(19) United States
(12) Patent Application Publication
(10) Pub. No.: US 2008/0300298 A1
(43) Pub. Date: Dec. 4, 2008

(54) Honokiol Derivates For the Treatment of Proliferative Disorders

(76) Inventors: Jack L. Arbiser, Atlanta, GA (US); Frank Amblard, Atlanta, GA (US)

Correspondence Address: INTELLECTUAL PROPERTY / TECHNOLOGY LAW, PO BOX 14329, RESEARCH TRIANGLE PARK, NC, 27709, US

(21) Appl. No.: 11/884,989

(22) PCT Filed: Feb. 23, 2006
(86) PCT No.: PCT/US2006/006494

§ 371 Date: Jul. 16, 2008

Related U.S. Application Data
(60) Provisional application No. 60/655,346, filed on Feb. 23, 2005.

(51) Int. Cl. A61K 31/357; C07C 43/23; C07C 39/12; C07C 25/18; A61K 31/135; A61K 31/03; A61P 35/04; A61K 31/05; A61K 31/075; C07C 211/50; C07D 321/10

(52) U.S. Cl. 514/450; 568/650; 568/717; 570/123; 564/305; 549/349; 514/721; 514/646; 514/734; 514/754

(57) ABSTRACT

The present invention provides novel honokiol derivatives, as well as pharmaceutical compositions containing the honokiol derivatives. These compounds and pharmaceutical compositions can be used in the prevention and/or treatment of cancer. In particular, honokiol derivatives, pharmaceutical compositions comprising the derivatives, and methods for their use in the treatment of myeloma are provided.

### RELATED APPLICATION

“This application is the national phase entry of PCT 2006/006494, filed Feb. 23, 2006, which claims priority to U.S. Ser. No. 60/655,346, filed Feb. 23, 2005, the contents of both of which are hereby incorporated by reference in their entirety.

### TECHNICAL FIELD

This application describes honokiol related compounds and compositions for the treatment of disorders associated with angiogenesis, cell proliferation, tumor growth and tumorogenesis and for example in the treatment of myeloma.

### BACKGROUND

Cancer is an abnormal growth of cells. Cancer cells rapidly reproduce despite restriction of space, nutrients shared by other cells, or signals sent from the body to stop reproduction. Cancer cells are often shaped differently from healthy cells, do not function properly, and can spread into many areas of the body. Abnormal growths of tissue, called tumors, are clusters of cells that are capable of growing and dividing uncontrollably. Tumors can be benign (noncancerous) or malignant (cancerous). Benign tumors tend to grow slowly and do not spread. Malignant tumors can grow rapidly, invade and destroy nearby normal tissues, and spread throughout the body.

Malignant cancers can be both locally invasive and metastatic. Locally invasive cancers can invade the tissues surrounding it by sending out “fingers” of cancerous cells into the normal tissue. Metastatic cancers can send cells into other tissues in the body, which may be distant from the original tumor.

Cancers are classified according to the kind of fluid or tissue from which they originate, or according to the location in the body where they first developed. In addition, some cancers are of mixed types. Cancers can be grouped into five broad categories, carcinomas, sarcomas, lymphomas, leukemias, and myelomas, which indicate the tissue and blood classifications of the cancer. Carcinomas are cancers found in body tissue known as epithelial tissue that covers or lines surfaces of organs, glands, or body structures. For example, a cancer of the lining of the stomach is called a carcinoma. Many carcinomas affect organs or glands that are involved with secretion, such as breasts that produce milk. Carcinomas account for approximately eighty to ninety percent of all cancer cases. Sarcomas are malignant tumors growing from connective tissues, such as cartilage, fat, muscle, tendons, and bones. The most common sarcoma, a tumor on the bone, usually occurs in young adults. Examples of sarcoma include osteosarcoma (bone) and chondrosarcoma (cartilage). Lymphoma refers to a cancer that originates in the nodes or glands of the lymphatic system, whose job it is to produce white blood cells and clean body fluids, or in organs such as the brain and breast. Lymphomas are classified into two categories: Hodgkin's lymphoma and non-Hodgkin's lymphoma. Leukemia, also known as blood cancer, is a cancer of the bone marrow that keeps the marrow from producing normal red and white blood cells and platelets. White blood cells are needed to resist infection. Red blood cells are needed to prevent anemia. Platelets keep the body from easily bruising and bleeding. Examples of leukemia include acute myelogenous leukemia, chronic myelogenous leukemia, acute lymphocytic leukemia, and chronic lymphocytic leukemia. The terms myelogenous and lymphocytic indicate the type of cells that are involved. Finally, myelomas grow in the plasma cells of bone marrow. In some cases, the myeloma cells collect in one bone and form a single tumor, called a plasmacytoma. However, in other cases, the myeloma cells collect in many bones, forming many bone tumors. This is called multiple myeloma.

Current treatments of cancer and related diseases have limited effectiveness and numerous serious unintended side effects. Cancer therapy can be divided into five subspecialties: (1) surgery, (2) radiation therapy, (3) chemotherapy, (4) immunotherapy, and (5) antiangiogenic therapy. These treatments have progressed only incrementally during more than thirty years of intensive research to discover the origins of cancer and devise improved therapies for cancer and related diseases. Current research strategies emphasize the search for effective therapeutic modes with less risk, including the use of natural products and biological agents. This change in emphasis has been stimulated by the fact that many of the consequences, to both patients and their offspring, of conventional cancer treatment result from their actions on genetic material. Efforts continue to discover both the genetic origins of cancer as well as new treatments.

### Honokiol

The root and stem bark of Magnolia has been used as a traditional Chinese medicine for the treatment of thrombotic stroke, gastrointestinal complaints, and anxiety. Honokiol (HNK), a substituted biphenyl and an active component isolated and purified from Magnolia, has anti-oxidant, antithrombosis, antibacterial, neurotrophic, xanthine oxidase inhibitory, and anxiolytic effects (Taira et al., Free Radic Res Commun. 1993; 19 Suppl 1:S71-77; Teng et al. Thromb Res. 1988; 50:757-765; Clark et al., J. Pharm. Sci. 1981; 70:951-952; Chang et al., Anticancer Res. 1994; 14:501-506; Kuribara et al., J. Pharm Pharmacol. 1998; 50:819-826; Esumi et al., Bioorg & Medicinal Chem Let 2004, 14: 2621-25).

In the early 1990s, reports of HNK's anticancer effects were published. In 1994, Hirano et al (Life Sci. 1994; 55(13):1061-9) examined the anti leukemic-cell efficacy of 28 naturally occurring and synthetic flavonoids and 11 naturally occurring ligands on human promyelocytic leukemic cell line HL-60, and cytotoxicity of these compounds was compared with four clinical anti-cancer agents. HNK was identified as one of the most potent compounds in this screen, with an IC₅₀ value less than 100 ng/ml. In 1998, Hibasami et al. demonstrated that HNK induced apoptosis in human lymphoid leukemia Molt 4B cells (Hibasami et al., Int. J. Mol. Med. 1998).

HNK has also been found to induce apoptosis in human squamous cell lung cancer CH27 cells (Yang S E, et al Biochem Pharmacol. 2002; 63:1641-1651) and in human colorectal RKO cells (Wang et al World J Gastroenterol. 2004; 10:2205-2208). In 2004, Chen et al. (World J Gastroenterol. 2004; 10: 3459-3463) reported that HNK was effective in an in vivo animal model of human colon cancer by inhibiting tumor growth and prolonging the lifespan of tumor bearing mice.

Honokiol is an inhibitor of angiogenesis and antitumor activity in vivo. HNK can cause apoptosis in tumor cells and inhibit angiogenesis through blocking phosphorylation of vascular endothelial growth factor receptor 2 (VEGFR2), the major mitogenic and chemoattractant endothelial growth factor (Bai et al. (2003) J. Biol. Chem. 278, 35501-35507). Honokiol also exhibits direct antitumor activity through induction of apoptosis through tumor necrosis factor apoptosis-inducing ligand (TRAIL/Apo2L) signaling and has been found to be highly effective against angiosarcoma in nude mice in vivo (Bai et al. (2003) J. Biol. Chem. 278, 35501-35507).

Esumi et al. (Biorganic & Medicinal Chemistry Letters (2004) 14: 2621-2625) describe a synthesis method to produce HNK. This report also evaluates the structure activity relationship of O-methylated and/or its hydrogenated analogs of HNK in an in vitro neurotrophic assay. Esumi et al. conclude that the 5-allyl and 4-hydroxyl groups are essential for the neurotroplic activity of HNK.

PCT Publication No. WO 02/076393 and U.S. Publication No. 2004/0105906 to Emory University describe pharmaceutical compositions and methods of treating conditions such angiogenic-, neoplastic-, and cancer-related conditions and skin conditions by administration of honokiol-type and/or magnolol-type compounds, as shown in FIGS. 1-4. For example, such compositions comprise at least one compound of formula A1:

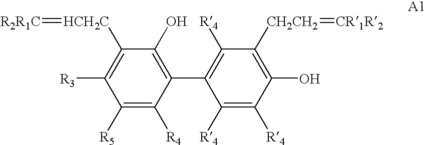

wherein R₁, R₂, R₃, R₄, R₅, R′₁, R′₂, R′₃, R′₄, and R′₅ can be independently selected from groups that include, but are not limited to, hydrogen, hydroxyl groups, amides, amines, hydrocarbons, halogenated hydrocarbons, cyclic hydrocarbons, cyclic heterocarbons, halogenated cyclic heterocarbons, benzyl, halogenated benzyl, organo selenium compounds, Sulfides, carbonyl, thiol, ether, dinitrogen ring compounds, thiophenes, pyridines, pyrroles, imidazoles, and pyrimidines. Honokiol-type and magnolol-type compounds are shown to inhibit SVR cell proliferation.

In November of 2004, Arbiser et al. reported that honokiol inhibited the growth of multiple myeloma cell lines via induction of G1 growth arrest, followed by apoptosis with IC50 values at 48 h of 5 to 10 μg/mL. It was also reported that honokiol inhibited growth of doxorubin (Dox)-resistant (RPMI-Dox40), mephalan resistant (RPMI-LR5) and dexamethiasone (Dex)-resistant (MM.1R) cell lines. It was suggested that the mechanism of honokiol triggered cytotoxicity is the honokiol induced increased expressin of Bax and Bad, down-regulated Mc-1 protein expression, followed by caspase-8/9/3 cleavage, (Arbiser, J. et al. Poster at the American Society of Hematology Annual Meeting, 2004. Abstract published online Nov. 4, 2004).

In July of 2005, Battle et al. reported that honokiol induces caspase-dependent apoptosis in B-cell chronic lymphocytic leukemia (B-CLL) cells (Blood. July 2005; 106:690-697). Honokiol induced caspase-dependent cell death in all of the B-CLL cells examined, which were primary tumor cells derived from B-CLL patients, and was more toxic toward B-CLL cells than to normal mononuclear cells. The honokiol-induced apoptosis was characterized by the activation of caspase-3, -8, and -9 and cleavage of poly(adenosine diphosphate-ribose) polymerase (PARP). It was also reported that honokiol enhanced cytotoxicity induced by fludarabine, cladribine, or chlorambucil.

In September 2005, Ishitsuka et al reported that honokiol overcomes conventional drug resistance in human multiple myeloma by induction of caspase-dependent and -independent apoptosis (Blood, 1 Sep. 2005, Vol. 106, No. 5, pp. 1794-1800). HNK induced cytotoxicity in human multiple myeloma (MM) cell lines and tumor cells from patients with relapsed refractory MM through induction of apoptosis via both caspase-dependent and -independent pathways. HNK also enhanced MM cell cytotoxicity and apoptosis induced by bortezomib.

It is an object of the present invention to provide new compounds, compositions, methods and uses for the treatment of disorders associated with angiogenesis, cell proliferation, tumor growth, tumorogenesis, and myeloma.

### SUMMARY OF THE INVENTION

The present invention provides honokiol derivatives, as well as pharmaceutical compositions containing the honokiol derivatives, and methods of use thereof. The compounds and compositions can be used to inhibit angiogenesis, cell proliferation and tumorogenesis and tumor growth. These compounds and pharmaceutical compositions can be used in the prevention and/or treatment of cancer, for example, myeloma, including multiple myeloma.

In a particular embodiment, honokiol and honokiol derivatives are provided that are useful for the treatment of myeloma, and in particular, multiple myeloma. In another particular embodiment, these compounds can be used to treat leukemia, such as chronic lymphocytic leukemia. In particular, the compounds described herein can be used to treat chronic lymphocytic leukemia cells (CLL), including, but not limited to those with mutant p53.

One aspect of the present invention is based on the discovery that honokiol can induce apoptosis in cancer cells through a caspase independent mechanism. The present invention also covers the treatment of noncancerous tumors and other proliferative conditions.

Cancer cell lines with low levels of certain caspases, such as caspase-3 and caspase-8, can be associated with cancer drug resistance. The honokiol and honokiol derivatives as disclosed herein can be used to treat cancers resistant to one or more drugs, including the embodiments of cancers and drugs disclosed herein. In one embodiment, honokiol or a derivative thereof as disclosed herein is administered in an effective amount for the treatment of a patient with a drug resistant tumor, for example, multidrug resistant tumors, including but not limited to those resistant to doxorubicin, As₂O₃, melphalan, dexamethasone, bortezomib and revlimid. In one particular embodiment, honokiol or a derivative thereof can be used to treat doxorubicin resistant multiple myeloma.

The honokiol or derivative thereof can be administered alone or in combination with an additional therapeutic or chemotherapeutic agent. In a particular embodiment, honokiol or a derivative can be administered in an effective amount for the treatment of drug resistant multiple myeloma. In one embodiment, the additional chemotherapeutic agent can be a P-glycoprotein inhibitor, such as verapamil, cyclosporin (such as cyclosporin A), tamoxifen, calmodulin antagonists, dexverapamil, dexniguldipine, valspodar (PSC 833), biricodar (VX-710), tariquidar (XR9576), zosuquidar (LY335979), laniquidar (R101933), and/or ONT-093. In another embodiment, the additional chemotherapeutic agent can be a histone deacetylase inhibitor. In a particular embodiment, the histone deacetylase inhibitor can be suberoylaanilide hydroxamic acid (SAHA).

In one embodiment, a method for the treatment of cancer in a host is provided, comprising administering an effective amount of a honokiol derivative disclosed herein to the host. The cancer can be, for example, carcinoma, sarcoma, lymphoma, leukemia, or myeloma.

In a particular embodiment, a method is provided for the treatment of myeloma in a host, the method comprising administering to the host an effective amount of honokiol or a honokiol derivative compound disclosed herein to the host. The myeloma can be, for example, multiple myeloma, macroglobulinemia, isolated plasmacytoma of bone, extramedullary plasmacytoma, waldenstrom's macroglobulinemia, monoclonal gammapathy, or a refractory plasma cell neoplasm.

In another embodiment, the compound is administered in combination or alternation with at least one additional therapeutic agent for the treatment of cancer, including myeloma.

In a particular embodiment, there is provided a method for the treatment of a condition characterized by angiogenesis, tumorogenesis, tumor growth, a neoplastic condition, cancer or a skin disorder in a host, the method comprising administering to the host an effect amount of a compound disclosed herein optionally in combination with a pharmaceutically acceptable carrier.

In a further embodiment, methods are provided for the treatment of a condition characterized by inflammation by administering to the host an effect amount of a compound disclosed herein optionally in combination with a pharmaceutically acceptable carrier. In a particular embodiment, methods for the treatment of arthritis by administering an effective amount of a compound disclosed herein are also provided.

In another embodiment, methods are provided for the treatment of a bone disorder, including, but not limited to osteoporosis, by administering to the host an effect amount of a compound disclosed herein optionally in combination with a pharmaceutically acceptable carrier.

An additional object of the present invention provides methods to identify tumors and cancers that are particularly susceptible to the toxic effects of honokiol and/or related compounds as described herein. One aspect of the present invention is based on the discovery that tumors that express phospholipase D (PLD), nuclear factor-κB (NKκB), and/or adenosine monophosphate kinase activated protein kinase (AMPK) are particularly susceptible to the toxic effects of honokiol or derivatives thereof. In one embodiment, methods are provided for treating a tumor in a mammal, particularly a human, which includes (i) obtaining a biological sample from the tumor; (ii) determining whether the tumor expresses or overexpresses an phospholipase D (PLD), nuclear factor-κB (NKκB), and/or adenosine monophosphate kinase activated protein kinase (AMPK), and (iii) treating the tumor that expresses or overexpresses phospholipase D (PLD), nuclear factor-κB (NKκB), and/or adenosine monophosphate kinase activated protein kinase (AMPK) with honokiol or a related compound as described herein. In one embodiment, the level of NFκB and/or AMPK expression can be determined by assaying the tumor or cancer for the presence of a phosphorylated NFκB and/or AMPK, for example, by using an antibody that can detect the phosphorylated form. In another embodiment, the level of PLD, NFκB and/or AMPK expression can be determined by assaying a tumor or cancer cell obtained from a subject and comparing the levels to a control tissue. In certain embodiments, the PLD, NFκB and/or AMPK can be overexpressed at least 2, 2.5, 3 or 5 fold in the cancer sample compared to the control.

Exemplary compounds include the compounds of FIGS. 1-4 and compounds disclosed herein, including compounds of Ia, or a salt, ester or prodrug thereof:

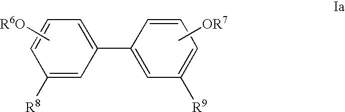

wherein R⁶ and R⁷ are independently H, alkyl, alkenyl, alkynyl, or aryl, which is optionally substituted, and are independently, for example a C₁₋₁₀ alkyl, alkenyl or alkynyl, e.g. methyl, ethyl or propyl;

wherein R⁸ and R⁹ are independently alkyl, alkenyl, alkynyl, or aryl, which is optionally substituted and are independently, for example, C₁₋₁₀ alkyl or alkenyl, such as vinyl or allyl; and

wherein optionally at least one of R⁸ and R⁹ are alkyl, such as C₁₋₅ alkyl.

In one subembodiment of Formula Ia:

R⁶ and R⁷ are independently H or C₁₋₅ alkyl, e.g. methyl, ethyl or propyl;

R⁸ and R⁹ are independently C₁₋₅ alkyl or alkenyl, such as vinyl or allyl; and

at least one of R⁸ and R⁹ are C₁₋₅ alkyl, such as methyl, ethyl, propyl or butyl.

In another embodiment, the compound has the Formula Ib, or a salt, ester or prodrug thereof:

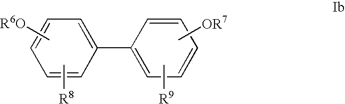

wherein R⁶ and R⁷ are independently H, alkyl, alkenyl, alkynyl, or aryl, which is optionally substituted, and are independently, for example a C₁₋₁₀ alkyl, alkenyl or alkynyl, e.g., methyl, ethyl or propyl;

wherein R⁸ and R⁹ are independently alkyl, alkenyl, alkynyl, or aryl, which is optionally substituted and are independently, for example, C₁₋₁₀ alkyl or alkenyl, such as vinyl or allyl; and

wherein optionally at least one of R⁶ and R⁷ are not H.

In one subembodiment of Formula Ib:

R⁶ and R⁷ are independently H, alkyl, such as C₁₋₅ alkyl, alkyl, alkenyl or alkynyl, e.g., methyl, ethyl or propyl;

R⁸ and R⁹ are independently C₁₋₅ alkyl or alkenyl, such as vinyl or allyl; and

at least one of R⁶ and R⁷ are not H.

Also provided are compounds of Formula Ic, Id, Ie or If, or a salt, ester or prodrug thereof:

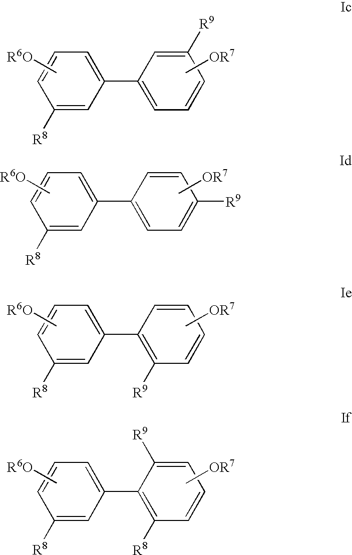

wherein R⁶ and R⁷ are independently H, alkyl, alkenyl, alkynyl, or aryl, which is optionally substituted, and are independently, for example a C₁₋₁₀ alkyl, alkenyl or alkynyl; e.g. methyl, ethyl or propyl;

wherein R⁸ and R⁹ are independently alkyl, alkenyl, alkynyl, or aryl, which is optionally substituted and are independently, for example, C₁₋₁₀ alkyl or alkenyl, such as vinyl or allyl; and

wherein optionally at least one of R⁸ and R⁹ are alkyl, such as C₁₋₅ alkyl; and

wherein optionally at least one of R⁶ and R⁷ are not H.

In one subembodiment of Formulas Ic, Id, Ie or If:

R⁶ and R⁷ are independently H or C₁₋₅ alkyl, alkenyl or alkynyl, e.g. methyl, ethyl or propyl; and

R⁸ and R⁹ are independently alkyl, such as C₁₋₅ alkyl, alkyl or alkenyl, such as vinyl or allyl.

In another embodiment, the compound has the formula D2:

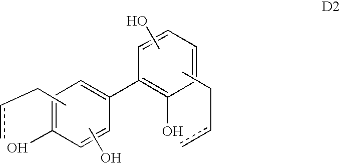

wherein the allyl group is in oxidized or reduced form.

In another embodiment, the compound has the formula D3:

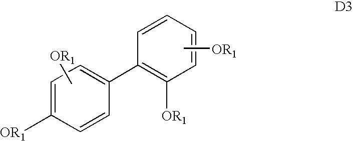

wherein the OR₁ substituent denotes an ether or ester linkage, and for example, each R₁ is independently alkyl, e.g., C₁₋₁₀ alkyl, or acyl, e.g., C₁₋₁₀ acyl.

In another embodiment, the compound is a compound of one of the following formulas:

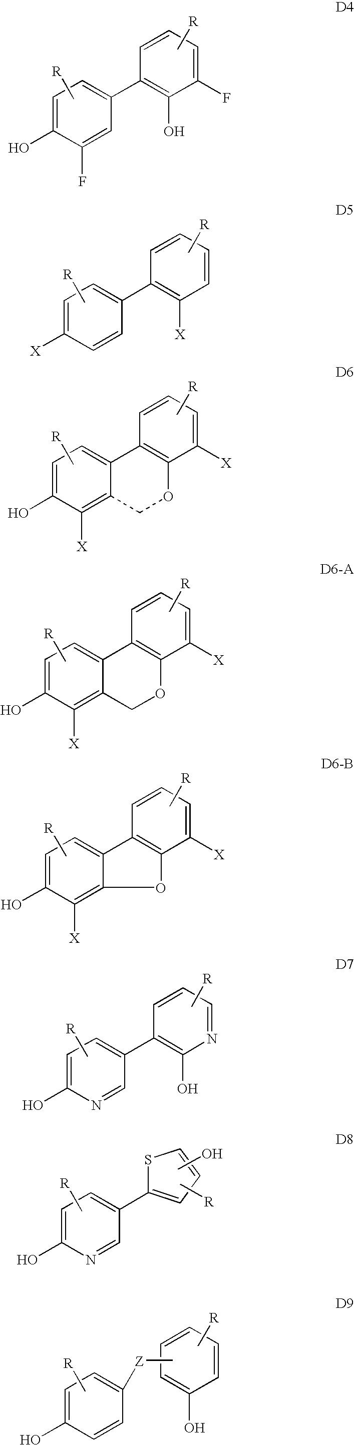

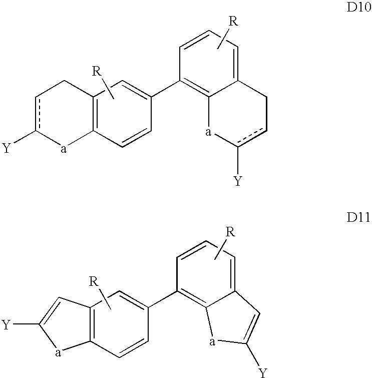

wherein each R is independently alkyl, alkenyl, aryl, or vinyl which is optionally straight, branched, or cyclic and is optionally substituted. Optionally each R is independently C₁₋₁₀ alkyl, C₁₋₁₀ alkenyl or C₁₋₁₀ alkynyl. For example, each R may be independently selected from the following groups:

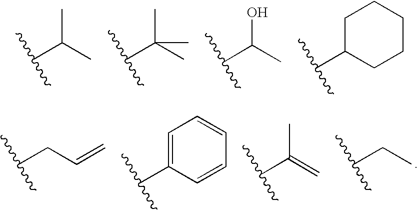

In Formula D5, each X is independently, for example, halogen (e.g., F), N(R¹)₂, SH or SR¹, where each R¹ is independently, e.g., H or alkyl.

In Formula D6, D6-A and D6-B, each X is independently H, alkyl (e.g., methyl or C₁₋₁₀ alkyl) or halogen, e.g., F. In Formula D6, the dashed line shows either the presence or absence of a CH₂ group thus making the ring either five or six membered, as shown in D6-A and D6-B.

In Formula D9, Z is O, S, SO₂, CO, or (CH₂)ₙ where n is 1-8.

In Formula D10 and D11, each Y is independently H, OH or alkyl, and each a is independently O, NR¹ or S, where each R¹ is independently, e.g., H or alkyl, e.g., C₁₋₅ alkyl. In D10, the dotted line shows a double or single bond.

In a particular embodiment, the compounds disclosed above can be administered in an effective amount for the treatment of myeloma.

In certain embodiments, a method is provided including administering to a host in need thereof an effective amount of a compound disclosed herein, or pharmaceutical composition comprising the compound, in an effective amount for the treatment of a condition characterized by angiogenesis, tumorogenesis, a neoplastic condition, cancer, or a skin disorder.

In one embodiment, a method for the treatment of cancer is provided including administering an effective amount of a compound disclosed herein, or a salt, isomer, prodrug or ester thereof, to an individual in need thereof, wherein the cancer is for example, carcinoma, sarcoma, lymphoma, leukemia, or myeloma. The compound, or salt, isomer, prodrug or ester thereof, is optionally provided in a pharmaceutically acceptable composition including the appropriate carriers, such as water, which is formulated for the desired route of administration to an individual in need thereof. Optionally the compound is administered in combination or alternation with at least one additional therapeutic agent for the treatment of cancer or in particular myeloma.

Also within the scope of the invention is the use of a compound disclosed herein or a salt, prodrug or ester thereof in the treatment of cancer, and in particular, myeloma, optionally in a pharmaceutically acceptable carrier; and the use of a compound disclosed herein or a salt, prodrug or ester thereof in the manufacture of a medicament for the treatment of cancer, and in particular, myeloma, optionally in a pharmaceutically acceptable carrier.

In one embodiment, the compounds of the present invention can be used to prevent and/or treat a carcinoma, sarcoma, lymphoma, leukemia, and/or myeloma. In other embodiments of the present invention, the compounds disclosed herein can be used to treat solid tumors. In still further embodiments, the compounds and compositions disclosed herein can be used for the treatment of cancer, such as, but not limited to cancer of the following organs or tissues: breast, prostate, bone, lung, colon, including, but not limited to colorectal, urinary, bladder, non-Hodgkin lymphoma, melanoma, kidney, renal, pancreas, pharnx, thyroid, stomach, brain, and/or multiple myeloma. In further embodiments of the present invention, the compounds disclosed herein can be used in the treatment of angiogenesis-related diseases.

In certain particular aspects of the present invention, the compounds described herein can be used in the treatment of myeloma. In one embodiment, honokiol can be used in the treatment of myeloma. In another embodiment of the present invention, honokiol or any of the compounds or compositions described herein can be used to treat a plasma cell neoplasm, such as, but not limited to mycloma, multiple myeloma, macroglobulinemia, isolated plasmacytoma of bone, extramedullary plasmacytoma, waldenstrom's macroglobulinemia or Lymphoplasmacytic leukemia, monoclonal gammapathy, and/or refractory plasma cell neoplasm.

In one aspect, the compounds and compositions can be administered in combination or alternation with at least one additional chemotherapeutic agent. The drugs can form part of the same composition, or be provided as a separate composition for administration at the same time or a different time. In one embodiment, compositions of the invention can be combined with anti-angiogenic agents. In other embodiments of the present invention, the compounds and compositions disclosed herein can be used in combination or alternation with the following types of drugs, including, but not limited to: antiproliferative drugs, antimitotic agents, antimetabolite drugs, alkylating agents or nitrogen mustards, drugs which target topoisomerases, drugs which target signal transduction in tumor cells, gene therapy and antisense agents, antibody therapeutics, steroids, steroid analogues, anti-emetic drugs and/or nonsteroidal agents.

### BRIEF DESCRIPTION OF THE DRAWINGS

FIG. 1 is an illustration of honokiol-type compound and magnolol-type compound structures.

FIG. 2 is a diagram that illustrates representative functional groups of the honokiol-type compound and magnolol-type compound structures shown in FIGS. 1, 3 and 4.

FIGS. 3 and 4 illustrate representative structures that are structurally similar to the honokiol-type compound and magnolol-type compound structures of FIG. 1.

FIG. 5 is a graph that illustrates the inhibition of SVR cell proliferation of honokiol-type and magnolol-type compounds.

FIG. 6 depicts honokiol (HNK) induced cytotoxicity in multiple myeloma (MM) cell lines and tumor cells from MM patients, but not in normal peripheral blood mononuclear cells (PBMNCs). A and B show growth inhibition in MM cell lines by HNK as assessed by colorimetric assay after 48 h-culture.

FIG. 7 depicts honokiol (HNK) induced apoptosis in MM cells. A shows MM.1S and RPM18226 cells that were treated with 8 ug/ml HNK for 48 hours. In B cleavage of caspases and PARP was determined by Western blotting of MM.1S whole cell lysates after 10 ug/ml HNK treatment for 12 and 24 h, with or without z-VAD-fmk (25 uM) pre-incubation for 1.5 h. C shows MM.1S cells that were treated with HNK or As₂O₃, with or without 25 uM z-VAD-fmk pre-treatment for 1.5 hours. In D, MM cells were treated with HNK or As₂O₃ for 24 h, with or without 25 uM z-VAD-fmk pre-treatment for 1.5 h, and expression of APO₂O₇ was determined by flow cytometry. E shows the cytotoxicity as determined by trypan blue exclusion staining. In F, MM.1S cells were treated with HNK (10 ug/ml for 0, 4, 8 and 12 h). G shows MM.1S cells that were treated with HNK (10 ug/ml for 24 h), with or without pre-treatment by z-VAD-fmk.

FIG. 8 illustrates that the combination of honokiol (HNK) with bortezomib enhances cytotoxicity against MM.1S cells. In A, MM.1S cells were treated with HNK and bortezomib for 48 h and cell growth was determined by colorimetric assay. B shows MM.1S cells that were treated with HNK and bortezomib and induction of apoptosis was determined by APO2.7. In C, MM.1S cells were treated with HNK and bortezomib for 8 h.

FIG. 9 illustrates that HNK can overcome the protective effects of IL-6, IGF-1 and adherence to patient bone marrow stromal cell (BMSCs) cultures. MM.1S cells were treated for 48 h with indicated concentrations of HNK in the presence or absence of IL-6 (shown in A), IGF (shown in B) or BMSCs derived from 2 MM patients (shown in C and D).

FIG. 10 illustrates that HNK modulates growth and survival signaling pathways in MM.1S cells. A shows MM.1S cells that were pretreated with HNK (10 ug/ml) in FCS 2.5% containing media for 3 and 6 h, cells were then stimulated with IL-6 (10 ng/ml) for 10 and 20 min. In B, MM.1S cells were pretreated with HNK (10 ug/ml) in FCS 2.5% containing media for 3 and 6 h, and then stimulated with IGF-1 (25 ng/ml) for 10 and 20 min. C shows MM.1S cells that were pretreated with HNK (10 ug/ml) in FCS 2.5% containing media for 3 and 6 h.

FIG. 11 depicts HNK inhibition of angiogenesis of HUVEC. HUVEC were cultured with (depicted in B) or without (depicted in A) 8 ug/ml of HNK for 6 h, and tube formation was assessed. Original magnification is ×40.

FIG. 12 shows the effect of inhibition of MAPKK by a dominant negative MAPKK gene or by the chemical inhibitor PD98059 on morphology of endothelial cells. MS1 represents endothelial cells containing only SV40 large T antigen; SVR represents MS1 cells transformed with ras; SVR+ PD98059 represents SVR cells treated with PD98059 (5 μg/ml); and SVRA221a represents cells stably expressing the dominant negative A221 allele of MAPKK.

FIG. 13 illustrates the effect of honokiol and magnolol on apoptosis. The light columns represent SVR cells treated with magnolol, and the dark columns represent SVR cells treated with honokiol. The control lanes represent cells immediately after treatment compared with 18 and 48 h of treatment.

FIG. 14 depicts the effects of honokiol on the phosphorylation of various intracellular proteins. A shows that honokiol inhibits phosphorylation of AKT, p44/42 MAPK, and Src. SVR. B shows that honokiol inhibits phosphorylation of Akt at low concentrations but not p44/42 MAPK or Src.

FIG. 15 shows that honokiol inhibition of endothelial proliferation is TRAIL-dependent. The green bars represent endothelial cells treated with honokiol alone, the dark blue bars represent cells treated with honokiol and TRAIL antibody, and the light blue bars represent cells treated with honokiol and isotype control antibody.

FIG. 16 illustrates that honokiol induces apoptosis in multiple myeloma cells (MM) through caspase8/caspase9/PARP mediated apoptosis.

FIG. 17 shows the effect of honokiol on VEGF phosphorylation. In A, the effect of honokiol on VEGF-induced KDR autophosphorylation in HUVECs is illustrated. In B, the effect of honokiol on VEGF-induced Rac activation was determined. Top, representative immunoblot of GTP-bound Rac. Bottom, densitometric analysis (mean ±S.E.) of immunoblots from three experiments expressed as fold increase over control.

FIG. 18 depicts the effect of honokiol on in vivo growth of SVR angiosarcoma in nude mice. This data shows that honokiol is effective against tumors in vivo.

FIG. 19 shows the induction of apoptosis in MM.1S and SU-DHL-4 cells.

FIG. 20 illustrates that honokiol activates AMP kinase (AMPK). PC3 cells were treated with honokiol under nontoxic and hypoxic conditions. The top blot shows increased phosphorylation (activation) of AMP kinase by honokiol. The bottom blot shows total AMP kinase protein, serving as a loading control.

FIG. 20b illustrates the effects of honokiol on HIF-1a in the prostate cancer cell line. Honokiol activated HIF-1a in prostate cancer cells in a dose dependent manner.

FIG. 21 depicts that honokiol can mimic the effect of wild type tuberin. Treatment with tiberin causes downregulation of S6kinase phosphorylation in a time and dose dependent fashion, as well as downregulation of akt, which indicates that honokiol can mimic several of the activities of wild type tuberin.

FIG. 22 shows that honokiol inhibits the activity of phospholipase D in both 0.5% and 10% serum in SVR cells.

FIG. 23 illustrates a proposed mechanism of action of honokiol. Honokiol can block PLD activity and activate AMP kinase. Honokiol can block the activity of phospholipase, resulting in decreased production of phosphatidic acid. Decreased phosphatidic acid can result in decreased activation of mTOR (mammalian target of rapamycin), which can result in downregulation of NFkB. Phosphatidic acid can have direct effects on mTOR activation, and like akt activation, phosphatidic acid production can result in phosphorylation and inactivation of tuberin (tsc2) (Tee et al., 2003; Chen et al., 2005; Hui et al., 2004). Similarly, AMPK activation can result in dephosphorylation and activation of tuberin (Joseph et al., 2001). Activation of p53 can activate AMPK in certain systems, honokiol induction of AMPK does not appear to require p53, as it occurs in the p53 deficient PC3 cell line (Feng et al., 2005; Wang et al., 2001).

FIG. 24 illustrates that honokiol can block NFκB activation and sensitize tumor cells to conventional chemotherapeutic agents. In (A) KBM-5 cells (2×10⁶/ml) were serum starved for 24 h and then incubated with TNF alone or in combination with honokiol as indicated for 24 h. Cell death was determined by calcein AM based live/dead assay. The red color highlights dead cells, and green color highlights live cells. In (B) cells were pretreated with 30 μM honokiol for 12 h and then incubated with 1 nM TNF for 16 h. Cells were incubated with anti-annexin V antibody conjugated with FITC and then analyzed with a flow cytometer for early apoptotic effects. In (C) Cells were pretreated with 30 μM honokiol for 12 h and then incubated with 1 nM TNF for the indicated times. Whole-cell extracts were prepared and subjected to Western blot analysis using anti-PARP antibody. (D) KBM-5 cells (5000 cells/0.1 ml) were incubated at 37° C. with TNF, Taxol, 5-FU or doxorubicin in the presence and absence of 30 μM honokiol, as indicated for 72 h duration, and the viable cells were assayed using MTT reagent. The results are shown as the mean ±s.d. from triplicate cultures.

FIG. 25 demonstrates that honokiol can repress TNF-induced NF-κB-dependent expression of anti-apoptosis-, proliferation-, and metastasis-related gene products. (A) shows proliferative and metastatic proteins and (B) shows anti-apoptosis proteins. KBM-5 cells were incubated with 30 μM honokiol for 12 h and then treated with 1 nm TNF for the indicated times. Whole-cell extracts were prepared and subjected to Western blot analysis using the relevant antibodies.

FIG. 26 shows that honokiol potentiates the apoptotic effects of TNF and chemotherapeutic drugs. (A) shows the structure of honokiol. (B) is a bar graph showing that honokiol enhances apoptosis induced by TNF and chemotherapeutic agents. KBM-5 cells (5000 cells/0.1 ml) were incubated at 37° C. with TNF, paclitaxel or doxorubicin in the presence and absence of 5 mM honokiol as indicated for 72 h, and the viable cells were assayed using the MTT reagent. The results are expressed as mean cytotoxicity ±SD from triplicate cultures. (C) shows that honokiol enhances TNF-induced PARP cleavage. KBM-5 cells (2×10⁶/ml) were serum starved for 24 h and then incubated with TNF (1 nM) alone or in combination with honokiol (25 mM) for the indicated times, and PARP cleavage was determined by Western blot analysis as described Example 8. Values at the bottom indicate the densitometric analysis of the 87-kDa band. (D) depicts that honokiol enhances TNF-induced cell death. KBM-5 cells (2×10⁶/ml) were serum starved for 24 h and then incubated with TNF (1 nM) alone or in combination with honokiol (10 mM) as indicated for 24 h. Cell death was determined by the calcein-AM based live/dead assay as described in Example 8. Data are for a representative experiment out of 3 independent ones showing similar results. (E) shows that honokiol upregulates TNF-induced early apoptosis. Cells were pretreated with 25 mM honokiol for 12 h and then incubated with 1 nM TNF for 16 h. Cells were incubated with anti-annexin V antibody conjugated with FITC and then analyzed with a flow cytometer for early apoptotic effects.

FIG. 27 demonstrates that Honokiol suppresses RANKL-induced osteoclastogenesis and TNF-induced invasive activity. (A) RAW 264.7 cells (1×10⁴) were plated overnight, pretreated with 5 mM honokiol for 12 h, and then treated with 5 nM RANKL. At 4 and 5 days later, cells were stained for TRAP and evaluated for osteoclastogenesis. Photographs were taken after 5 days of incubation with RANKL. (B) The numbers of TRAP-positive multinucleated osteoclasts (>3 nuclei) per well were counted. (C) H1299 cells (2.5×10⁴) were seeded into the upper wells of a Matrigel invasion chamber overnight in the absence of serum, pretreated with 10 mM honokiol for 12 h, treated with 1 nM TNF for 24 h in the presence of 1% serum, and then subjected to invasion assay. The value for no honokiol and no TNF was set to 1.0.

FIG. 28 demonstrates that Honokiol inhibits NF-kB. (A) Honokiol blocks NF-kB activation induced by TNF, cigarette smoke condensate, PMA, okadaic acid, and H₂O₂. H1299 cells (2×10⁶/ml) were preincubated for 12 h at 37° C. with 25 mM honokiol and then treated with TNF (0.1 nM), PMA (100 ng/ml, 1 h), okadaic acid (500 nM, 4 h) cigarette smoke condensate (10 mg/ml, 30 min), or H₂O₂ (250 mM, 1 h). Nuclear extracts were prepared and tested for NF-kB activation. Data are for a representative experiment out of 3 independent ones showing similar results. (B) Honokiol inhibits TNF-dependent NF-kB activation in a dose-dependent manner. H1299 cells (2×10⁶/ml) were preincubated with the indicated concentrations of honokiol for 12 h at 37° C. and then treated with 0.1 nM TNF for 30 min. Nuclear extracts were prepared and tested for NF-kB activation. (C) Honokiol inhibits TNF-dependent NF-kB activation in a time-dependent manner. H11299 cells (2×10⁶/ml) were preincubated with 25 mM honokiol for the indicated times at 37° C. and then treated with 0.1 nM TNF for 30 min at 37° C. Nuclear extracts were prepared and then tested for NF-kB activation. (D) Suppression of inducible activation by honokiol is not cell-type specific. Two million A293 or Jurkat cells were pretreated with the indicated concentrations of honokiol for 24 h and then treated with 0.1 nM TNF for 30 min. The nuclear extracts were then prepared and assayed for NF-kB by EMSA. (E) Honokiol suppresses constitutive NF-kB activation in multiple myeloma U266 and squamous cell carcinoma SCC4. Cells were incubated with the indicated concentrations of honokiol for 24 h and then with 0.1 nM TNF for 30 min. Nuclear extracts were prepared and analyzed for NF-kB activation by EMSA. (F) Honokiol does not modulate the ability of NF-kB to bind to the DNA. Nuclear extracts from H11299 cells (2×10⁶/ml) treated or not treated with 0.1 nM TNF for 30 min were treated with the indicated concentrations of honokiol for 2 h at room temperature and then assayed for DNA binding by EMSA. Data are of a representative experiment out of 3 independent ones showing similar results.

FIG. 29 demonstrates that (A) Honokiol inhibits TNF-induced NF-kB activation, IkBaphosphorylation, and IkBa degradation. Honokiol inhibits TNF-induced activation of NF-kB. H1299 cells were incubated with 25 mM honokiol for 12 h, treated with 0.1 nM TNF for the indicated times, and then analyzed for NF-kB activation by EMSA. (B) H1299 cells (2×10⁶/ml) were incubated with 25 mM honokiol for 12 h at 37° C., treated with 0.1 nM TNF for the indicated times at 37° C., and then tested for IkBa (upper panel) in cytosolic fractions by Western blot analysis. Equal protein loading was evaluated by b-actin (lower panel). (C) Honokiol blocks the phosphorylation and ubiquitination (D) of IkBaby TNF. Cells were preincubated with 25 mM honokiol for 12 h, incubated with 50 mg/ml N-acetyl-leucyl-leucyl-norleucinal (ALLN) for 30 min, and then treated with 0.1 nM TNF for 10 min. Cytoplasmic extracts were fractionated and then subjected to Western blot analysis using phospho-specific anti-IkBa antibody. The same membrane was reblotted with anti-IkBa antibody. (E) Honokiol inhibits TNF-induced IkBakinase activity. H1299 cells (2×10⁶/ml) were treated with 25 mM honokiol for 12 h and then treated with 0.1 nM TNF for the indicated time intervals. Whole-cell extracts were prepared, and 200 mg of extract was immunoprecipitated with antibodies against IKKa and IKKb. The immune complex kinase assay was then performed as described in Materials and Methods. To examine the effect of honokiol on the level of expression of IKK proteins, 30 mg of whole-cell extract was analyzed on 10% SDS-PAGE, electrotransferred, and immunoblotted with the indicated antibodies as described in Materials and Methods. (F) Honokiol inhibits TNF-induced nuclear translocation of p65. H1299 cells (2×10⁶/ml) were either untreated or pretreated with 25 mM honokiol for 12 h at 37° C. and then treated with 0.1 nM TNF for the indicated times. Nuclear extracts were prepared and analyzed by Western blotting using antibodies against p65. (G) honokiol inhibits TNF-induced nuclear translocation of p65. H1299 cells (1×10⁶/ml) were first treated with 25 mM honokiol for 12 h at 37° C. and then exposed to 0.1 nM TNF. After cytospin, immunocytochemical analysis was performed as described in Materials and Methods. Data are for a representative experiment out of 3 independent ones showing similar results. (H) Honokiol inhibits TNF-induced phosphorylation of p65. H1299 cells (2×10⁶/ml) were incubated with 25 mM honokiol for 12 h and then treated with 0.1 nM TNF for the indicated times. The cytoplasmic and nuclear extracts were analyzed by Western blotting using antibodies against the phosphorylated form of p65.

FIG. 30 demonstrates that (A) Honokiol inhibits TNF-induced NF-kB-dependent reporter gene (SEAP) expression. A293 cells were transiently transfected with an NF-kB-containing plasmid linked to the SEAP gene and then treated with the indicated concentrations of honokiol. After 24 h in culture with 0.1 nM TNF, cell supernatants were collected and assayed for SEAP activity. Results are expressed as fold activity over the activity of the vector control. (B) Honokiol inhibits NF-kB-dependent reporter gene expression induced by TNFR, TRADD, TRAF, NIK, and IKKb. A293 cells were transiently transfected with the indicated plasmids along with an NF-kB-containing plasmid linked to the SEAP gene and then left either untreated or treated with 25 mM honokiol for 12 h. Cell supernatants were assayed for secreted alkaline phosphatase activity. Results are expressed as fold activity over the activity of the vector control. Bars indicate standard deviation. (C) Honokiol inhibits TNF-induced COX2 promoter activity. H1299 cells were transiently transfected with a COX-2 promoter plasmid linked to the luciferase gene and then treated with the indicated concentrations of honokiol. After 24 h in culture with 0.1 nM TNF, cell supernatants were collected and assayed for luciferase activity. Results are expressed as fold activity over the activity of the vector control. Values are means ±SD (indicated as error bars) of triplicate cultures for a representative experiment out of 3 independent ones showing similar results. (D) Structure of magnolol. (E) The honokiol analogue magnolol inhibits TNF-induced NFkB activation. H1299 cells were treated with the indicated concentrations of magnolol for 12 h and then stimulated with 0.1 nM TNF for 30 min. Nuclear extracts were prepared and analyzed for NF-kB activation by EMSA.

FIG. 31 demonstrates that honokiol inhibits TNF-induced NF-kB-regulated gene products. (A) Honokiol inhibits COX-2, MMP-9, ICAM-1, and VEGF expression induced by TNF. H1299 cells (2×10⁶/ml) were left untreated or incubated with 25 mM honokiol for 12 h and then treated with 0.1 nM TNF for different times. Whole-cell extracts were prepared, and 50 mg of the whole-cell lysate was analyzed by Western blotting using antibodies against VEGF, MMP-9, and COX-2. (B) Honokiol inhibits cyclin D1 and c-myc expression induced by TNF. H1299 cells (2×10⁶/ml) were left untreated or incubated with 25 mM honokiol for 12 h and then treated with 0.1 nM TNF for different times. Whole-cell extracts were prepared, and 50 mg of the whole-cell lysate was analyzed by Western blotting using antibodies against cyclin D1 and c-myc. Data are for a representative experiment out of 3 independent ones showing similar results. (C) Honokiol inhibits the expression of anti-apoptotic gene products cIAP1, cIAP2 Bcl-x1, Bcl-2, cFLIP, TRAF2, and survivin. H1299 cells (2×10⁶/ml) were left untreated or incubated with 25 mM honokiol for 12 h and then treated with 0.1 nM TNF for different times. Whole-cell extracts were prepared, and 50 mg of the whole-cell lysate was analyzed by Western blotting using antibodies against IAP1, IAP2, bcl-x1, bcl-2, cFLIP, and survivin as indicated.

FIG. 32 shows a schematic representation of the effect of honokiol on TNF-induced NF-kB activation and apoptosis.

FIG. 33 (A) depicts the chemical structure of honokiol (also referred to herein as FINK). (B-F) are graphs of the viability of breast cancer cell lines, which were cultured in medium containing the indicated doses of HNK, after 24 hours of treatment, using the MTT assay. The results indicate that HNK inhibits proliferation in breast cancer cells.

FIGS. 34 (A and B) are graphs of the viability of Glioblastoma multifone cell lines, which were cultured in medium containing the indicated doses of HNK, after 24 hours of treatment, using the MTT assay. The results indicate that Glioblastoma multifone cell lines are resistant to HNK treatment.

FIG. 35 (A-G) are bar graphs which depict the viability of MCF-7 and MDA-MB-231 cell lines, which were cultured in medium containing the indicated doses of HNK either alone or in combination with a second drug, after 24 hours of treatment, using the MTT assay. The secondary drugs used in the study: A and B. SAHA (2 μM); C. 4-HT (100 nM); D and E. doxorubicin (ADR, 300 nM); F and G paclitaxel (PAC, 250 nM). The results indicate that HNK enhances the growth inhibitory activity of SAHA.

FIG. 36 is a graph of tumor volume over weeks of tumors in mice. MDA-MB-231 cells were injected into both flanks of athymic nude mice. The mice were treated with daily I.P. injections of either a vehicle (n=5) or HNK (2 mg/d, n=5) for four weeks. Tumor volume was measured weekly and the tumors in the experimental mice were significantly smaller (p<0.02) by two weeks to the conclusion of the study.

FIG. 37 (A) shows stains of MCF-7 cells were treated with HNK (60 μM) for the indicated time. Following treatment, the cells were harvested and stained for PI and annexin V, as described in Example 9. (B) is a bar graph in which the results of three independent experiments (HNK 60 μM, 24 h) are shown. Asterix indicates p<0.05. (C) is a series of photographs of the Western blots in which MCF-7 cells were treated with HNK (20 or 40 μM, 24 h), lysed and analyzed by Western blotting for the expression of apoptosis-related proteins. The results indicate that HNK induces apoptosis in breast cancer cells.

FIG. 38 (A) shows graphs of MDA-MB-231 cells, which were treated with HNK (30 μM, 24 hours) and analyzed for cell cycle using PI staining, as described in Example 9. (B) is a bar graph of the % cells versus concentration of HNK. The results of three independent experiments are shown. P>0.05 for the percentage of cells in S phase in the control compared to those treated with 30 μM HNK. (C) shows graphs of MCF-7 cells, which were treated with HNK (30 μM, 24 hours) and analyzed for cell cycle using PI staining. (D) is a bar graph of the % cells versus concentration of HNK. The results of three independent experiments are shown. (E) shows a series of photographs of Western blots of MDA-MB-231 cells, which were treated with HNK (20, 40 or 60 μM, for 24 h), lysed and analyzed by Western blotting for the expression of cell cycle related proteins. (F) shows a series of photographs of Western blots of MDA-MB-231 cells, which were treated with HNK (20, 40 or 60 μM, for 24 h), lysed and analyzed by Western blotting for the expression of EGFR and total and phosphorylated ERK2, as well as β-actin. The results indicate that HNK slows cell cycle in breast cancer cells.

FIG. 39 (A) is a bar graph showing the correlation of honokiol concentration with % apoptosis in Rat1a and Rat1a-mAkt fibroblast cell lines after treatment with 0 to 40 μg/ml honokiol in the absence of growth factors; (B) is a bar graph showing the correlation of honokial concentration with mitochondrial HK activity in Rat1a and Rat1a-mAkt fibroblast cell lines were withdrawn from growth factors in the presence or absence of Honokiol and the percentage of total cellular hexokinase activity associated with the mitochondria was determined; (C) is a bar graph showing the correlation of honokiol concentration with % apoptosis in wildtype and Bax/Bak DKO MEF cell lines after treatment with 0 to 40 μg/ml honokiol in the absence of growth factors; (D) is a bar graph showing the correlation of honokial concentration with mitochondrial HK activity in wildtype and Bax/Bak DKO MEF cell lines were withdrawn from growth factors in the presence or absence of Honokiol and the percentage of total cellular hexokinase activity associated with the mitochondria was determined.

FIG. 40 shows that in vivo honokiol treatment stablizes collagen induced arthritis (CIA) pathology in both C57Bl/6 and LMP1 transgenic mice mice, but does not inhibit to level of negative control.

FIG. 41 shows the effect of honokiol treatment on IL-6 and TNF-alpha production in CH12.hCD40-LMP1 B cells.

FIG. 42 shows that NFkB activation was inhibited by honokiol in a dose dependent manner in mouse M12.4.1 cells.

FIG. 43 illustrates the effects of the combination of TSA and honokiol treatment on cancer cells.

### DETAILED DESCRIPTION

Definitions

The term “alkyl”, as used herein, unless otherwise specified, includes a saturated straight, branched, or cyclic, primary, secondary, or tertiary hydrocarbon, including those of C₁₋₂₂ or C₁₋₁₀ and specifically includes methyl, ethyl, CF₂CF₂CF₃, propyl, isopropyl, cyclopropyl, butyl, isobutyl, secbutyl, t-butyl, pentyl, cyclopentyl, isopentyl, neopentyl, hexyl, isohexyl, cyclohexyl, cyclohexylmethyl, heptyl, cycloheptyl, octyl, cyclo-octyl, dodecyl, tridecyl, pentadecyl, icosyl, hemicosyl, and decosyl. The alkyl group may be optionally substituted with, e.g., halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl”, as used herein, and unless otherwise specified, includes a C₁ to C₄ saturated straight, branched, or if appropriate, a cyclic (for example, cyclopropyl) alkyl group, which is optionally substituted.

The term “amino” includes an “—N(R)₂” group, and includes primary amines, and secondary and tertiary amines which is optionally substituted for example with alkyl, aryl, heterocycle, and or sulfonyl groups. Thus, (R)₂ may include, but is not limited to, two hydrogens, a hydrogen and an alkyl, a hydrogen and an aryl, a hydrogen and an alkenyl, two alkyls, two aryls, two alkenyls, one alkyl and one alkenyl, one alkyl and one aryl, or one aryl and one alkenyl.

Whenever a range of carbon atoms is referred to, it includes independently and separately every member of the range. As a nonlimiting example, the term “C₁-C₁₀ alkyl” is considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities.

The term “amido” includes a moiety represented by the structure “—C(O)N(R)2”, wherein R may include alkyl, alkenyl and aryl that is optionally substituted.

The term “protected” as used herein and unless otherwise defined includes a group that is added to an atom such as an oxygen, nitrogen, or phosphorus atom to prevent its further reaction or for other purposes. A wide variety of oxygen and nitrogen protecting groups are known to those skilled in the art of organic synthesis.

The term “aryl”, as used herein, and unless otherwise specified, includes a stable monocyclic, bicyclic, or tricyclic carbon ring with Lip to 8 members in each ring, and at least one ring being aromatic. Examples include, but are not limited to, benzyl, phenyl, biphenyl, or naphthyl. The aryl group can be substituted with one or more moieties including, but not limited to, halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “halo”, as used herein, specifically refers to chloro, bromo, iodo, and fluoro.

The term “alkenyl” refers to a straight, branched, or cyclic unsaturated hydrocarbon including one of C₂₋₂₂ with at least one double bond. Examples include, but are not limited to, vinyl, allyl, and methyl-vinyl. The alkenyl group can be optionally substituted in the same manner as described above for the alkyl groups.

The term “alkynyl” refers to a straight or branched hydrocarbon with at least one triple bond, including one of C₂₋₂₂. The alkynyl group can be optionally substituted in the same manner as described above for the alkyl groups.

The term “alkoxy” includes a moiety of the structure —O-alkyl.

The term “heterocycle” or “heterocyclic” includes a saturated, unsaturated, or aromatic, monocyclic (for example, stable 5 to 7 membered monocyclic) or bicyclic heterocyclic (for example, 8 to 11 membered bicyclic) ring that consists of carbon atoms and from one to three heteroatoms including but not limited to O, S, N, and P; and wherein the nitrogen and sulfur heteroatoms may optionally be oxidized, and/or the nitrogen atoms quarternized and including any bicyclic group in which any of the above-defined heterocyclic rings is fused to a benzene ring. The heterocyclic ring may be attached at any heteroatom or carbon atom which results in the creation of a stable structure. Nonlimiting examples or heterocyclic groups include pyrrolyl, pyrimidyl, pyridinyl, imidazolyl, pyridyl, furanyl, pyrazole, oxazolyl, oxirane, isooxazolyl, indolyl, isoindolyl, thiazolyl, isothiazolyl, quinolyl, tetrazolyl, bonzofuranyl, thiophrene, piperazine, and pyrrolidine.

The term “acyl” includes a group of the formula R′C(O), wherein R′ is a straight, branched, or cyclic, substituted or unsubstituted alkyl or aryl.

The term “host”, as used herein, unless otherwise specified, includes mammals (e.g., cats, dogs, horses, mice, etc.), humans, or other organisms in need of treatment. Hosts that are “predisposed to” conditions such as cancer-related conditions can be defined as hosts that do not exhibit overt symptoms of one or more of these conditions but that are genetically, physiologically, or otherwise at risk of developing one or more of these conditions. Thus, compositions of the present invention can be used prophylactically as chemopreventative agents for these conditions. Further, a “composition” can include one or more chemical compounds, as described herein.

The phrase “treatment with an effective amount” as used herein includes administration of an amount sufficient for prevention, treatment, or amelioration of one or more of the symptoms of diseases or disorders, for example, an angiogenic disease (for example, to limit tumor growth, decrease tumor volume or to slow or block tumor metastisis), and includes a amount which results in the effect that one or more of the symptoms of a disease or disorder are ameliorated or otherwise beneficially altered.

The term “pharmaceutically acceptable salt” as used herein, unless otherwise specified, includes those salts which are, within the scope of sound medical judgment, suitable for use in contact with the tissues of hosts without undue toxicity, irritation, allergic response and the like, and are commensurate with a reasonable benefit/risk ratio and effective for their intended use. The salts can be prepared in situ during the final isolation and purification of one or more compounds of the composition, or separately by reacting the free base function with a suitable organic acid. Non-pharmaceutically acceptable acids and bases also find use herein, as for example, in the synthesis and/or purification of the compounds of interest. Nonlimiting examples of such salts are (a) acid addition salts formed with inorganic salts (for example hydrochloric acid, hydrobromic acid, sulfuric acid, phosphoric acid, nitric acid, and the like), and salts formed with organic salts such as acetic acid, oxalic acid, tartaric acid, succinic acid, ascorbic acid, benzoic acid, tannic acid, and the like; (b) base addition salts formed with metal cations such as zinc, calcium, magnesium, aluminum, copper, nickel and the like; (c) combinations of (a) and (b).

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

Representative alkali or alkaline earth metal salts that may be used as the pharmaceutically acceptable salts include, but are not limited to, sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pharmaceutically acceptable esters” as used herein, unless otherwise specified, includes those esters of one or more compounds of the composition, which are, within the scope of sound medical judgment, suitable for use in contact with the tissues of hosts without undue toxicity, irritation, allergic response and the like, are commensurate with a reasonable benefit/risk ratio, and are effective for their intended use.

The term “pharmaceutically acceptable prodrugs” as used herein, unless otherwise specified, includes those prodrugs of one or more compounds of the composition which are, with the scope of sound medical judgment, suitable for use in contact with the tissues of hosts without undue toxicity, irritation, allergic response and the like, are commensurate with a reasonable benefit/risk ratio, and are effective for their intended use. Pharmaceutically acceptable prodrugs also include zwitterionic forms, where possible, of one or more compounds of the composition. The term “prodrug” includes compounds that are rapidly transformed in vivo to yield the parent compound, for example by hydrolysis in blood.

The term “pharmaceutically acceptable carrier and/or excipient” as used herein, unless otherwise specified, includes any carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, surfactants, colorants, flavorants, or sweeteners and the like, as suited to the particular dosage form desired.

### I. Compounds

The compounds disclosed herein may be used in the methods and compositions, which in one embodiment have useful activity, for example, against cancer, and in particular, multiple myeloma. The term “honokiol derivatives” is intended to include honokiol-type and magnolol-type compounds, or other compounds described herein with a desired activity of honokiol.

In one embodiment, the compound is of Formula Ia, or a salt, ester or prodrug thereof:

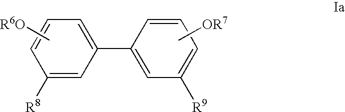

wherein R⁶ and R⁷ are independently H, alkyl, alkenyl, alkynyl, or aryl, which is optionally substituted, and are independently, for example a C₁₋₁₀ alkyl, alkenyl or alkynyl, e.g. methyl, ethyl or propyl;

wherein R⁸ and R⁹ are independently alkyl, alkenyl, alkynyl, or aryl, which is optionally substituted and are independently, for example, C₁₋₁₀ alkyl or alkenyl, such as vinyl or allyl; and

wherein optionally at least one of R⁸ and R⁹ are alkyl, such as C₁₋₅ alkyl.

In one subembodiment of Formula Ia:

R⁶ and R⁷ are independently H or C₁₋₅ alkyl, e.g. methyl, ethyl or propyl;

R⁸ and R⁹ are independently C₁₋₅ alkyl or alkenyl, such as vinyl or allyl; and

at least one of R⁸ and R⁹ are C₁₋₅ alkyl, such as methyl, ethyl, propyl or butyl.

In another embodiment, the compound has the Formula Ib, or a salt, ester or prodrug thereof:

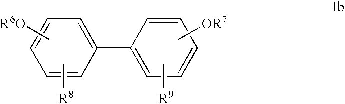

wherein R⁶ and R⁷ are independently H, alkyl, alkenyl, alkynyl, or aryl, which is optionally substituted, and are independently, for example a C₁₋₁₀ alkyl, alkenyl or alkynyl, e.g., methyl, ethyl or propyl;

wherein R⁸ and R⁹ are independently alkyl, alkenyl, alkynyl, or aryl, which is optionally substituted and are independently, for example, C₁₋₁₀ alkyl or alkenyl, such as vinyl or allyl; and

wherein optionally at least one of R⁶ and R⁷ are not H.

In one subembodiment of Formula Ib:

R⁶ and R⁷ are independently H, alkyl, such as C₁₋₅ alkyl, alkyl, alkenyl or alkynyl, e.g., methyl, ethyl or propyl;

R⁸ and R⁹ are independently C₁₋₅ alkyl or alkenyl, such as vinyl or allyl; and

at least one of R⁶ and R⁷ are not H.

Also provided are compounds of Formula Ic, Id, Ie or If, or a salt, ester or prodrug thereof:

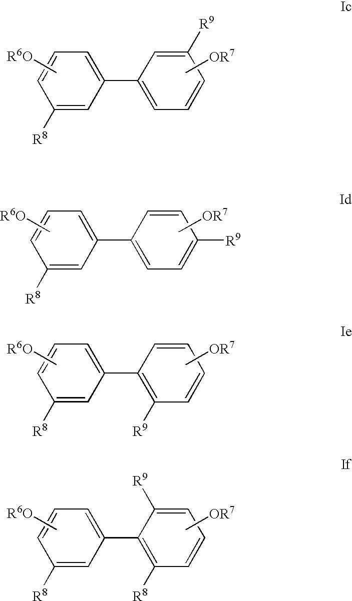

wherein R⁶ and R⁷ are independently H, alkyl, alkenyl, alkynyl, or aryl, which is optionally substituted, and are independently, for example a C₁₋₁₀ alkyl, alkenyl or alkynyl, e.g. methyl, ethyl or propyl;

wherein R⁸ and R⁹ are independently alkyl, alkenyl, alkynyl, or aryl, which is optionally substituted and are independently, for example, C₁₋₁₀ alkyl or alkenyl, such as vinyl or allyl; and

wherein optionally at least one of R⁸ and R⁹ are alkyl, such as C₁₋₅ alkyl; and

wherein optionally at least one of R⁶ and R⁷ are not H.

In one subembodiment of Formulas Ic, Id, Ie or If:

R⁶ and R⁷ are independently H or C₁₋₅ alkyl, alkenyl or alkynyl, e.g. methyl, ethyl or propyl; and

R⁸ and R⁹ are independently alkyl, such as C₁₋₅ alkyl, alkyl or alkenyl, such as vinyl or allyl.

In one embodiment, the compound is honokiol or magnolol:

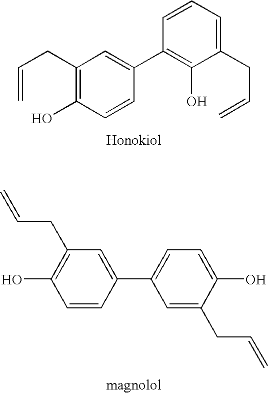

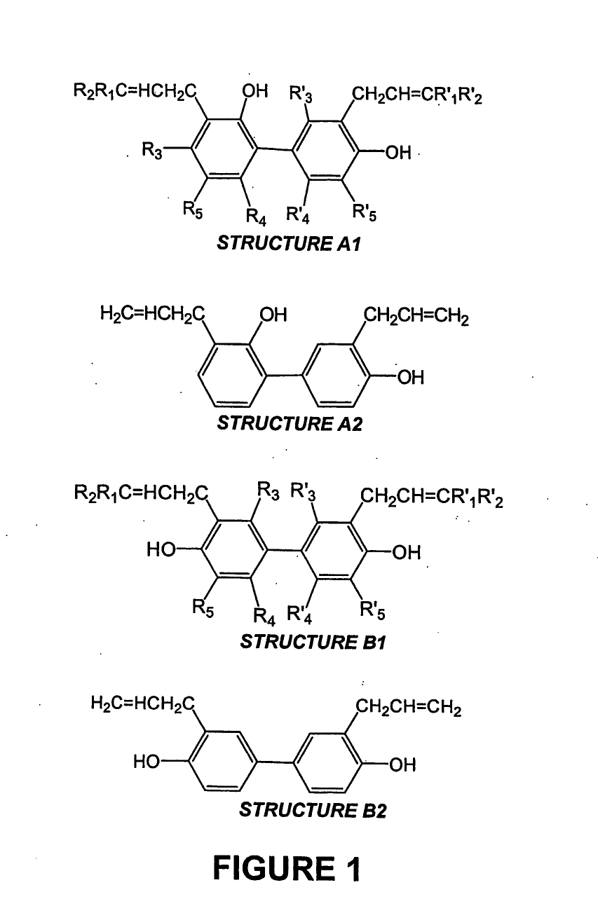

Other exemplary honokiol derivatives include compounds of FIGS. 1, 2, 3 and 4 as described in U.S. Patent Appl. Publ. No. 2004/0105906, published Jun. 3, 2004, the disclosure of which is incorporated herein by reference. The honokiol derivative may be e.g., a honokiol-type compound or a magnolol-type compound and various other derivatives with the desired honokiol activity as disclosed herein.

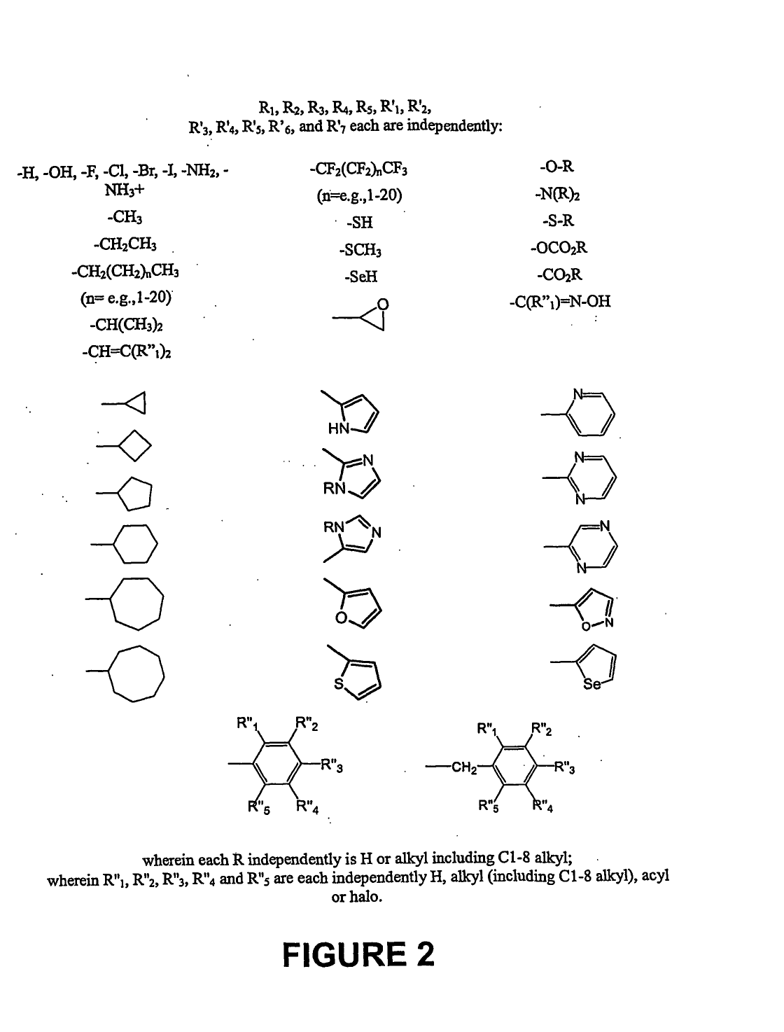

Honokiol-type compounds include, but are not limited to, structure A1 illustrated in FIG. 1. More particularly, honokiol-type compounds can include structure A2 illustrated in FIG. 1. The functional groups of the honokiol-type compounds are indicated as R₁, R₂, R₃, R₄, R₅, R′₁, R′₂, R′₃, R′₄, and R′₅. The functional groups can be independently selected from groups that include, but are not limited to, hydrogen, hydroxyl groups, amides, amines, hydrocarbons, halogenated hydrocarbons, cyclic hydrocarbons, cyclic heterocarbons, halogenated cyclic heterocarbons, benzyl, halogenated benzyl, organo selenium compounds, sulfides, carbonyl, thiol, ether, dinitrogen ring compounds, thiophenes, pyridines, pyrroles, imidazoles, and pyrimidines. FIG. 2 is a diagram that illustrates exemplary functional groups including R₁, R₂, R₃, R₄, R₅, R′₁, R′₂, R′₃, R′₄, and R′₅ which may be independently selected.

In addition, honokiol-type compounds include pharmaceutically acceptable salts, esters, and prodrugs of the compounds described or referred to above.

Magnolol-type compounds include, but are not limited to, structure B1 illustrated in FIG. 1. More particularly, magnolol-type compounds can include structure B2 illustrated in FIG. 1. The functional groups of the magnolol-type compounds are indicated as R₁, R₂, R₃, R₄, R₅, R′₁, R′₂, R′₃, R′₄, and R′₅. The functional groups may be independently selected and include, but are not limited to, hydrogen, hydroxyl groups, amides, amines, hydrocarbons, halogenated hydrocarbons, cyclic hydrocarbons, cyclic heterocarbons, halogenated cyclic heterocarbons, benzyl, halogenated benzyl compounds, organo selenium compounds, sulfide compounds, cabonyl compounds, thiol compounds, ether compounds, dinitrogen ring compounds, thiophene compounds, pyridine compounds, pyrrole compounds, imidazole compounds, and pyrimidine compounds. FIG. 2 is a diagram that illustrates exemplary functional groups of R₁, R₂, R₃, R₄, R₅, R′₁, R′₂, R′₃, R′₄, and R′₅ that may be independently selected.

Analogues, homologues, isomers, or derivatives of the honlokiol-type compounds also may be used, such as those that function to treat angiogenic-, neoplastic-, and cancer-related conditions in a host and/or function prophylactically as a chemopreventative composition, as well as pharmaceutically acceptable salts, esters, and prodrugs of the compounds described herein.

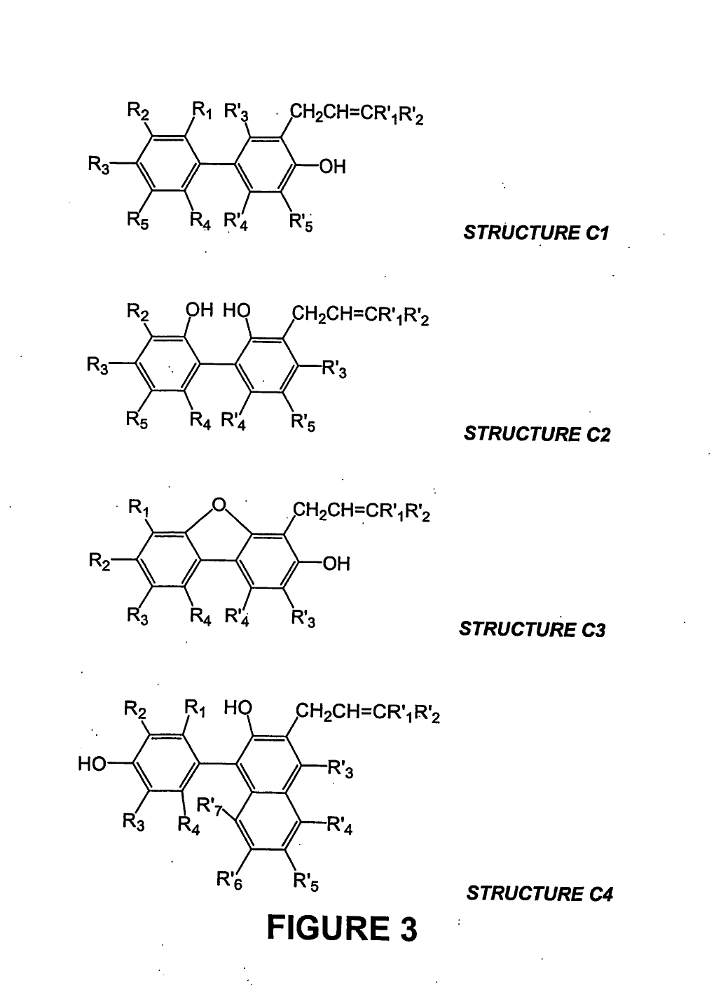

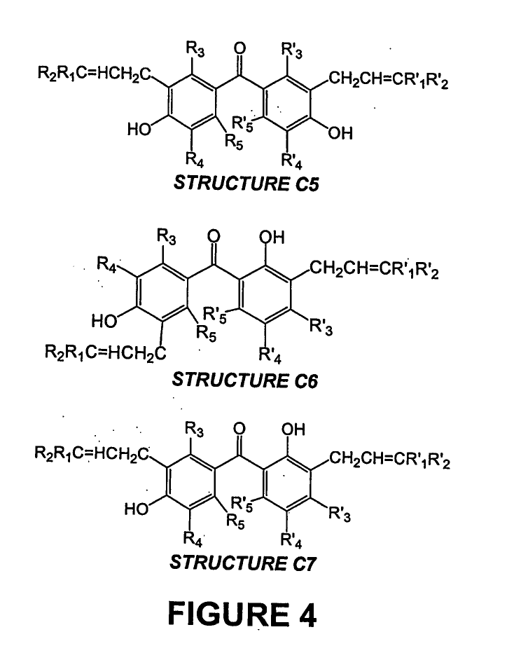

FIGS. 3 and 4 illustrate additional structures C₁₋₇ that are examples of other useful compounds. These compounds may be used instead of or in addition to the honokiol-type and/or magnolol-type compounds described above. In this regard, functional groups R′₆ and R′₇ can independently be any of the functional groups described or referred to above in FIG. 2.

Other compounds that are useful include compounds of formula D2:

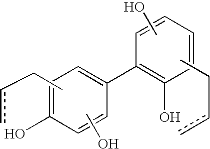

wherein the allyl group is in oxidized or reduced form.

In another embodiment, the compound has the formula D3:

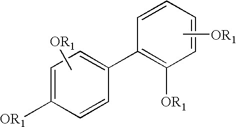

wherein the OR₁, substituent denotes an ether or ester linkage, and for example, each R₁ is independently alkyl, e.g., C₁₋₁₀ alkyl, or acyl, e.g. C₁₋₁₀ acyl.

In another embodiment, the compound is a compound of one of the following formulas:

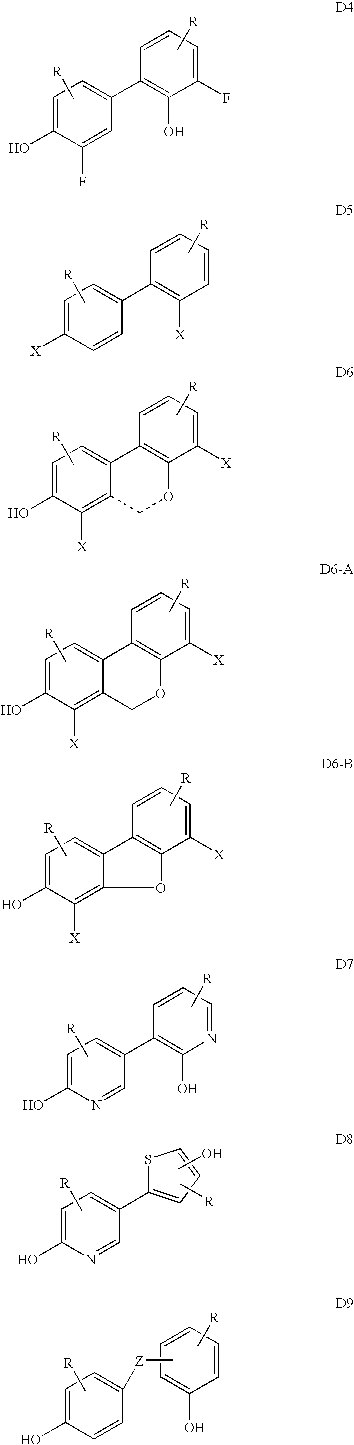

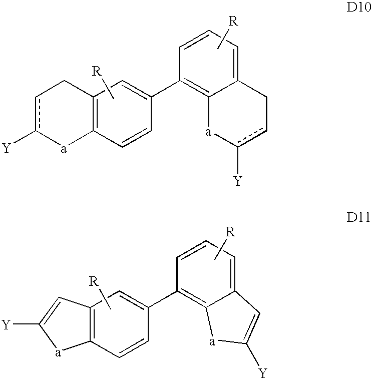

wherein each R is independently alkyl, alkenyl, aryl, or vinyl which is optionally straight, branched, or cyclic and is optionally substituted. Optionally each R is independently C₁₋₁₀ alkyl, C₁₋₁₀ alkenyl or C₁₋₁₀ alkynyl. For example, each R may be independently selected from the following groups:

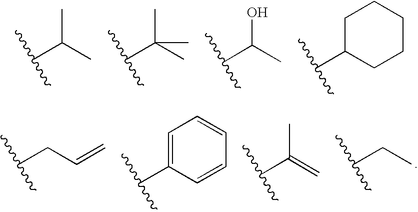

In Formula D5, each X is independently, for example, halogen (e.g., F), N(R¹)₂, SH or SR¹, where each R¹ is independently, e.g., H or alkyl.

In Formula D6, D6-A and D6-B, each X is independently H, alkyl (e.g., Methyl or C1-10 alkyl) or halogen, e.g., F. In Formula D6, the dashed line shows either the presence or absence of a CH₂ group thus making the ring either five or six membered, as shown in D6-A and D6-B.

In Formula D9, Z is O, S, SO₂, CO, or (CH₂)N where n is 1-8.

In Formula D10 and D11, each Y is independently H, OH or alkyl, and each a is independently O, NR¹ or S, where each R¹ is independently, e.g., H or alkyl, e.g., C₁₋₅ alkyl. In D10, the dotted line shows a double or single bond.

In one embodiment, the compound is one of the following compounds:

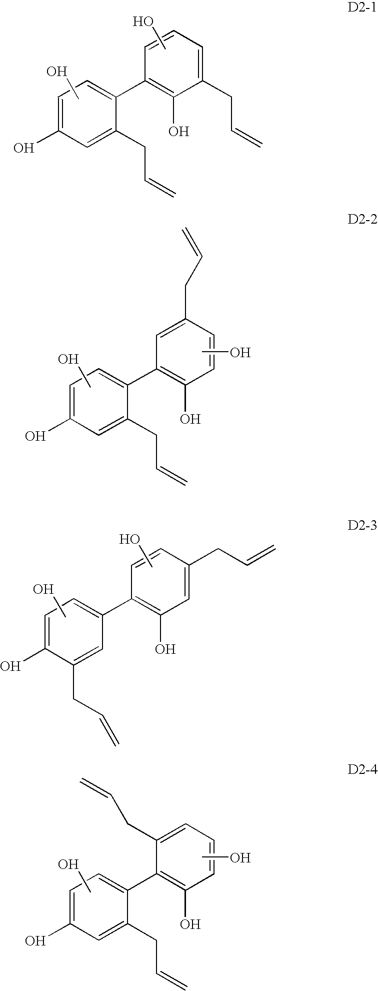

In another embodiment the compound has the formula:

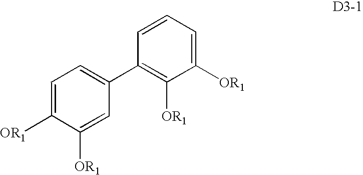

wherein the OR₁, substituent denotes an ether or ester linkage, and for example, each R₁ is independently alkyl, e.g., C₁₋₁₀ alkyl, or acyl, e.g., C₁₋₁₀ acyl.

In another embodiment, the compound has one of the following formulas:

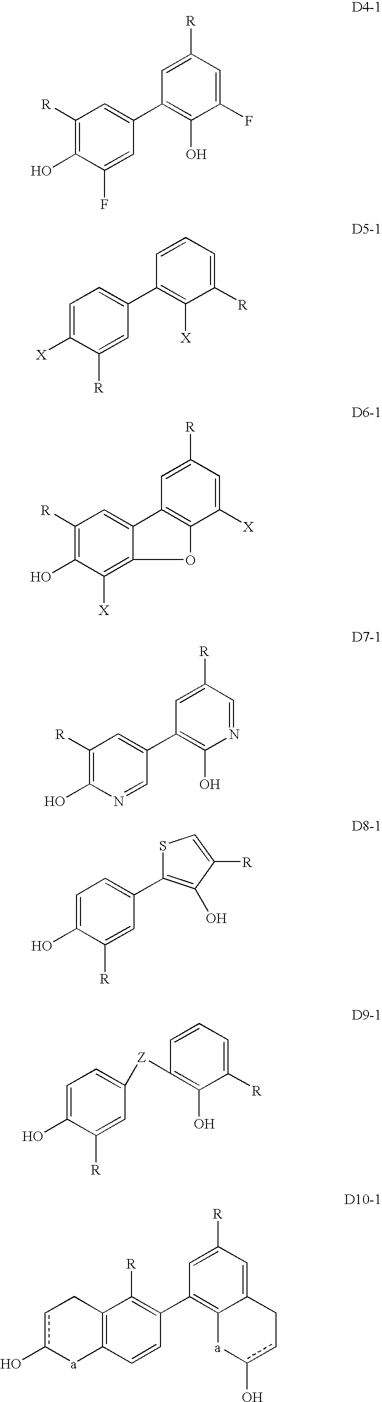

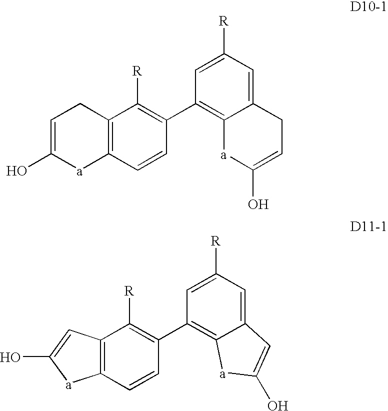

wherein each R is independently alkyl, alkenyl, aryl, or vinyl which is optionally straight, branched, or cyclic and is optionally substituted. Optionally each R is independently C₁₋₁₀ alkyl, C₁₋₁₀ alkenyl or C₁₋₁₀ alkynyl. For example, each R may be independently selected from the following groups:

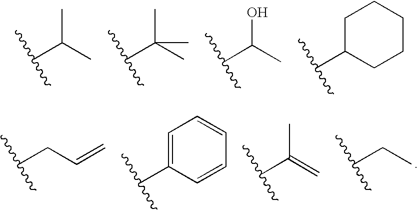

In Formula D5-1, each X is independently, for example, halogen (e.g., F), N(R¹)₂, SH or SR¹, where each R¹ is independently, e.g., H or alkyl.

In Formula D6-1, each X is independently H, alkyl (e.g., methyl) or halogen, e.g., F.

In Formula D9-1, Z is O, S, SO₂, CO, or (CH₂)ₙ where n is e.g., 1-8.

In Formula D10-1, D0-2 and D11-1, each Y is independently H, OH or alkyl, and each “a” is independently O, NR¹ or S, where each R¹ is independently, e.g., H or alkyl, e.g., C₁₋₅ alkyl. In D10, the dotted line shows a double or single bond.

In another embodiment, the compound has the formula:

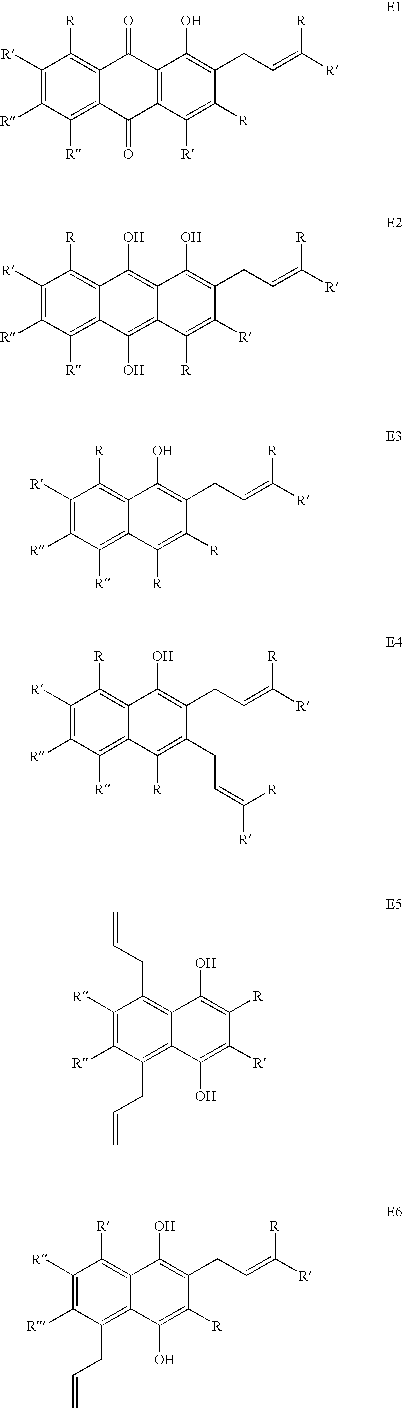

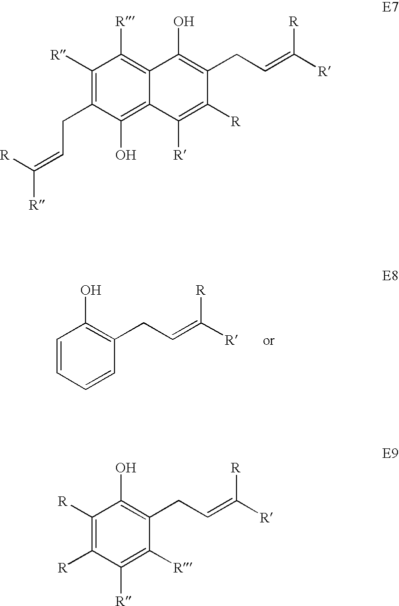

wherein each R, R′, R″, and R′″ are independently H, OH, F, Cl, 1, Br, CH₃, —(CH₂)ₙCH₃ (where n is e.g. 1-10),

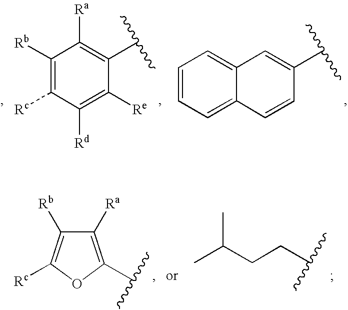

wherein R^{a}, R^{b}, R^{c}, R^{d} and R^{e} each are independently H, OH, Oalkyl, alkyl (including C₁₋₈ alkyl), alkenyl, or halogen.

In another embodiment, the compound is valproic acid (Depakote, Depakene) or a pharmaceutically acceptable salt, ester or prodrug thereof. Valproic acid is an antiepileptic agent and is known to inhibit hepatic glucuronidase and epoxide hydrolase.

Other useful compounds are those shown in Scheme 1A, as well as salts, esters, isomers and prodrugs thereof.

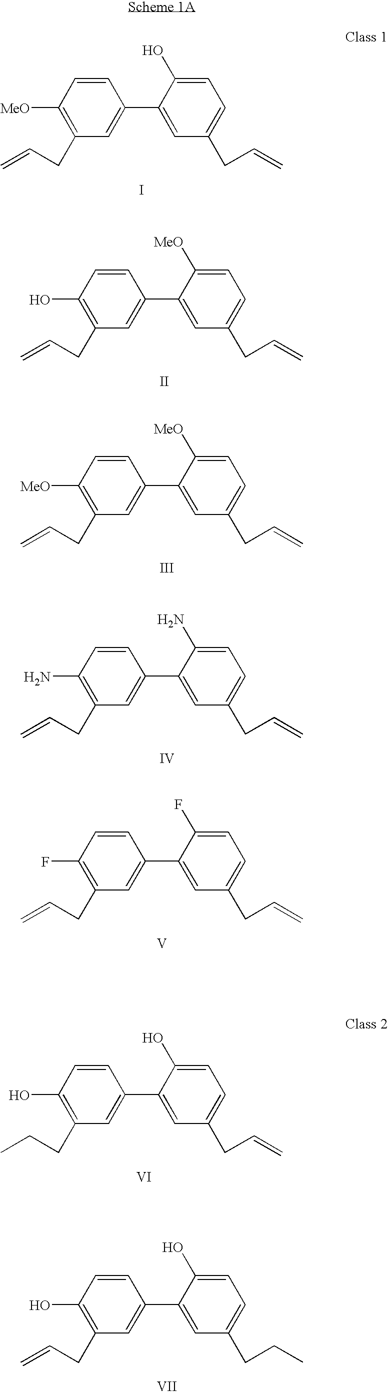

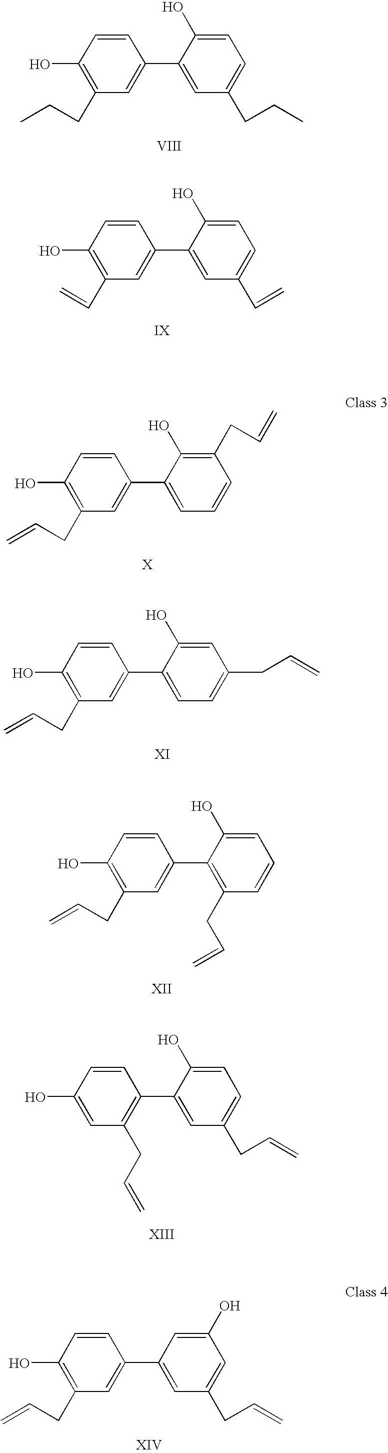

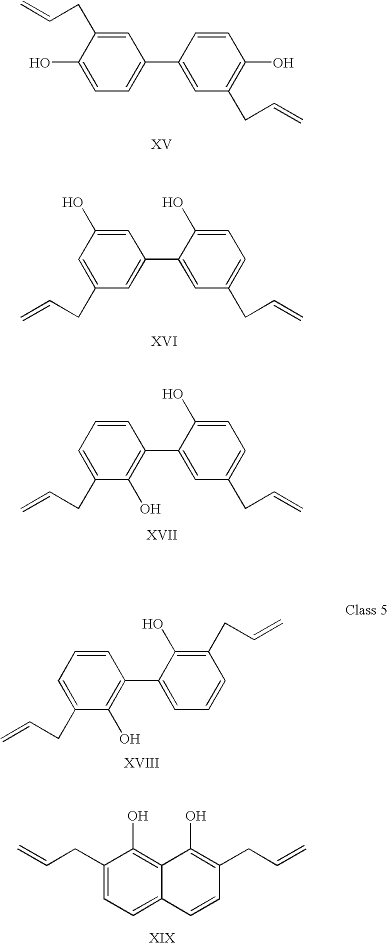

Further examples of useful compounds include those listed in Table 1 as well as salts, isomers, esters and prodrugs thereof.

It is to be understood that the compounds disclosed herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the disclosure of a compound herein encompasses any racemic, optically active, polymorphic, or steroisomeric form, or mixtures thereof, which preferably possesses the useful properties described herein, it being well known in the art how to prepare optically active forms and how to determine activity using the standard tests described herein, or using other similar tests which are will known in the art. Examples of methods that can be used to obtain optical isomers of the compounds include the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce assymetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

### II. Methods of Treatment

The compounds and pharmaceutical compositions provided herein can be used in the treatment of a condition characterized by angiogenesis, tumorogenesis, a neoplastic condition, cancer, a skin disorder, an inflammatory disorder and/or a bone disorder, such as osteoporosis.

### Cancers

In one embodiment, the compounds of the present invention can be used to treat a carcinoma, sarcoma, lymphoma, leukemia, and/or myeloma. In other embodiments of the present invention, the compounds disclosed herein can be used to treat solid tumors.

The compounds of the present invention can be used for the treatment of cancer, such as, but not limited to cancer of the following organs or tissues: breast, prostate, lung, bronchus, colon, urinary, bladder, non-Hodgkin lymphoma, melanoma, kidney, renal, pancreas, pharnx, thyroid, stomach, brain, multiple myeloma, esophagus, liver, intrahepatic bile duct, cervix, larynx, acute myeloid leukemia, chronic lymphatic leukemia, soft tissue, such as heart, Hodgkin lymphoma, testis, small intestine, chronic myeloid leukemia, acute lymphatic leukemia, anus, anal canal, anorectal, thyroid, vulva, gallbladder, pleura, eye, nose nasal cavity, middle ear, nasopharnx, ureter, peritoneum, omentum, mesentery, and gastrointestinal, high grade glioma, glioblastoma, colon, rectal, pancreatic, gastric cancers, hepatocellular carcinoma; head and neck cancers, carcinomas; renal cell carcinoma; adenocarcinoma; sarcomas; hemangioendothelioma; lymphomas; leukemias, mycosis fungoides. In additional embodiments, the compounds of the present invention can be used to treat skin diseases including, but not limited to, the malignant diseases angiosarcoma, hemangioendothelioma, basal cell carcinoma, squamous cell carcinoma, malignant melanoma and Kaposi's sarcoma, and the non-malignant diseases or conditions such as psoriasis, lymphangiogenesis, hemangioma of childhood, Sturge-Weber syndrome, verruca vulgaris, neurofibromatosis, tuberous sclerosis, pyogenic granulomas, recessive dystrophic epidermolysis bullosa, venous ulcers, acne, rosacea, eczema, molluscum contagious, seborrheic keratosis, and actinic keratosis.

Compositions of this invention can be used to treat these cancers and other cancers at any stage from the discovery of the cancer to advanced stages. In addition, compositions of this invention can be used in the treatment of the primary cancer and metastases thereof.

In other embodiments of the present invention, the compounds described herein can be used for the treatment of cancer, including, but not limited to the cancers listed in Table 2 below.

TABLE 2
Types of Cancer

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult (Primary) |
| Acute Myeloid Leukemia, Adult | Hepatocellular (Liver) Cancer, Childhood |
| Acute Myeloid Leukemia, Childhood | (Primary) |
| Adrenocortical Carcinoma | Hodgkin's Lymphoma, Adult |
| Adrenocortical Carcinoma, Childhood | Hodgkin's Lymphoma, Childhood |
| AIDS-Related Cancers | Hodgkin's Lymphoma During Pregnancy |
| AIDS-Related Lymphoma | Hypopharyngeal Cancer |
| Anal Cancer | Hypothalamic and Visual Pathway Glioma, |
| Astrocytoma, Childhood Cerebellar | Childhood |
| Astrocytoma, Childhood Cerebral | Intraocular Melanoma |
| Basal Cell Carcinoma | Islet Cell Carcinoma (Endocrine Pancreas) |
| Bile Duct Cancer, Extrahepatic | Kaposi's Sarcoma |
| Bladder Cancer | Kidney (Renal Cell) Cancer |
| Bladder Cancer, Childhood | Kidney Cancer, Childhood |
| Bone Cancer, Osteosarcoma/Malignant | Laryngeal Cancer |
| Fibrous Histiocytoma | Laryngeal Cancer, Childhood |
| Brain Stem Glioma, Childhood | Leukemia, Acute Lymphoblastic, Adult |
| Brain Tumor, Adult | Leukemia, Acute Lymphoblastic, Childhood |
| Brain Tumor, Brain Stem Glioma, | Leukemia, Acute Myeloid, Adult |
| Childhood | Leukemia, Acute Myeloid, Childhood |
| Brain Tumor, Cerebellar Astrocytoma, | Leukemia, Chronic Lymphocytic |
| Childhood | Leukemia, Chronic Myelogenous |
| Brain Tumor, Cerebral | Leukemia, Hairy Cell |
| Astrocytoma/Malignant Glioma, | Lip and Oral Cavity Cancer |
| Childhood | Liver Cancer, Adult (Primary) |
| Brain Tumor, Ependymoma, Childhood | Liver Cancer, Childhood (Primary) |
| Brain Tumor, Medulloblastoma, | Lung Cancer, Non-Small Cell |
| Childhood | Lung Cancer, Small Cell |
| Brain Tumor, Supratentorial Primitive | Lymphoma, AIDS-Related |
| Neuroectodermal Tumors, Childhood | Lymphoma, Burkitt's |
| Brain Tumor, Visual Pathway and | Lymphoma, Cutaneous T-Cell, see Mycosis |
| Hypothalamic Glioma, Childhood | Fungoides and Sézary Syndrome |
| Brain Tumor, Childhood | Lymphoma, Hodgkin's, Adult |
| Breast Cancer | Lymphoma, Hodgkin's, Childhood |
| Breast Cancer, Childhood | Lymphoma, Hodgkin's During Pregnancy |
| Breast Cancer, Male | Lymphoma, Non-Hodgkin's, Adult |
| Bronchial Adenomas/Carcinoids, | Lymphoma, Non-Hodgkin's, Childhood |
| Childhood | Lymphoma, Non-Hodgkin's During Pregnancy |
| Burkitt's Lymphoma | Lymphoma, Primary Central Nervous System |
| Carcinoid Tumor, Childhood | Macroglobulinemia, Waldenström's |
| Carcinoid Tumor, Gastrointestinal | Malignant Fibrous Histiocytoma of |
| Carcinoma of Unknown Primary | Bone/Osteosarcoma |
| Central Nervous System Lymphoma, | Medulloblastoma, Childhood |
| Primary | Melanoma |
| Cerebellar Astrocytoma, Childhood | Melanoma, Intraocular (Eye) |
| Cerebral Astrocytoma/Malignant Glioma, | Merkel Cell Carcinoma |
| Childhood | Mesothelioma, Adult Malignant |
| Cervical Cancer | Mesothelioma, Childhood |
| Childhood Cancers | Metastatic Squamous Neck Cancer with Occult |
| Chronic Lymphocytic Leukemia | Primary |
| Chronic Myelogenous Leukemia | Multiple Endocrine Neoplasia Syndrome, |
| Chronic Myeloproliferative Disorders | Childhood |
| Colon Cancer | Multiple Myeloma/Plasma Cell Neoplasm |
| Colorectal Cancer, Childhood | Mycosis Fungoides |
| Cutaneous T-Cell Lymphoma, see | Myelodysplastic Syndromes |
| Mycosis Fungoides and Sézary | Myelodysplastic/Myeloproliferative Diseases |
| Syndrome | Myelogenous Leukemia, Chronic |
| Endometrial Cancer | Myeloid Leukemia, Adult Acute |
| Ependymoma, Childhood | Myeloid Leukemia, Childhood Acute |
| Esophageal Cancer | Myeloma, Multiple |
| Esophageal Cancer, Childhood | Myeloproliferative Disorders, Chronic |
| Ewing's Family of Tumors | Nasal Cavity and Paranasal Sinus Cancer |
| Extracranial Germ Cell Tumor, | Nasopharyngeal Cancer |
| Childhood | Nasopharyngeal Cancer, Childhood |
| Extragonadal Germ Cell Tumor | Neuroblastoma |
| Extrahepatic Bile Duct Cancer | Non-Hodgkin's Lymphoma, Adult |
| Eye Cancer, Intraocular Melanoma | Non-Hodgkin's Lymphoma, Childhood |
| Eye Cancer, Retinoblastoma | Non-Hodgkin's Lymphoma During Pregnancy |
| Gallbladder Cancer | Non-Small Cell Lung Cancer |
| Gastric (Stomach) Cancer | Oral Cancer, Childhood |
| Gastric (Stomach) Cancer, Childhood | Oral Cavity Cancer, Lip and |
| Gastrointestinal Carcinoid Tumor | Oropharyngeal Cancer |
| Germ Cell Tumor, Extracranial, | Osteosarcoma/Malignant Fibrous Histiocytoma |
| Childhood | of Bone |
| Germ Cell Tumor, Extragonadal | Ovarian Cancer, Childhood |
| Germ Cell Tumor, Ovarian | Ovarian Epithelial Cancer |
| Gestational Trophoblastic Tumor | Ovarian Germ Cell Tumor |
| Glioma, Adult | Ovarian Low Malignant Potential Tumor |
| Glioma, Childhood Brain Stem | Pancreatic Cancer |
| Glioma, Childhood Cerebral | Pancreatic Cancer, Childhood |
| Astrocytoma | Pancreatic Cancer, Islet Cell |
| Glioma, Childhood Visual Pathway and | Paranasal Sinus and Nasal Cavity Cancer |
| Hypothalamic | Parathyroid Cancer |
| Skin Cancer (Melanoma) | Penile Cancer |
| Skin Carcinoma, Merkel Cell | Pheochromocytoma |
| Small Cell Lung Cancer | Pineoblastoma and Supratentorial Primitive |
| Small Intestine Cancer | Neuroectodermal Tumors, Childhood |
| Soft Tissue Sarcoma, Adult | Pituitary Tumor |
| Soft Tissue Sarcoma, Childhood | Plasma Cell Neoplasm/Multiple Myeloma |
| Squamous Cell Carcinoma, see Skin | Pleuropulmonary Blastoma |
| Cancer (non-Melanoma) | Pregnancy and Breast Cancer |
| Squamous Neck Cancer with Occult | Pregnancy and Hodgkin's Lymphoma |
| Primary, Metastatic | Pregnancy and Non-Hodgkin's Lymphoma |
| Stomach (Gastric) Cancer | Primary Central Nervous System Lymphoma |
| Stomach (Gastric) Cancer, Childhood | Prostate Cancer |
| Supratentorial Primitive | Rectal Cancer |
| Neuroectodermal Tumors, Childhood | Renal Cell (Kidney) Cancer |
| T-Cell Lymphoma, Cutaneous, see | Renal Cell (Kidney) Cancer, Childhood |
| Mycosis Fungoides and Sézary | Renal Pelvis and Ureter, Transitional Cell |
| Syndrome | Cancer |
| Testicular Cancer | Retinoblastoma |
| Thymoma, Childhood | Rhabdomyosarcoma, Childhood |
| Thymoma and Thymic Carcinoma | Salivary Gland Cancer |
| Thyroid Cancer | Salivary Gland Cancer, Childhood |
| Thyroid Cancer, Childhood | Sarcoma, Ewing's Family of Tumors |
| Transitional Cell Cancer of the Renal | Sarcoma, Kaposi's |
| Pelvis and Ureter | Sarcoma, Soft Tissue, Adult |
| Trophoblastic Tumor, Gestational | Sarcoma, Soft Tissue, Childhood |
| Unknown Primary Site, Carcinoma of, | Sarcoma, Uterine |
| Adult | Sezary Syndrome |
| Unknown Primary Site, Cancer of, | Skin Cancer (non-Melanoma) |
| Childhood | Skin Cancer, Childhood |
| Unusual Cancers of Childhood | |
| Ureter and Renal Pelvis, Transitional Cell | |
| Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

### Myeloma

In certain particular aspects of the present invention, the compounds described herein can be used in the treatment of myeloma. In one embodiment, honokiol can be used in the treatment of myeloma. In another embodiment of the present invention, honokiol or any of the compounds or compositions described herein can be used to treat a plasma cell neoplasm, such as, but not limited to multiple myeloma, macroglobulinemia, isolated plasmacytoma of bone, extramedullary plasmacytoma, waldenstrom's macroglobulinemia or Lymphoplasmacytic leukemia, monoclonal gammapathy, smoldering myeloma, stage I multiple myeloma, stage II multiple myeloms, and/or refractory plasma cell neoplasm.

Myeloma or plasma cell neoplasms are diseases in which certain cells in the blood (called plasma cells) become cancer. Plasma cells are made by white blood cells called lymphocytes. The plasma cells make antibodies, which fight infection and other harmful things in the body. When these cells become cancer, they may make too many antibodies and a substance called M-protein is found in the blood and urine. There are several types of plasma cell neoplasms. The most common type is called multiple myeloma. In multiple myeloma, cancerous plasma cells are found in the bone marrow. The bone marrow is the spongy tissue inside the large bones in the body. The bone marrow makes red blood cells (which carry oxygen and other materials to all tissues of the body), white blood cells (which fight infection), and platelets (which make the blood clot). The cancer cells can crowd out normal blood cells, causing anemia (too few red blood cells). The plasma cells also may cause the bone to break down. The plasma cells can collect in the bone to make small tumors called plasmacytomas. Plasma cell neoplasms also can appear only as growths of plasma cells (plasmacytomas) in the bone and soft tissues, without cancer cells in the bone marrow or blood. Macroglobulinemia is a type of plasma cell neoplasm in which lymphocytes that make an M-protein build up in the blood. Lymph nodes and the liver and spleen may be swollen.

Multiple myeloma (MM) is a B-cell malignancy characterized by proliferation of monoclonal plasma cell in bone marrow. Despite clinical efficacy of high dose therapy as well as novel agents including thalidomide, revlimid and bortezomib in patients with relapsed and refractory MM, responses are not durable and few, if any, patients are cured. Therefore new therapeutic strategies are needed to improve patient outcome.

In one aspect, the present invention is based on the surprising discovery that HNK is effective against multiple myeloma. HNK can inhibit growth and induce apoptosis of MM cells, via both caspase-dependent and -independent pathways, overcome conventional drug resistance, inhibit angiogenesis in the BM milieu, and/or enhance MM cell cytotoxicity of bortezomib.

HNK significantly induces cytotoxicity in human multiple myeloma (MM) cell lines and tumor cells from patients with relapsed refractory MM. Neither co-culture with bone marrow stromal cells nor cytokines (interleukin-6 and insulin-like growth factor-1) protect against HNK-induced cytotoxicity. Although activation of caspases 3, 7, 8 and 9 was triggered by HNK, the pan-caspase inhibitor z-VAD-fmk does not abrogate HNK-induced apoptosis. Importantly, release of an executioner of caspase-independent apoptosis AIF from mitochondria was induced by HNK treatment. HNK induces apoptosis in SU-DHL4 cell line, which has low levels of caspase-3 and -8 associated with resistance to both conventional and novel drugs. While not being limited to any theory, these results suggest that HNK induces apoptosis via both caspase-dependent and -independent pathways. Furthermore, HNK can enhance MM cell cytotoxicity and apoptosis induced by other drugs such as bortezomib. In addition to its direct cytotoxicity to MM cells, HNK also represses tube formation by endothelial cells, suggesting that HNK inhibits neovascurization in the bone marrow microenvironment. Thus, HNK and its derivatives can be used to improve patient outcome in MM.

### Drug Resistant Cancers

One aspect of the present invention is based on the discovery that honokiol can induce apoptosis in cancer cells through a caspase independent mechanism. Cancer cell lines with low levels of certain caspases, such as caspase-3 and caspase-8, can be associated with cancer drug resistance. Drug resistance is a problem in cancer. The invention provides honoliol and honokiol derivatives that can be used to treat drug resistant cancer, including the embodiments of cancers and drugs disclosed herein. In one embodiment, the honoliol or derivative is co-administered with a second drug.

Multidrug resistance (MDR) occurs in human cancers and can be a significant obstacle to the success of chemotherapy. Multidrug resistance is a phenomenon whereby tumor cells in vitro that have been exposed to one cytotoxic agent develop cross-resistance to a range of structurally and functionally unrelated compounds. In addition, MDR can occur intrinsically in some cancers without previous exposure to chemotherapy agents. Thus, in one embodiment, the present invention provides methods for the treatment of a patient with a drug resistant cancer, for example, multidrug resistant cancer, by administration of honokiol or derivative thereof.

In one embodiment, honokiol or derivatives thereof can be used for the treatment of drug resistant cancers of the colon, bone, kidney, adrenal, pancreas, liver and/or any other cancer known in the art or described herein. In a particular embodiment, honoliol or a derivative thereof, including the derivatives described herein can be administered in an effective amount for the treatment of drug resistant multiple myeloma. In one embodiment, honokiol or a derivative thereof can be administered in an amount effective to treat multiple mycloma that is resistant to doxorubicin, As₂O₃, melphalan, dexamethasone, bortezomib and/or revlimid.

### Angiogenesis-Related Diseases

In further embodiments of the present invention, the compounds disclosed herein can be used in the treatment of angiogenesis-related diseases.

Dr. Folkman demonstrated that all growing tumors are angiogenic, and that in multiple animal models, tumor growth is angiogenesis dependent (O'Reilly et al. (1994) Cell 79, 315-328). The discovery of tumor angiogenesis led to the discovery of tumor derived angiogenic growth factors, such as basic fibroblast growth factors and vascular endothelial growth factor (VEGF). In addition, tumor cells and tumor derived inflammatory cells are capable of releasing pro-angiogenic cytokines such as interleukin 6, 8, and corticotropin releasing hormone (CRH) (Ezekowitz, R. A. et al (1992) N. EngI. J. Med. 326, 1456-1463; Lu et al Proc. Natl. Acad. Sci. U.S.A 89, 9215-9219, Arbiser et al (1999) J. Invest Dermatol. 113, 838-842). The development of highly malignant tumors occurs through what is known as the angiogenic switch, in which tumors go from a balance of endogenous angiogenesis inhibitors (thrombospondins, interferons, and tissue inhibitors of matrix metalloproteinases) and stimulators (VEGF, FGF, CRH, and matrix metalloproteinases), to an imbalance in favor of angiogenic stimulation. Both activation of dominant oncogenes and loss of tumor suppressor genes contribute to the angiogenic switch (Arbiser et al. (1997) Proc. Natl. Acad. Sci. U.S.A 94, 861-866; Hanahan, D. & Folkman, J. (1996) Cell 86, 353-364). Analysis of the angiogenic switch has led to two strategies which may be synergistic in the treatment of cancer. First, direct inhibitors of angiogenesis, which block specific interactions of endothelial growth factors and their receptors, have been shown to demonstrate efficacy in animal models and humans (Arbiser, J. L. (1996) J. Am. Acad. Dermatol. 34, 486-497; Arbiser et al (1998) Mol. Med. 4, 376-383). Direct angiogenesis inhibitors include angiostatin, endostatin, and VEGF inhibitors (O'Reilly et al (1997) Cell 88, 277-285; Wen et al (1999) Cancer Res. 59, 6052-6056). Indirect inhibitors of angiogenesis prevent tumor cells from making proangiogenic factors and possibly augment production of angiogenesis inhibitors. Examples of indirect angiogenesis inhibitors include signaling antagonists, such as tyrosine kinase inhibitors, farnesyltransferase inhibitors, and inhibitors of signaling pathways such as MAP kinase, phosphoinositol-3 kinase, reactive oxygen, and nuclear factor kappa beta (NFkB) (Arbiser et al. (1997) Proc. Natl. Acad. Sci. U.S.A 94, 861-866, LaMontagne et al (2000) Am. J. Pathol. 157, 1937-1945, Arbiser, J. L. (2003) Nat. Med. 9, 1103., Arbiser, J. L. et al. (2002) Proc. Natl. Acad. Sci. U.S.A 99, 715-720, Huang, S. et al (2000) Clin Cancer Res. 6, 2573-2581). Honokiol, a small molecular weight compound, has both direct antiangiogenic properties, in that it inhibits phosphorylation of VEGFR2, the primary receptor mediating pathologic angiogenesis, and has direct antitumor activity by mediating apoptosis through tumor necrosis factor-related apoptosis inducing ligand (TRAIL) mediated apoptosis (Bai et al. (2003) J. Biol. Chem. 278, 35501-35507).

Angiogenesis inhibitors inhibit endothelial growth and angiogenesis through a wide variety of mechanisms. Interferon alpha/beta, the first described naturally occurring angiogenesis inhibitor, has been shown to activate synthesis of the cell cycle inhibitor p21 (Brouty-Boye, D. & Zetter, B. R. (1980) Science 208, 516-518, Chin, Y. E. et al. (1996) Science 272, 719-722). Angiostatin and endostatin have been shown to act through binding to the endothelial cell surface, activating apoptosis by interfering with integrin-mediated endothelial survival signals (Rehn, M. et al. (2001) Proceedings of the National Academy of Sciences of the United States of America 98, 1024-1029, Karumanchi, S. A. et al. (2001) Molecular Cell 7, 811-822). Thrombospondin-1 binds to a cellular receptor present on endothelial cells, CD36, resulting in endothelial apoptosis. Tissue inhibitor of matrix metalloproteinases (TIMPs) inhibit the enzymatic activity of matrix metalloproteinases, preventing breakdown of basement membrane in a 1/1 stoichiometric fashion, and recently, a separate antiangiogenic fragment of 24 amino acids has been isolated from TIMP2 (Fernandez, et al (2003) Journal of Biological Chemistry 278, 40989-40995). Previously discovered antiangiogenic small molecules include thalidomide, which acts in part by inhibiting NFkB, 2-methoxyestradiol, which influences microtubule activation and hypoxia inducing factor (HIF1a) activation, cyclo-oxygenase 2 (COX2) inhibitors, and low doses of conventional chemotherapeutic agents, including cyclophosphamide, taxanes, and vinca alkaloids (vincristine, vinblastine) (D'Amato, R. J. et al. (1994) Proc. Natl. Acad. Sci. U.S.A 91, 3964-3968, D'Amato, R. J. et al. (1994) Proc. Natl. Acad. Sci. U.S.A 91, 4082-4085). In addition, certain tyrosine kinase inhibitors indirectly decrease angiogenesis by decreasing production of VEGF and other proangiogenic factors by tumor and stromal cells. These drugs include Herceptin, imatinib (Glivec), and Iressa (Bergers, G. et al. (2003) Journal of Clinical Investigation 111, 1287-1295, Ciardiello, F. et al. (2001) Clinical Cancer Research 7, 1459-1465, Plum, S. M. et al. (2003) Clinical Cancer Research 9, 4619-4626).

Recently, angiogenesis inhibitors have moved from animal models to human patients. Angiogenesis inhibitors represent a promising treatment for a variety of cancers. Recently, Avastin a high affinity antibody against vascular endothelial growth factor (VEGF), has been shown to prolong life as a single agent in advanced renal cell carcinoma and prolong life in combination with chemotherapy in advanced colon cancer (Yang, J. C. et al. (2003) New England Journal of Medicine 349, 427-434, Kabbinavar, F. et al. (2003) Journal of Clinical Oncology 21, 60-65).

Angiogenesis-related diseases include, but are not limited to, inflammatory, autoimmune, and infectious diseases; angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; eczema; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation. In addition, compositions of this invention can be used to treat diseases such as, but not limited to, intestinal adhesions, atherosclerosis, scleroderma, warts, and hypertrophic scars (i.e., keloids). Compositions of this invention can also be used in the treatment of diseases that have angiogenesis as a pathologic consequence such as cat scratch disease (Rochele minalia quintosa), ulcers (Helobacter pylori), tuberculosis, and leprosy.

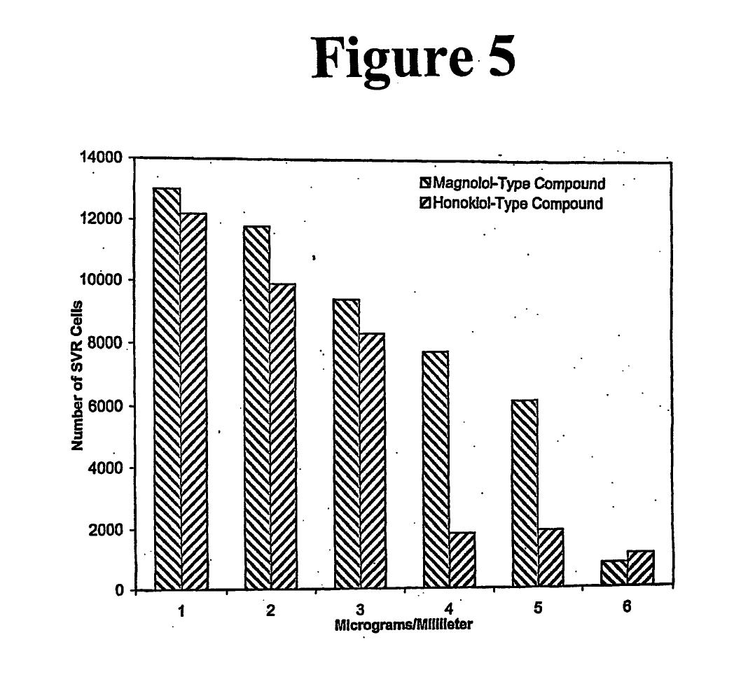

As illustrated in FIG. 5, honokiol-type compounds and magnolol-type compounds have been shown to be effective at decreasing the proliferation of SVR cells. In this regard, using inhibition of transformed SVR endothelial cells as a bioassay, honokiol-type compounds and magnolol-type compounds show enhanced activity in the SVR inhibition assay. Previously, bioassays of transformed SVR endothelial cells have been used to accurately predict in vivo responses to known angiogenesis inhibitors (Arbiser et al., Proc. Natl. Acad. Sci. 94: 861-866 and Arbiser et al., J. Am. Acad. of Dermatol., 40:959-929, incorporated herein by reference). Therefore, honokiol-type compounds and magnolol-type compounds may be used to inhibit angiogenesis, as discussed in Bai et al., J Biol Chem. (2003) Sep. 12; 278(37):35501-7, incorporated herein by reference.

### Viral Infections

The compounds disclosed herein, in one embodiment, may be adminstered to a host in an effective amount for the treatment of a viral infection, such as HIV, Hepatitis-B (HBV), or Hepatitis-C (HCV), alone or in combination, for example with a second antiviral.

### Inflamatory Diseases

In further embodiments of the present invention, the compounds disclosed herein can be used in the treatment of inflammatory diseases.

Examples of inflammatory diseases include, but are not limited to, arthritis, asthma, dermatitis, psoriasis, cystic fibrosis, post transplantation late and chronic solid organ rejection, multiple sclerosis, systemic lupus erythematosis, inflammatory bowel diseases, gastrointestinal conditions (e.g., gastritis, irritable bowel syndrome, ulcerative colitis), Crohn's disease, headache, asthma, bronchitis, tuberculosis, chronic cholecystitis, Hashimoto's thyroiditis, menstrual cramps, tendonitis, bursitis, rhinitis, ischemia-reperfusion injury, post-angioplasty restenosis, chronic obstructive pulmonary disease (COPD), Psoriasis, glomerulonephritis, Graves disease, gastrointestinal allergies, sarcoidosis, disseminated intravascular coagulation, vasculitis syndromes, atherosclerosis, coronary artery disease, angina, small artery disease, conjunctivitis. In addition, as readily recognized by those of skill in the art, inflammation-related conditions can also be associated with a variety of conditions, such as, for example, vascular diseases, periarteritis nodosa, thyroidiris, aplastic anemia, Hodgkin's disease, sclerodoma, rheumatic fever, type I diabetes, myasthenia gravis, colorectal cancer, sarcoidosis, nephrotic syndrome, Behcet's syndrome, potymyositis, gingivitis, hypersensitivity, conjunctivitis, swelling occurring after injury, myocardial ischemia, and the like, which can also be treated by the compounds of the present invention.

In a particular embodiment, the compounds disclosed herein can be used to treat arthritis or arthritic condition. Examples of arthritis and arthritic conditions include, but are not limited to rheumatoid (such as soft-tissue rheumatism and non-articular rheumatism), fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, spondyloarthropaties (ankylosing spondylitis), osteoarthritis, hyperuricemia and arthritis associated with acute gout, chronic gout and systemic lupus erythematosus.

### Bone-Related Diseases

In other embodiments of the present invention, the compounds disclosed herein can be used in the treatment of bone-related diseases, including, but not limited to osteoporosis.

In a particular embodiment, the compounds of the present invention can be used for the treatment of osteoporosis or a related condition. In additional embodiments, the compounds disclosed herein can be used to treat bone tumors, craniosynostosis, enchrondroma, fibrous dysplasia, Klippel-Feil Syndrome, Osteitis Condensans IIii, Osteochonidritis Dissecans (OCD), Osteomyelitis (Cleveland Clinic Foundation), Osteonecrosis, Osteopenia, Renal Osteodystrophy, Unicameral (Simple) Bone Cyst and/or Osteomalacia.

### Combination Therapy

In one aspect of the present invention, the compounds and compositions disclosed herein can be combined with at least one additional chemotherapeutic agent. The additional agents can be administered in combination or alternation with the compounds disclosed herein. The drugs can form part of the same composition, or be provided as a separate composition for administration at the same time or a different time.

In one particular embodiment, the compounds of the present invention can be administered in combination and/or alternation with a histone deacetylase inhibitor. In one embodiment, the histone deacetylase inhibitor can be suberoylaanilide hydroxamic acid (SAHA) (see, for example, Butler, L. M. et al., Proc. Natl. Acad. Sci., USA 99, 11700-11705, 2002). In another embodiment, the histone deacetylase inhibitor can be a phosphorus-based SAHA analog, such as Apicidin (see, for example, Mai, A. et al., J. Med. Chem., 45, 1778-1784 (2002). In another embodiment, the histone deacetylase inhibitor can be selected from, but not limited to the following: sodium butyrate; (−)-Depudecin (see, for example, Kwon, et al., Proc. Natl. Acad. Sci. USA, 95, 3356, 1998); Scriptaid (see, for example, Su, G., et al., Cancer Res., 60, 3137-3142, 2000); Sirtinol (2-[(2-Hydroxynaphthalen-1-ylmethylene)amino]-N-91-phenetheyl)benzamide; and/or trichostatin A (see, for example, Yoshida, M., et al., Bioessays, 17, 423-430, 1995).

In one embodiment, compounds disclosed herein can be combined with antiangiogenic agents to enhance their effectiveness, or combined with other antiangiogenic agents and administered together with other cytotoxic agents. In another embodiment, the compounds and compositions, when used in the treatment of solid tumors, can be administered with the agents selected from, but not limited to IL-12, retinoids, interferons, angiostatin, endostatin, thalidomide, thrombospondin-1, thrombospondin-2, captopryl, anti-neoplastic agents such as alpha interferon, COMP (cyclophosphamide, vincristine, methotrexate and prednisone), etoposide, mBACOD (methortrexate, bleomycin, doxorubicin, cyclophosphamide, vincristine and dexamethasone), PRO-MACE/MOPP (prednisone, methotrexate (w/leucovin rescue), doxorubicin, cyclophosphamide, taxol, etoposide/mechlorethamine, vincristine, prednisone and procarbazine), vincristine, vinblastine, angioinhibins, TNP-470, pentosan polysulfate, platelet factor 4, angiostatin, LM-609, SU-101, CM-101, Techgalan, thalidomide, SP-PG and radiation. In another particular embodiment, the compound of the present invention can be administered in combination or alternation with trichostatin A (TSA). In further embodiments, the compounds and compositions disclosed herein can be administered in combination or alternation with, for example, drugs with antimitotic effects, such as those which target cytoskeletal elements, including microtubule modulators such as taxane drugs (such as taxol, paclitaxel, taxotere, docetaxel), podophylotoxins or vinca alkaloids (vincristine, vinblastine); antimetabolite drugs (such as 5-fluorouracil, cytarabine, gemcitabine, purine analogues such as pentostatin, methotrexate); alkylating agents or nitrogen mustards (such as nitrosoureas, cyclophosphamide or ifosphamide); drugs which target DNA such as the antracycline drugs adriamycin, doxorubicin, pharmorubicin or epirubicin; drugs which target topoisomerases such as etoposide; hormones and hormone agonists or antagonists such as estrogens, antiestrogens (tamoxifen and related compounds) and androgens, flutamide, leuprorelin, goserelin, cyprotrone or octreotide; drugs which target signal transduction in tumour cells including antibody derivatives such as herceptin; alkylating drugs such as platinum drugs (cis-platin, carbonplatin, oxaliplatin, paraplatin) or nitrosoureas; drugs potentially affecting metastasis of tumours such as matrix metalloproteinase inhibitors; gene therapy and antisense agents; antibody therapeutics; other bioactive compounds of marine origin, notably the dideimins such as aplidine; steroid analogues, in particular dexamethasone; anti-inflammatory drugs, including nonsteroidal agents (such as acetaminophen or ibuprofen) or steroids and their derivatives in particular dexamethasone; anti-emetic drugs, including 5HT-3 inhibitors (such as gramisetron or ondasetron), and steroids and their derivatives in particular dexamethasone. In still further embodiments, the compounds and compositions can be used in combination or alternation with the chemotherapeutic agents disclosed below in Table 3.

TABLE 3
Chemotherapeutic Agents

| | |
|---|---|
| 13-cis-Retinoic Acid | Neosar |
| 2-Amino-6- | Neulasta |
| Mercaptopurine | Neumega |
| 2-CdA | Neupogen |
| 2-Chlorodeoxyadenosine | Nilandron |
| 5-fluorouracil | Nilutamide |
| 5-FU | Nitrogen Mustard |
| 6-TG | Novaldex |
| 6-Thioguanine | Novantrone |
| 6-Mercaptopurine | Octreotide |
| 6-MP | Octreotide acetate |
| Accutane | Oncospar |
| Actinomycin-D | Oncovin |
| Adriamycin | Ontak |
| Adrucil | Onxal |
| Agrylin | Oprevelkin |
| Ala-Cort | Orapred |
| Aldesleukin | Orasone |
| Alemtuzumab | Oxaliplatin |
| Alitretinoin | Paclitaxel |
| Alkaban-AQ | Pamidronate |
| Alkeran | Panretin |
| All-transretinoic acid | Paraplatin |
| Alpha interferon | Pediapred |
| Altretamine | PEG Interferon |
| Amethopterin | Pegaspargase |
| Amifostine | Pegfilgrastim |
| Aminoglutethimide | PEG-INTRON |
| Anagrelide | PEG-L-asparaginase |
| Anandron | Phenylalanine Mustard |
| Anastrozole | Platinol |
| Arabinosylcytosine | Platinol-AQ |
| Ara-C | Prednisolone |
| Aranesp | Prednisone |
| Aredia | Prelone |
| Arimidex | Procarbazine |
| Aromasin | PROCRIT |
| Arsenic trioxide | Proleukin |
| Asparaginase | Prolifeprospan 20 with Carmustine implant |
| ATRA | Purinethol |
| Avastin | Raloxifene |
| BCG | Rheumatrex |
| BCNU | Rituxan |
| Bevacizumab | Rituximab |
| Bexarotene | Roveron-A (interferon alfa-2a) |
| Bicalutamide | Rubex |
| BiCNU | Rubidomycin hydrochloride |
| Blenoxane | Sandostatin |
| Bleomycin | Sandostatin LAR |
| Bortezomib | Sargramostim |
| Busulfan | Solu-Cortef |
| Busulfex | Solu-Medrol |
| C225 | STI-571 |
| Calcium Leucovorin | Streptozocin |
| Campath | Tamoxifen |
| Camptosar | Targretin |
| Camptothecin-11 | Taxol |
| Capecitabine | Taxotere |
| Carac | Temodar |
| Carboplatin | Temozolomide |
| Carmustine | Teniposide |
| Carmustine wafer | TESPA |
| Casodex | Thalidomide |
| CCNU | Thalomid |
| CDDP | TheraCys |
| CeeNU | Thioguanine |
| Cerubidine | Thioguanine Tabloid |
| cetuximab | Thiophosphoamide |
| Chlorambucil | Thioplex |
| Cisplatin | Thiotepa |
| Citrovorum Factor | TICE |
| Cladribine | Toposar |
| Cortisone | Topotecan |
| Cosmegen | Toremifene |
| CPT-11 | Trastuzumab |
| Cyclophosphamide | Tretinoin |
| Cytadren | Trexall |
| Cytarabine | Trisenox |
| Cytarabine liposomal | TSPA |
| Cytosar-U | VCR |
| Cytoxan | Velban |
| Dacarbazine | Velcade |
| Dactinomycin | VePesid |
| Darbepoetin alfa | Vesanoid |
| Daunomycin | Viadur |
| Daunorubicin | Vinblastine |
| Daunorubicin | Vinblastine Sulfate |
| hydrochloride | Vincasar Pfs |
| Daunorubicin liposomal | Vincristine |
| DaunoXome | Vinorelbine |
| Decadron | Vinorelbine tartrate |
| Delta-Cortef | VLB |
| Deltasone | VP-16 |
| Denileukin diftitox | Vumon |
| DepoCyt | Xeloda |
| Dexamethasone | Zanosar |
| Dexamethasone acetate | Zevalin |
| dexamethasone sodium | Zinecard |
| phosphate | Zoladex |
| Dexasone | Zoledronic acid |
| Dexrazoxane | Zometa |
| DHAD | Gliadel wafer |
| DIC | Glivec |
| Diodex | GM-CSF |
| Docetaxel | Goserelin |
| Doxil | granulocyte —colony stimulating factor |
| Doxorubicin | Granulocyte macrophage colony stimulating |
| Doxorubicin liposomal | factor |
| Droxia | Halotestin |
| DTIC | Herceptin |
| DTIC-Dome | Hexadrol |
| Duralone | Hexalen |
| Efudex | Hexamethylmelamine |
| Eligard | HMM |
| Ellence | Hycamtin |
| Eloxatin | Hydrea |
| Elspar | Hydrocort Acetate |
| Emcyt | Hydrocortisone |
| Epirubicin | Hydrocortisone sodium phosphate |
| Epoetin alfa | Hydrocortisone sodium succinate |
| Erbitux | Hydrocortone phosphate |
| Erwinia L-asparaginase | Hydroxyurea |
| Estramustine | Ibritumomab |
| Ethyol | Ibritumomab Tiuxetan |
| Etopophos | Idamycin |
| Etoposide | Idarubicin |
| Etoposide phosphate | Ifex |
| Eulexin | IFN-alpha |
| Evista | Ifosfamide |
| Exemestane | IL-2 |
| Fareston | IL-11 |
| Faslodex | Imatinib mesylate |
| Femara | Imidazole Carboxamide |
| Filgrastim | Interferon alfa |
| Floxuridine | Interferon Alfa-2b (PEG conjugate) |
| Fludara | Interleukin-2 |
| Fludarabine | Interleukin-11 |
| Fluoroplex | Intron A (interferon alfa-2b) |
| Fluorouracil | Leucovorin |
| Fluorouracil (cream) | Leukeran |
| Fluoxymesterone | Leukine |
| Flutamide | Leuprolide |
| Folinic Acid | Leurocristine |
| FUDR | Leustatin |
| Fulvestrant | Liposomal Ara-C |
| G-CSF | Liquid Pred |
| Gefitinib | Lomustine |
| Gemcitabine | L-PAM |
| Gemtuzumab ozogamicin | L-Sarcolysin |
| Gemzar | Meticorten |
| Gleevec | Mitomycin |
| Lupron | Mitomycin-C |
| Lupron Depot | Mitoxantrone |
| Matulane | M-Prednisol |
| Maxidex | MTC |
| Mechlorethamine | MTX |
| Mechlorethamine | Mustargen |
| Hydrochlorine | Mustine |
| Medralone | Mutamycin |
| Medrol | Myleran |
| Megace | Iressa |
| Megestrol | Irinotecan |
| Megestrol Acetate | Isotretinoin |
| Melphalan | Kidrolase |
| Mercaptopurine | Lanacort |
| Mesna | L-asparaginase |
| Mesnex | LCR |
| Methotrexate | |
| Methotrexate Sodium | |
| Methylprednisolone | |
| Mylocel | |
| Letrozole | |

In certain embodiments, the compounds and compositions described herein can be used in combination with a therapeutic agent used to treat multiple myeloma. In one embodiment, honokiol can be used in combination with an agent used to treat multiple myeloma. Drugs used in the treatment of multiple myeloma include, but are not limited to, erythropoietin, genasense, panzem, PI-88, revlimid, thalidomide, Thalidomid, trisenox, velcade, zarnestra, zoledronic acid, zometa, 2ME2, Aredia, arsenic trioxide, Bcl-2 antisense, bisphosphonates, and colony stimulating factors. In a particular embodiment, the honokiol or derivative thereof can be administered in combination with bortezomib for the treatment of multiple myeloma.

In one embodiment, honokiol or derivatives thereof can be used in combination or alternation with additional chemotherapeutic agents, such as those described herein or in Table 3, for the treatment of drug resistant cancer, for example multiple drug resistant cancer. Drug resistant cancers can include cancers of the colon, bone, kidney, adrenal, pancreas, liver and/or any other cancer known in the art or described herein. In one embodiment, the additional chemotherapeutic agent can be a P-glycoprotein inhibitor. In certain non-limiting embodiments, the P-glycoprotein inhibitor can be selected from the following drugs: verapamil, cyclosporin (such as cyclosporin A), tamoxifen, calmodulin antagonists, dexverapamil, dexniguldipine, valspodar (PSC 833), biricodar (VX-710), tariquidar (XR9576), zosuquidar (LY335979), laniquidar (R101933), and/or ONT-093. In another embodiment, honokiol or a derivative thereof, including the derivatives described herein, can be administered alone or in combination or alternation with other therapeutic agents to treat multiple myeloma. In a particular embodiment, the honokiol or derivative thereof can be administered in combination with bortezomib for the treatment of drug resistant cancer, including drug resistant myeloma.

### Screening of Patient Populations

An additional object of the present invention provides methods to identify tumors and cancers that are particularly susceptible to the toxic effects of honokiol and/or related compounds as described herein. One aspect of the present invention is based on the discovery that tumors that express phospholipase D (PLD), nuclear factor-κB (NKκB), and/or adenosine monophosphate kinase activated protein kinase (AMPK) are particularly susceptible to the toxic effects of honokiol or derivatives thereof. In one embodiment, methods are provided for treating a tumor in a mammal, particularly a human, which includes (i) obtaining a biological sample from the tumor; (ii) determining whether the tumor expresses or overexpresses an phospholipase D (PLD), nuclear factor-κB (NKκB), and/or adenosine monophosphate kinase activated protein kinase (AMPK), and (iii) treating the tumor that expresses or overexpresses phospholipase D (PLD), nuclear factor-κB (NKκB), and/or adenosine monophosphate kinase activated protein kinase (AMPK) with honokiol or a related compound as described herein. In one embodiment, the level of NFκB and/or AMPK expression can be determined by assaying the tumor or cancer for the presence of a phosphorylated NFκB and/or AMPK, for example, by using an antibody that can detect the phosphorylated form. In another embodiment, the level of PLD, NFκB and/or AMPK expression can be determined by assaying a tumor or cancer cell obtained from a subject and comparing the levels to a control tissue. In certain embodiments, the PLD, NFκB and/or AMPK can be overexpressed at least 2, 2.5, 3 or 5 fold in the cancer sample compared to the control. In one embodiment, the biological sample can be a biopsy. In other embodiments, the biological sample can be fluid, cells and/or aspirates obtained from the tumor or cancer. In one embodiment the tumor or cancer can be assayed for the expression or overexpression of phospholipase D (PLD). In another embodiment the tumor or cancer can be assayed for the expression or overexpression of nuclear factor-κB (NKκB). In a further embodiment the tumor or cancer can be assayed for the expression or overexpression of adenosine monophosphate kinase activated protein kinase (AMPK).

The biological sample can be obtained according to any technique known to one skilled in the art. In one embodiment, a biopsy can be conducted to obtain the biological sample. A biopsy is a procedure performed to remove tissue or cells from the body for examination. Some biopsies can be performed in a physician's office, while others need to be done in a hospital setting. In addition, some biopsies require use of an anesthetic to numb the area, while others do not require any sedation. In certain embodiments, an endoscopic biopsy can be performed. This type of biopsy is performed through a fiberoptic endoscope (a long, thin tube that has a close-focusing telescope on the end for viewing) through a natural body orifice (i.e., rectum) or a small incision (i.e., arthroscopy). The endoscope is used to view the organ in question for abnormal or suspicious areas, in order to obtain a small amount of tissue for study. Endoscopic procedures are named for the organ or body area to be visualized and/or treated. The physician can insert the endoscope into the gastrointestinal tract (alimentary tract endoscopy), bladder (cystoscopy), abdominal cavity (laparoscopy), joint cavity (arthroscopy), mid-portion of the chest (mediastinoscopy), or trachea and bronchial system (laryngoscopy and bronchoscopy).

In another embodiment, a bone marrow biopsy can be performed. This type of biopsy can be performed either from the sternum (breastbone) or the iliac crest hipbone (the bone area on either side of the pelvis on the lower back area). The skin is cleansed and a local anesthetic is given to numb the area. A long, rigid needle is inserted into the marrow, and cells are aspirated for study; this step is occasionally uncomfortable. A core biopsy (removing a small bone ‘chip’ from the marrow) may follow the aspiration.

In a further embodiment, an excisional or incisional biopsy can be performed on the mammal. This type of biopsy is often used when a wider or deeper portion of the skin is needed. Using a scalpel (surgical knife), a full thickness of skin is removed for further examination, and the wound is sutured (sewed shut with surgical thread). When the entire tumor is removed, it is referred to as an excisional biopsy technique. If only a portion of the tumor is removed, it is referred to as an incisional biopsy technique. Excisional biopsy is often the method usually preferred, for example, when melanoma (a type of skin cancer) is suspected.

In still further embodiments, a fine needle aspiration (FNA) biopsy can be used. This type of biopsy involves using a thin needle to remove very small pieces from a tumor. Local anesthetic is sometimes used to numb the area, but the test rarely causes much discomfort and leaves no scar. FNA is not, for example, used for diagnosis of a suspicious mole, but may be used, for example, to biopsy large lymph nodes near a melanoma to see if the melanoma has metastasized (spread). A computed tomography scan (CT or CAT scan) can be used to guide a needle into a tumor in an internal organ such as the lung or liver.

In other embodiments, punch shave and/or skin biopsies can be conducted. Punch biopsies involve taking a deeper sample of skin with a biopsy instrument that removes a short cylinder, or “apple core,” of tissue. After a local anesthetic is administered, the instrument is rotated on the surface of the skin until it cuts through all the layers, including the dermis, epidermis, and the most superficial parts of the subcutis (fat). A shave biopsy involves removing the top layers of skin by shaving it off. Shave biopsies are also performed with a local anesthetic. Skin biopsies involve removing a sample of skin for examination under the microscope to determine if, for example, melanoma is present. The biopsy is performed under local anesthesia.

In particular embodiment, methods are provided to determine whether the tumor expresses or overexpresses PLD, NFκB and/or AMPK. In one embodiment, a tumor biopsy can be compared to a control tissue. The control tissue can be a normal tissue from the mammal in which the biopsy was obtained or a normal tissue from a healthy mammal. PLD, NFκB and/or AMPK expression or overexpression can be determined if the tumor biopsy contains greater amounts of PLD, NFκB and/or AMPK than the control tissue, such as, for example, at least approximately 1.5, 2, 2.25, 2.5, 2.75, 3, 3.25, 3.5, 3.75, 4, 4.25, 4.5, 4.75, 5, 5.5, 6, 7, 8, 9, or 10-fold greater amounts of PLD, NFκB and/or AMPK than contained in the control tissue.

In one embodiment, the present invention provides a method to detect aberrant PLD, NFκB and/or AMPK expression in a subject or in a biological sample from the subject by contacting cells, cell extracts, serum or other sample from the subjects or said biological sample with an immunointeractive molecule specific for PLD, NFκB and/or AMPK or antigenic portion thereof and screening for the level of immunointeractive molecule—complex formation, wherein an elevated presence of the complex relative to a normal cell is indicative of an aberrant cell that expresses or overexpresses PLD, NFκB and/or AMPK. In one example, cells or cell extracts can be screened immunologically for the presence of elevated levels of PLD, NFκB and/or AMPK.

In an alternative embodiment, the aberrant expression of PLD, NFκB and/or AMPK in a cell is detected at the genetic level by screening for the level of expression of a gene encoding PLD, NFκB and/or AMPK wherein an elevated level of a transcriptional expression product (i.e. mRNA) compared to a normal cell is indicative of an aberrant cell. In certain embodiments, real-time PCR as well as other PCR procedures can be used to determine transcriptional activity. In one embodiment, mRNA can be obtained from cells of a subject or from a biological sample from a subject and cDNA optionally generated. The mRNA or cDNA can then be contacted with a genetic probe capable of hybridizing to and/or amplifying all or part of a nucleotide sequence encoding PLD, NFκB and/or AMPK or its complementary nucleotide sequence and then the level of the mRNA or cDNA can be detected wherein the presence of elevated levels of the mRNA or cDNA compared to normal controls can be assessed.

Yet another embodiment of the present invention contemplates the use of an antibody, monoclonal or polyclonal, to PLD, NFκB and/or AMPK in a quantitative or semi-quantitative diagnostic kit to determine relative levels of PLD, NFκB and/or AMPK in suspected cancer cells from a patient, which can include all the reagents necessary to perform the assay. In one embodiment, a kit utilizing reagents and materials necessary to perform an ELISA assay is provided. Reagents can include, for example, washing buffer, antibody dilution buffer, blocking buffer, cell staining solution, developing solution, stop solution, anti-phospho-protein specific antibodies, anti-Pan protein specific antibodies, secondary antibodies, and distilled water. The kit can also include instructions for use and can optionally be automated or semi-automated or in a form which is compatible with automated machine or software.

### Diagnostic Assays

### Immunological Assays

In one embodiment, a method is provided for detecting the expression or overexpression of PLD, NFκB and/or AMPK in a cell in a mammal or in a biological sample from the mammal, by contacting cells, cell extracts or serum or other sample from the mammal or biological sample with an immunointeractive molecule specific for PLD, NFκB and/or AMPK or antigenic portion thereof and screening for the level of immunointeractive molecule—PLD, NFκB and/or AMPK complex formations and determining whether an elevated presence of the complex relative to a normal cell is present.

The immunointeractive molecule can be a molecule having specificity and binding affinity for PLD, NFκB and/or AMPK or its antigenic parts or its homologs or derivatives thereof. In one embodiment, the immunointeractive molecule can be an immunglobulin molecule. In other embodiments, the immunointeractive molecules can be an antibody fragments, single chain antibodies, and/or deimmunized molecules including humanized antibodies and T-cell associated antigen-binding molecules (TABMs). In one particular embodiment, the antibody can be a monoclonal antibody. In another particular embodiment, the antibody can be a polyclonal antibody. The immunointeractive molecule can exhibit specificity for PLD, NFκB and/or AMPK or more particularly an antigenic determinant or epitope on PLD, NFκB and/or AMPK. An antigenic determinant or epitope on PLD, NFκB and/or AMPK includes that part of the molecule to which an immune response is directed. The antigenic determinant or epitope can be a B-cell epitope or where appropriate a T-cell epitope.

One embodiment of the present invention provides a method for diagnosing the presence of cancer or cancer-like growth in a mammal, in which PLD, NFκB and/or AMPK activity is present, by contacting cells or cell extracts from the mammal or a biological sample from the subject with an PLD, NFκB and/or AMPK-binding effective amount of an antibody having specificity for the PLD, NFκB and/or AMPK or an antigenic determinant or epitope thereon and then quantitatively or qualitatively determining the level of an PLD, NFκB and/or AMPK-antibody complex wherein the presence of elevated levels of said complex compared to a normal cell is determined.

Antibodies can be prepared by any of a number of means known to one skilled in the art. For example, for the detection of human PLD, NFκB and/or AMPK, antibodies can be generally but not necessarily derived from non-human animals such as primates, livestock animals (e.g. sheep, cows, pigs, goats, horses), laboratory test animals (e.g. mice, rats, guinea pigs, rabbits) and/or companion animals (e.g. dogs, cats). Antibodies may also be recombinantly produced in prokaryotic or eukaryotic host cells. Generally, antibody based assays can be conducted in vitro on cell or tissue biopsies. However, if an antibody is suitably deimmunized or, in the case of human use, humanized, then the antibody can be labeled with, for example, a nuclear tag, administered to a patient and the site of nuclear label accumulation determined by radiological techniques. The PLD, NFκB and/or AMPK antibody can be a cancer targeting agent. Accordingly, another embodiment of the present invention provides deimmunized forms of the antibodies for use in cancer imaging in human and non-human patients.

In general, for the generation of antibodies to PLD, NFκB and/or AMPK, the enzyme is required to be extracted from a biological sample whether this be from animal including human tissue or from cell culture if produced by recombinant means. The PLD, NFκB and/or AMPK can be separated from the biological sample by any suitable means. For example, the separation may take advantage of any one or more of the PLD, NFκB and/or AMPK surface charge properties, size, density, biological activity and its affinity for another entity (e.g. another protein or chemical compound to which it binds or otherwise associates). Thus, for example, separation of the PLD, NFκB and/or AMPK from the biological fluid can be achieved by any one or more of ultra-centrifugation, ion-exchange chromatography (e.g. anion exchange chromatography, cation exchange chromatography), electrophoresis (e.g. polyacrylamide gel electrophoresis, isoelectric focussing), size separation (e.g., gel filtration, ultra-filtration) and affinity-mediated separation (e.g. immunoaffinity separation including, but not limited to, magnetic bead separation such as Dynabead (trademark) separation, immunochromatography, immuno-precipitation). The separation of PLD, NFκB and/or AMPK from the biological fluid can preserve conformational epitopes present on the protein and, thus, suitably avoids techniques that cause denaturation of the protein. In a further embodiment, the protein can be separated from the biological fluid using any one or more of affinity separation, gel filtration and/or ultra-filtration.

Immunization and subsequent production of monoclonal antibodies can be carried out using standard protocols known in the art, such as, for example, described by Kohler and Milstein (Kohler and Milstein, Nature 256: 495-499, 1975; Kohler and Milstein, Eur. J. Immunol. 6(7): 511-519, 1976), Coligan et al. (“Current Protocols in Immunology, John Wiley & Sons, Inc., 1991-1997) or Toyama et al. (Monoclonal Antibody, Experiment Manual”, published by Kodansha Scientific, 1987). Essentially, an animal is immunized with an PLD, NFκB and/or AMPK-containing biological fluid or fraction thereof or a recombinant form of PLD, NFκB and/or AMPK by standard methods to produce antibody-producing cells, particularly antibody-producing somatic cells (e.g. B lymphocytes). These cells can then be removed from the immunized animal for immortalization. In certain embodiment, a fragment of PLD, NFκB and/or AMPK can be used to the generate antibodies. The fragment can be associated with a carrier. The carrier can be any substance of typically high molecular weight to which a non- or poorly immunogenic substance (e.g. a hapten) is naturally or artificially linked to enhance its immunogenicity.

Immortalization of antibody-producing cells can be carried out using methods which are well-known in the art. For example, the immortalization may be achieved by the transformation method using Epstein-Barr virus (EBV) (Kozbor et al., Methods in Enzymology 121: 140, 1986). In another embodiment, antibody-producing cells are immortalized using the cell fusion method (described in Coligan et al., 1991-1997, supra), which is widely employed for the production of monoclonal antibodies. In this method, somatic antibody-producing cells with the potential to produce antibodies, particularly B cells, are fused with a myeloma cell line. These somatic cells may be derived from the lymph nodes, spleens and peripheral blood of primed animals, preferably rodent animals such as mice and rats. In a particular embodiment, mice spleen cells can be used. In other embodiments, rat, rabbit, sheep or goat cells can also be used. Specialized myeloma cell lines have been developed from lymphocytic tumours for use in hybridoma-producing fusion procedures (Kohler and Milstein, 1976, supra; Shulman et al., Nature 276: 269-270, 1978; Volk et al., J. Virol. 42(1): 220-227, 1982). Many myeloma cell lines can also be used for the production of fused cell hybrids, including, e.g. P3.times.63-Ag8, P3.times.63-AG8.653, P3/NS1-Ag-4-1 (NS-1), Sp2/0-Ag14 and S194/5.XXO.Bu.1. The P3.times.63-Ag8 and NS-1 cell lines have been described by Kohler and Milstein (1976, supra). Shulman et al. (1978, supra) developed the Sp2/0-Ag14 myelonia line. The S194/5.XXO.Bu.1 line was reported by Trowbridge (J. Exp. Med. 148(1): 313-323, 1978). Methods for generating hybrids of antibody-producing spleen or lymph node cells and myeloma cells usually involve mixing somatic cells with myeloma cells in a 10:1 proportion (although the proportion may vary from about 20:1 to about 1:1), respectively, in the presence of an agent or agents (chemical, viral or electrical) that promotes the fusion of cell membranes. Fusion methods have been described (Kohler and Milstein, 1975, supra; Kohler and Milstein, 1976, supra; Gefter et al., Somatic Cell Genet. 3: 231-236, 1977; Volk et al., 1982, supra). The fusion-promoting agents used by those investigators were Sendai virus and polyethylene glycol (PEG). In certain embodiments, means to select the fused cell hybrids from the remaining unfused cells, particularly the unfused myeloma cells, are provided. Generally, the selection of fused cell hybrids can be accomplished by culturing the cells in media that support the growth of hybridomas but prevent the growth of the unfused myeloma cells, which normally would go on dividing indefinitely. The somatic cells used in the fusion do not maintain long-term viability in in vitro culture and hence do not pose a problem. Several weeks are required to selectively culture the fused cell hybrids. Early in this time period, it is necessary to identify those hybrids which produce the desired antibody, so that they may subsequently be cloned and propagated. Generally, around 10% of the hybrids obtained produce the desired antibody, although a range of from about 1 to about 30% is not uncommon. The detection of antibody-producing hybrids can be achieved by any one of several standard assay methods, including enzyme-linked immunoassay and radioimmunoassay techniques as, for example, described in Kennet et al. (Monoclonal Antibodies and Hybridomas: A New Dimension in Biological Analyses, pp 376-384, Plenum Press, New York, 1980) and by FACS analysis (O'Reilly et al., Biotechniques 25: 824-830, 1998).

Once the desired fused cell hybrids have been selected and cloned into individual antibody-producing cell lines, each cell line may be propagated in either of two standard ways. A suspension of the hybridoma cells can be injected into a histocompatible animal. The injected animal will then develop tumours that secrete the specific monoclonal antibody produced by the fused cell hybrid. The body fluids of the animal, such as serum or ascites fluid, can be tapped to provide monoclonal antibodies in high concentration. Alternatively, the individual cell lines may be propagated in vitro in laboratory culture vessels. The culture medium containing high concentrations of a single specific monoclonal antibody can be harvested by decantation, filtration or centrifugation, and subsequently purified.

The cell lines can then be tested for their specificity to detect the PLD, NFκB and/or AMPK of interest by any suitable immunodetection means. For example, cell lines can be aliquoted into a number of wells and incubated and the supernatant from each well is analyzed by enzyme-linked immunosorbent assay (ELISA), indirect fluorescent antibody technique, or the like. The cell line(s) producing a monoclonal antibody capable of recognizing the target protein but which does not recognize non-target epitopes are identified and then directly cultured in vitro or injected into a histocompatible animal to form tumours and to produce, collect and purify the required antibodies.

The present invention provides, therefore, a method of detecting in a sample PLD, NFκB and/or AMPK or fragment, variant or derivative thereof comprising contacting the sample with an antibody or fragment or derivative thereof and detecting the level of a complex containing the antibody and PLD, NFκB and/or AMPK or fragment, variant or derivative thereof compared to normal controls wherein elevated levels of PLD, NFκB and/or AMPK is determined. Any suitable technique for determining formation of the complex may be used. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) immunochromatographic techniques (ICTs), and Western blotting which are well known to those of skill in the art. Immunoassays can also include competitive assays. The present invention encompasses qualitative and quantitative immunoassays.

Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labeled antigen-binding molecule to a target antigen.

The invention further provides methods for quantifying PLD, NFκB and/or AMPK protein expression and activation levels in cells or tissue samples obtained from an animal, such as a human cancer patient or an individual suspected of having cancer. In one embodiment, the invention provides methods for quantifying PLD, NFκB and/or AMPK protein expression or activation levels using an imaging system quantitatively. The imaging system can be used to receive, enhance, and process images of cells or tissue samples, that have been stained with PLD, NFκB and/or AMPK protein-specific stains, in order to determine the amount or activation level of PLD, NFκB and/or AMPK proteins expressed in the cells or tissue samples from such an animal. In embodiments of the methods of the invention, a calibration curve of PLD, NFκB and/or AMPK protein expression can be generated for at least two cell lines expressing differing amounts of PLD, NFκB and/or AMPK protein. The calibration curve can then used to quantitatively determine the amount of PLD, NFκB and/or AMPK protein that is expressed in a cell or tissue sample. Analogous calibration curves can be made for activated PLD, NFκB and/or AMPK proteins using reagents specific for the activation features. It can also be used to determine changes in amounts and activation state of PLD, NFκB and/or AMPK before and after clinical cancer treatment.

In one particular embodiment of the methods of the invention, PLD, NFκB and/or AMPK protein expression in a cell or tissue sample can be quantified using an enzyme-linked immunoabsorbent assay (ELISA) to determine the amount of PLD, NFκB and/or AMPK protein in a sample. Such methods are described, for example, in U.S. Patent Publication No. 2002/0015974.

In other embodiments enzyme immunoassays can be used to detect the PLD, NFκB and/or AMPK. In such assays, an enzyme is conjugated to the second antibody, generally by means of glutaraldehyde or periodate. The substrates to be used with the specific enzymes are generally chosen for the production of, upon hydrolysis by the corresponding enzyme, a detectable colour change. It is also possible to employ fluorogenic substrates, which yield a fluorescent product rather than the chromogenic substrates. The enzyme-labeled antibody can be added to the first antibody-antigen complex, allowed to bind, and then the excess reagent washed away. A solution containing the appropriate substrate can then be added to the complex of antibody-antigen-antibody. The substrate can react with the enzyme linked to the second antibody, giving a qualitative visual signal, which may be further quantitated, usually spectrophotometrically, to give an indication of the amount of antigen which was present in the sample. Alternately, fluorescent compounds, such as fluorescein, rhodamine and the lanthanide, europium (EU), can be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic colour visually detectable with a light microscope. The fluorescent-labeled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules can also be employed.

In a particular embodiment, antibodies to PLD, NFκB and/or AMPK can also be used in ELISA-mediated detection of PLD, NFκB and/or AMPK especially in serum or other circulatory fluid. This can be accomplished by immobilizing anti-PLD, NFκB and/or AMPK antibodies to a solid support and contacting these with a biological extract such as serum, blood, lymph or other bodily fluid, cell extract or cell biopsy. Labeled anti-PLD, NFκB and/or AMPK antibodies can then be used to detect immobilized PLD, NFκB and/or AMPK. This assay can be varied in any number of ways and all variations are encompassed by the present invention and known to one skilled in the art. This approach can enable rapid detection and quantitation of PLD, NFκB and/or AMPK levels using, for example, a serum-based assay.

In one embodiment, PLD, NFκB and/or AMPK Elisa assay kit may be used in the present invention. Elisa assay kit containing an anti-PLD, NFκB and/or AMPK antibody and additional reagents, including, but not limited to, washing buffer, antibody dilution buffer, blocking buffer, cell staining solution, developing solution, stop solution, secondary antibodies, and distilled water.

### Nucleotide Detection

In another embodiment, a method to detect PLD, NFκB and/or AMPK is provided by detecting the level of expression in a cell of a polynucleotide encoding an PLD, NFκB and/or AMPK. Expression of the polynucleotide can be determined using any suitable technique known to one skilled in the art. In one embodiment, a labeled polynucleotide encoding an PLD, NFκB and/or AMPK can be utilized as a probe in a Northern blot of an RNA extract obtained from the cell. In other embodiments, a nucleic acid extract from an animal can be utilized in concert with oligonucleotide primers corresponding to sense and antisense sequences of a polynucleotide encoding the kinase, or flanking sequences thereof, in a nucleic acid amplification reaction such as RT PCR. A variety of automated solid-phase detection techniques are also available to one skilled in the art, for example, as described by Fodor et al. (Science 251: 767-777, 1991) and Kazal et al. (Nature Medicine 2: 753-759, 1996).

In other embodiments, methods are provided to detect PLD, NFκB and/or AMPK encoding RNA transcripts. The RNA can be isolated from a cellular sample suspected of containing PLD, NFκB and/or AMPK RNA, e.g. total RNA isolated from human cancer tissue. RNA can be isolated by methods known in the art, e.g. using TRIZOL reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs can then amplified with sequence-specific oligonucleotides in PCR reactions to yield an amplified product.

Polymerase chain reaction or “PCR” refers to a procedure or technique in which amounts of a preselected fragment of nucleic acid, RNA and/or DNA, are amplified as described, for example, in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al. (Quant. Biol. 51: 263, 1987; Erlich, eds., PCR Technology, Stockton Press, NY, 1989). Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian PLD, NFκB and/or AMPK genes. For example, one primer is prepared which is predicted to anneal to the antisense strand and another primer prepared which is predicted to anneal to the sense strand of a cDNA molecule which encodes PLD, NFκB and/or AMPK. To detect the amplified product, the reaction mixture is typically subjected to agarose gel electrophoresis or other convenient separation technique and the relative presence of the PLD, NFκB and/or AMPK specific amplified DNA detected. For example, PLD, NFκB and/or AMPK amplified DNA may be detected using Southern hybridization with a specific oligonucleotide probe or comparing its electrophoretic mobility with DNA standards of known molecular weight. Isolation, purification and characterization of the amplified PLD, NFκB and/or AMPK DNA can be accomplished by excising or eluting the fragment from the gel (for example, see references Lawn et al., Nucleic Acids Res. 2: 6103, 1981; Goeddel et al., Nucleic Acids Res. 8: 4057-1980), cloning the amplified product into a cloning site of a suitable vector, such as the pCRII vector (Invitrogen), sequencing the cloned insert and comparing the DNA sequence to the known sequence of PLD, NFκB and/or AMPK. The relative amounts of PLD, NFκB and/or AMPK mRNA and cDNA can then be determined.

In one embodiment, real-time PCR can be used to determine transcriptional levels of PLD, NFκB and/or AMPK nucleotides. Determination of transcriptional activity also includes a measure of potential translational activity based on available mRNA transcripts. Real-time PCR as well as other PCR procedures use a number of chemistries for detection of PCR product including the binding of DNA binding fluorophores, the 5′ endonuclease, adjacent liner and hairpin oligoprobes and the self-fluorescing amplicons. These chemistries and real-time PCR in general are discussed, for example, in Mackay et al., Nucleic Acids Res 30(6): 1292-1305, 2002; Walker, J. Biochem. Mol. Toxicology 15(3): 121-127, 2001; Lewis et al., J. Pathol. 195: 66-71, 2001.

In an alternate embodiment, the expression of PLD, NFκB and/or AMPK can be identified by contacting a nucleotide sequences isolated from a biological sample with an oligonucleotide probe having a sequence complementary to PLD, NFκB and/or AMPK sequence. The hybridization of the probe to the biological sample can be detected by labeling the probe using any detectable agent. The probe can be labeled for example, with a radioisotope, or with biotin, fluorescent dye, electron-dense reagent, enzyme, hapten or protein for which antibodies are available. The detectable label can be assayed by any desired means, including spectroscopic, photochemical, biochemical, immunochemical, radioisotopic, or chemical means. The probe can also be detected using techniques such as an oligomer restriction technique, a dot blot assay, a reverse dot blot assay, a line probe assay, and a 5′ nuclease assay. Alternatively, the probe can be detected using any of the generally applicable DNA array technologies, including macroarray, microarray and DNA microchip technologies. The oligonucleotide probe typically includes approximately at least 14, 15, 16, 18, 20, 25 or 28 nucleotides that hybridize to the nucleotides. It is generally not preferred to use a probe that is greater than approximately 25 or 28 nucleotides in length. The oligonucleotide probe is designed to identify a PLD, NFκB and/or AMPK nucleotide sequence.

### Mode of Action

In certain embodiments of the present action, methods are provided to treat any of the diseases and/or disorders disclosed herein by administering the compounds disclosed herein in a manner such that they modulate the target biological pathways to treat a disorder of that pathway. The present invention is based on the discovery that honokiol and/or derivatives thereof can have the following effects on cells: inhibition of VEGFR2 phosphorylation, stimulation of TRAIL mediated apoptosis, stimulation of AMPK activation, inhibition of phospholipase D activity and/or inhibition of NFκB activation. Thus, in certain embodiments of the present invention, methods are provided to inhibit VEGFR2 phosphorylation, stimulate TRAIL mediated apoptosis, stimulate AMPK activation, inhibit phospholipase D activity and/or inhibit NFκB activation to treat the diseases and/or disorders disclosed herein via administration of honokiol or derivatives thereof.

In a further embodiment, the present invention provides methods to treat individuals with cancers that exhibit low levels of caspase-3 and/or caspase-8 by administering honokiol or a derivative thereof. In still further embodiments, the present invention provides methods for the treatment of an individual with drug resistant cancer by (i) obtaining a population of cancer cells from the patient, (ii) identifying the levels of caspase-3 and/or caspase-8 in the cancer cells, (iii) determining whether there are low levels of caspase-3 and/or caspase-8; (iv) if low levels of caspase-3 and/or caspase-8 are identified, treating the patient with honokiol or a derivative thereof.

### III. Biological Assays

The biological activity compounds and compositions can be screened in in vitro or in vivo biological assays. Non limiting examples of such assays include, but are not limited to: cellular proliferation assays; evaluation of inhibition of VEGF receptor function, such as VEGF receptor-2 phosphorylation; MAP kinase kinase assays; apoptotic assays, such as TRAIL mediated apoptotic assays; cell viability assays using representative human tumors, such as primary human samples and cell lines; AMP kinase (AMPK) assays; Phospholipase D (PLD) assays; NFκB assays and in vivo assays against xenografts in animals, such as immunocompromised mice.

Cellular proliferation assays known in the art can be used as competition assays. This assay can be used as a direct measure of the candidate compound to serve as an antiangiogenic and/or antitumor agent. In one embodiment compounds that have an IC₅₀ of, for example, approximately 10 μM or less, can be selected for further study. Cells which have activity in this initial assay can then be tested for their ability to preferentially inhibit endothelial proliferation versus fibroblast proliferation using primary human endothelial cells and fibroblasts.

The inhibition of VEGF receptor phosphorylation assay can be done using human dermal microvascular endothelial cells. These cells can be stimulated with recombinant human VEGF (for example, at 10 ng/ml) in the presence or absence of the test compound for a period of time, such as one hour. Protein can then be harvested and immunoprecipitated with anti-phosphotyrosine antibodies. Western blot analysis can then be used to probe for receptor phosphorylation.

Apoptotic assays are commonly known to those skilled in the art. Compounds of the present invention can be tested for their actions in a TRAIL (tumor necrosis factor apoptosis-inducing ligand) apoptosis assay. This assay can be used to determine whether or not the compound can induce apoptosis through TRAIL by using TRAIL neutralizing antibodies to potentially block the effects of the compound. tumor necrosis factor-related apoptosis inducing ligand (TRAIL) mediated apoptosis (Bai et al. (2003) J. Biol. Chem. 278, 35501-35507).

TRAIL/Apo2L is a peptide which has been shown to induce apoptosis in a number of tumor cell lines, while exhibiting no toxicity towards normal cells (Ravi, R. & Bedi, A. (2002) Cancel Res. 62, 4180-4185). TRAIL has two signaling receptors, TRAILR1/DR4 and TRAILR2/DR5 (Ravi, R. & Bedi, A. (2002) Cancer Res. 62, 4180-4185, Schneider, P. et al (1997) FEBS Lett. 416, 329-334, Bodmer, J. L. et al. (2000) Nat. Cell Biol. 2, 241-243). One potential mechanism observed on malignant cells is higher expression of these TRAIL receptors compared with benign counterparts (Ghosh et al. (2002) Blood). TRAIL has been shown to cause apoptosis due to involvement of both membrane receptor induced apoptosis, through activation and trimerization of TRAIL receptors, leading to activation of caspase 8, and activation of mitochondrial mediated apoptosis through Apaf-1/caspase 9/cytochrome c (Bodmer, J. L. et al. (2000) Nat. Cell Biol. 2, 241-243, Li, J. (2003) Journal of Immunology 171, 1526-1533). A potential mechanism of crosstalk between these two pathways occurs through caspase 8 mediated cleavage/activation of the proapoptotic protein Bid, which then translocates to the mitochondria, resulting in mitochondrial based apoptosis. TRAIL peptide itself has been shown to have antitumor activity in preclinical models, and synergy has been observed of combinations of TRAIL or compounds which stimulate TRAIL signaling and conventional chemotherapeutic agents (Mitsiades, C. S. et al. (2001) Blood 98, 795-804).

Compounds of the present invention can be tested for their actions in adenosine monophosphate kinase (AMPK) assays. This assay can be used to determine whether or not the compound can induce or inhibit AMPK activation. Any phosphorylation assay known in the art can be used to detect activation of AMPK. In addition, western blots can be run test for stimulation of AMPK activation.

The AMP pathway is activated by a high ratio of AMP to ATP, indicating a low energy state. In addition to nutrient deprivation, AMP kinase is activated by hypoxia and honokiol, leading to growth arrest and apoptosis. AMP kinase activation has recently been shown to exert potent antiproliferative effects in tumor cells. AMPK has been shown to be a physiologic antagonist to akt, a serine-threonine kinase which is a major downstream effector of phosphoinositol-3 kinase in terms of tumor proliferation and apoptosis. In addition, AMPK has been shown to antagonize the inactivation of tuberin (tsc2) by akt, thus providing an additional mechanism of antitumor activity. As a consequence of this activity, AMPK activation can result in downregulation of mammalian target of rapamycin (mTOR), leading to decreased protein synthesis in tumor cells. Upstream of AMPK is LKB, an AMP kinase kinase, which is mutated in the tumor prone Peutz Jeghers Syndrome. In a recent in vivo study, intraperitoneal administration of aminoimidazole carboxamide riboside (AICAR) led to a significant inhibition of C6 glioma growth in rats. This inhibition was accompanied in part through upregulation of the tumor suppressor genes p53, p21, and p27. In addition, AICAR inhibited the activation of PI3K/akt signaling. The laboratory of Arnold Levine at Princeton has demonstrated that p53, when activated by cellular stresses such as chemotherapy, undergoes phosphorylation at ser-15, resulting in upregulation of AMPK, and subsequent downregulation of mTOR. Pharmacologic inhibition of AMPK desensitizes cells to chemotherapy (Feng, Z. H et al (2005) Proceedings of the National Academy of Sciences of the United States of America 102, 8204-8209). Thus activation of AMPK can be beneficial in the treatment of tumors through both p53 dependent and independent pathways.

Compounds of the present invention can also be tested for their effect on phospholipase D activity (PLD) to determine whether or not the compound can induce or inhibit PLD. Cells that are known to express high levels of PLD can be used to assay PLD activity. The cells can be treated for a period of time with the compounds, the lipids can then be extracted from the cells, and the effect on PLD activity ascertained. Compounds can also be tested for their effects on downstream targets of PLD, such as mTOR, S6 kinase and/or S6.

Phospholipase D (PLD) is a lipase which cleaves phosphatidylcholine to phosphatidic acid and choline. Phosphatidic acid can be converted to other biologically active lipids, including lysophosphatidic acid, which activated protein kinase D, while phosphatidic acid itself can activate isoforms of protein kinase C. The net consequences of these activities are increased cellular proliferation and decreased apoptosis, similar to what has been observed by akt. Virtually all studied tyrosine kinase receptors can stimulate PLD activation upon exposure to appropriate ligands. PLD thus serves as a nonoverlapping yet parallel signaling pathway to akt. Two major PLD genes in humans have been isolated, PLD1 and PLD2. Both of these genes have pleckstrin homology domains (PH), which bind phosphate pairs of phosphatidylinositol (PtdIns) PtdIns(4,5)P2 and PtdIns(3,4)P2. PLD1 has been shown to be activated by small G proteins (Rho, rac, cdc42), while PLD2 has been found to associate with PtdIns-4-P-5 kinase 1a, and has been more closely associated with cell shape changes and tumorigenesis (Joseph, T et al (2001) Biochemical and Biophysical Research Communications 289, 1019-1024; Chen, Y. H et al. (2005) Oncogene 24, 672-679).

In addition to activation of known kinases, PLD activation has been shown to stabilize the oncogenic c-myc protein. Interestingly, in breast cancer, there appears to be an inverse relationship between PLD activation and akt activation. Breast cancer cells that have elevated akt elevation appear to be most sensitive to rapamycin, while PLD breast cancer cells, which are highly undifferentiated, appear to be less sensitive to rapamycin. This may be due to competitive binding of rapamycin and phosphatidic acid to mTOR. Decreased production of phosphatidic acid through inhibition of PLD by honokiol might sensitize tumor cells to rapamycin (Chen, Y. H et al. (2005) Oncogene 24, 672-679; (Rodrik, V et al (2005) Molecular and Cellular Biology 25, 7917-7925; Chen, Y. H et al. (2003) Oncogene 22, 3937-3942).

Compounds of the present invention can be tested for their effect on Nuclear factor kappa beta (NFkB) to determine whether or not the compound can induce or inhibit NFkB. Any assay known in the art to assay for NFkB activity can be used.

Nuclear factor kappa beta (NFkB) is a family of transcription factors that are vital to the survival of a large number of tumor types. Constitutive overexpression of NFkB has been observed in multiple myeloma, virtually all types of leukemias, melanoma, glioblastoma, epithelial malignancies, and sarcomas. NFkB is pivotal in apoptosis prevention in a number of ways, and can impact on both intrinsic and extrinsic apoptotic pathways. NFkB inhibition sensitizes tumor cells to both chemotherapy, as well as apoptosis due to ligands such as TRAIL, FAS, and TNFa (Mitsiades, et al (2001) Blood 98, 795-804; Bernard, et al (2001) Journal of Biological Chemistry 276, 27322-27328). Inhibition of NFkB has been used clinically in humans through the use of proteasome inhibitors, such as velcade, for multiple mycloma. Other drugs for myeloma, including thalidomide, and prednisone, also downregulate NFkB activity, possibly accounting for their clinical activity in multiple myeloma.

NFkB is regulated at a number of levels. It consists of several family members including p50 and p65 as the most commonly observed members. These proteins are capable of either homo or heterodimerization, and these dimers mediate transcription. In addition, nuclear localization of NFkB is required for activation. Cellular localization of NFkB is regulated by IKK, which binds NFkB, and prevents nuclear localization, and causes subsequent degradation by ubiquitination and proteasome mediated degradation. NFkB activity can also be modulated by interaction with p300 (Arany, Z. et al (1996) PNAS 93, 12969-12973; Gerritsen, M. E. et al (1997) Proceedings of the National Academy of Sciences of the United States of America 94, 2927-2932; Ravi, R. et al. (1998) Cancer Research 58, 4531-4536).

Assays to test activity against representative human tumors (such as, for example, multiple myeloma) can be conducted using both primary human samples and cell lines. Such samples and cell lines can also be resistant to certain chemotherapeutic agents, for example, myeloma cells that are resistant to doxorubicin can be used.

In vivo models of cancer growth include xenografts of human tumor cells injected into animals, such as mice, particularly immunocomprimised mice. Toxicity of the compounds can also be assessed in these assays through daily administration of the compounds to the animals combined with daily monitoring of the animals. The animals can also be monitored for two weeks to look for weight loss, as well as other common toxicities known to one skilled in the art, such as altered grooming, decreased movement of mice, and tremor. The compounds can also be tested for their ability to inhibit tumor growth in animals. SVR angiosarcoma tumor cells, for example, can be injected into animals, such as immunocompromised mice. Mice can be injected with approximately one million SVR cells subcutaneously, and when tumors become palpable, can be treated with a beginning dose (such as 120 mg/kg intraperitoneally) once daily. Drugs that are active at 120 mg/kg can then be retested at lower doses of 80 mg/kg, 40 mg/kg, etc. Tumor volume can be calculated using the formula (w2×L)0.52, where w (width) represents the smallest dimension of the tumor. Mice can be treated for a period of time such as approximately 30 days, or until tumor growth reaches 1 cm³.

### V. Pharmaceutical Compositions

An effective amount of any of the compounds described herein can be used to treat any of the disorders described herein.

Pharmaceutical carriers suitable for administration of the compounds provided herein include any such carriers known to those skilled in the art to be suitable for the particular mode of administration. The compounds may be formulated as the sole pharmaceutically active ingredient in the composition or may be combined with other active ingredients.

Compositions comprising the compounds disclosed herein may be suitable for oral, rectal, nasal, topical (including buccal and sublingual), vaginal, or parenteral (including subcutaneous, intramuscular, subcutaneous, intravenous, intradermal, intraocular, intratracheal, intracisternal, intraperitoneal, and epidural) administration.

The compositions may conveniently be presented in unit dosage form and may be prepared by conventional pharmaceutical techniques. Such techniques include the step of bringing into association one or more compositions of the present invention and one or more pharmaceutical carriers or excipients.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof may be mixed with one or more suitable pharmaceutical carriers. The compounds may be derivatized as the corresponding salts, esters, enol ethers or esters, acetals, ketals, orthoesters, hemiacetals, hemiketals, acids, bases, solvates, hydrates or prodrugs prior to formulation. The concentrations of the compounds in the compositions are effective for delivery of an amount, upon administration, that treats, prevents, or ameliorates one or more of the symptoms of the target disease or disorder. In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

Compositions suitable for oral administration may be presented as discrete units such as, but not limited to, tablets, caplets, pills or dragees capsules, or cachets, each containing a predetermined amount of one or more of the compositions; as a powder or granules; as a Solution or a suspension in an aqueous liquid or a non-aqueous liquid; or as an oil-in-water liquid emulsion or a water-in-oil emulsion or as a bolus, etc.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents, preservatives, flavoring agents, and the like, for example, acetate, sodium citrate, cyclodextrine derivatives, sorbitan monolaurate, triethanolamine sodium acetate, triethanolamine oleate, and other such agents. Methods of preparing such dosage forms are known, or will be apparent, to those skilled in this art; for example, see Remington's Pharmaceutical Sciences, Mack Publishing Company, Easton, Pa., 15th Edition, 1975.

Compositions of the present invention suitable for topical administration in the mouth include for example, lozenges, having the ingredients in a flavored basis, usually sucrose and acacia or tragacanth; pastilles, having one or more of the compositions of the present invention in an inert basis such as gelatin and glycerin, or sucrose and acacia; and mouthwashes, having one or more of the compositions of the present invention administered in a suitable liquid carrier.

The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

Compositions suitable for topical administration to the skin may be presented as ointments, creams, gels, and pastes, having one or more of the compositions administered in a pharmaceutical acceptable carrier.

Compositions for rectal administration may be presented as a suppository with a suitable base comprising, for example, cocoa butter or a salicylate.

Compositions suitable for nasal administration, when the carrier is a solid, include a coarse powder having a particle size, for example, in the range of 20 to 500 microns which is administered in the manner in which snuff is taken, (i.e., by rapid inhalation through the nasal passage from a container of the powder held close up to the nose). When the carrier is a liquid (for example, a nasal spray or as nasal drops), one or more of the compositions can be admixed in an aqueous or oily solution, and inhaled or sprayed into the nasal passage.

Compositions suitable for vaginal administration may be presented as pessaries, tampons, creams, gels, pastes, foams or spray formulations containing one or more of the compositions and appropriate carriers.

Compositions suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The compositions may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules, and tablets of the kind previously described above.

Pharmaceutical organic or inorganic solid or liquid carrier media suitable for enteral or parenteral administration can be used to fabricate the compositions. Gelatin, lactose, starch, magnesium stearate, talc, vegetable and animal fats and oils, gum, polyalkylene glycol, water, or other known carriers may all be suitable as carrier media.

Compositions may be used as the active ingredient in combination with one or more pharmaceutically acceptable carrier mediums and/or excipients. As used herein, “pharmaceutically acceptable carrier medium” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Additionally, the compositions may be combined with pharmaceutically acceptable excipients, and, optionally, sustained-release matrices, such as biodegradable polymers, to form therapeutic compositions. A “pharmaceutically acceptable excipient” includes a non-toxic solid, semi-solid or liquid filler, diluent, encapsulating material or formulation auxiliary of any type.

It will be understood, however, that the total daily usage of the compositions will be decided by the attending physician within the scope of sound medical judgment. The specific therapeutically effective dose level for any particular host will depend upon a variety of factors, including for example, the disorder being treated and the severity of the disorder; activity of the specific composition employed; the specific composition employed, the age, body weight, general health, sex and diet of the patient; the time of administration; route of administration; rate of excretion of the specific compound employed; the duration of the treatment; drugs used in combination or coincidential with the specific composition employed; and like factors well known in the medical arts. For example, it is well within the skill of the art to start doses of the composition at levels lower than those required to achieve the desired therapeutic effect and to gradually increase the dosage until the desired effect is achieved.

Compositions are preferably formulated in dosage unit form for ease of administration and uniformity of dosage. “Dosage unit form” as used herein refers to a physically discrete unit of the composition appropriate for the host to be treated. Each dosage should contain the quantity of composition calculated to produce the desired therapeutic affect either as such, or in association with the selected pharmaceutical carrier medium.

Preferred unit dosage formulations are those containing a daily dose or unit, daily sub-dose, or an appropriate fraction thereof, of the administered ingredient. For example, approximately 1-5 mg per day of a honokiol-type compound can reduce the volume of a solid tumor in mice. In particular, administration of 3 mg daily of the honokiol-type compound reduces the tumor more than 50% as discussed in Bai et al., J Biol Chem. (2003) Sep. 12; 278(37):35501-7. These results can be used to estimate the human dose of a compound.

The dosage will depend on host factors such as weight, age, surface area, metabolism, tissue distribution, absorption rate and excretion rate. In one embodiment, approximately 0.5 to 7 grams per day of a compound disclosed herein may be administered to humans. Optionally, approximately 1 to 4 grams per day of the compound can be administered to humans. In certain embodiments 0.001-5 mg/day is administered to a human. The therapeutically effective dose level will depend on many factors as noted above. In addition, it is well within the skill of the art to start doses of the composition at relatively low levels, and increase the dosage until the desired effect is achieved.

Compositions comprising a compound disclosed herein may be used with a sustained-release matrix, which can be made of materials, usually polymers, which are degradable by enzymatic or acid-based hydrolysis or by dissolution. Once inserted into the body, the matrix is acted upon by enzymes and body fluids. A sustained-release matrix for example is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The compounds may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The liposome can contain, in addition to one or more compositions of the present invention, stabilizers, preservatives, excipients, and the like. Examples of lipids are the phospholipids and the phosphatidyl cholines (lecithins), both natural and synthetic. Methods to form liposomes are known in the art.

The compounds may be formulated as aerosols for application, such as by inhalation. These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

Compositions comprising the compounds disclosed herein may be used in combination with other compositions and/or procedures for the treatment of the conditions described above. For example, a tumor may be treated conventionally with surgery, radiation, or chemotherapy combined with one or more compositions of the present invention and then one or more compositions of the present invention may be subsequently administered to the patient to extend the dormancy of micrometastases and to stabilize, inhibit, or reduce the growth of any residual primary tumor.

### IV. Synthesis

The compounds disclosed herein can be synthesized using methods known in the art. For example, five classes of honokiol analogues can be synthesized, shown in Scheme 1A.

Methods available in the art for the synthesis of honokiol and its derivatives can be modified for the synthesis of the compounds disclosed herein. For example, the synthesis described in Esumi, T., et al., (2004), Bioorg. Med. Chem. Lett. 14, 2621-2625 can be used. In one embodiment, a Suzuki coupling reaction is used for the construction of the biphenyl scaffold. This reaction can produce good product yields as described in Miyaura, N. and Suzuki, A. (1995), Chem. Rev. 95, 2457-2483; and Suzuki, A. (1999), J. Organometal. Chem. 576, 147-168. This reaction can be used for the introduction of the allyl moiety to arylphenol or biphenyl, using the appropriate intermediates. Optionally the allyl group is introduced before the formation of the bisphenol, based on the differential reactivities of iodo and bromo functionalities. See, e.g., Toyota, S., Woods, C. R., Benaglia, M., Siegel, J. S. (1998), Tetrahedron Lett. 39, 2697-2700; and Bahl, A., Grahn, W., Stadler, S., Feiner, F., Bourhill, G., Brauchle, C., Reisner, A., Jones, P. G. (1995), Angew. Chem. Int. Ed. Engl. 34, 1485-1488.

In one embodiment, the intermediates for the synthesis of honokiol are 3-allyl-4-hydroxybenzeneboronate 5 and 4-allyl-2-bromophenol 9. The boronate 5 can be prepared from 2-iodophenol 1 by bromination, followed by Suzuki coupling to introduce the allyl group, and boronation under Suzuki conditions. Compound 9 can be prepared from 4-iodophenol 6 by bromination and allylation (Suzuki coupling). The coupling of 5 and 9 under Suzuki conditions can yield honokiol from Suzuki coupling, not other allyl-oriented products from the Heck reaction, as it was shown that Suzuki coupling can succeed in the presence of C═C double bond (see Miyaura, N.; Suzuki, A. (1995), Chem. Rev. 95, 2457-2483; and Suzuki, A. (1999), J. Organometal. Chem. 576, 147-168, and the references cited therein).

Thus, honokiol and derivatives can be synthesized from commercially available starting materials in 6 steps (Scheme 1).

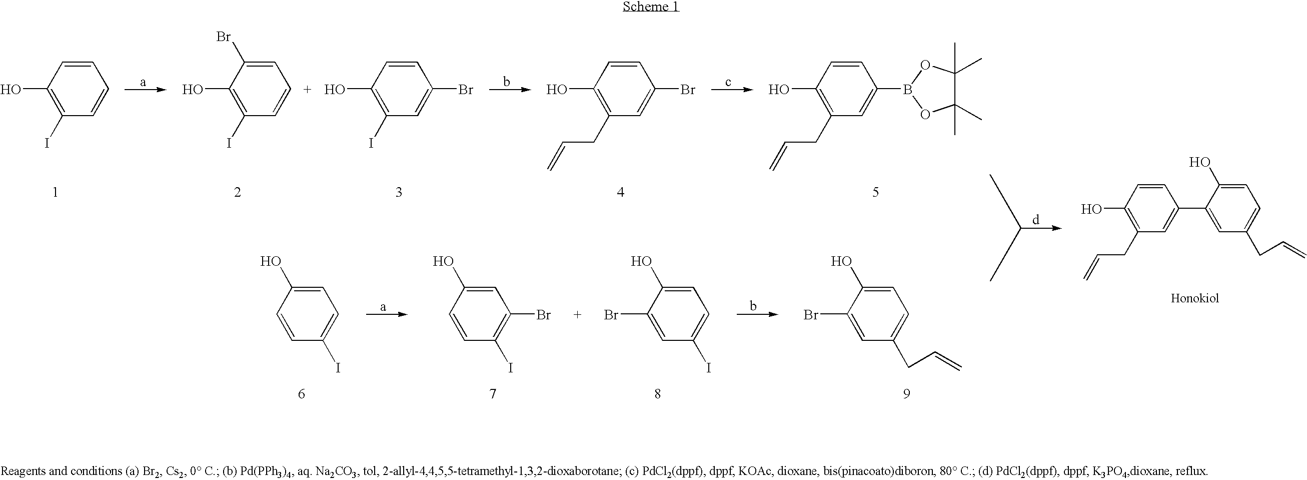

Treatment of honokiol with TMS-diazomethane in methanol results in mono- and di-methylated compounds I-III, and hydrogenation of honokiol with Wilkinson's catalyst yields di- and tetrahydrohonokiols VI-VIII, as reported by Esumi, T. et al. (2004), Bioorg. Med. Chem. Lett. 14, 2621-2625. The amino and fluoro analogues (IV and V) can be constructed from iodoacetanilide under Suzuki coupling conditions. From 2-iodoacetanilide 10, after bromination, allylation, and boronation, the boronated intermediate 13 can be prepared. The other bromo intermediate 16 can be prepared from 4-iodoacetanilide 14 via bromination and allylation. The coupling of boronate 13 and bromide 16 under Suzuki conditions can afford, after deprotection, the compound IV. Diazotization followed by Schiemann reaction can convert the amino analogue IV to fluoro analogue V (Scheme 2).

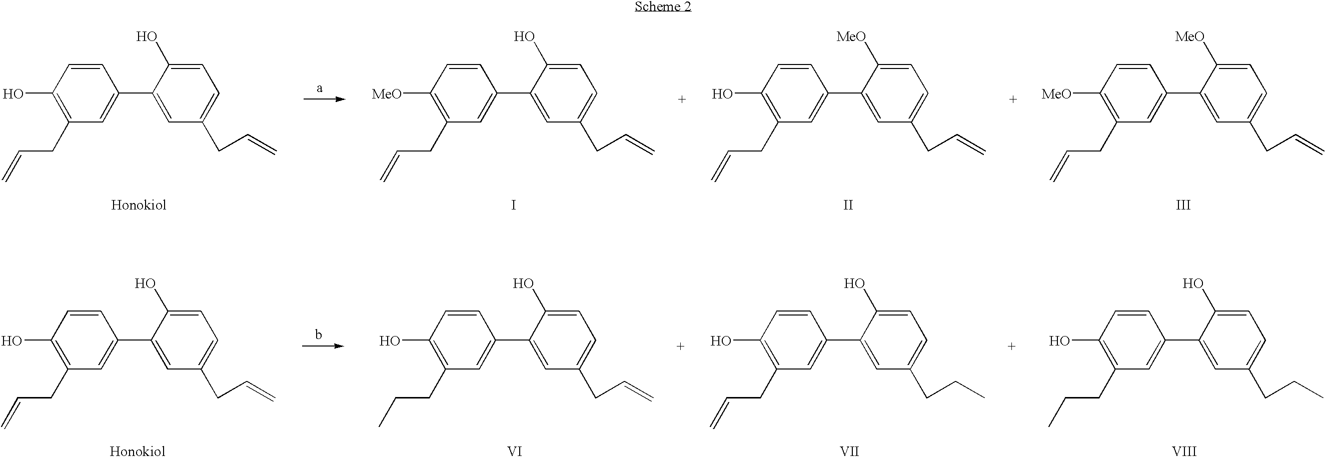

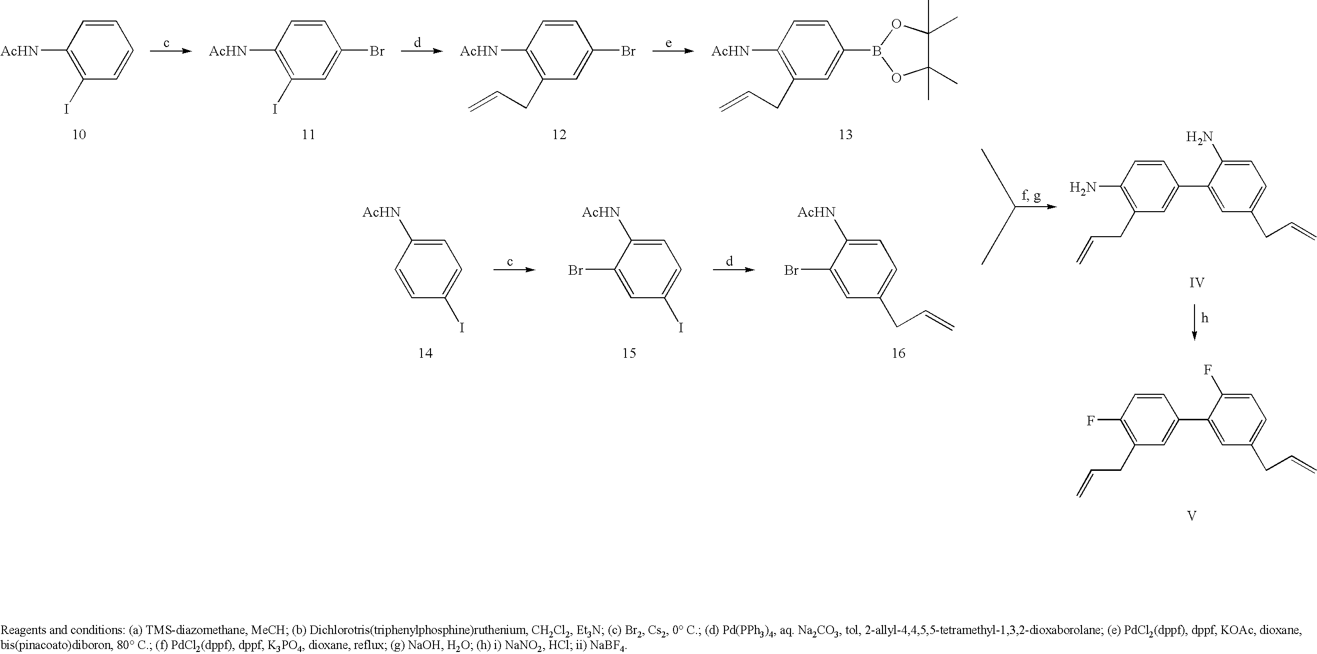

The dimethoxy honokiol derivative, III, can also be prepared, for example, by the treatment of honokiol with potassium carbonate, iodomethane. (Scheme 2a). The hydrogenated honokiol analog can alternatively be prepared by the hydrogenation of honokiol with sodium borohydride and nickel(II) chloride to yields tetrahydrohonokiols VI-VIII. (Scheme 2a).

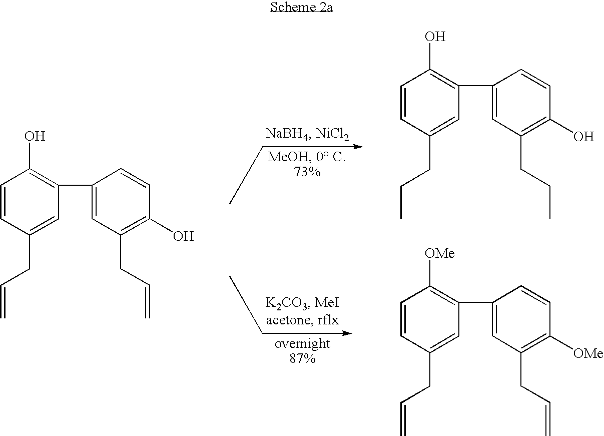

The preparation of the vinyl analogue IX is based on combining the Wittig reaction with Suzuki coupling. The intermediate aldehyde 18 can be prepared from 4-iodophenol 17 via the Reimer-Tiemann reaction, while 3-bromo-4-hydroxybenzenealdehyde 23 can be prepared from para-hydroxybenzoic ester 21 via bromination and reduction. The Wittig reaction of these two aldehydes can yield the corresponding vinyl substituted benzenes 19 and 24. Compound 19 can afford the boronate 20, which can be coupled with 24, to yield the compound IX (Scheme 3).

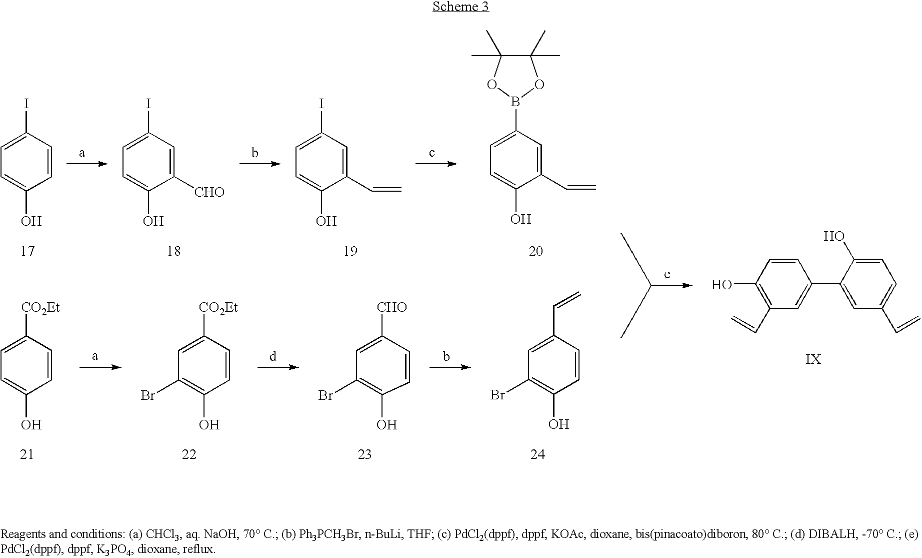

For the synthesis of honokiol analogues with changed positions of the allyl or hydroxyl groups, the boronate 5, and the bromophenols 4 and 9 can be used as intermediates. Suzuki coupling of one of these intermediates with an appropriate halide or boronate can provide the compounds X-XVII. Compounds X-XII and XIV-XV can be prepared by Suzuki coupling of boronate 5 with an appropriate halide. Halide 25, needed for compound X, can be prepared from 2-bromo-6-iodophenol 2 via allylation, while the intermediate, 5-allyl-2-bromophenol 29 for compound XI, can be furnished from 3-iodophenol 26 via bromination and allylation. The preparation of halide 5-allyl-3-bromophenol 33, an intermediate for the synthesis of compound XIV, requires an organothallium reagent. The thallation of 3-bromophenol 30 followed by treatment with iodide can yield 3-bromo-5-iodophenol 32. After allylation, the allyl-substituted intermediate 33 can be prepared. The synthesis of compound XII can begin with 2-iodoacetanilide 10, via sulfonation, nitration, and reduction to obtain the intermediate 36. Aniline 36, after diazotization, followed by acid and base treatments, will afford 2-amino-3-iodophenol 37. Diazotization, Sandmeyer reaction, and allylation of compound 37 will yield halide 39. By a coupling reaction of these halides (25, 29, 33, and 39) with boronate 5, these compounds (X-XII, and XIV) can be prepared. Compound XV can be synthesized by Suzuki coupling of halide 4 with boronate 5 (Scheme 4).

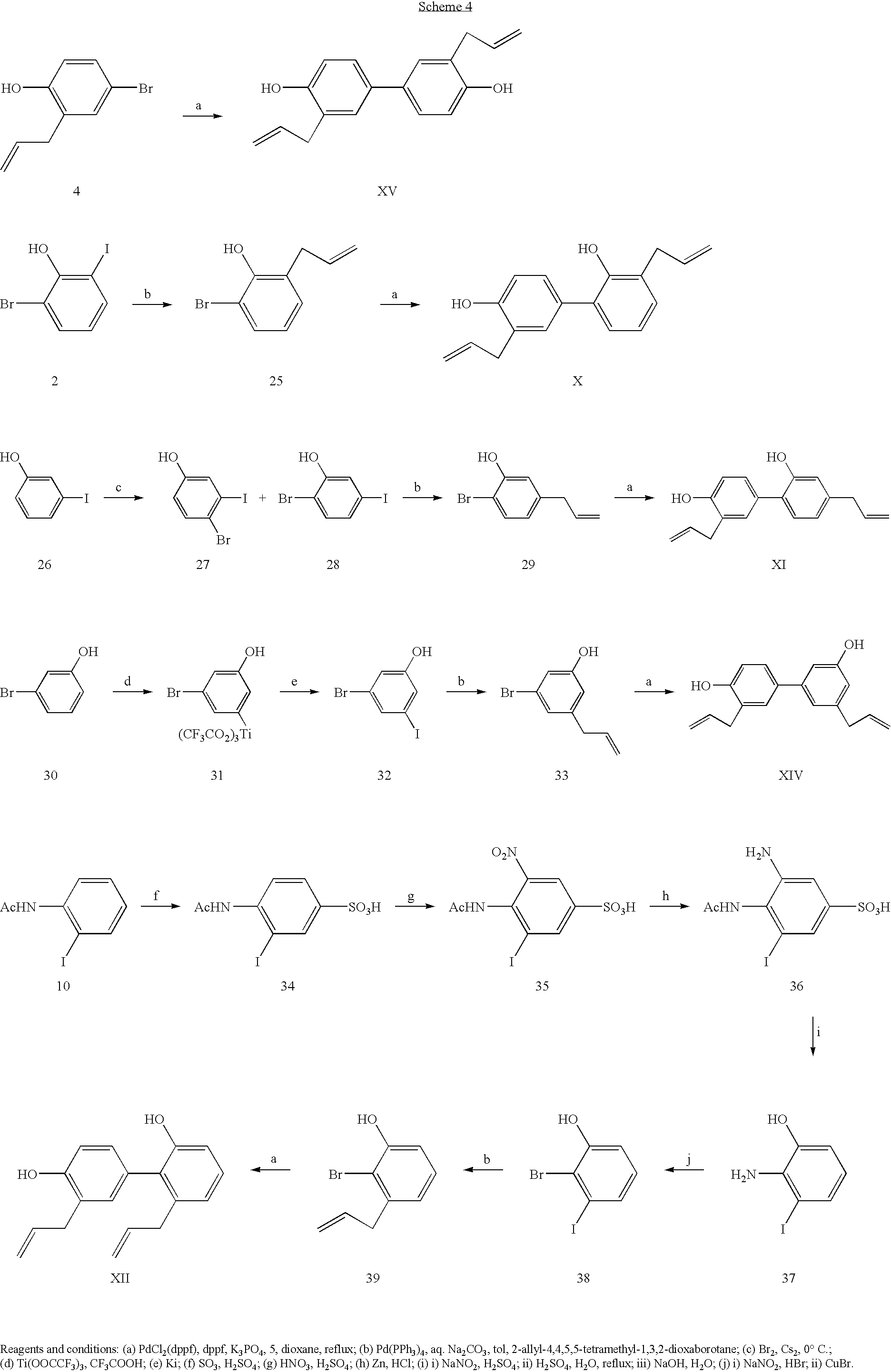

Alternatively, compounds X, XV, and XVII can be synthesized by an allylation-Claisen pathway. Biphenol compounds can be reacted first with potassium carbonate and allyl bromide, followed by reaction with BCl₃ to yield honokiol-like compounds, for example, X, XV, and XVII. (Scheme 4a). To a cooled solution (0° C. with an ice bath) of diallyl starting material (1 eq.) in dry dichloromethane (Concentration of the solution: 0.1 mol.L⁻¹) was added dropwise a solution of BCl₃ (1M in dichloromethane; 1.5 eq.=0.75 eq for each allyl group). The reaction is then stirred at 0° C. until disappearance of the starting material on TLC (If after 15 minutes, the reaction is not complete, 1 more equivalent of BCl₃ can be added). After hydrolysis with water (about same volume than dichloromethane), the two layers are separated. The organic layer is washed again with water, dried under MgSO₄ then evaporated under vacuum. The residue is finally purified by column chromatography to give the di-hydroxyderivative.

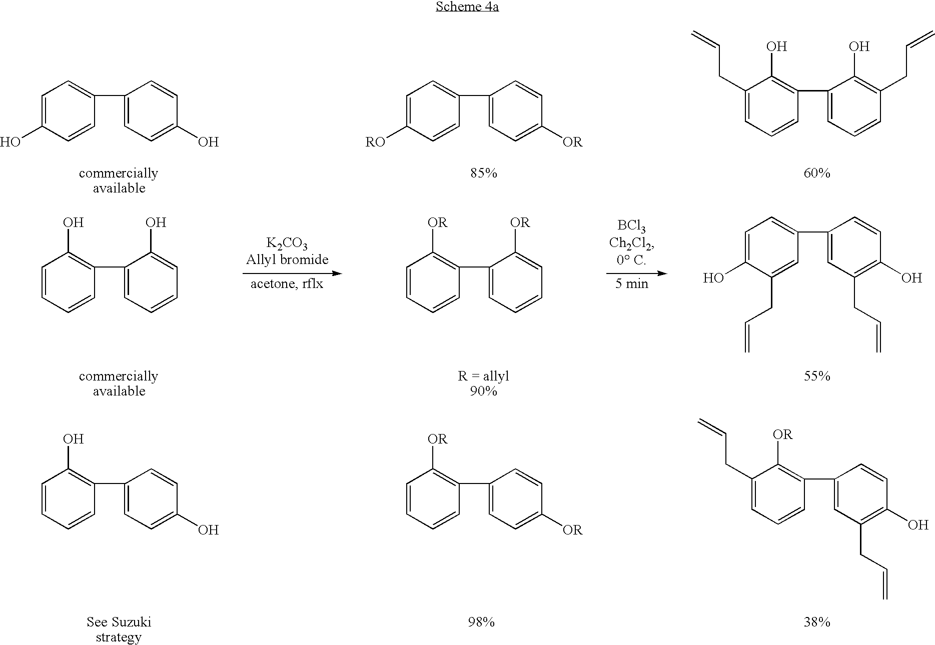

Bromide 9 is also a useful intermediate for coupling with some boronates. For example, Suzuki coupling of bromide 9 with boronate 42, which is prepared from 4-bromo-3-iodophenol 40 via allylation and boronation, can yield the compound XIII. Similarly, the coupling between bromide 9 and boronate 43 can afford the compound XVI. The compound XVII can be prepared from 4-allyl-2-bromophenol 9 via boronation followed by Suzuki coupling with 2-allyl-6-bromophenol 25 (Scheme 5).

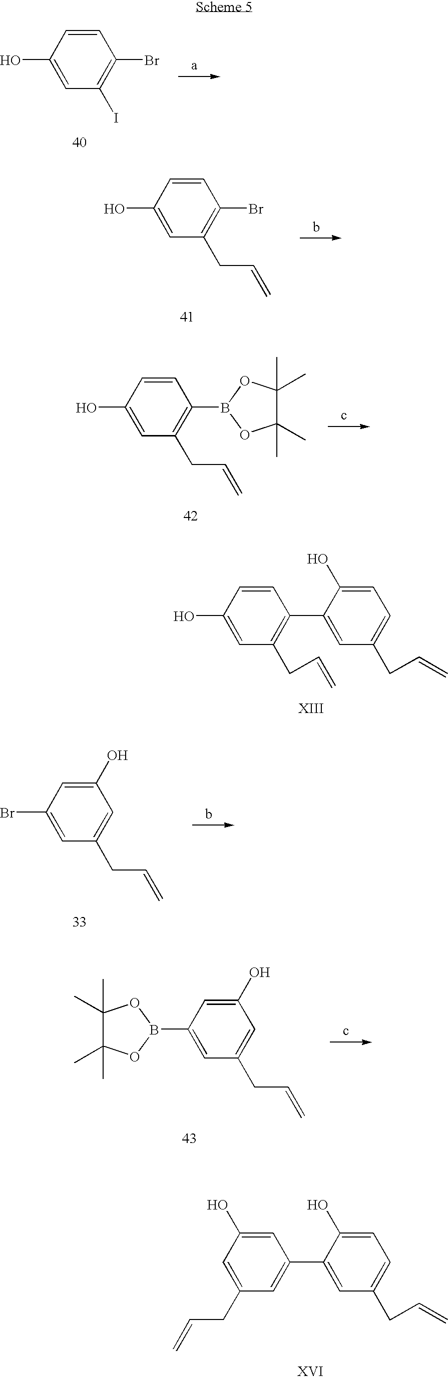

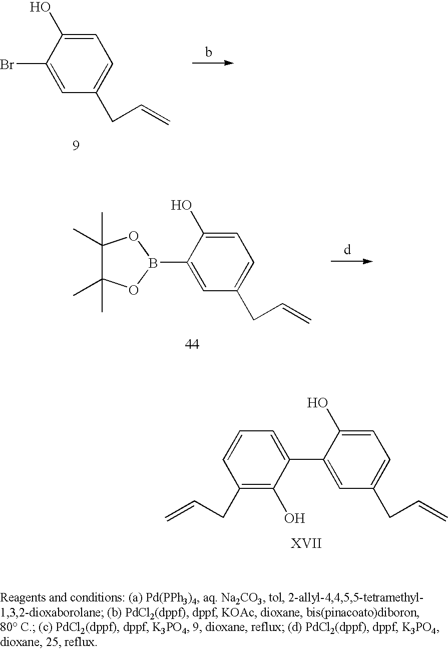

The compounds XVIII and XIX can be synthesized from commercially available bisphenol 45 and the dihydroxynaphthalene-disulfuric acid salt 47. Thus, the bisphenol 45, through the Williamson reaction and Claisen rearrangement, can be converted to compound XVIII. Similarly, desulfonation of dihydroxynaphthalene-disulfuric acid salt 47, followed by the Williamson reaction and Claisen rearrangement, can produce the compound XIX (Scheme 6).

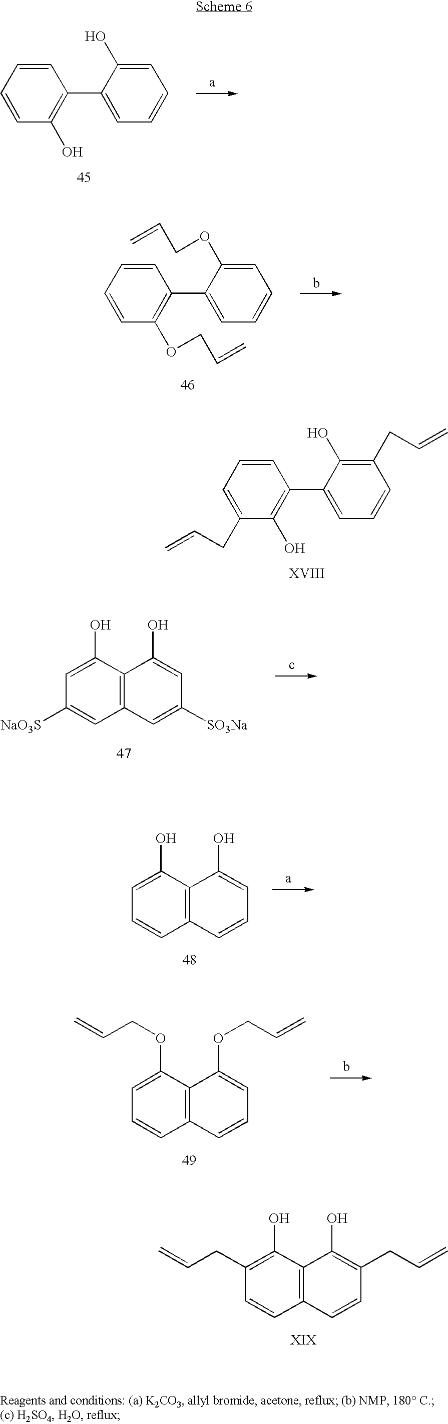

Dioxolane compounds can be prepared from magnoliol by reaction of magnoliol with 2,2-dimethoxypropane and p-toluenesulfonic acid. (Scheme 7). This synthesis also provides a method of separating mixtures of honokiol and magnoliol.

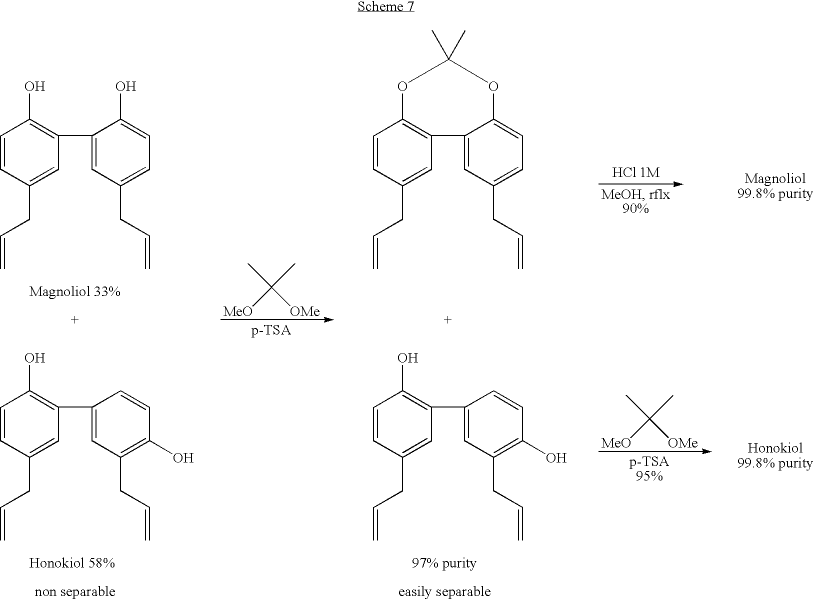

The following examples are offered by way of illustration and not by way of limitation.

### EXAMPLES

### Example 1

### MAPKK Screen

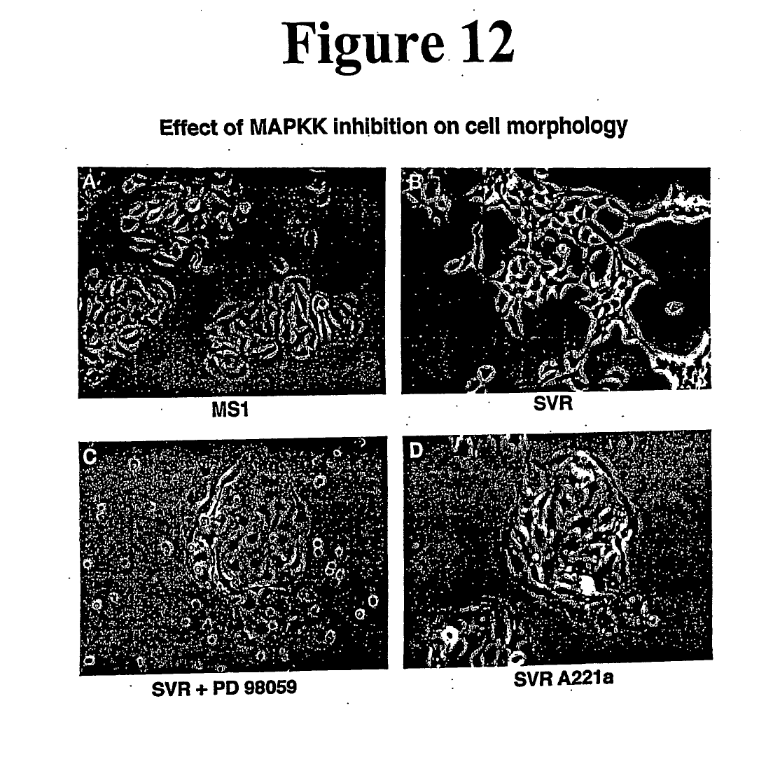

FIG. 12 exhibits the effect of inhibition of MAPKK by a dominant negative MAPKK gene or by the chemical inhibitor PD98059 on morphology of endothelial cells. MS1 represents endothelial cells containing only SV40 large T antigen; SVR represents MS1 cells transformed with ras; SVR+PD98059 represents SVR cells treated with PD98059 (5 μg/ml); and SVRA221a represents cells stably expressing the dominant negative A221 allele of MAPKK. The morphology of SVRAnd SVRbag4 cells are identical. Original magnifications, ×40. This figure illustrates the distinctive response of SVR cells to MAP kinase inhibition, which can be used in a visual high throughput assay to find inhibitors of MAP kinase and related inhibitors (see also, LaMontagne et al (2000) Am. J. Pathol. 157, 1937-1945).

### Example 2

### Intracellular Effects of Honokiol

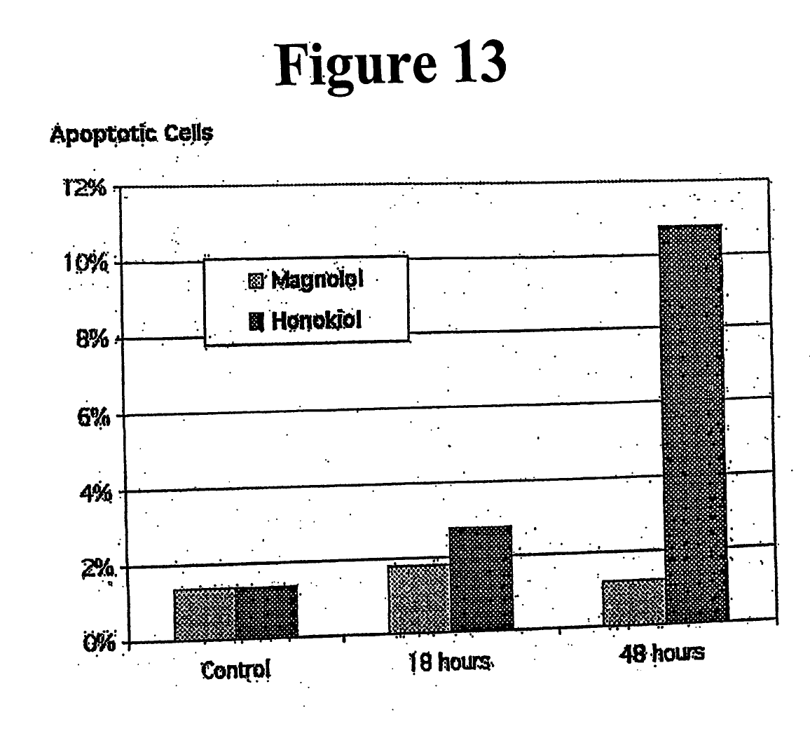

FIG. 13 illustrates the effect of honokiol and magnolol on apoptosis. The light columns represent SVR cells treated with magnolol, and the dark columns represent SVR cells treated with honokiol. The control lanes represent cells immediately after treatment compared with 18 and 48 h of treatment. This figure shows that honokiol is more effective in the induction of apoptosis than magnolol.

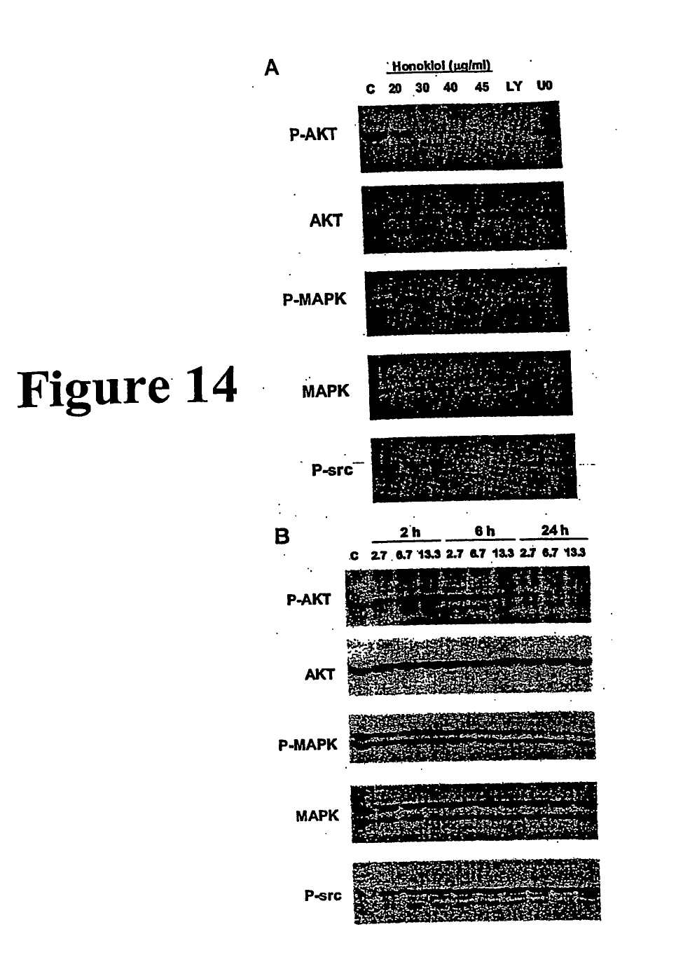

FIG. 14 depicts the effects of honokiol on the phosphorylation of various intracellular proteins. A shows that honokiol inhibits phosphorylation of AKT, p44/42 MAPK, and Src. SVR cells were incubated with 20 (75 μM), 30 (112.5 μM), 40 (150 μM), or 45 μg/ml (169 μM) honokiol for 1 h. SVR cells were also incubated with 50 μM LY294002 (LY) or 50 μM U0126 (U0) for 2 h. Cells were lysed and analyzed by Western blotting using antibodies specific for the phosphorylated (P-AKT, P-MAPK, and P-Src) or unphosphorylated forms of AKT and MAPK. B, honokiol inhibits phosphorylation of Akt at low concentrations but not p44/42 MAPK or Src. SVR cells were incubated with 2.7 (10 μM), 6.7 (25 μM), or 13.3 μg/ml (50 μM) honokiol for 2, 6, or 24 h. Cells were lysed and analyzed by Western blotting using antibodies specific for the phosphorylated (P-Akt, P-MAPK, and P-Src) or unphosphorylated forms of Akt and MAPK. These mechanistic studies indicate that a primary site of action of honokiol is at the level of phosphoinostol 3 kinase activation or an upstream event.

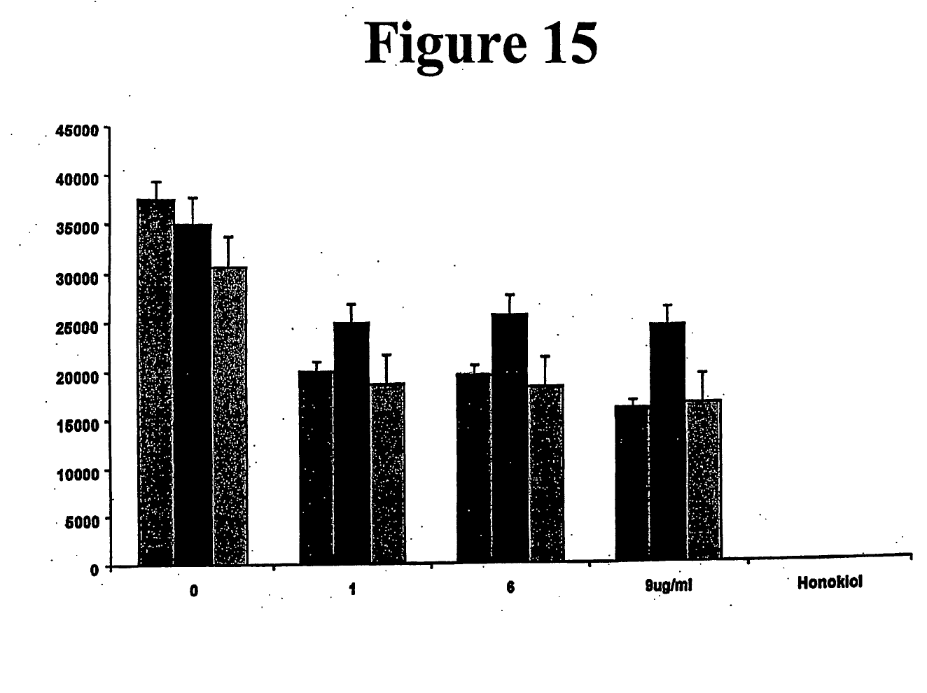

FIG. 15 demonstrates that honokiol inhibition of endothelial proliferation is TRAIL-dependent. 10⁴/well microvascular endothelial cells were cultured in 24-well plates for 24 h. The next day, cells were washed by PBS and pretreated with 0.5 ml/well fresh MEC medium with 0, 1, 6, or 9 μg/ml honokiol for 30 min before addition of TRAIL or isotype control antibody (30 μg/well). Cells were incubated for 48 h after the addition of reagents and were counted with a Coulter Counter. The green bars represent endothelial cells treated with honokiol alone, the dark blue bars represent cells treated with honokiol and TRAIL antibody, and the light blue bars represent cells treated with honokiol and isotype control antibody. The differences in honokiol-treated endothelium in the presence or absence of TRAIL antibody are significant (p<0.05). These findings indicate that honokiol stimulates apoptosis through activation of TRAIL signaling, suggesting that honokiol may be synergistic with other chemotherapeutic and radiation therapies.

### Example 3

### Effect of Honokiol on Multiple Myeloma Cells

### Materials and Methods

Cells: Dexamethasone (Dex)-sensitive MM.1S (wild-type p53) and Dex-resistant MM.1R, RPMI 8226-Dox40 (doxorubicin resistant) and RPMI 8226-LR5 (melphalan resistant) human multiple myeloma (MM) cell lines were used. RPMI-8226 and U266 cells were obtained from the American Type Culture Collection (Rockville, Md.). SU-DHL-4 cells were also utilized. Fresh peripheral blood mononuclear cells (PBMNCs) were obtained from healthy subjects after informed consent. The PBMNC were separated from heparinized peripheral blood by Ficoll-Hipaque density sedimentation. BM specimens were acquired from patients with MM after obtaining informed consent and mononuclear cells were separated by Ficoll-Hipaque density sedimentation. Cells were cultured at 37° C. in RPMI 1640 containing 10% fetal bovine serum (FBS; Sigma, St Louis, Mo.), 2 μM L-glutamine, 100 U/mL penicillin, and 100 μg/mL streptomycin (Gibco, Grand Island, N.Y.).

MNCs in BM specimens were also used to establish long-term bone marrow stromal cell (BMSC) cultures, as described in, for example, Uchiyama et al Blood. 1993; 82:3712-3720 and Hideshmia et al Oncogene. 2001; 20:4519-4527.

Reagents: Honokiol (HNK; Calbiochem, San Diego, Calif.) was dissolved in ethanol at 20 mg/ml stock solution. Recombinant human interleukin-6 (IL-6), vascular endothelial growth factor (VEGF) and insulin-like growth factor-1 (IGF-1) (R&D Systems, Minneapolis, Minn.) were reconstituted with sterile PBS containing 0.1% FBS (IL-6 and VEGF) and 10 mM of acetic acid containing 0.1% FBS (IGF-1), respectively. Pan-caspase inhibitor z-VAD-fmk (Bachem, Bubendorf, Switzerland) was dissolved in methanol. These reagents were stored at −20° C. and diluted by media just before the use. Doxorubicin (Sigma, St Louis, Mo.) was dissolved in sterile water at the concentration of 3.45 mM. As₂O₃ (5 mM in PBS) was provided by Cell Therapeutics Inc. (Seattle, Wash.). Bortezomib (Millennium Pharmaceuticals, Cambridge, Mass.) was dissolved in DMSO at 1 mM and stored at −20° C.
Cellular proliferation and DNA synthesis assays: Colorimetric assays were performed to evaluate drug activity. MM cell lines and BMSCs were treated with indicated concentration of HNK in 96-well culture plates for 48 hours (h) in 100 ul of media and pulsed with 10 μL of 2-(2-methoxy-4-nitrophenyl)-3-(4-nitrophenyl)-5-(2,4-disulfophenyl)-2H-tetrazolium (WST-8, Cell Counting Kit-8, Dojindo, Kumamoto, Japan) to each well for 4 h. WST-8 is converted to WST-8-formazan upon bioreduction in the presence of an electron carrier 1-Methoxy-5-methylphenazinium methylsulfate that is abundant in viable cells. Absorbance readings at a wavelength of 450 nm were taken on a spectrophotometer (Molecular Devices Corp., Sunnyvale, Calif.).

DNA synthesis was measured as previously described, see, for example, Hideshima et al Blood. 2000; 96:2943-2950. Cells in 96-well culture plates were pulsed with 0.5 μCi/well of [³H]-thymidine (Perkin Elmer, Boston, Mass.) during the last 8 h of culture, harvested onto glass filters with an automatic cell harvester (Cambridge Technology, Cambridge, Mass.), and counted using the LKB Betaplate scintillation counter (Wallac, Gaithersburg, Md.). All experiments were performed in triplicate.

Assessment of HNK-induced cytotoxicity against patient MM cells: Cytotoxicity of HNK against fresh MM cells was determined as previously determined, see, for example, Mitsiades et al Cancer Cell. 2004; 5:221-230. Fresh MNCs separated from bone marrow samples derived from patients with MM were incubated with PE-conjugated anti-CD138 antibody and/or FITC-conjugated anti-CD38 antibody (BD Biosciences, San Diego, Calif.) for 30 minutes on ice and washed, followed by analysis using EPICS XL flow cytometer (Beckman Coulter, Hialeah, Fla.). A CD38^{high} fraction enriched for MM cells was determined in side and forward scatter panel in each case. The expression of CD138 on gated cells was also evaluated. After HNK-treatment for 48 h, cells were harvested and percentage of CD38^{high} cells with or without HNK-treatment was evaluated.
Cell cycle analysis: MM cells cultured with HNK were harvested, fixed with 70% ethanol, and pretreated with 250 μg/mL of RNAse (Sigma, St Louis, Mo.). Cells were stained with propidium iodide (PI; 50 μg/mL; Sigma, St Louis, Mo.), and cell cycle profile was determined by using the program M software on an EPICS XL flow cytometer (Beckman Coulter, Hialeah, Fla.).
Detection of apoptosis and caspase-3 activity: TdT-mediated d-UTP nick end labeling (TUNEL) assay (MBL, Nagoya, Japan) and APO 2.7 staining (Immunotech, Marseille, France) were used to determine apoptosis. In brief, cells were fixed and permeabilzed by 4% paraformaldehyde and 70% ethanol, respectively, and incubated with a mixture of FITC-dUTP and TdT for 1 h at 37° C. for TUNEL assay. For detection of mitochondrial membrane protein 7A6 expression on apoptotic cells, cells were incubated with APO 2.7 reagent for 20 minutes. Fluorescence intensity of TUNEL and APO 2.7 staining was determined using on EPICS XL flow cytometer. Cytotoxicity was determined by trypan blue exclusion assay. To evaluate activation of caspase 3, flow cytometric analysis was done using FITC-conjugated monoclonal active caspase 3 antibody apoptosis kit I (BD Biosciences, San Diego, Calif.).
Western blotting: MM cells cultured under indicated conditions were harvested, washed twice with ice-cold PBS, and lysed in lysis buffer; 50 mM Tris-HCl (pH 7.4), 150 mM NaCl, 1% NP-40, 5 mM EDTA, 5 mM NaF, 2 mM Na₃V₄, 1 mM PMSF, 5 ug/ml leupeptine, and 5 ug/ml aprotinin for immunoblotting of whole cell lysate. Subcellular proteins from 1×10⁷ of HNK treated cells were extracted using Nuclear/Cytosol fractionation kit (BioVision, Mountain View, Calif.). Cell lysates or fractionated proteins were subjected to SDS-PAGE, transferred to nitrocellulose membrane, and immunoblotted with these antibodies: anti-caspase 3, -caspase 6, -caspase 7, -caspase 8, -caspase 9, Bad, phosphorylated (p)-Bad (Ser112), Bax, Bak, XIAP, AIF, p-Akt, Akt, p38MAPK, p-p38MAPK, heat shock protein (Hsp) 27 and p-Hsp27 (Cell Signaling, Beverly, Mass., USA); anti-ERK2, p-ERK, STAT3, p-STAT3, bcl-2, Mcl-1, gp80, and Hsp70 (Santa Cruz Biotechnology, Santa Cruz, Calif.); anti-Bid (Biosource International, Camarillo, Calif.); anti-EndoG (Axxora, San Diego, Calif.); and anti-gp130 (Upstate Biotechnology, Lake Placid, N.Y.). Immunoblotting with anti-alpha-tublin Ab (Sigma, St Louis, Mo.) confirmed equivalent protein loading.
Combination with bortezomib: MM.1S cells were cultured with HNK and bortezomib for 48 h. Cell growth and induction of apoptosis were determined both by colorimetric assay and now cytometric detection of APO2.7 after 48 h treatment.
Effect of IL-6, IGF-1 and BMSCs on HNK induced growth inhibition: MM-1S cells were incubated for 48 h with HNK, in the presence or absence of IL-6 or IGF-1. Proliferation of MM cells was then assessed by [³H]-thymidine uptake. To evaluate growth stimulation in MM cells adherent to BMSCs, MM.1S cells were cultured in BMSC-coated 96-well plates for 48 h, in the presence or absence of HNK. DNA synthesis was measured by [³H]-thymidine uptake. To elucidate the modulation of growth signaling induced by IL-6 or IGF-1 in HNK-treated cells, MM.1S cells were cultured in media containing 2.5% of FCS with 10 ug/ml of HNK for 3 and 6 h, followed by stimulation of IL-6 (10 ng/ml) or IGF-1 (25 ng/ml) for 10 and 20 minutes (min). Cell lysates were prepared as described for Western blotting.
Angiogenesis assay: The anti-angiogenic effect of HNK was determined using an In Vitro Angiogenesis Assay Kit (Chemicon, Temecula, Calif.). Human umbilical vein endothelial cells (HUVEC) were cultured in the presence or absence of HNK on polymerized matrix gel at 37° C. After 6 h, tube formation by endothelial cells was evaluated. Direct toxicity of HNK against HUVEC was determined by calorimetric assay.

### Results

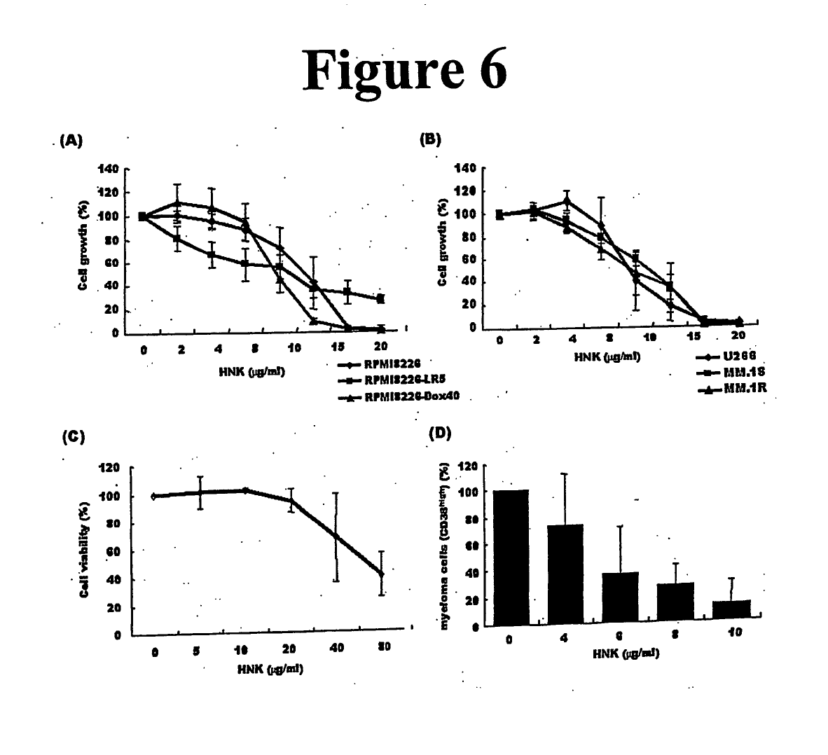

HNK inhibits growth of MM cell lines. To identify the therapeutic potential of HNK, MM cell lines and normal PBMNCs were cultured at indicated concentration with HNK for 48 h, and growth was determined by calorimetric assays. HNK inhibited the growth of drug sensitive RPM18226, U266 and MM.1S cells, with fifty percent inhibition (IC₅₀) at 48 h of 8 to 10 ug/ml. HNK also inhibited growth of drug resistant RPM18226-Dox40, RPM18226-LR5 and MM.1R cells, with IC₅₀ values similar to parental drug-sensitive cell lines (FIGS. 6A and B). While, up to 20 ug/ml of HNK did not inhibit the viability of normal PBMNCs at 48 h (FIG. 6C). FIGS. 6A and B show growth inhibition in MM cell lines by HNK as assessed by calorimetric assay after 48 h-culture. Data represent mean ±SD (standard deviation) of 3 independent experiments. FIG. 6C shows viability of PBMNCs derived from 3 healthy subjects as assessed by calorimetric assay after 48 h-culture. Data represent mean ±SD of triplicate cultures.

HNK is cytotoxic to patient MM cells. Cytotoxicity of HNK against tumor cells isolated from 6 patients with relapsed refractory MM was evaluated. The percentage of CD38^{high} tumor cells was determined by flow cytometry: the percentage of CD38^{high} cells was decreased to 26.2±15.8% after treatment with 8 ug/ml of HNK at 48 h compared to control cultures (FIG. 6D). FIG. 6D illustrates the cytotoxicity of HNK against patient MM cells as determined by comparison of percentage of CD38^{high} cells after culture with or without HNK for 48 h (N=6, Values represent the mean ±SD).

HNK directly inhibits growth of MM cell lines. Growth inhibition of MM cell lines, including Melphalan (MeI)-, Doxorubicin (Dox)-, and Dex-resistant cell lines, was observed at an IC₅₀ of <10 ug/ml. Furthermore, MM cells from patients with relapsed/refractory MM were also significantly reduced by HNK treatment. The IC₅₀ of HNK in normal PBMNCs was 40 to 80 ug/ml, markedly higher than IC₅₀ for MM cell lines and patient MM cells. These data demonstrate that HNK effectively induces cytotoxicity in MM cell lines, including drug resistant cell lines and patient MM cells, without toxicity to normal PBMNCs.

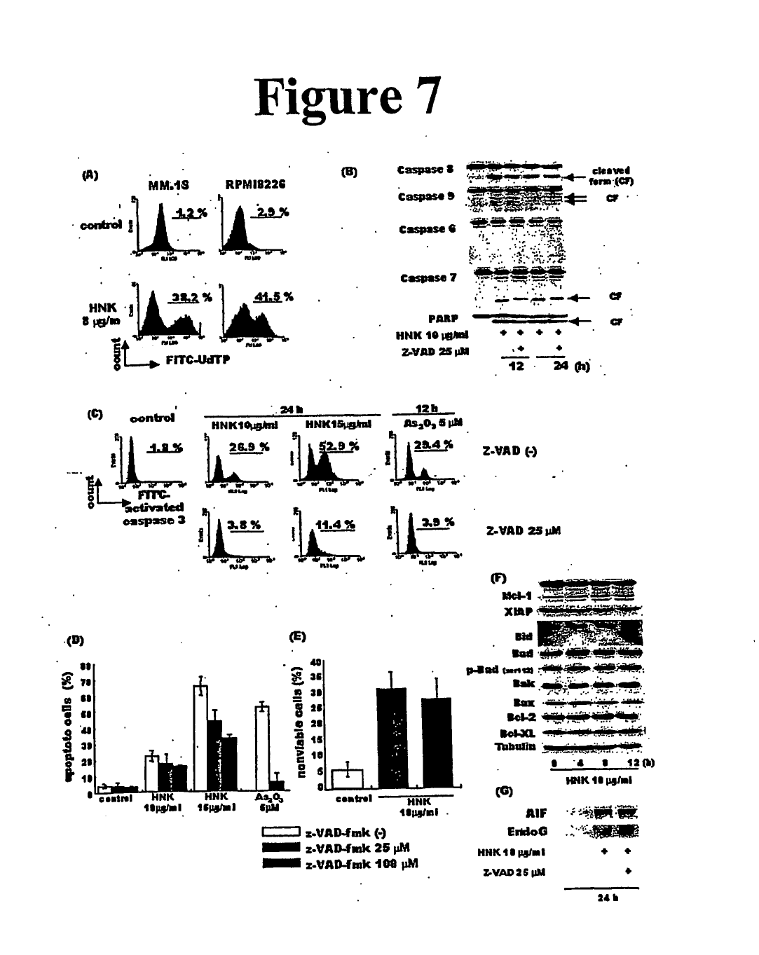

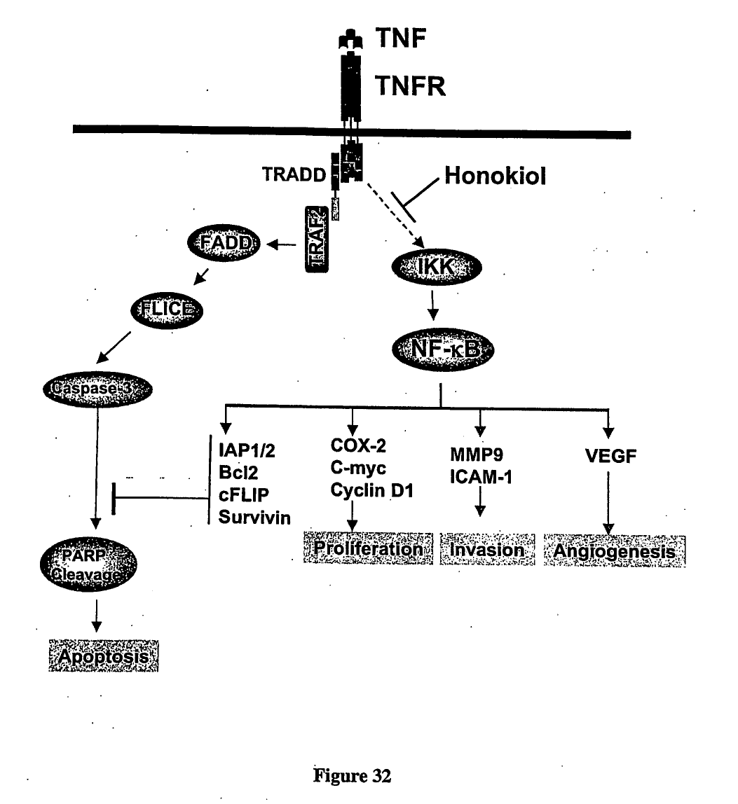

HNK induces apoptosis in MM cell lines. The cytotoxicity of HNK against MM cell lines was analyzed by evaluating the cell cycle profile of MM.1S and RPM18226 cells cultured with 10 ug/ml of HNK for 24 h. HNK treatment significantly augmented sub-G₀/G₁ cells. Moreover, treatment of MM.1S and RPM18226 cells with 10 ug/ml of HNK for 48 h induced 38.2% and 41.5% TUNEL positive cells, respectively (FIG. 7A). Treatment of MM.1S and RPM18226 cells with 10 ug/ml of HNK for 24 h induced 21.7±3.4% (FIG. 7D) and 32.9±0.6% APO2.7 positive cells, respectively, whereas 15 ug/ml HNK-treatment for 48 h did not induce APO2.7 positive cells in normal PBMNCs (n=3).

HNK induces both caspase-dependent and independent apoptosis. The apoptotic pathway induced by HNK was examined. MM.1S cells were treated with 10 ug/ml of HNK for 12 and 24 h. Protein expression of caspase 6, 7, 8, 9, and PARP was then determined by WB, and activated caspase 3 was measured using a flow cytometric assay.

FIG. 7 depicts honokiol (HNK) induced apoptosis in MM cells. A shows MM.1S and RPM18226 cells that were treated with 8 ug/ml HNK for 48 hours. Apoptosis was assessed using TUNEL assay. In B cleavage of caspases and PARP was determined by Western blotting of MM.1S whole cell lysates after 10 ug/ml HNK treatment for 12 and 24 h, with or without z-VAD-fmk (25 uM) pre-incubation for 1.5 h. C shows MM.1S cells that were treated with HNK or As₂O₃, with or without 25 uM z-VAD-fmk pre-treatment for 1.5 hours. Activation of caspase 3 was determined by flow cytometry. In 7D, MM cells were treated with HNK or As₂O₃ for 24 h, with or without 25 uM z-VAD-fmk pre-treatment for 1.5 h, and expression of APO2.7 was determined by flow cytometry. Values represent the mean ±SD of triplicate cultures. E shows the cytotoxicity as determined by trypan blue exclusion staining. Values represent the mean ±SD for 3 independent experiments. In F, MM.1S cells were treated with HNK (10 ug/ml for 0, 4, 8 and 12 h). Whole cell lysates were subjected to Western blotting to assess the expression of Bcl-2 family proteins. G shows MM.1S cells that were treated with HNK (10 ug/ml for 24 h), with or without pre-treatment by z-VAD-fmk. Proteins in cytosolic fraction were subjected to immunoblotting of AIF and EndoG.

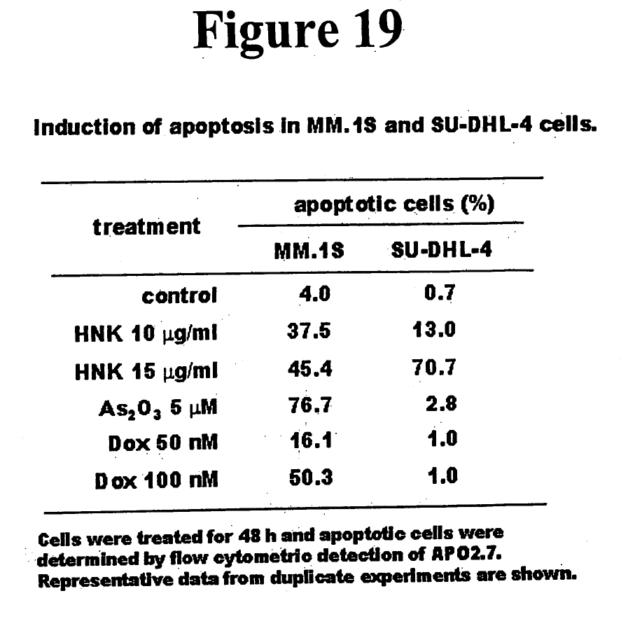

Cleavage of caspases 7, 8, 9 and PARP were induced by HNK (FIG. 7B). Activation of caspase 3 induced by HNK or As₂O₃ was completely blocked by pretreatment with 25 uM of z-VAD-fmk in MM.1S cells (FIG. 7C). However, in contrast to the complete block of As₂O₃-induced apoptosis by z-VAD-fmk, inhibition of HNK-induced apoptosis by z-VAD-fmk was only partial, evidenced by PARP cleavage and APO2.7 assay (FIGS. 7B and D). Pre-treatment with 100 uM of z-VAD-fink completely inhibited HNK-induced cleavage of caspase 7, but HNK-induced apoptosis was still observed (FIG. 7D). Moreover, cytotoxicity against MM.1S cells was not significantly reduced by z-VAD-fmk pre-treatment: the percentage of nonviable cells by trypan blue exclusion was 5.9±2.4%, 30.7±5.5%, and 27.6±6.4% in control cultures, treated with HNK 10 ug/ml for 24 h, and cultured with z-VAD-fmk 25 uM for 1.5 h followed by HNK 10 ug/ml for 24 h, respectively (FIG. 7E). Of bcl-2 family proteins, Mcl-1 was cleaved and XIAP was downregulated; Bad was markedly up-regulated; and Bid, p-Bad, Bak, Bax, Bcl-2, and Bcl-xL were unchanged after HNK treatment (FIG. 7F). HNK also induced release of mitochondrial pro-apoptotic protein AIF to cytosol (FIG. 7G). Finally, HNK also induced apoptosis in SU-DHL-4 cells, which are resistant to doxorubicin and As₂O₃-induced apoptosis (FIG. 19), without associated activation of caspase 3.

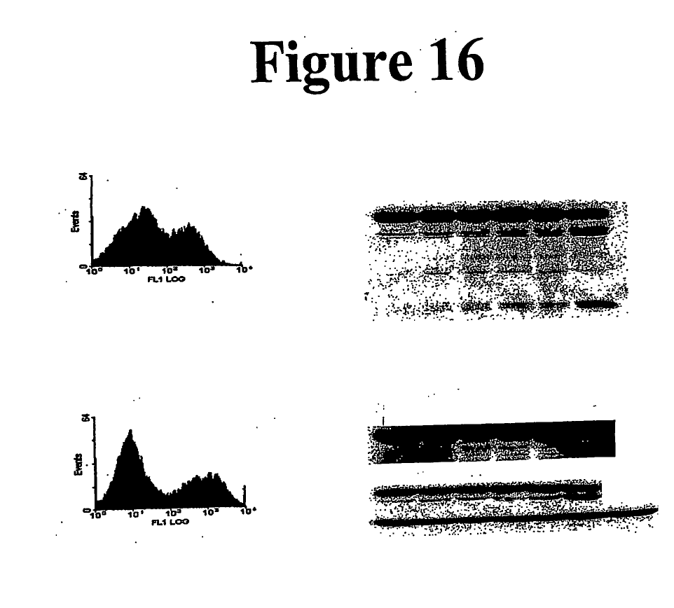

FIG. 16 provides additional data that honokiol induces apoptosis in multiple myeloma cells (MM) through caspase8/caspase9/PARP mediated apoptosis. The panel on left shows that honokiol (HNK) increases the subG1 fraction of apoptosis from control levels (1.2% in RPM18226) and (2.9% in MM.1S), to 41.5% in RPMI cells and 38.2% in MM.1s by TUNEL assay. The right panel represents Western analysis, showing that honokiol induces apoptosis through the activation of caspase8/caspase9/PARP.

HNK induced apoptosis in MM cell lines was associated with significant activation of caspase 3, 7, 8 and 9. Although pre-treatment with z-VAD-fmk almost completely inhibited HNK-induced activation of caspase 3, inhibition of HNK-induced cytotoxicity and apoptosis was only partial. In contrast, pre-treatment with z-VAD-fmk completely inhibited both caspase 3 activation and apoptosis in MM.1S cell induced by As₂O₃. HNK also induced apoptosis in caspase 3 deficient MCF-7 cells. Caspase 7, which is an executioner caspase in MCF-7 cells (Janicke R U et al J Biol Chem. 1998; 273:9357-9360; Fattman et al Oncogene. 2001; 20:2918-2926; Mc Gee M M et al FEBS Lett. 2002; 515:66-70), was also cleaved in HNK-treated MM.1S cells. These results indicate that HNK induces apoptosis in both caspase 3-dependent and independent pathway.

Bad, a proapoptotic Bcl-2 family member protein, can displace Bax from binding to Bcl-2 and Bcl-xL, thereby promoting apoptosis (Zha et al Cell. 1996; 87:619-628). On the other hand, phosphorylated Bad prevents the binding of Bad to Bcl-2 and Bcl-xL, thereby inhibiting induction of apoptosis (Zha et al J Biol Chem. 1997; 272:24101-24104; Yan et al J Biol Chem. 2003; 278:45358-45367). In this study, HNK significantly enhanced Bad expression with modest phosphorylation, but did not significantly change Bcl-2, Bcl-xL, Bax, and Bid. The expression of XIAP was decreased and Mcl-1 was cleaved during HNK-induced apoptosis. XIAP is a well-characterized IAP family member in terms of its caspase inhibitory mechanism (Chawla-Sarkar et al Cell Death Differ. 2004; 11:915-923). Although, XIAP is negatively regulated by nuclear factor (NF)-κB (Mitsiades et al Blood. 2002; 99:4079-4086), phoshorylation IκBα and p65 NF-κB were not modulated in MM.1S cells by HNK. Mcl-1 is an anti-apoptotic member of Bcl-2 family; cleavage of Mcl-1 by caspases yields cleaved Mcl-1 which counteracts function of residual intact Mcl-1 (Herrant et al Oncogene. 2004; 23:7863-7873). Taken together, these results suggest that HNK induces apoptosis via both extrinsic pathway with caspase 8 activation and intrinsic pathway, due to enhanced Bad expression leading to activation of mitochondrial apoptotic pathway. Moreover, drug-induced down-regulation of XIAP prevents the inhibition of effecter caspases; and conversely, activation of caspases is further enhanced by cleaved Mcl-1.

Most drugs used to treat MM as well as other malignant diseases induce tumor cell death by activation of caspases and apoptosis. However, recent studies suggest that caspase activation is not the sole pathway for inducing apoptosis by death stimuli (Jaattela et al. Nat Immunol. 2003; 4:416-423; Abraham et al Trends Cell Biol. 2004; 14:184-193; Lockshin et al Oncogene. 2004; 23:2766-2773). Caspase-independent apoptosis in vitro can be induced by the following clinically available drugs: acute myelogenous leukemia cells treated with cytosine arabinoside or paclitaxel (Carter et al Blood. 2003; 102:4179-4186); B-lymphoid cell lines and chronic lymphocytic leukemia cells treated with rituximab and alemtuzuniab (Stangimaier et al Ann Hematol. 2004); BCR-ABL-positive human leukemic cells treated with imatinib mesylate (Okada et al Blood. 2004; 103:2299-2307); and ovarian carcinoma cell lines treated with taxol (Ahn et al J Cell Biochem. 2004; 91:1043-1052). In MM, apoptosis in arsenic RPM18226 cells and patient MM cells induced by As₂O₃ is caspase-independent (McCafferty-Grad et al Mol Cancer Ther. 2003; 2:1155-1164). HNK also induces apoptosis SU-DHL4 cells, which express low levels of caspase-8 and -3. the HNK-induced caspase-independent apoptotic pathway was further examined. Several molecular pathways to induce caspase-independent AIF/Endo G pathway (Ahn et al J Cell Biochem. 2004; 91:1043-1052; Penninger et al Nat Cell Biol. 2003; 5:97-99; Joza et al. Nature. 2001; 410:549-554; Daugas et al. Faseb J. 2000; 14:729-739; Cregan et al. Oncogene. 2004; 23:2785-2796; Cande et al Cell Death Differ. 2004; 11:591-595). In this pathway, death stimuli induce release of AIF and/or Endo G from mitochondria to the cytosol and nucleus, with subsequent chromatin condensation and cell death. During HNK induced apoptosis AIF, but not Endo G, was significantly released from mitochondria to cytosol. There are few reports that apoptosis induced via AIF/Endo G pathway can also be caspase-dependent by showing the release of AIF and Endo G from mitochondria is blocked by z-VAD-fmk (Cande et al Cell Death Differ. 2004; 11:591-595; Arnoult et al Embo J. 2003; 22:4385-4399). The release of AIF protein was caspase-independent, since HNK effects on AIF were not blocked by z-VAD-fmk. Finally, since pre-treatment with serine protease inhibitor 4-(2-aminoethyl)benzenesulfonyl fluoride (AEBSF) did not inhibit HNK induced apoptosis, thus caspase-dependent and -independent cell death pathway induced by serine protease activity (Okada et al. Blood. 2004; 103:2299-2307; de Bruin et al. Cell Death Differ. 2003; 10:1204-1212; Liu et al. FEBS Lett. 2004; 569:49-53) is not likely to mediate HNK-induced apoptosis.

These results indicate HNK induces apoptosis in MM cells via both caspase-dependent and -independent pathways. HNK induces apoptosis in SU-DHL4 cells, which express low levels of caspase-8 and -3 and are resistant to doxorubicin, As₂O₃, melphalan, dexamethasone, bortezomib (Chauhan et al. Cancer Res. 2003; 63:6174-6177), and revlimid. Therefore, agents such as HNK which kill MM cells via both caspase-dependent and caspase-independent pathways may be particularly useful to overcome drug resistance.

Combined with HNK and Bortezomib Augments Inhibition MM.1S Cell Growth.

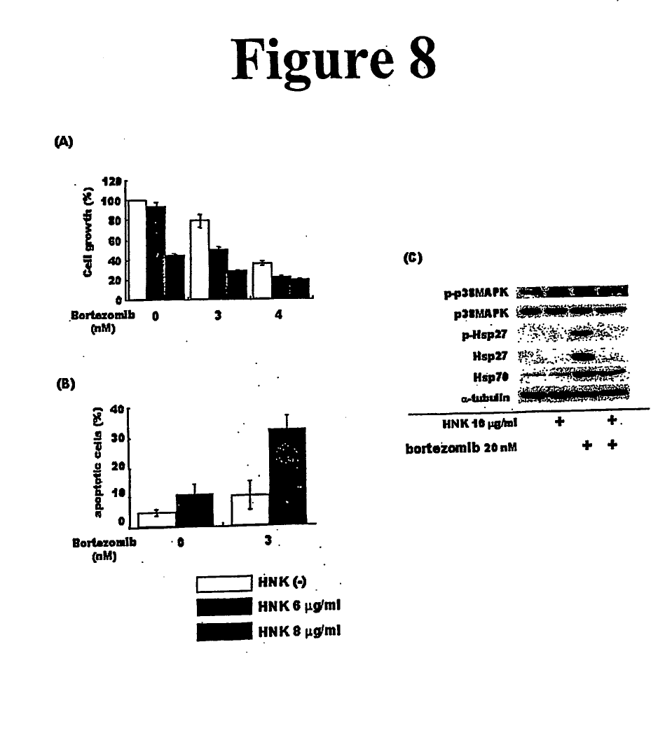

Combined treatment of MM.1S cells with HNK and bortezomib enhanced the cytotoxicity and induction of apoptosis compared to each drug alone (FIGS. 8A and B). In FIG. 8A, MM.1S cells were treated with HNK and bortezomib for 48 h and cell growth was determined by colorimetric assay. Values represent the mean ±SD of triplicate cultures. FIG. 8B shows MM.1S cells that were treated with HNK and bortezomib and induction of apoptosis was determined by APO2.7. Values represent the mean ±SD of two independent cultures. To elucidate the mechanism of the enhanced cytotoxicity of combined HNK and bortezomib, MM.1S cells were treated with HNK for 8 h, alone and together with bortezomib. Bortezomib-induced up-regulation of Hsp27, p-Hsp27 and Hsp70 was significantly blocked by HNK (FIG. 8C). In FIG. 8C, MM.1S cells were treated with HNK and bortezomib for 8 h. Whole cell lysates were subjected to Western blotting to assess phosphorylation and protein expression of p38MAPK, Hsp27 and Hsp70.

The combination of HNK with bortezomib enhanced cytotoxicity and induction of apoptosis, compared to either drug alone. Hsp27 and Hsp70 are upregulated after bortezomib treatment in MM cells (Mitsiades et al Proc Natl Acad Sci USA. 2002; 99:14374-14379; Hideshima et al Blood. 2003; 101:1530-1534); since Hsps inhibit apoptotic signaling at several levels (Creagh et al Leukemia. 2000; 14:1161-1173; Jolly et al J Natl Cancer Inst. 2000; 92:1564-1572; Xanthoudakis et al Nat Cell Biol. 2000; 2:E163-165.), this upregulation of Hsps in PS-341-treated MM cells induces resistance to bortezomib-induced apoptosis. HNK significantly downregulated bortezomib-induced expression of Hsp27 and Hsp70, thereby enhancing cytotoxicity of bortezomib.

Effect of HNK on MM Cells Cultured with Exogenous IL-6, IGF-1 and BMSCs.

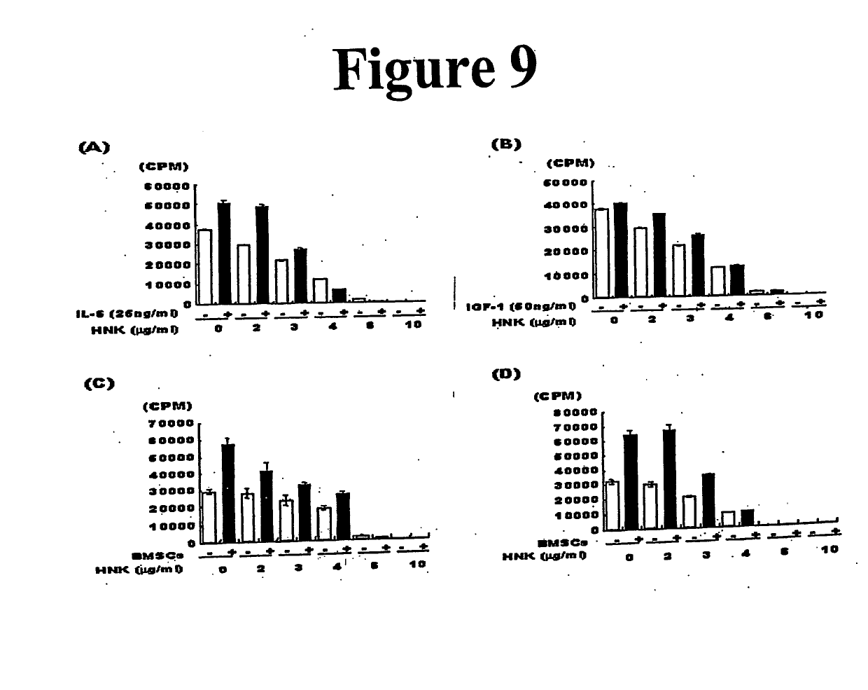

The effect of HNK on MM cells in the presence of exogenous IL-6 and IGF-1, as well as BMSCs was evaluated. Neither IL-6 nor IGF-1 protected against HNK induced growth inhibition (FIGS. 9A and B). FIG. 9 shows that HNK can overcome the protective effects of IL-6, IGF-1 and adherence to patient BMSCs. MM.1S cells were treated for 48 h with indicated concentrations of HNK in the presence or absence of IL-6 (shown in A), IGF (shown in B) or BMSCs derived from 2 mM patients (shown in C and D). DNA synthesis was determined by measuring [³H]-thymidine incorporation during the last 8 h of 48 h cultures. Values represent the mean ±SD of triplicate cultures. Binding of MM cells to BMSCs derived from 2 mM patients triggers DNA synthesis, which was also abrogated by HNK (FIGS. 9C and D). Importantly, at similar concentrations HNK did not affect the viability of BMSCs, as determined by colorimetric assay.

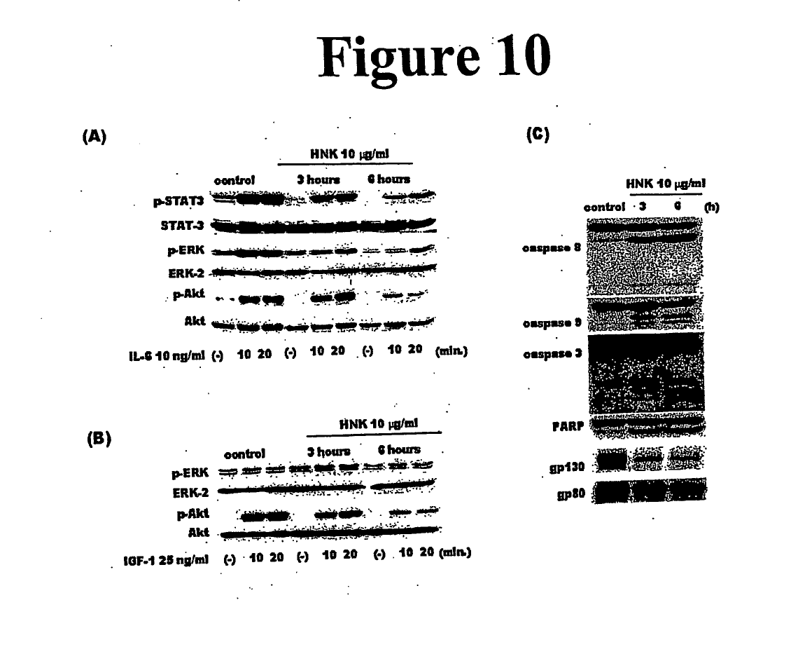

To further delineate the effect of HNK on growth signaling, MM.1S cells were stimulated by IL-6 (10 ng/ml) or IGF-1 (25 ng/ml) for 10 and 20 min following pre-treatment with 10 ug/ml of HNK for 3 and 6 h in 2.5% FCS. HNK significantly reduced phosphorylation of STAT-3, ERK and Akt induced by IL-6, as well as ERK and Akt induced by IGF-1 (FIGS. 10A and B). FIG. 10A shows MM.1S cells that were pretreated with FINK (10 ug/ml) in FCS 2.5% containing media for 3 and 6 h, cells were then stimulated with IL-6 (10 ng/ml) for 10 and 20 min. Whole cell lysates were subjected to Western blotting to assess phosphorylation and protein expression of STAT3, ERK1/2, and Akt. In FIG. 10B, MM.1S cells were pretreated with HNK (10 ug/ml) in FCS 2.5% containing media for 3 and 6 h, and then stimulated with IGF-1 (25 ng/ml) for 10 and 20 min. Whole cell lysates were subjected to Western blotting for phosphorylation and protein expression of ERK1/2 and Akt. Downregulation of gp130 and gp80 were also observed after HNK-treatment (FIG. 10C). FIG. 10C shows MM.1S cells that were pretreated with HNK (10 ug/ml) in FCS 2.5% containing media for 3 and 6 h. Whole cell lysates were subjected to Western blotting to determine cleavage of caspases and expression of gp80 and gp130.

Since the BM microenvironment confers drug resistance in MM cells (Damiano et al Blood. 1999; 93: 1658-1667), the BM microenvironment was mimicked. The effect of exogenous IL-6, IGF-1 and co-culture of MM cells with BMSCs on HNK cytotoxicity was studied. Adherence to BMSCs, IL-6 or IGF-1 did not protect against HNK-induced MM cell death. HNK triggered modulation of signaling pathways induced by IL-6 and IGF-1 were also further elucidated. STAT-3, ERK and Akt signaling induced by IL-6 as well as ERK and Akt signaling triggered by IGF-1, were blocked by HNK. Downregulation of the cytoplasmic domain of IL-6 receptor gp130 by activated caspases during bortezomib-treated apoptosis in MM cells was reported (Hideshima et al Oncogene. 2003; 22:8386-8393). Downregulation of gp130 as well as gp80, was also observed in HNK-treated cells, which thereby abrogates IL-6-induced signaling.

### HNK Inhibits Angiogenesis of HUVEC.

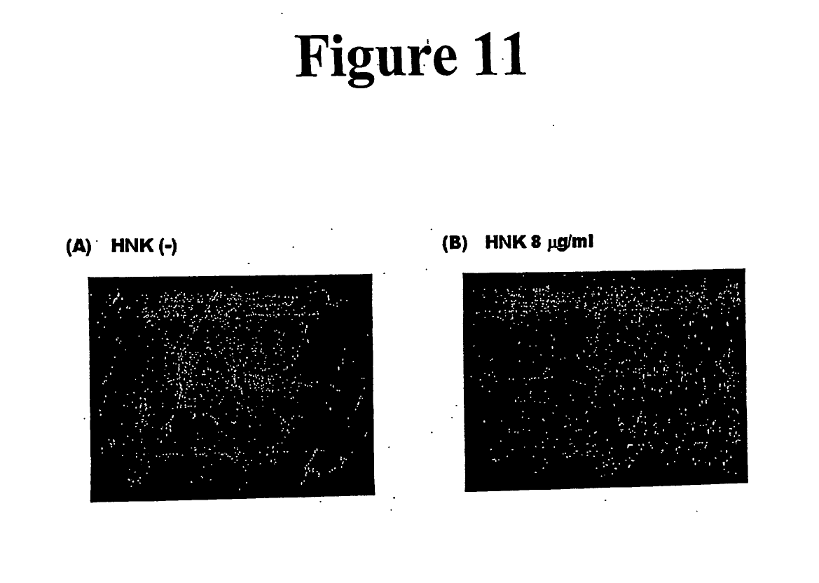

HUVEC were cultured with 8 ug/ml of HNK for 6 h, and tube formation by endothelial cells was evaluated. HNK significantly inhibited the tube formation (FIGS. 11A and B), but at this concentration did not affect the viability of HUVEC cells. FIG. 11 depicts HNK inhibition of angiogenesis of HUVEC. HUVEC were cultured with (depicted in B) or without (depicted in A) 8 ug/ml of HNK for 6 h, and tube formation was assessed. Original magnification is ×40.

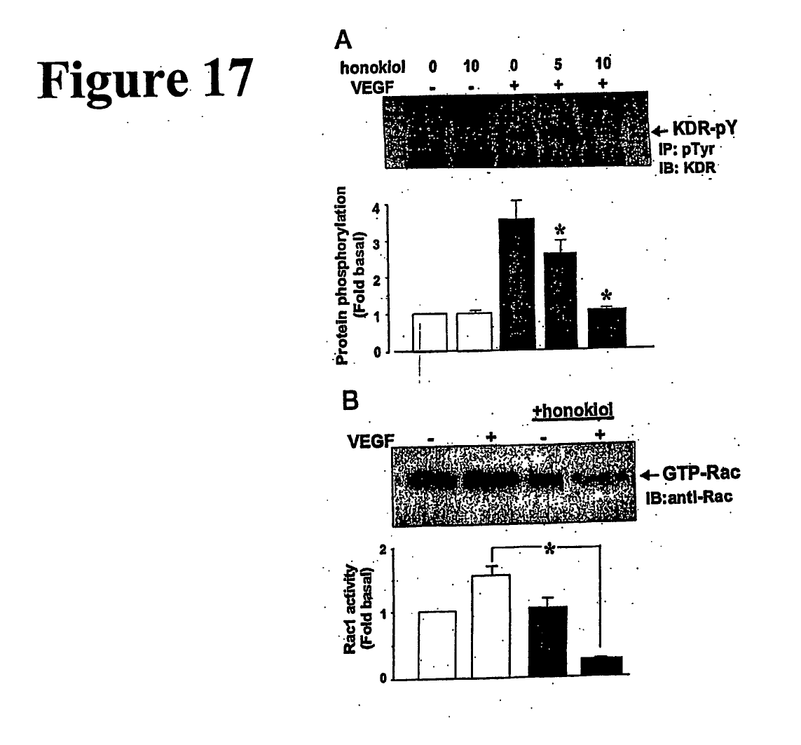

FIG. 17A also demonstrates effect of honokiol on VEGF-induced KDR autophosphorylation in HUVECs. HUVECs were preincubated with vehicle or honokiol (5 and 10 μg/ml) for 60 min and then stimulated with 20 ng/ml VEGF for 5 min. Lysates were immunoprecipitated (IP) with anti-phosphotyrosine (pTyr) antibody followed by immunoblotting (IB) with anti-KDR antibody (top panel). Bottom panel represents averaged data expressed as fold change over basal (the ratio in untreated cells was set to 1). Values are the means ±S.E. for three independent experiments. *, p<0.05 for increase in phosphorylation by VEGF in the presence of inhibitor versus VEGF alone. In FIG. 17B, the effect of honokiol on VEGF-induced Rae activation was analyzed. HUVECs were preincubated with vehicle or honokiol (10 μg/ml) for 60 min and then stimulated with 20 ng/ml VEGF for 3 min. Rae activity was measured by p21-activated kinase-1-protein binding domain affinity precipitation Top, representative immunoblot of GTP-bound Rac. Bottom, densitometric analysis (mean ±S.E.) of immunoblots from three experiments expressed as fold increase over control. *, p<0.01 compared with VEGF alone. This data indicates that honokiol acts as a direct inhibitor of angiogenesis in addition to its antitumor activities.

Anti-angiogenesis activity of HNK, evidenced by blocking of VEGF-induced VEGF receptor 2 autophosphorylation and growth inhibition in HUVEC, has been reported (Bai et al J Biol Chem. 2003; 278:35501-35507). In this study, it was also shown that sub-toxic doses of HNK induced inhibition of tube formation of HUVEC, suggesting that HNK inhibits vascular formation in the BM microenvironments.

### Example 4

### In Vivo Effects of Honokiol

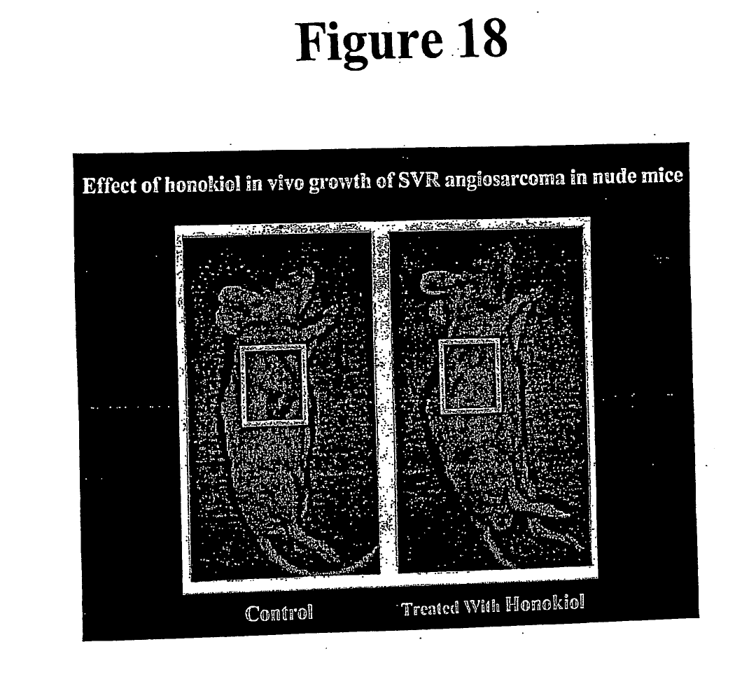

FIG. 18 illustrates the effect of honokiol on in vivo growth of SVR angiosarcoma in nude mice. This data shows that honokiol is effective against tumors in vivo and is nontoxic to the host animal.

### Example 5

### Functional Analysis of Honokiol Analog Candidates Against Biological Targets, Including AMPK, PLD, and NFkB

### Cellular Proliferation Assay

The SVR (a transformed endothelial cell line) proliferation assay can be used as a direct measure of antiangiogenic and antitumor activity. This assay serves as a high throughput screen that compares the effects of a compound on proliferation of SVR cells versus an immortalized endothelial cell line, MS1. Compounds that have an IC₅₀ of 10 μM in this assay can be considered active. Compounds that show activity in this initial assay can be tested for their ability to preferentially inhibit endothelial proliferation versus fibroblast proliferation using primary human endothelial cells and fibroblasts, as previously demonstrated with honokiol (Bai, X. et al. (2003) J. Biol. Chem. 278, 35501-35507).

SVR cells were plated in 24-well dishes. The next day, the medium was replaced with fresh medium containing the inhibitors or vehicle controls. Cells were incubated at 37° C. for 72 h (Arbiser, J. L., et al. 1999 J. Am. Acad. Dermatol. 40, 925-929; LaMontagne, K. R., et al. 2000 Am. J. Pathol. 157, 1937-1945), and cell number was determined in triplicate using a Coulter Counter (Hialeah, Fla.). Immortalized and K-Ras transformed rat epithelial cells (RIEpZip and RIEpZipK-Ras12V) and fibroblasts (NIH3T3 pZip and NIH3T3 pZipK-Ras12V) were maintained at 37° C., 10% CO₂, in Dulbecco's modified Eagle's medium supplemented with 5% fetal calf serum (RIE) or 10% calf serum (NIH3T3) (Oldham, S. M., et al. 1996 Proc. Natl. Acad. Sci. U.S.A. 93, 6924-6928; Pruitt, K., et al. 2000 J. Biol. Chem. 275, 40916-40924). Cells were plated at 10⁵/well in six-well plates. Vector and Ras-transformed NIH3T3 and RIE cells were treated with either vehicle (20 μl of Me₂SO) or increasing concentrations (5, 10, 20, and 40 μg/ml) of honokiol (from a 2 mg/ml Me₂SO stock) and observed for morphology changes after 24 h.

### Stimulation of AMPK Activation

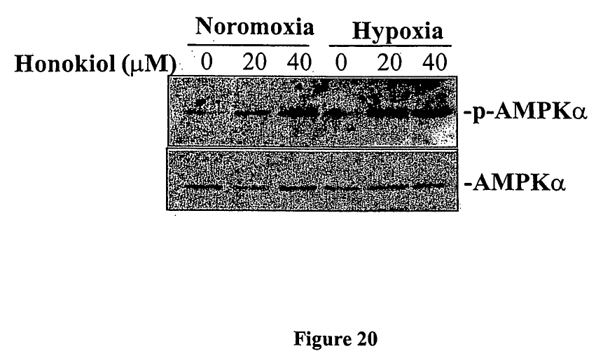

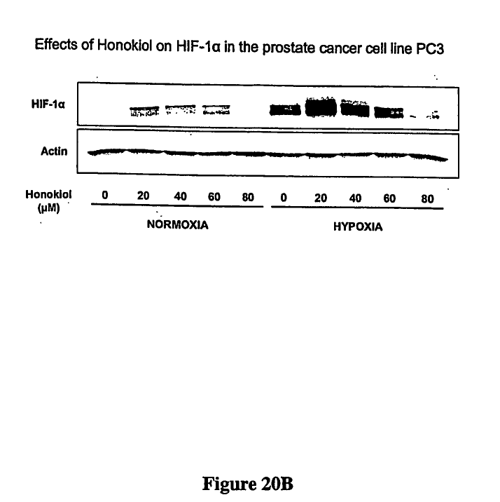

Honokiol can activate AMPK (FIG. 20), which has been shown to decrease proliferation of tumor cells through both p53 dependent and independent pathways (Jones, R. G. et al (2005) Molecular Cell 18, 283-293; Bharti, A. C. et al (2004) Blood 103, 3175-3184; Arbiser, J. L et al (1998) Mol. Med. 4, 376-383; Woods, A. et al (2003) Current Biology 13, 2004-2008; Shaw, R. J. et al (2004) PNAS 101, 3329-3335; Buzzai, M. et al (2005) Oncogene 24, 4165-4173). As shown in FIG. 20, PC3 cells were treated with honokiol under normoxic and hypoxic conditions. The top blot shows increased phosphorylation (activation) of AMP kinase by honokiol. The bottom blot shows total AMP kinase protein, serving as a loading control. Honokiol activated HIF-1a in prostate cancer cells in a dose dependent manner, as shown in FIG. 20b. PC3 cells were treated with honokiol in normoxia (left) or hypoxia (right). In both cases, HIF-1a induction is dose dependent, and in the case of hypoxia, at least additive.

Compounds can be tested on the p53 deficient PC3 human prostate cancer cell line, to see whether treatment results in activation of AMPK as in FIG. 20. PC3 cells can be treated with compound or vehicle for 24 hours, then proteins harvested and analyzed by Western blot for phosphorylation of the alpha subunit of AMPK, a marker of AMPK activation. In addition, phosphorylation of a substrate of AMPK, acetyl CoA carboxylase (ACC) can be monitored by Western blot. If a compound causes phosphorylation of AMPK and ACC, the ability of the compound to stimulate AMPK activity directly can be assessed by adding the compound to an AMPK enzymatic assay as described by Winder and Hardie ((1996) American Journal of Physiology-Endocrinology and Metabolism 33, E299-E304). In addition, dominant negative AMPK cells can be used to test the ability of honokiol and honokiol analogs to inhibit the proliferation of these cells (Jones, R. G. et al (2005) Molecular Cell 18, 283-293.) Honokiol does not directly activate heart AMPKK in vitro. Honokiol potentiates glucose uptake by insulin, similar to adiponectin, in rat papillary muscles.

### Inhibition of Phospholipase D Activity

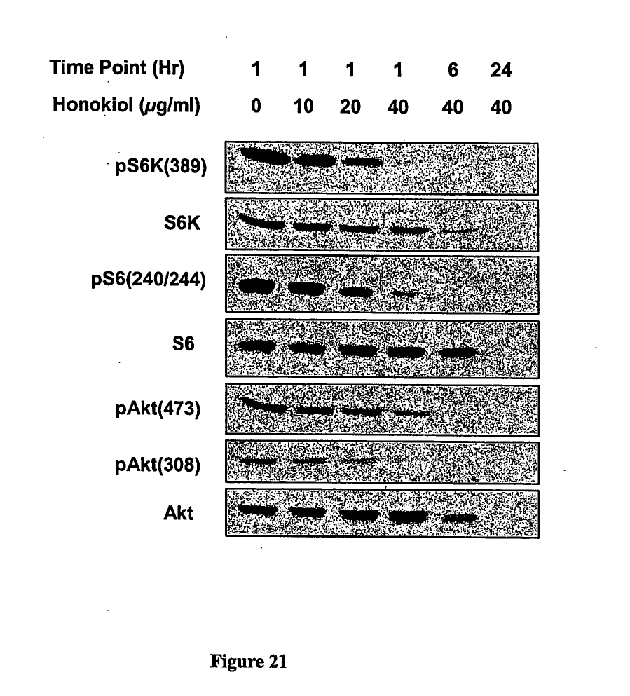

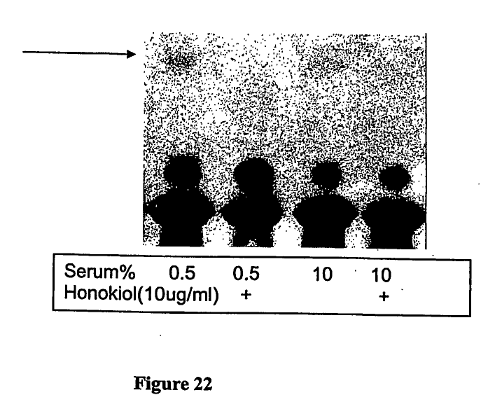

Honokiol analogs can stimulate tumor and endothelial cell apoptosis through inhibition of PLD activity. Preliminary data shows that SVR cells, especially under serum free conditions, express high levels of PLD and thus serve as an excellent assay of PLD activity (FIGS. 21, 22). FIG. 21 shows that honokiol can mimic the effect of wild type tuberin. Treatment with tuberin causes downregulation of S6kinase phosphorylation in a time and dose dependent fashion, as well as downregulation of akt. Thus, honokiol mimics several of the activities of wild type tuberin. FIG. 22 shows that honokiol inhibits the activity of phospholipase D in both 0.5% and 10% serum in SVR cells.

Cells can be treated for 24 hours with honokiol analogs, and lipids can be extracted according to the methods of Foster et al ((2001) Biochemical and Biophysical Research Communications 289, 1019-1024). Compounds that show inhibitory activity against PLD can be tested for their ability to inhibit downstream activation of PLD targets such as mTOR, S6 kinase, and S6 (FIG. 21).

Inhibition of NFkB Activation

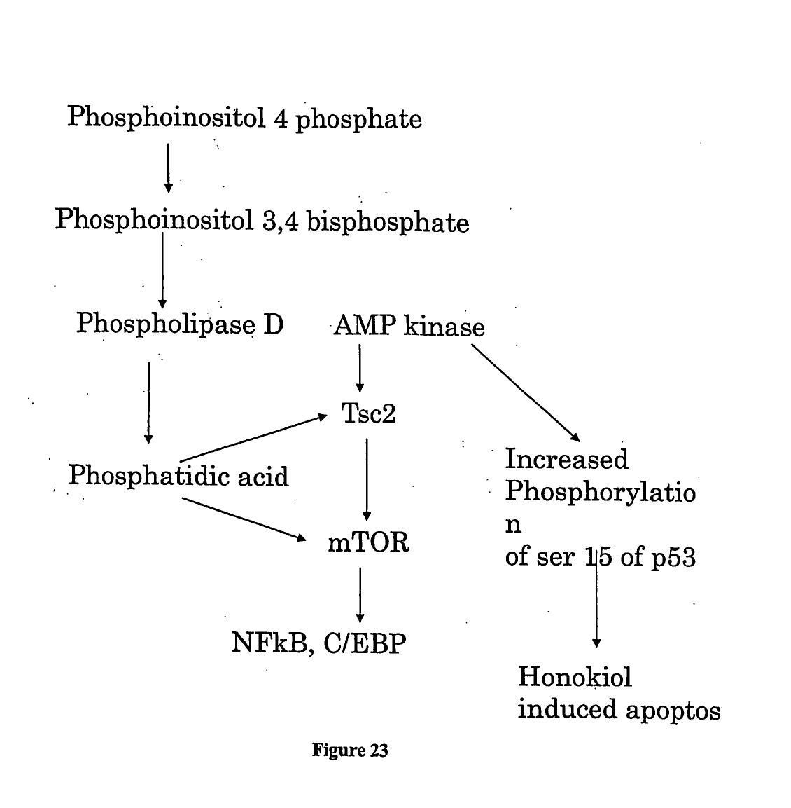

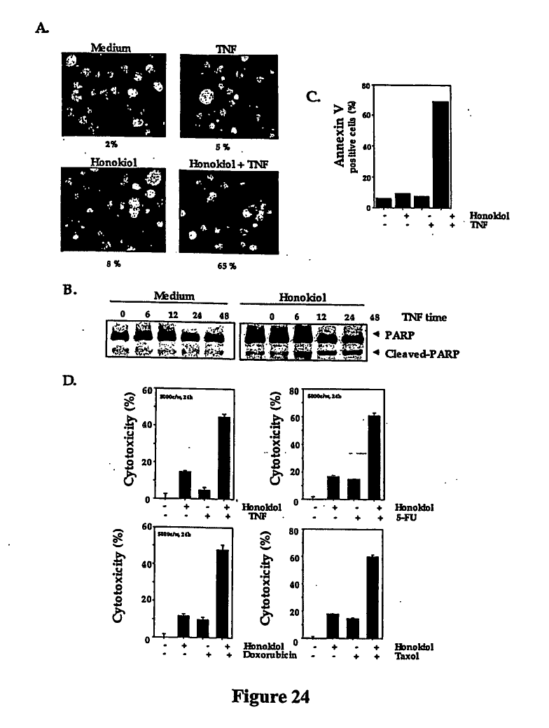

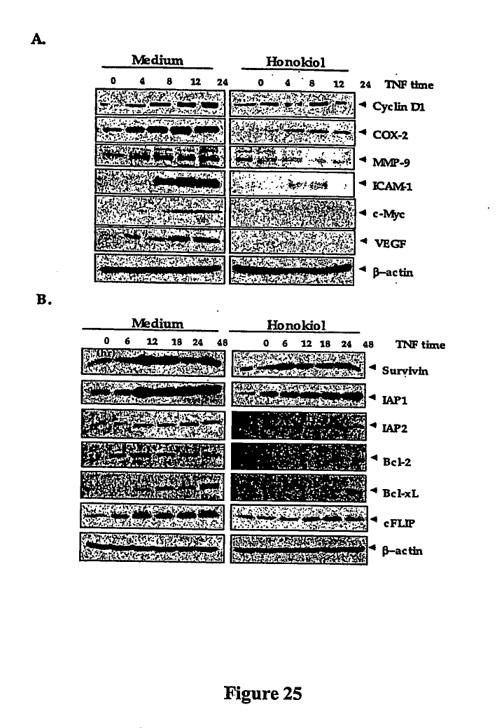

NFkB is a major survival mechanism of many tumor cells, including multiple myeloma. Honokiol can augment the activity of velcade, possibly through inhibition of NFkB through velcade independent pathways. FIGS. 23 and 24 show that honokiol blocks NFkB activation and sensitizes tumor cells to conventional chemotherapeutic agents._ As a rapid assay of honokiol activity on NFkB, the phosphorylation of IkBα can be examined. Phosphorylation of IkBα is reduced by honokiol treatment. Cells can be treated with honokiol analogs, and lysates can be prepared after 24 hours incubation. Western blot analysis of both total and phosphorylated p65 can be carried out by standard protocols (Singh, S. & Aggarwal, B. B. (1995) Journal of Biological Chemistry 270, 24995-25000; Bharti, A. C., Shishodia, S., Reuben, J. M., Weber, D., Alexanian, R., Raj-Vadhan, S., Estrov, Z., Talpaz, M. & Aggarwal, B. B. (2004) Blood 103, 3175-3184.

### Example 6

### Anti-HIV-1 Activity in PBM Cells and Cytotoxicity Assays of Honokiol and Honokiol-Like Compounds

### HIV Assay in PBM Cells

The antiviral activity of the synthesized compounds and honokiol were evaluated against HIV-1 in human peripheral blood mononuclear (PBM) cells (Table 4).

TABLE 4

| | Activity anti-VIH-1 on | | Cytotoxicity | | |
|---|---|---|---|---|---|
| | PBM cells | | (EC₅₀-μM) in: | | |
| Formula | EC₅₀, μM | EC₉₀, μM | PBM | CEM | VERO |
| AZT | 0.014 | 0.049 | >100 | 14.3 | 50.6 |
| 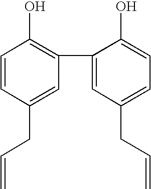 | 69.3 | >100 | 38.6 | 99.6 | 79.9 |
| 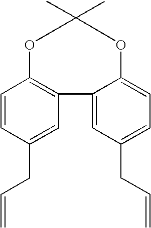 | 13.6 | 43.3 | 75.3 | 11.6 | >100 |
| 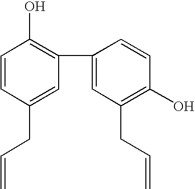 | 3.3 | 22.7 | 16.1 | 10.9 | 22.5 |
| 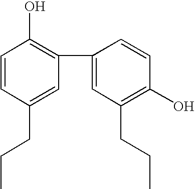 | 16.9 | 69.4 | 45.9 | 10.4 | 19.8 |
| 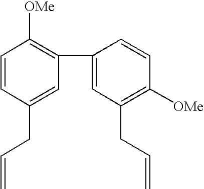 | 54.3 | >100 | >100 | 19.1 | >100 |
| 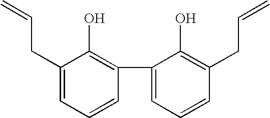 | 34.8 | 63.0 | >100 | >100 | >100 |
| 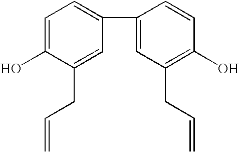 | 19.4 | 66.4 | >100 | 23.0 | 33.2 |

Human peripheral blood mononuclear (PBM) cells (which can be obtained from Atlanta Red Cross) can be isolated by Ficoll-Hypaque discontinuous gradient centrifugation from healthy seronegative donors. Cells can be stimulated with phytohemagglutinin A (Difco, Sparks, Md.) for 2-3 days prior to use. HIV-1, such as HIV-1_{LA}I can be obtained from the Centers for Disease Control and Prevention (Atlanta, Ga.), and can be used as the standard reference virus for the antiviral assays. The molecular infectious clones HIV-1_{xxBru} and HIV-1_{M184Vpin} can be obtained from Dr. John Mellors (University of Pittsburgh). Infections can be done in bulk for one hour, either with 100 TCID₅₀/1×10⁷ cells for a flask (T25) assay or with 200 TCID₅₀/6×10⁵ cells/well for a 24 well plate assay. Cells can be added to a plate or flask containing a ten-fold serial dilution of the test compound. Assay medium can be RPMI-1640 supplemented with heat inactivated 16% fetal bovine serum, 1.6 mM L-glutamine, 80 IU/ml penicillin, 80 μg/ml streptomycin, 0.0008% DEAE-Dextran, 0.045% sodium bicarbonate, and 26 IU/ml recombinant interleukin-2 (Chiron Corp, Emeryville, Calif.). AZT can be used as a positive control for the assay. Untreated and uninfected PBM cells can be grown in parallel at equivalent cell concentrations as controls. The cell cultures can be maintained in a humidified 5% CO₂-air at 37° C. for 5 days and supernatants can be collected for reverse transcriptase (RT) activity.

Supernatants can be centrifuged at 12,000 rpm for 2 hours to pellet the virus. The pellet can be solubilized with vortexing in 100 μl virus solubilization buffer (VSB) containing 0.5% Triton X-100, 0.8 M NaCl, 0.5 mM phenylmethylsulfonyl fluoride, 20% glycerol, and 0.05 M Tris, pH 7.8. Ten μL of each sample can be added to 75 μL RT reaction mixture (0.06 M Tris, pH 7.8, 0.012 M MgCl₂, 0.006 M dithiothreitol, 0.006 mg/ml poly (rA)ₙ, oligo (dT)₁₂₋₁₈, 96 μg/ml dATP, and 1 μM of 0.08 mCi/ml ³H-thymidine triphosphate (Moravek Biochemicals, Brea, Calif.) and can be incubated at 37° C. for 2 hours. The reaction can be stopped by the addition of 100 μL 10% trichloroacetic acid containing 0.05% sodium pyrophosphate. The acid insoluble product can be harvested onto filter paper using a Packard Harvester (Meriden, Conn.), and the RT activity can be read on a Packard Direct Beta Counter (Meriden, Conn.). The RT results can be expressed in counts per minute (CPM) per milliliter. The antiviral 50% effective concentration (EC₅₀) and 90% effective concentration (EC₉₀) can be determined from the concentration-response curve using the median effect method (Belen'kii, S. M.; Schinazi, R. S. Multiple drug effect analysis with confidence interval. Antiviral. Res. 1994, 25, 1-11).

### Cytotoxicity Assays

The cytotoxicity of the honokiol and analogues thereof were assessed in human PBM, CEM and Vero cells (Table 4).

Compounds can be evaluated for their potential toxic effects on uninfected human PBM cells, in CEM (T-lymphoblastoid cell line obtained from American Type Culture Collection, Rockville, Md.) and Vero (African green monkey kidney) cells. PBM cells can be obtained from whole blood of healthy seronegative donors (HIV-1) by single-step Ficoll-Hypaque discontinous gradient centrifugation. Log phase Vero, CEM and PBM cells can be seeded at a density of 5×10³, 2.5×10³ and 5×10⁴ cells/well respectively. All of the cells can be plated in 96-well cell culture plates containing ten-fold serial dilutions of the test drug. The cultures can be incubated for 3, 4 and 5 days for Vero, CEM, and PBM cells, respectively in a humidified 5% CO₂-air at 37° C. At the end of incubation, MTT tetrazolium dye solution (Cell titer 96®, Promega, Madison, Wis.) can be added to each well and incubated overnight. The reaction can be stopped with stop solubilization solution (Promega, Madison, Wis.). The plates can be incubated for 5 hours to ensure that the formazan crystals can be dissolved. The plates can be read at a wavelength of 570 nm using an ELISA plate reader (Bio-tek instruments, Inc., Winooski, Vt., Model #EL 312e). The 50% inhibition concentration (IC₅₀) can be determined from the concentration-response curve using the median effect method.

### Example 7

### Cellular Proliferation Assay of Honokiol Analogs

Cellular Proliferation Assay

The SVR (a transformed endothelial cell line) proliferation assay can be used as a direct measure of antiangiogenic and antitumor activity. This assay serves as a high throughput screen that compares the effects of a compound on proliferation of SVR cells versus an immortalized endothelial cell line, MS1. Compounds that have an IC₅₀ of 10 μM in this assay can be considered active. Compounds that show activity in this initial assay can be tested for their ability to preferentially inhibit endothelial proliferation versus fibroblast proliferation using primary human endothelial cells and fibroblasts, as previously demonstrated with honokiol (Bai, X. et al. (2003) J. Biol. Chem. 278, 35501-35507). Results of this assay for several compounds are shown in Table 5.

SVR cells were plated in 24-well dishes. The next day, the medium was replaced with fresh medium containing the inhibitors or vehicle controls. Cells were incubated at 37° C. for 72 h (Arbiser, J. L., et al. 1999 J. Am. Acad. Dermatol. 40, 925-929; LaMontagne, K. R., et al. 2000 Am. J. Pathol. 157, 1937-1945), and cell number was determined in triplicate using a Coulter Counter (Hialeah, Fla.). Immortalized and K-Ras transformed rat epithelial cells (RIEpZip and RIEpZipK-Ras12V) and fibroblasts (NIH3T3 pZip and NIH3T3 pZipK-Ras12V) were maintained at 37° C., 10% CO₂, in Dulbecco's modified Eagle's medium supplemented with 5% fetal calf serum (RIE) or 10% calf serum (NIH3T3) (Oldham, S. M., et al. 1996 Proc. Natl. Acad. Sci. U.S.A. 93, 6924-6928; Pruitt, K., et al. 2000 J. Biol. Chem. 275, 40916-40924). Cells were plated at 10⁵/well in six-well plates. Vector and Ras-transformed NIH3T3 and RIE cells were treated with either vehicle (20 μl of Me₂SO) or increasing concentrations (10 or 15 μg/ml) of honokiol or the compound of interest (from a 2 mg/ml Me₂SO stock) and observed for morphology changes after 24 h.

TABLE 5

| | | % | % | |
|---|---|---|---|---|
| | Molecular | inhibition | inhibition | |
| | weight | at 10 | at 15 | |
| Compound | (g/mol) | μg/mL | μg/mL | Mice |
| 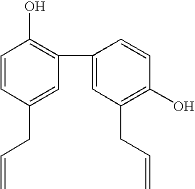 | 266 | 59.85 | 85.04 | |
| 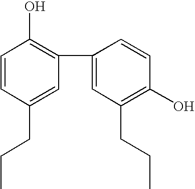 | 270 | 79.16 | 89.56 | |
| 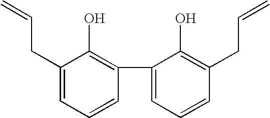 | 266 | 40.77 | 57.22 | |
| 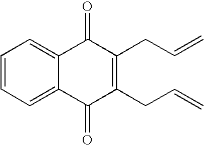 | 238 | 87.55 | 91.46 | Toxic |
| 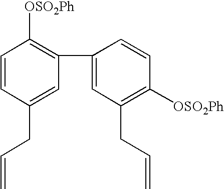 | 546 | 35.29 | 37.90 | |
| 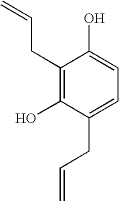 | 190 | 37.21 | 35.96 | |
| 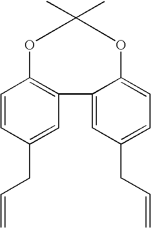 | 306 | 61.85 | 59.73 | |
| 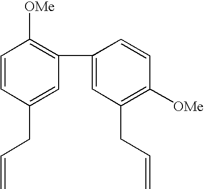 | 294 | 40.56 | 47.19 | |
| 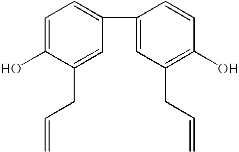 | 266 | 44.86 | 56.2 | Toxic |
| 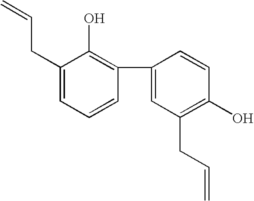 | 266 | 74.17 | 81.64 | |
| 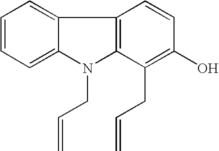 | 263 | 69.68 | 78.67 | |
| 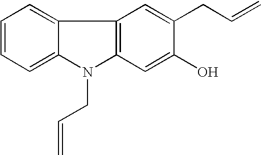 | 263 | 68.92 | 76.07 | |
| 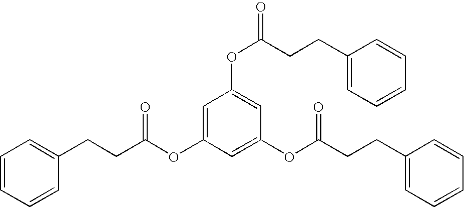 | 522 | 5.00 | 25.00 | |
| 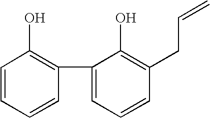 | 226 | 47.1 | 59.34 | |
| 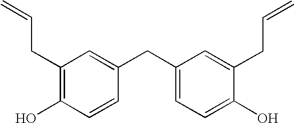 | 280 | 75.54 | 73.70 | Toxic |
| 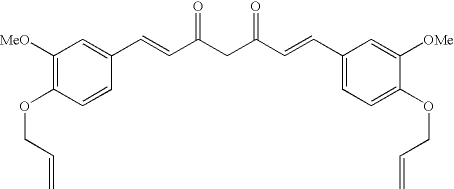 | 448 | 69.97 | 67.91 | |
| 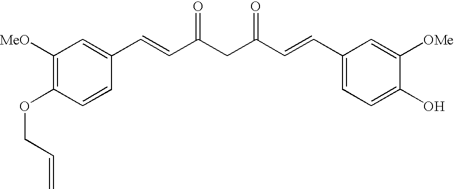 | 408 | 75.21 | 78.84 | |
| 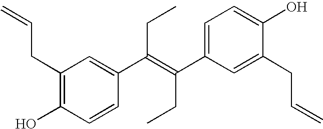 | 348 | 68.36 | 89.32 | Toxic |
| 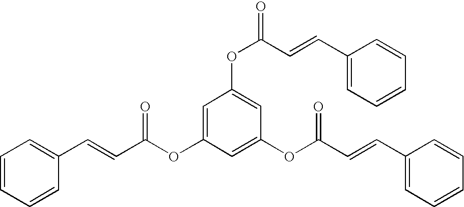 | 516 | 13.36 | 8.60 | |
| 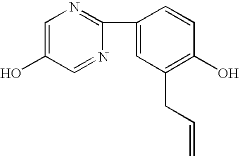 | 228 | 4.40 | 10.00 | |
| 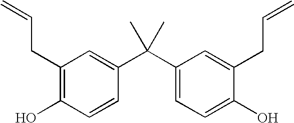 | 308 | 32.90 | 57.10 | |
| 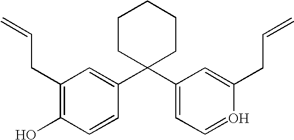 | 348 | 63.00 | 79.40 | |
| 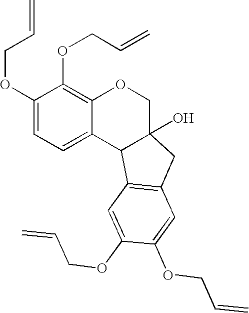 | 462 | 13.82 | 41.82 | |
| 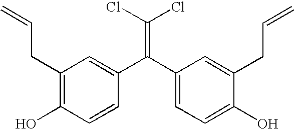 | 360 | 53.65 | 75.56 | |
| 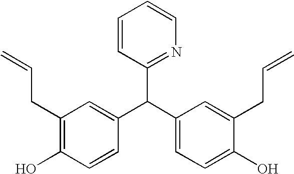 | 357 | 25.25 | 46.09 | |

### Example 8

### Honokiol Potentiates Apoptosis, Suppresses Osteoclastogenesis, and Inhibits Invasion Through Downregulation of IkBa Kinase and NF-kB-Regulated Gene Products

### Materials and Methods

Honokiol and magnolol were isolated as described previously (Bai, X., et al. 2003 J. Biol. Chem. 278:35501-35507). A 50 mM solution of honokiol was prepared in 100% dimethyl sulfoxide, stored as small aliquots at −20° C., and then diluted as needed in cell culture medium. Bacteria-derived recombinant human TNF, purified to homogeneity with a specific activity of 5×10⁷ U/mg, was kindly provided by Genentech (South San Francisco, Calif.). Cigarette smoke condensate, was prepared as previously described (Anto, R. J., et al. 2002 Carcinogenesis. 23:1511-1518). Penicillin, streptomycin, IMDM medium, and FBS were obtained from Invitrogen (Grand Island, N.Y.). PMA, okadaic acid, H₂O₂, and anti-b-actin antibody were obtained from Aldrich-Sigma (St. Louis, Mo.). Antibodies against p65, p50, IkBa, cyclin D1, MMP-9, PARP, IAP1, IAP2, Bcl-2, Bcl-xL, VEGF, c-Myc, ICAM-1, and the annexin V staining kit were obtained from Santa Cruz Biotechnology (Santa Cruz, Calif.). Anti-COX-2 and anti-XIAP antibodies were obtained from BD Biosciences (San Diego, Calif.). Phospho-specific anti-IkBa (serine 32) and phospho-specific anti-p65 (serine 529) antibodies were purchased from Cell Signaling (Beverly, Mass.). Anti-IKK-a, anti-IKK-b, and anti-FLIP antibodies were kindly provided by Imgenex (San Diego, Calif.).

Cell lines. Human myeloid KBM-5 cells, mouse macrophage Raw 264.7 cells, human lung adenocarcinoma H1299 cells human multiple myeloma U266 cells, squamous cell carcinoma SCC4, and human embryonic kidney A293 cells were obtained from American Type Culture Collection (Manassas, Va.). KBM-5 cells were cultured in IMDM medium supplemented with 15% FBS. Raw 264.7 cells were cultured in DMEM/F-12 medium, H1299 cells and U266 were cultured in RPMI 1640 medium, and A293 cells were cultured in DMEM supplemented with 10% FBS. SCC-4 cells were cultured in DMEM containing 10% FBS, nonessential amino acids, pyruvate, glutamine, and vitamins. All media were also supplemented with 100 U/ml penicillin and 100 mg/ml streptomycin.

Cytotoxicity assay Cytotoxicity was assayed by the modified tetrazolium salt 3-(4-5-dimethylthilozol-2-yl)-2-5-diphenyl-tetrazolium bromide (MTT) assay as described previously (Bharti, A. C., et al. 2004 J. Biol. Chem. 279:6065-6076).

PARP cleavage assay. For detection of cleavage products of PARP, whole-cell extracts were prepared by subjecting honokiol-treated cells to lysis in lysis buffer (20 mM Tris, pH 7.4; 250 mM NaCl; 2 mM EDTA, pH 8.0; 0.1% TritonX-100; 0.01 mg/ml aprotinin; 0.005 mg/ml leupeptin; 0.4 mM PMSF; and 4 mM NaVO₄). Lysates were spun at 14000 rpm for 10 min to remove insoluble material, resolved by 10% SDS PAGE, and probed with PARP antibodies.

Live and dead assay. The Live and Dead assay (Molecular Probes), which determines intracellular esterase activity and plasma membrane integrity, was used to measure apoptosis. This assay uses calcein, a polyanionic dye, which is retained within live cells and provides green fluorescence (Bharti, A. C., et al. 2004 J. Biol. Chem. 279:6065-6076). It also uses the ethidium monomer dye (red fluorescence), which can enter cells only through damaged membranes and bind to nucleic acids but is excluded by the intact plasma membrane of live cells. Briefly, 1×10⁵ cells were incubated with 10 mM honokiol for 24 h and then treated with 1 nM TNF for 16 h at 37° C. Cells were stained with the Live and Dead reagent (5 mM ethidium homodimer, 5 mM calcein-AM) and then incubated at 37° C. for 30 min. Cells were analyzed under a fluorescence microscope (Labophot-2, Nikon, Tokyo, Japan).

Annexin V assay. One of the early indicators of apoptosis is the rapid translocation and accumulation of the membrane phospholipid phosphatidylserine from the cell's cytoplasmic interface to the extracellular surface. This loss of membrane asymmetry can be detected using the binding properties of annexin V. Annexin V antibody conjugated with the fluorescent dye FITC was used to detect apoptosis. Briefly, 1×10⁶ cells were pretreated with 30 mM honokiol for 12 h, treated with 1 nM TNF for 16 h, and then subjected to annexin V staining. Cells were washed, stained with FITC-conjugated anti-annexin V antibody, and then analyzed with a flow cytometer (FACSCalibur; BD Biosciences).

Invasion assay. The membrane invasion culture system was used to assess cell invasion because invasion through the extracellular matrix is a crucial step in tumor metastasis. The BD BioCoat Tumor Invasion system is a chamber that has a light-tight polyethelyene terephthalate membrane with 8-mm-diameter pores and is coated with a reconstituted basement membrane gel (BD Biosciences). A total of 2.5×10⁴ H1299 cells were suspended in serum-free medium and seeded into the upper wells. After incubation overnight, cells were treated with 10 mM honokiol for 12 h and then stimulated with 1 nM TNF for a further 24 h in the presence of 1% FBS and the honokiol. The cells that invaded through the Matrigel (i.e., those that migrated to the lower chamber during incubation) were stained with 4 mg/ml calcein-AM (Molecular Probes) in PBS for 30 min at 37° C. and scanned for fluorescence with a Victor 3 multi-plate reader (Perkin Elmer Life and Analytical Sciences, Boston, Mass.); fluorescent cells were counted.

Osteoclast differentiation assay. To determine the effect of honokiol on RANKL-induced osteoclastogenesis, RAW 264.7 cells, which can differentiate into osteoclasts by RANKL in vitro, were cultured. (Bharti, A. C., et al. 2004 J. Biol. Chem. 279:6065-6076). RAW 264.7 cells were cultured in 24-well dishes at a density of 1×10⁴ cells per well and allowed to adhere overnight. The medium was then replaced, and the cells were pretreated with 5 mM honokiol for 12 h and then treated with 5 nM RANKL. At days 4 and 5, the cells were stained for tartrate-resistant acid phosphatase (TRAP) expression, as previously described (18) using an acid phosphatase kit (Sigma-Aldrich), and the TRAP-positive multinucleated osteoclasts (>3 nuclei) per well were counted.

NF-kB activation. To determine NF-kB activation by TNF, which has a well-established role in inflammation, tumor proliferation, promotion, invasion, and metastasis (Aggarwal, B. B. 2003. Nat. Rev. Immunol. 3:745-756), EMSA (Chaturvedi, M. M., et al. 2000 Methods Enzymol. 319:585-602) was performed. Briefly, nuclear extracts prepared from TNF-treated cells (1×10⁶/ml) were incubated with ³²P-end-labeled 45-mer double-stranded NF-kB oligonucleotide (15 mg of protein with 16 fmol of DNA) from the human immunodeficiency virus long terminal repeat, 5′-TTGTTACAA GGGACTTTC CGCTG GGGACTTTC CAGGGAGGCGTGG-3′ (boldface indicates NF-kB-binding sites), for 30 min at 37° C., and the DNA-protein complex formed was separated from free oligonucleotide on 6.6% native polyacrylamide gels. A double-stranded mutated oligonucleotide, 5′-TTGTTACAA CTCACTTTC CGCTG CTCACTTTC CAGGGAGGCGTGG-3, was used to examine the specificity of binding of NF-kB to the DNA. The specificity of binding was also examined by competition with the unlabeled oligonucleotide. For supershift assays, nuclear extracts prepared from TNF-treated cells were incubated with antibodies against either the p50 or the p65 subunit of NF-kB for 30 min at 37° C. before the complex was analyzed by EMSA. Preimmune serum (PIS) was included as a negative control. The dried gels were visualized with a Storm820 and radioactive bands were quantified using Imagequant software (Amersham, Piscataway, N.J.).

Western blot analysis. To determine the effect of honokiol on TNF-dependent IkBa phosphorylation, IkBa degradation, p65 translocation, and p65 phosphorylation, cytoplasmic extracts were prepared (Shishodia, S., et al. 2003 Cancer Res. 63:4375-4383) from KBM-5 cells (2×10⁶/ml) that had been pretreated with 25 mM honokiol for 12 h and then exposed to 0.1 nM TNF for various times. Cytoplasmic protein (30 mg) was resolved on 10% SDS-PAGE gel, transferred to a nitrocellulose membrane, blocked with 5% non-fat milk, and probed with specific antibodies against IkBa, posphorylated IkBa, p65, and phosphorylated p65. To determine the expression of cyclin D1, COX-2, MMP-9, cIAP-1, TRAF1, Bcl-2, Bfl-1, cFLIP, and survivin in whole-cell extracts of treated cells (2×10⁶ cells in 2 ml of medium), 50 mg of protein was resolved on SDS-PAGE and probed by Western blot with specific antibodies as per the manufacturer's recommended protocol. The blots were washed, exposed to HRP-conjugated secondary antibodies for 1 h, and finally detected by ECL reagent (Amersham Pharmacia Biotechnology, Piscataway, N.J.). The bands were quantified using a Personal Densitometer Scan v1.30 using Imagequant software version 3.3 (Molecular Dynamics).

IKK assay. To determine the effect of honokiol on TNF-induced IKK activation, we analyzed IKK by a method essentially as described previously (Shishodia, S., et al. 2003 Cancer Res. 63:4375-4383). Briefly, the IKK complex from whole-cell extracts was precipitated with antibody against IKKa and IKKb and then treated with protein A/G-Sepharose beads (Pierce Chemical, Rockford, Ill.). After 2 h, the beads were washed with lysis buffer and then resuspended in a kinase assay mixture containing 50 mM HEPES, pH 7.4, 20 mM MgCl₂, 2 mM dithiothreitol, 20 mCi [g-³²P]ATP, 10 mM unlabeled ATP, and 2 mg of substrate GST-IkBa (aa 1-54). After incubation at 30° C. for 30 min, the reaction was terminated by boiling with SDS sample buffer for 5 min. Finally, the protein was resolved on 10% SDS-PAGE, the gel was dried, and the radioactive bands were visualized with a Storm820. To determine the total amounts of IKKa and IKKb in each sample, 50 mg of whole-cell proteins was resolved on 7.5% SDS-PAGE, electrotransferred to a nitrocellulose membrane, and then blotted with either anti-IKK-a or anti-IKK-b antibody.

Immunolocalization of NF-kB p65. The effect of honokiol on the TNF-induced nuclear translocation of p65 was examined by an immunocytochemical method using an epifluorescence microscope (Labophot-2; Nikon, Tokyo, Japan) and a Photometrics Coolsnap CF color camera (Nikon, Lewisville, Tex.) as described previously (Shishodia, S., et al. 2003 Cancer Res. 63:4375-4383).

NF-kB-dependent reporter gene transcription. The effect of honokiol on TNF-induced NF-kB dependent reporter gene transcription in A293 cells was measured as previously described (Shishodia, S., et al. 2003 Cancer Res. 63:4375-4383).

COX-2 promoter-dependent reporter luciferase gene expression. COX-2 promoter activity was examined as described elsewhere (Shishodia, S., et al. 2003 Cancer Res. 63:4375-4383). To further determine the effect of honokiol on COX-2 promoter, A293 cells were seeded at a concentration of 1.5×10⁵ cells per well in six-well plates. After overnight culture, the cells in each well were transfected with 2 mg of DNA consisting of COX-2 promoter-luciferase reporter plasmid, along with 6 ml of LIPOFECTAMINE 2000 according to the manufacturer's protocol. The COX-2 promoter (−375 to +59), which was amplified from human genomic DNA by using the primers 5′-GAGTCTCTTATTTATTTTT-3′ (sense) and 5′-GCTGCTGAGGAGTTCCTGGACGTGC-3′ (antisense). After a 6-h exposure to the transfection mixture, the cells were incubated in medium containing honokiol for 12 h. The cells were exposed to TNF (0.1 nM) for 24 h and then harvested. Luciferase activity was measured by using the Luclite luciferase assay system (Perkin-Elmer, Boston, Mass.) according to the manufacturer's protocol and detected by luminometer (Victor 3, Perkin-Elmer). All experiments were performed in triplicate and repeated at least twice to prove their reproducibility.

### Results

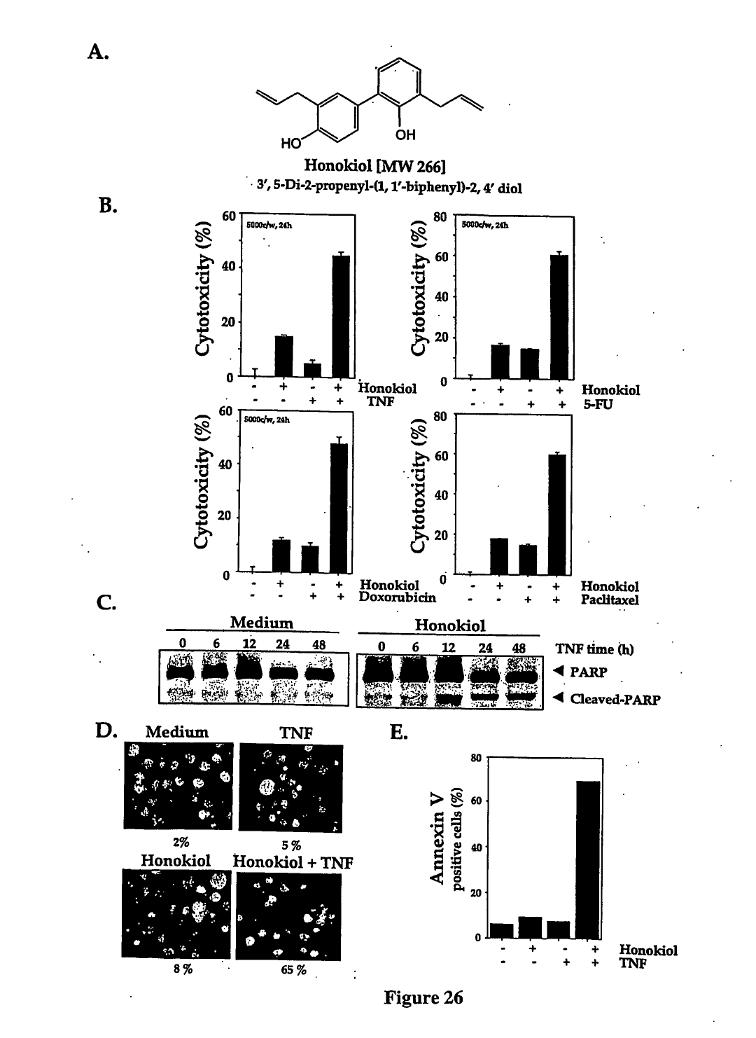

The goal of this study was to investigate the effect of honokiol on the transcription factor NF-kB signaling pathway, on NF-kB-regulated gene products, and on NF-kB-mediated cellular responses. The structure of this retinoid is shown in FIG. 26A. The concentration of honokiol used and the duration of exposure had minimal effect on the viability of cells, as determined by the trypan blue dye exclusion test. For most studies, human myeloid KBM5 cells were used because these cells have been shown to express both types of TNF receptors. To examine the effect of honokiol on the NF-kB activation pathway, most studies used TNF since the pathway activated by this agent is relatively well understood.

Honokiol potentiates the apoptotic effects of TNF and chemotherapeutic drugs. Because NF-kB activation has been shown to suppress the apoptosis induced by various agents (Van Antwerp, D. J., et al. 1996 Science. 274:787-789; Wang, C. Y., et al. 1996 Science 274:784-787), it was investigated whether honokiol would modulate the apoptosis induced by TNF-induced and chemotherapeutic agents in KBM5 cells. The effect of honokiol on TNF and chemotherapeutic agent-induced apoptosis was examined by the MTT assay. It was found that honokiol enhanced the cytotoxic effects of TNF, paclitaxel, and doxorubicin (FIG. 26B).

By using caspase-activated PARP cleavage, it was shown that the enhanced cytotoxicity was due to apoptosis. TNF-induced PARP cleavage was enhanced in the honokiol-treated cells (FIG. 26C). The Live/Dead assay, which measures intracellular esterase activity and plasma membrane integrity, also indicated that honokiol upregulated TNF-induced apoptosis from 5% to 65% (FIG. 26D). Similarly, annexin V staining also showed that honokiol is quite effective in enhancing the effects of TNF (FIG. 26E). The results of all these assays together suggest that honokiol enhances the apoptotic effects of TNF and chemotherapeutic agents.

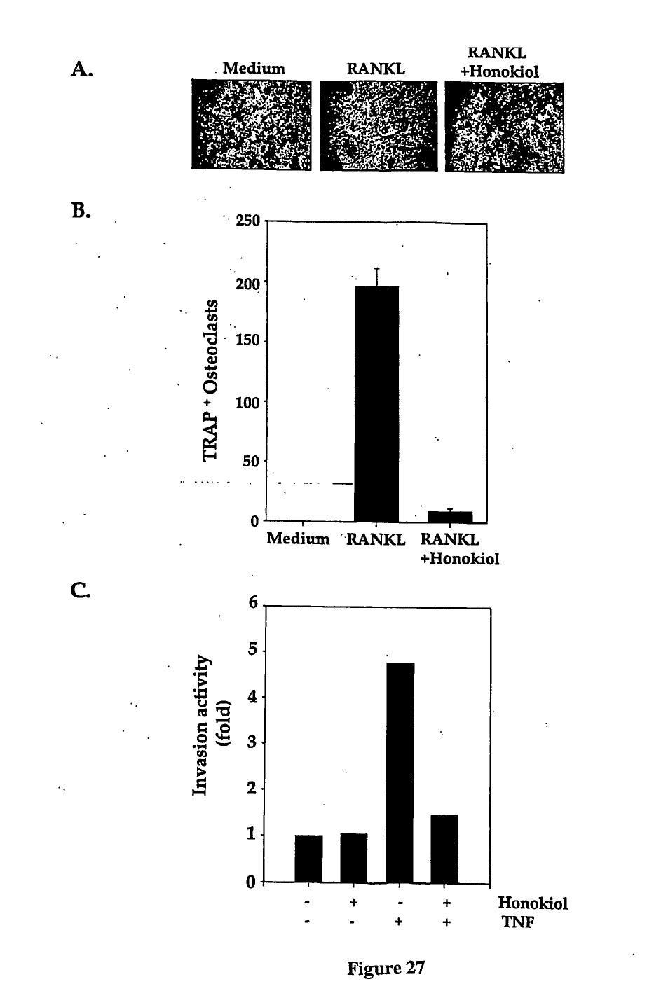

Honokiol suppresses RANKL-induced osteoclastogenesis. Because RANKL, a member of the TNF superfamily, induces osteoclastogenesis through the activation of NF-kB (Abu-Amer, Y., et al. 1997 Nat. Med. 3:1189-1190), whether honokiol can suppress RANKL-induced osteoclastogenesis was assessed. It was discovered that RANKL induced osteoclast differentiation, as indicated by the expression of TRAP, and that honokiol suppressed it (FIGS. 27A and 27B).

Honokiol suppresses TNF-induced tumor cell invasion activity. It is known that NF-kB regulates the expression of gene products (e.g., MMP-9) that mediate tumor cell invasion (Liotta, L. A., et al. 1982 Cancer Metastasis Rev. 1:277-288). Whether honokiol can modulate TNF-induced tumor cell invasion activity was investigated in vitro. To determine this, tumor cells were seeded to the top chamber of the Matrigel invasion chamber with TNF in the presence or absence of honokiol and then examined for invasion. As shown in FIG. 27C, TNF induced tumor cell invasion by almost 5-fold, and honokiol suppressed this activity. Honokiol alone had no invasion activity.

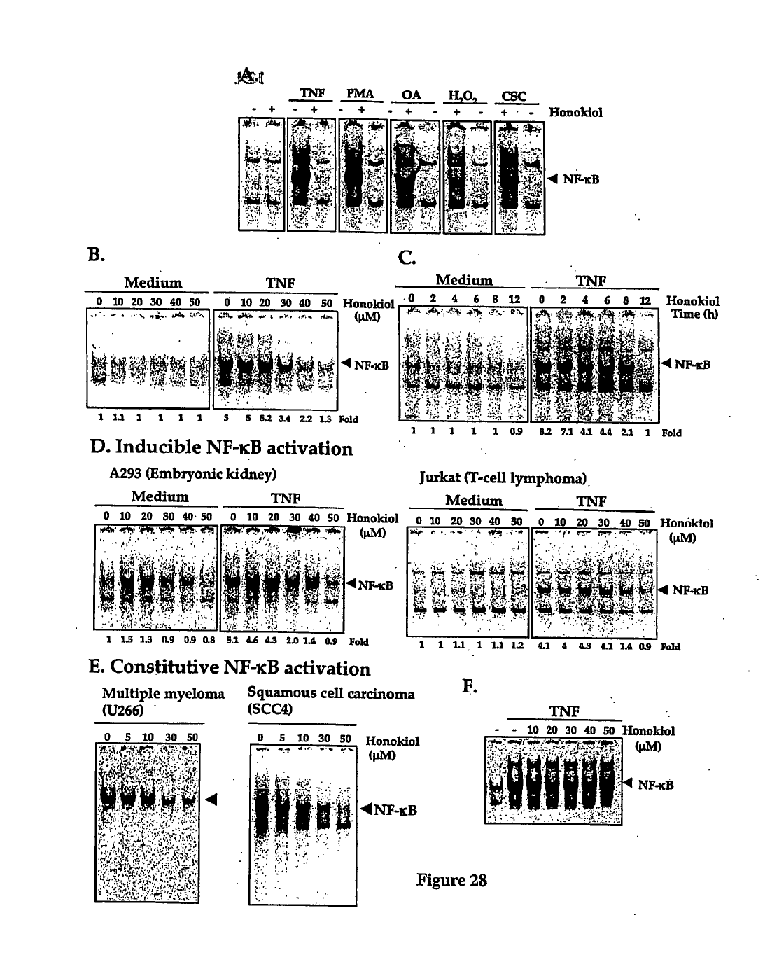

Honokiol blocks NF-kB activation induced by various agents. To assess whether honokiol modulates NF-kB activation, the effect of honokiol on the activation of NF-kB induced by various agents, including TNF, PMA, okadaic acid, cigarette smoke condensate, and H₂O₂ was examined. A DNA-binding assay (EMSA) showed that honokiol suppressed the NF-kB activation induced by all these agents (FIG. 28A). These results suggest that honokiol acted at a step in the NF-kB activation pathway that is common to all these agents.

Honokiol suppresses NF-kB activation in a dose- and time-dependent manner. The EMSA results showed that honokiol alone had no effect on NFkB activation. However, it inhibited TNF-mediated NF-kB activation in a dose-dependent manner (FIG. 28B). The suppression of NF-kB activation by honokiol was also found to be time dependent (FIG. 28C).

Inhibition of NF-kB activation by honokiol is not cell type specific. It has been reported that the NF-kB induction pathway in epithelial cells may differ from that in lymphoid cells (Bonizzi, G., et al. 1997 J. Immunol. 159:5264-5272). The ability of honokiol to inhibit NF-kB activation in different cell types was examined. Honokiol completely inhibited TNF-induced NF-kB activation in embryonic kidney cells (A293) and T cell leukemia (Jurkat) cells (FIG. 28D), indicating a lack of cell type specificity.

Honokiol inhibits constitutive NF-kB activation. The effect of honokiol on NF-kB activation in human multiple myeloma (U266) and head and neck squamous cell carcinoma (SCC4) tumor cells, which both express constitutively active NF-kB was tested. (Bharti, A. C., et al. 2003 Blood. 101:1053-1062; Giri, D. K. and Aggarwal, B. B. 1998. J. Biol. Chem. 273:14008-14014). U266 and SCC4 cells were treated with different concentrations of honokiol for 24 h and then analyzed NF-kB activation. Honokiol inhibited constitutively active NF-kB in both cells in a dose-dependent manner (FIG. 28E). These results indicated a lack of cell type specificity.

Honokiol does not directly affect binding of NF-kB to the DNA. Some NF-kB inhibitors, including TPCK (the serine protease inhibitor), herbimycin A (protein tyrosine kinase inhibitor), and caffeic acid phenethyl ester, directly modify NF-kB to suppress its DNA binding (Finco, T. S., et al. 1994 Proc. Natl. Acad. Sci. U.S.A. 91:11884-11888; Mahon, T. M., and O'Neill, L. A. 1995 J. Biol. Chem. 270:28557-28564; Natarajan, K., et al. 1996 Proc. Natl. Acad. Sci. U.S.A. 93:9090-9095). It was examined whether honokiol mediates its effect through similar mechanism. EMSA showed that honokiol did not modify the DNA-binding ability of NF-kB proteins prepared from TNF-treated cells (FIG. 28F). These results suggest that honokiol inhibits NF-kB activation by a mechanism different from that of TPCK, herbimycin A, or CAPE.

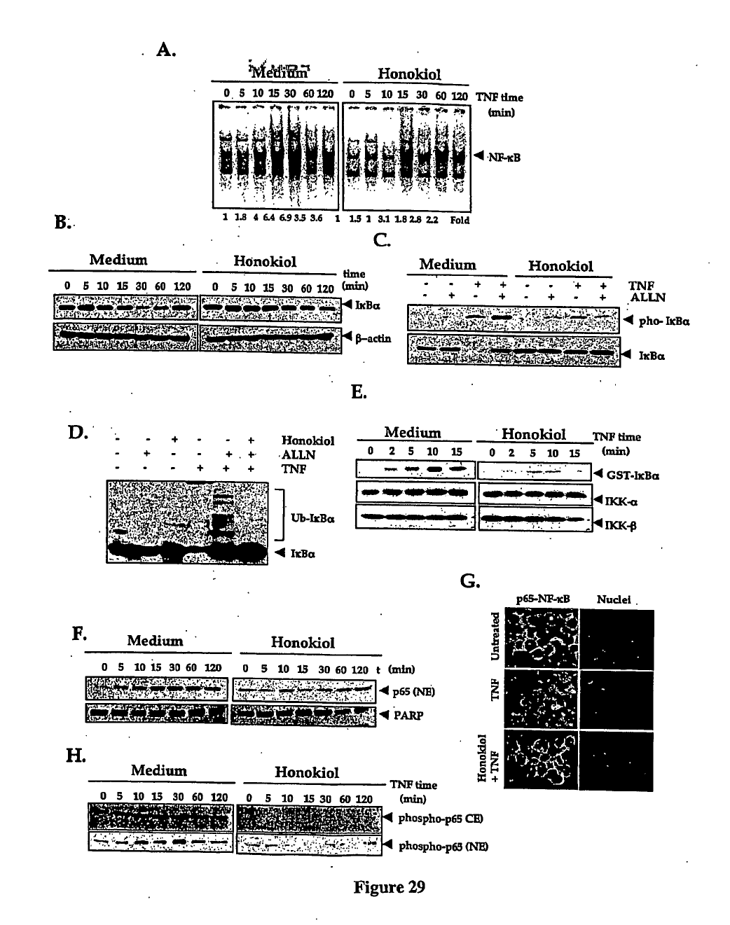

Honokiol inhibits TNF-dependent IkBa degradation. Because IkBa degradation is required for activation of NF-kB (Miyamoto, S., et al. 1994 Proc. Natl. Acad. Sci. U.S.A. 91:12740-12744), whether honokiol's inhibition of TNF-induced NF-kB activation was due to inhibition of IkBa degradation was examined. It was found that TNF induced IkBa degradation in control cells as early as 10 min, but in honokiol pretreated cells TNF had no effect on IkBa degradation (FIG. 29B).

Honokiol inhibits TNF-dependent IkBa phosphorylation. The effect of honokiol on the TNF-induced IkBa phosphorylation needed for IkBa degradation was assessed. ALLN, which prevents the degradation of phosphorylated IkBa, was used. Western blot analysis using antibody that detects only the serine-phosphorylated form of IkBa indicated that TNF induced IkBa phosphorylation and that honokiol completely suppressed it (FIG. 29C). Thus, honokiol inhibited TNF-induced NF-kB activation by inhibiting phosphorylation and degradation of IkBa.

Honokiol inhibits TNF-dependent ubiquitination of IkBa. The effect of honokiol on the TNF-induced IkBa ubiquitination that leads to IkBa degradation was examined. Western blot analysis using antibody that detects IkBa indicated that TNF induced IkBa ubiquitination, as indicated by high-molecular-weight bands, and honokiol completely suppressed it (FIG. 29D). Thus, honokiol inhibited TNF-induced NF-kB activation by inhibiting phosphorylation, ubiquitination, and degradation of IkBa.

Honokiol inhibits TNF-induced IKK activation. Because honokiol inhibits the phosphorylation of IkBa, the effect of honokiol on TNF-induced IKK activation, which is required for TNF-induced phosphorylation of IkBa, was tested. As shown in FIG. 29E (upper panel), honokiol completely suppressed TNF-induced activation of IKK. TNF or honokiol had no direct effect on the expression of IKK protein (bottom panel). In testing the effect of honokiol on IKK activity in vitro, it was found that honokiol did not directly interfere with the IKK activity. Because treatment of cells inhibits TNF-induced IKK activity, honokiol must suppress the activation of IKK.

Honokiol inhibits TNF-induced nuclear translocation of p65. The effect of honokiol on TNF-induced nuclear translocation of p65 was tested by Western blot analysis. As shown in FIG. 29F, honokiol suppressed nuclear translocation of the p65 subunit of NF-kB. Similarly, immunocytochemical analysis (FIG. 29G) indicated that honokiol abolished TNF-induced nuclear translocation of p65.

Honokiol inhibits TNF-induced phosphorylation of p65. The effect of honokiol on TNF-induced phosphorylation of p65 was also tested, since phosphorylation is also required for transcriptional activity of p65 (Zhong, H., et al. 1998 Mol. Cell. 1:661-671). As shown in FIG. 29H, honokiol suppressed p65 phosphorylation almost completely.

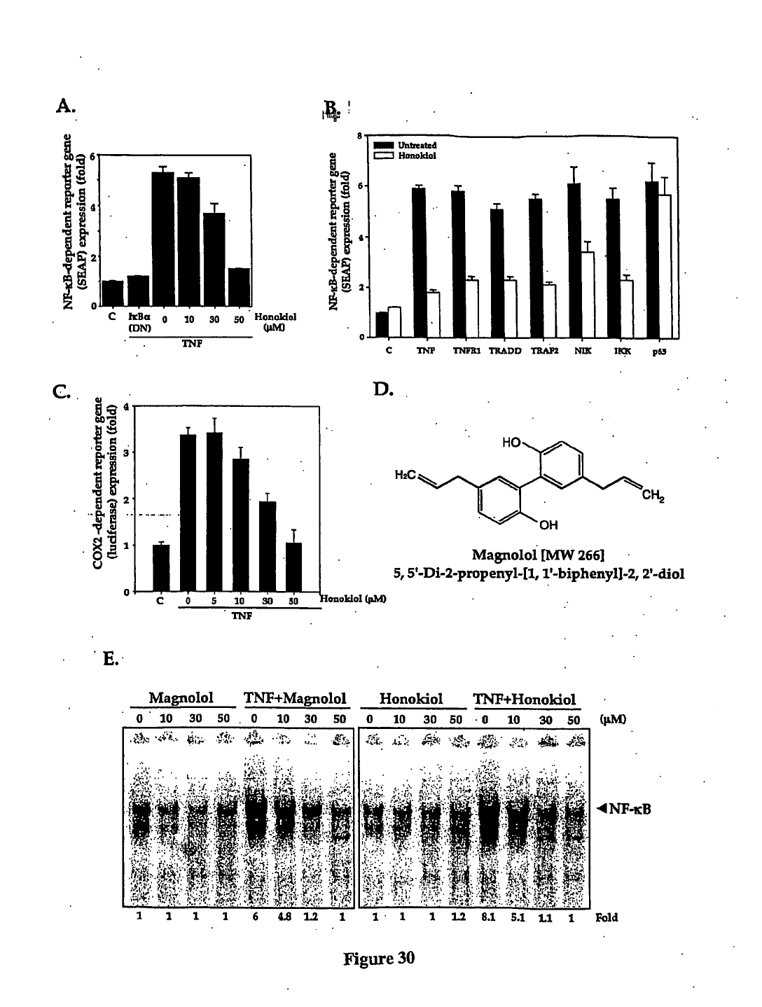

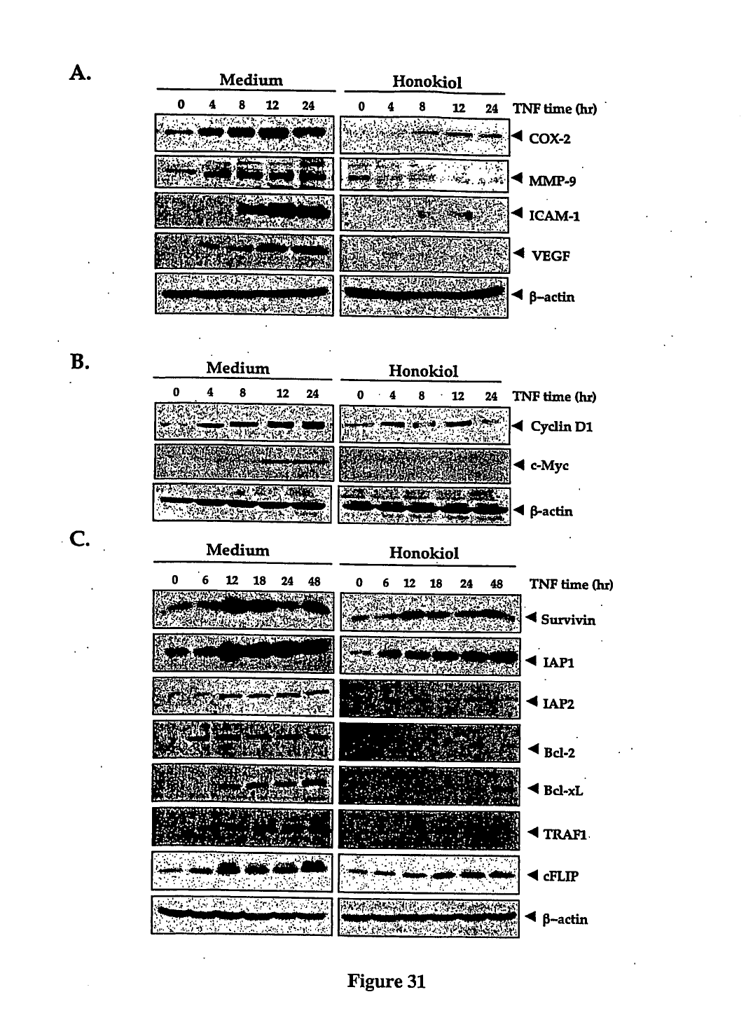

Honokiol represses TNF-induced NF-kB-dependent reporter gene expression. To test the effect of honokiol on NF-kB-dependent gene transcription, cells were transiently transfected with the NF-kB-regulated SEAP reporter construct and then stimulated with TNF. It was found that TNF produced an almost 5-fold increase in SEAP activity over vector control (FIG. 30A), which was inhibited by dominant-negative IkBa, indicating specificity. When the cells were pretreated with honokiol, TNF-induced NF-kB-dependent SEAP expression was inhibited in a dose-dependent manner. These results demonstrated that honokiol inhibits the NF-kB-dependent reporter gene expression induced by TNF.

Tests were also carried out to determine where honokiol acts in the sequence of TNFR1, TRADD, TRAF2, NIK, and IKK recruitment that characterizes TNF-induced NF-kB activation (Hsu, H., et al. 1996 Cell. 84:299-308). In cells transfected with TNFR1, TRADD, TRAF2, NIK, IKKb, and p65 plasmids, NF-kB-dependent reporter gene expression was induced; honokiol suppressed SEAP expression in all cells except those transfected with p65 (FIG. 30B).

Honokiol represses TNF-induced COX2 promoter activity. The effect of honokiol on COX2 promoter activity, which is regulated by NF-kB (Yamamoto, K., et al. 1995 J. Biol. Chem. 270:31315-31320). As shown in FIG. 30C, honokiol inhibited the TNF-induced COX2 promoter activity in a dose-dependent manner.

Magnolol also suppresses NF-kB activation in a dose-dependent manner. Since magnolol is a close structural homologue of honokiol (see FIG. 30D), the dose of magnolol required to suppress NF-kB activation was determined. EMSA results showed that magnolol alone had no effect on NF-kB activation. However, it inhibited TNF-mediated NF-kB activation in a dose-dependent manner (FIG. 30E). The suppression of NF-kB activation by magnolol was comparable with that of honokiol (FIG. 30E).

Honokiol inhibits TNF-induced COX-2, MMP-9, ICAM-1, and VEGF expression. The effect of honokiol on the inhibition of TNF-induced tumor cell invasion was investigated to determine whether these effects of honokiol are mediated through the suppression of COX-2, MMP-9, ICAM-1, and VEGF gene products. It was found that TNF treatment induced the expression of VEGF, COX-2, ICAM-1 and MMP-9 gene products and that honokiol abolished the expression (FIG. 31A).

Honokiol inhibits TNF-induced cyclin D1 and c-myc expression. Both cyclin D1 and c-myc regulate cellular proliferation and are regulated by NF-kB (Aggarwal, B. B. 2004 Cancer: Cell. 6:203-208). Whether honokiol controls the expression of these gene products was also examined. It was found that honokiol abolished, in a time-dependent fashion, the TNF-induced expression of cyclin D1 and c-myc (FIG. 31B).

Honokiol inhibits TNF-induced activation of anti-apoptotic gene products. The above results indicated that honokiol potentiates the apoptosis induced by TNF. Whether this effect of honokiol is through suppression of antiapoptotic gene products was investigated. NF-kB upregulates the expression of a number of genes implicated in facilitating tumor cell survival, including cIAP1, cIAP-2, BCl-2, Bcl-xL, cFLIP, TRAF1, and survivin (Aggarwal, B. B. 2004 Cancer. Cell. 6:203-208). Honokiol inhibited the TNF-induced expression of all of these proteins (FIG. 30C).

The present study was designed to investigate the effect of honokiol on the NF-kB activation pathway and on the NF-kB-regulated gene products that control tumor cell survival, proliferation, invasion, angiogenesis, and metastasis (see FIG. 32). It was found that honokiol potentiated the apoptosis induced by TNF and chemotherapeutic agents and inhibited TN F-induced invasion and RANKL-induced osteoclastogenesis. Honokiol suppressed NF-kB activated by carcinogens, tumor promoters, and inflammatory stimuli in a variety of cell lines. This inhibition was mediated through inhibition of IKK by honokiol, which led to suppression of phosphorylation and degradation of IkBa. Honokiol also inhibited the TNF-induced phosphorylation of p65, nuclear p65 translocation, and NF-kB-dependent reporter gene activity. The expressions of gene products involved in antiapoptosis (IAP1, IAP-2, s survivin, Bcl-2, Bcl-x1, TRAF1, and cFLIP), proliferation (cyclin D1 and c-Myc), and metastasis (MMP-9, COX2, and VEGF) were also downregulated by honokiol.

### Example 9

### Honokiol Induces Apoptosis and Cell Cycle Arrest, and Inhibits In Vitro and In Vivo Growth of Breast Cancer Cells

The in vitro and in vivo activity of honokiol against breast cancer was investigated.

### Materials and Methods

Chemicals, antibodies and constructs: Honokiol (also referred to herein as HNK) was dissolved in ethanol to form a stock solution of 75 mM, and further dissolved in culture medium to form a working solution at the required concentration. Benzoyloxycarbonyl-Val-Ala-Asp-fluoromethylketone (z-VAD-fmk) (BD Pharmingen, San Diego, Calif.) was dissolved in DMSO and used at a concentration of 50 μM. 4-hydroxytanioxifen (4HT, Sigma Chemical Co., St. Louis, Mo.), doxorubicin hydrochloride (Adriamycin®), vincristine (Oncovin®), paclitaxel (Taxol®), and SAHA (Merck & Co., Whitehouse Station, N.J.) were freshly diluted in growth media and immediately added to cells along with HNK, at the indicated concentrations. Cocktail of protease inhibitors (Comp) were obtained from Roche Diagnostic, Alameda, Calif. The antibodies used in this study were: anti-p21 (H-164), anti-p27Kip1 (C-19), anti-cyclin D1 (H-295) anti-PARP-1, anti-BCL-2 (N-19), anti-Bad and anti-Bax, all from Santa Cruz Biotechnology, Santa Cruz, Calif.); anti-ERK and anti-phospho ERK (BD Transduction Labs, San Jose, Calif.); anti-caspase 9 (9502), anti-caspase 8 (9746) and anti-caspase 3 (9668), (Cell Signaling, Danvers, Mass.); anti-Glyceraldehyde-3-phosphate dehydrogenase (GAPDH) (Research Diagnostic Inc., Concord, Mass.); anti-actin; Horseradish peroxidase-conjugated anti-mouse IgG and anti-rabbit IgG (Amersham Biosciences, Piscataway, N.J.); and horseradish peroxidase-conjugated anti-goat (sc-2020, Santa Cruz Biotechnology, CA).

Cell lines: All cell lines were obtained from American Type Culture Collection. The breast cancer cell lines used: MCF-7 and BT-474 cells were grown in DMEM medium containing 10% FCS; MDA-MB-231, SK-BR-3, MDA-MB-436 and T47-D cells were grown in RPMI medium containing 10% FCS. The glioblastoma multiforme cell lines (U343 and T98G) were maintained in DMEM modified medium containing 10% FCS.

Western blot analysis: Cells were harvested and lysed for total protein extraction in a buffer containing 50 mM Tris-Cl pH 7.4, 150 mM NaCl and 2% NP-40 together with a protease inhibitor cocktail (Comp). Approximately 50-150 μg of protein extract was loaded on a 4-15% polyacrylamide gels (Bio-Rad, Hercules, Calif.), separated electrophoretically and blotted from the gel onto PVDF membrane. The membranes were then blocked with a blocking buffer (5% non-fat dry milk in 1×TBST, i.e. 20 mM Tris-HCl, pH 7.6 containing 0.8% NaCl and 0.1% Tween-20) at room temperature for 1 h. The membranes were incubated with the primary antibodies in blocking buffer, followed by incubation with HRP-labeled secondary antibodies. Immunoactivity was detected with horseradish peroxidase-conjugated secondary antibody and visualized by Enhanced Chemiluminescence (Pierce, Rockford, Ill.). Quantification of the results was performed using AlphaImager 2000 (Alpha Innotech, San Leandro, Calif.).

3-(4,5-dimethylthiazol-2-yl)-2,5-diphenyltetrazolium bromide (MTT) proliferation assays: 3×10³ cells/well were plated in 96-well plates, cultured in the appropriate culture media containing 10% FCS, and treated with either control vehicle or various concentrations of HNK, alone or with a secondary drug, as indicated. All secondary drugs were freshly diluted in growth media and immediately added to cells along with the HNK. After 24 hours of incubation at 37° C., 5% CO2, the cells were cultured for four hours with 10% MTT reagent (5 mg/ml; Sigma-Aldrich, St. Louis, Mo.). The medium was aspirated, and the cells were dissolved by dimethyl sulfoxide (DMSO). Absorbance of the formazan product was measured by an enzyme-linked immunosorbent assay reader (Macintosh).

Cell cycle assays: 5×106 cells were cultured in the appropriate culture media containing 10% FCS, and treated with either control vehicle or various concentrations of HNK as indicated for 24 h. Following treatment, the cells were harvested, fixed in methanol and stained with propidium iodide (PI, Abcam, Cambridge, Mass.). Flow cytometry was performed at the Flow Cytometry Core facility of Cedars-Sinai Medical Center, using FACScan (Becton Dickinson, Franklin Lakes, N.J.).

Apoptosis analysis: 5×106 cells were placed in the appropriate culture media containing 10% FCS, and treated with either control vehicle or various concentrations of HNK, as indicated for 24 h. Following treatment, cells were harvested, and stained with PI and Annexin V, using the Annexin V-PE Apoptosis Detection Kit I (BD Pharmingen, San Diego, Calif.) according to the manufacturer protocol. Flow cytometry was performed at the Flow Cytometry Core facility of Cedars-Sinai Medical Center, using FACScan (Becton Dickinson, Franklin Lakes, N.J.). For studies using z-VAD-fmk to inhibit caspase activity, 5×106 cells were incubated with 50 μM z-VAD-fmk for 60 minutes prior to addition of HNK.

Animal studies: All animals were maintained and animal experiments were performed under NIH and institutional guidelines established for the Animal Core Facility at the Cedars-Sinai Medical Center. MDA-MB-231 cells were harvested, washed twice with sterile PBS, counted and re-suspended in Matrigel (BD Biosciences, San Jose, Calif.). Six-week-old female athymic nude mice were injected subcutaneously in both flanks with cells at a density of 1×106 viable cells/100 μl. The mice were treated with daily intra-peritoneal injections of either HNK (2 mg/day) or vehicle control, suspended in 20% intralipid (Baxter Healthcare, Deerfield, Ill.) in a total volume of 0.3 ml. Five mice were used in each group. Tumor size was measured with a linear caliper for up to 5 weeks, and the volume was estimated by using the equation V=(a×b2)×0.5236, where “a” is the larger dimension and “b” the perpendicular diameter.

Model for drug interactions in vitro: The analysis of interaction between two drugs was conducted using the additive model (Xu D., et al. Cancer Lett. 2006 Jan. 7; [Electronic publication; ahead of print]; Sutherland, R. L., et al. Cancer Res. 1983 September; 43(9):3998-4006). The model predicts the effect of a combination to be equal to the product of the effect of its constituents. For example, if a drug combination is composed of two single drugs producing viability of 40% and 60%, respectively, the combination would be expected to result in viability of 24% (0.4×0.6). Principally, an observed effect of a combination higher than predicted by the additive model indicates synergism, whereas a lower value represents a sub-additive effect. A ratio between the observed and the predicted viability by the additive model was calculated for all combinations. If the ratio exceeded 1.2 the interaction is classified as sub-additive; under 0.8, synergistic; and ratios between 0.8 and 1.2 are additive.

Statistical analysis: Results for continuous variables were presented as Mean ±Standard Deviation. Results for categorical variables were presented as Number (%). Two-group differences in continuous variables were assessed by the T-test. Two-group differences in categorical variables were determined by the chi-square. All significance tests are two-tailed. A P value of <0.05 is considered statistically significant.

### Results

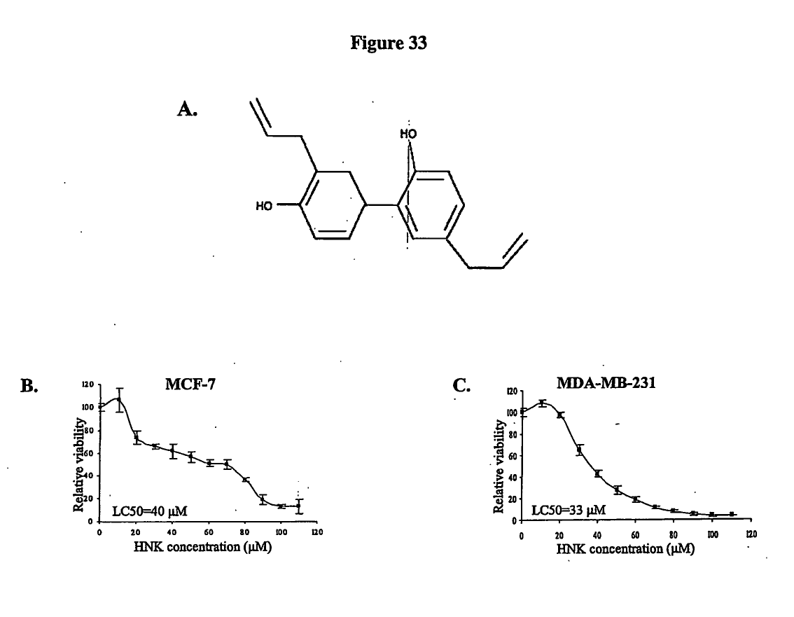

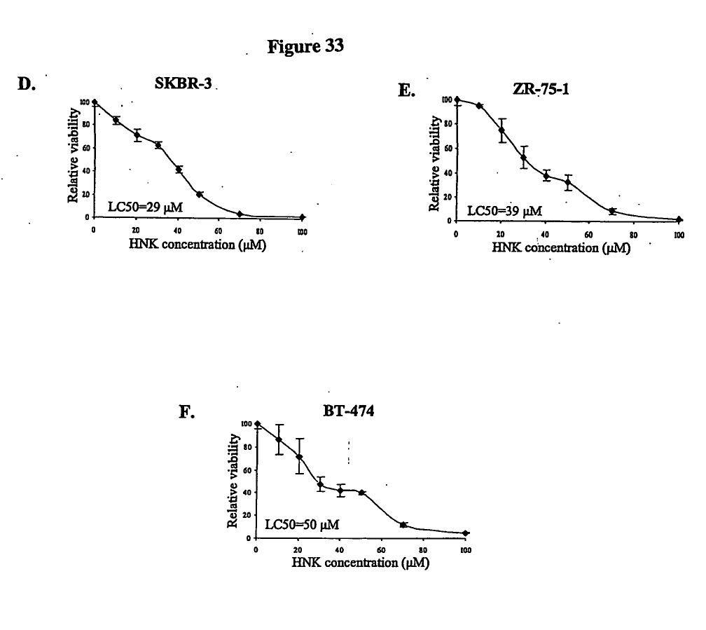

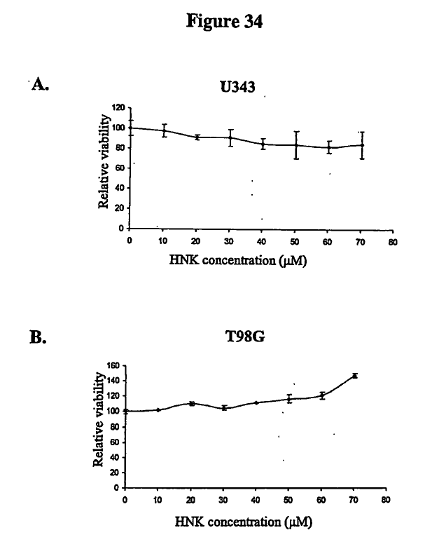

HNK inhibits growth of breast cancer cells. Breast cancer cells were treated with different concentrations of HNK for 24 hours, and MTT assays were conducted to assess viability. The selected cell lines have different phenotypes and different expression patterns of the estrogen receptor (ER), Her2 and p53, and thus represent various subclasses of breast cancer. All five cell lines showed dose-dependent reduction in viability in response to HNK (FIGS. 33B-F). The concentration that reduced viability by 50% (LC50) ranged from 50 μM for the ER-positive BT-474 cells to 29 μM for the poorly differentiated SKBR-3 cell. Similar analyses were also conducted for two glioblastoma multiforme cell lines, U343 and T98G. Over the same dose-range (10-70 μM), both cell lines were resistant to HNK (FIGS. 34 A, B).

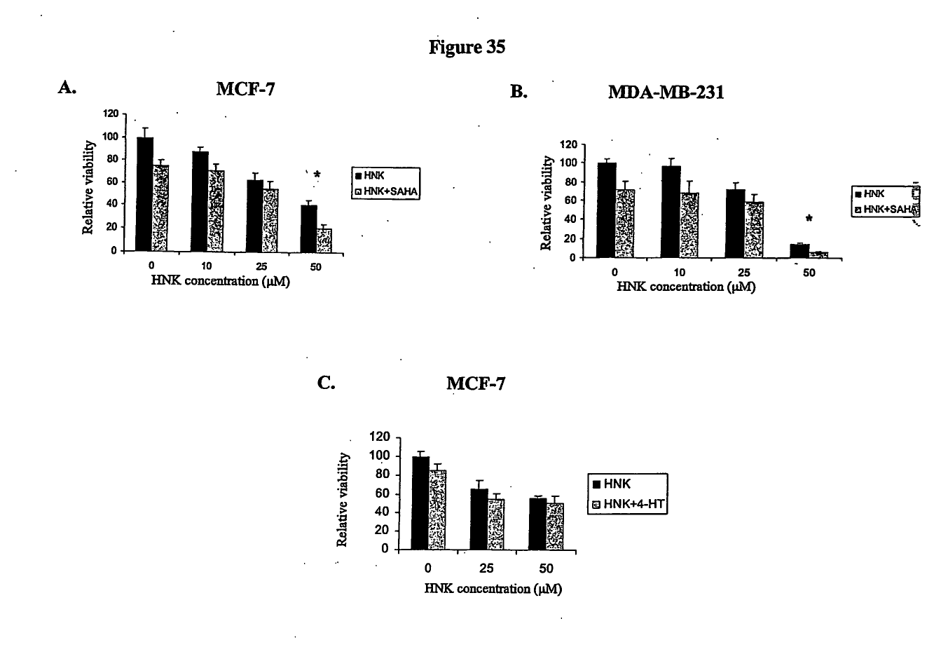

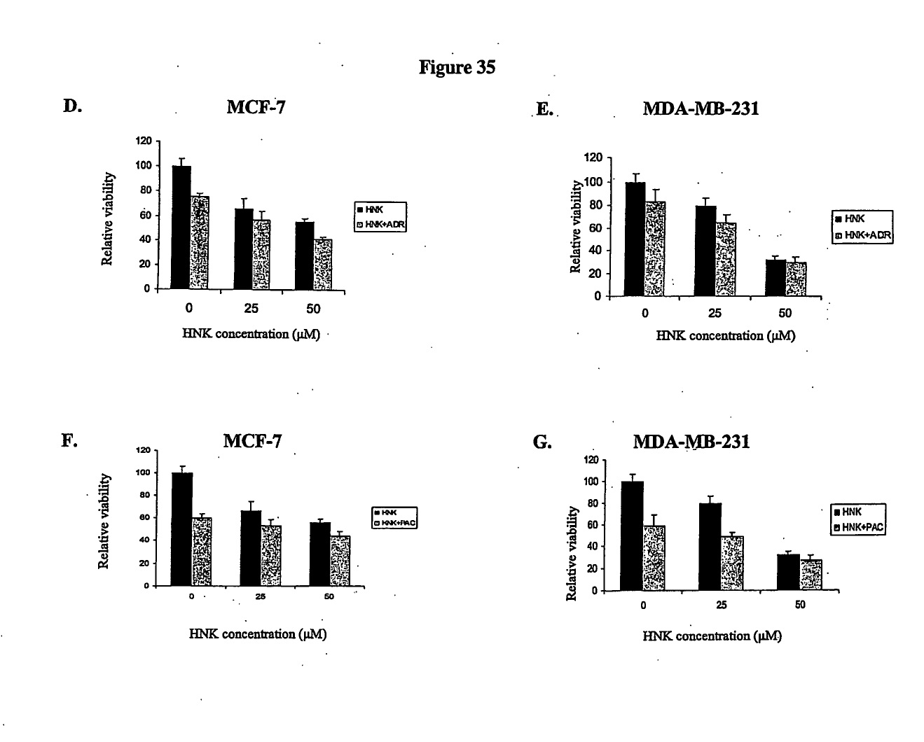

HNK enhances the growth inhibitory activity of SAHA. In B-CLL and in multiple myeloma, FINK has been reported to enhance toxicity and overcome resistance to cytotoxic chemotherapy. Recently, HNK has also been shown to overcome the multidrug resistance (MDR) of the breast cancer cell line MCF-7/ADR. The effects of HNK on the antiproliferative activity of five drugs with different mechanisms of action against two breast cancer cell lines, MCF-7 (ER-positive, p53 wild type) and MDA-MB-231 (ER-negative, p53 mutated), were examined. The cells were treated for 24 hours with various doses of HNK, at a range of 10-50 μM, together with either a control vehicle or a fixed dose of the additional drug; and viability was assessed by the MTT assay. The drugs included: cytotoxic chemotherapeutic drugs (paclitaxel, 250 nM; and doxorubicin 300 nM); 4-hydroxytamoxifen (4-HT, 100 nM), an inhibitor of the estrogen pathway; and the histone deacetylase inhibitor suberoyl anilide bishydroxamide (SAHA, 2 μM). All these drugs have known activity against breast cancer cells and were used at doses that cause less than 40% growth inhibition. Drug interactions were assessed using the additive model; a model that was validated to be a reliable tool for this analysis (Xu D., et al. Cancer Lett. 2006 Jan. 7; [Electronic publication; ahead of print]; Sutherland, R. L., et al. Cancer Res. 1983 September; 43(9):3998-4006). HNK enhanced the activity of all these drugs. However, a synergistic effect was observed only for the combination of HNK and SAHA, inhibitor of histone deacetylase (HDAC) (FIGS. 35 A, B). This effect was also observed with this combination against the SK-BR-3, ZR-75 and BT-474 breast cancer cell lines. Additive effect of was observed for the combination of HNK and the other drugs tested (FIGS. 35 C-G).

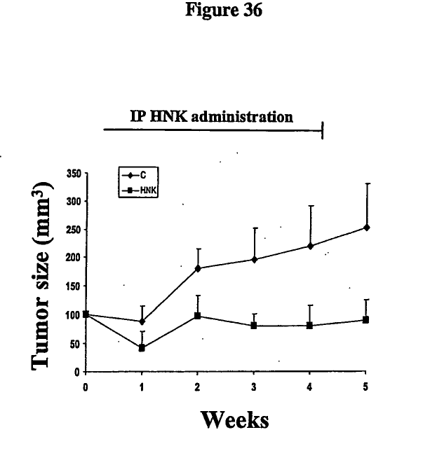

In vivo activity of HNK against human breast cancer. MDA-MB-231 cells were injected on both flanks of nude mice (1×106 cells per injection, five mice per group, two tumors in each mouse), and tumor growth was monitored weekly. These cells were chosen based on their ability easily to form tumors in nude mice (lacroix) and their sensitivity to HNK. The mice were treated with daily injections of either 2 mg HNK (100 mg/kg) or a control vehicle for four weeks; and the tumors were measured weekly. HNK treatment resulted in a complete arrest of tumor growth (p<0.02 from week 2, FIG. 36).

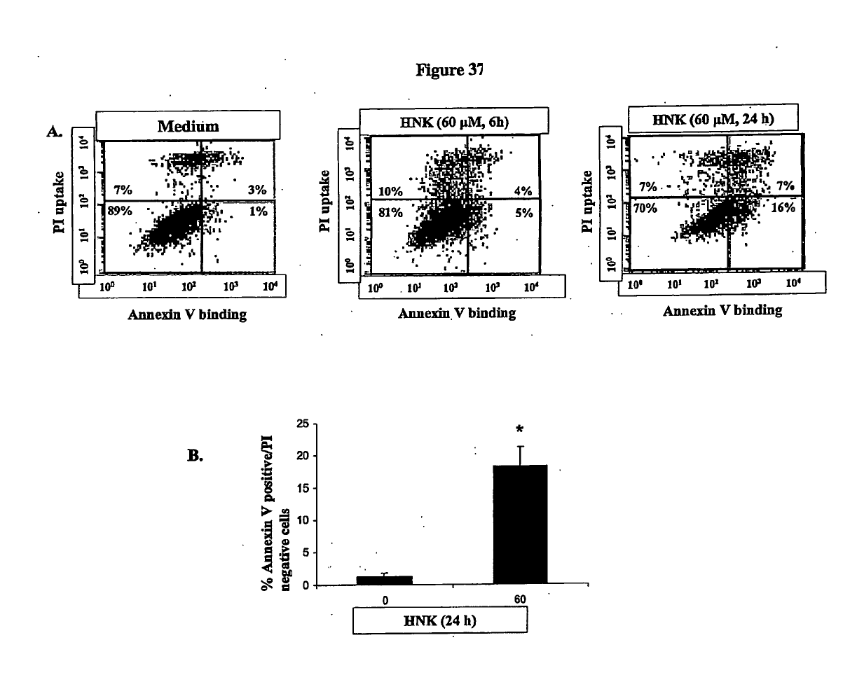

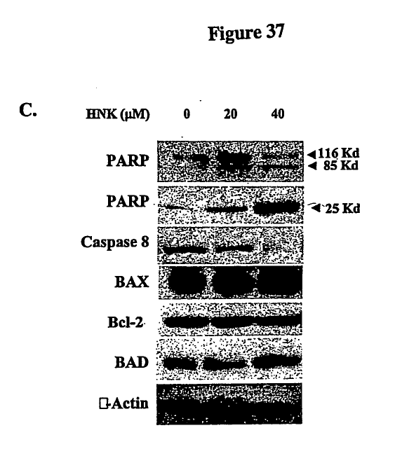

HNK induces apoptosis in breast cancer cell lines. HNK has been shown to induce apoptosis in a wide range of malignant call types. The ability of HNK to induce apoptosis and cell death in breast cancer cell lines was investigated. MCF-7 cells were treated with HNK (60 μM for six or 24 hours) and apoptosis and cell death were assessed using annexin V and PI staining (FIG. 5 A). After 24 hours of HNK treatment, the number of annexin V-positive, PI-negative cells increased significantly, from 1%±0.5% to 16%±3% (p<0.05, FIG. 37 B). Western blot analysis revealed degradation of poly (ADP-ribose) polymerase (PARP) and decreased levels of caspase 8 following HNK treatment (FIG. 37 C). Upregulation of BAX was also noticed, (85% increase at 40 μM compared to control, as analyzed by densitometry) but no significant changes in BCL-2 or BAD levels were observed. Only partial inhibition of apoptosis was observed following pretreatment with z-VAD-fmk.

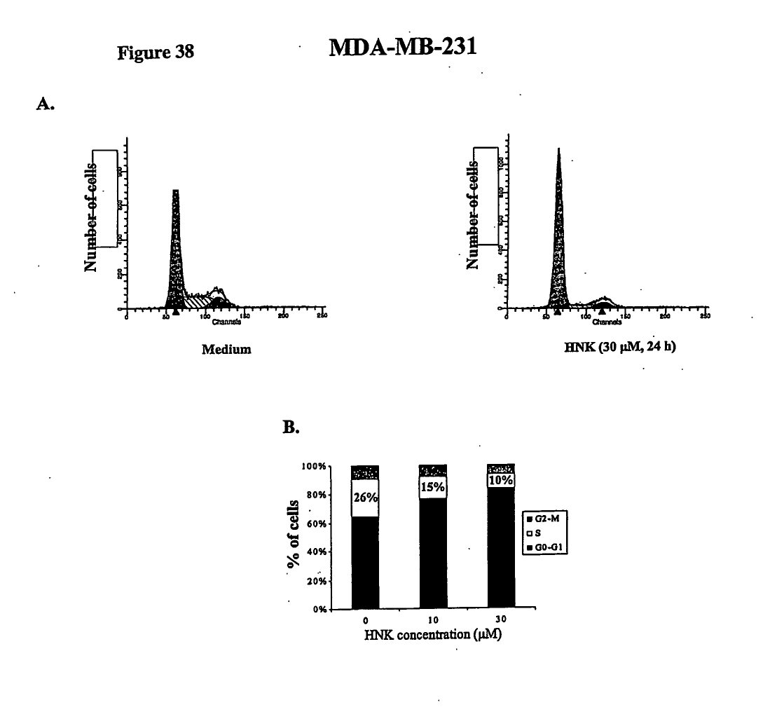

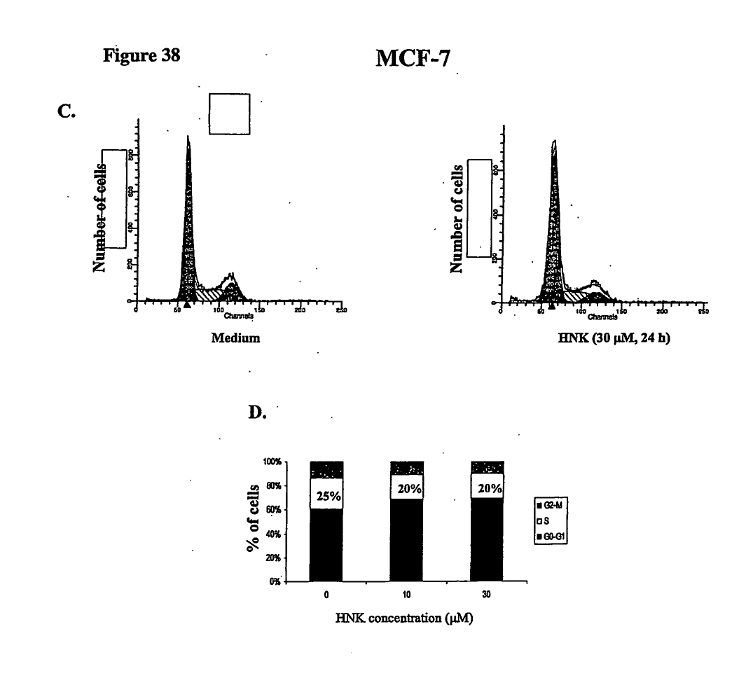

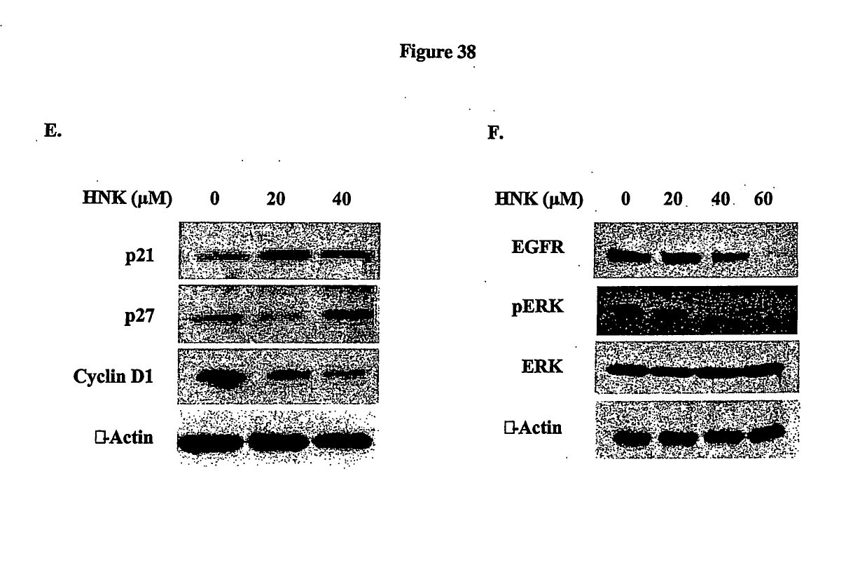

HNK slows cell cycle in breast cancer cell lines. The effects of HNK (10 μM or 30 μM HNK for 24 hours) on cell cycle were evaluated in MCF-7 and MDA-MB-231 cells. These doses of HNK are less than the LC50 for both cell lines. HNK at both doses significantly reduced the number of MDA-MB-231 cells in S-phase (26%, 15% and 10% in the control, 10 μM and 30 μM groups, respectively, p<0.005, FIGS. 38A-B). Less pronounced effect was observed for MCF-7 cells (26%, 20% and 20% in S-phase in the control, 10 μM or 30 μM groups, respectively, FIG. C-D). Expression of proteins involved in G1 cell-cycle regulation (massgue) was examined in MDA-MB-231 cells using Western analysis. HNK treatment (20, 40 and 60 μM HNK for 24 hours) reduced the levels of cyclin D1, and upregulated expression of the cyclin-dependent kinase inhibitors, p27Kip1 and p21Cip1/WAF1 (FIG. 38E).

HNK inhibits growth signaling pathways. In endothelial cells, HNK inhibits the activity of the KDR receptor and its downstream signaling cascades, the MAPK and the AKT pathways. In breast cancer, the PI3K and MAPK pathways are activated by the epidermal growth factor receptor (EGFR), which plays a major role in the pathogenesis of breast cancer. The EGFR mediated signaling is especially important in ER-negative breast cancer; and its inhibition slows the growth of ER-negative cells, such as MDA-MB-231. The effects of HNK on the expression of the EGFR and the activity of the PI3K and MAPK pathways in the MDA-MB-231 cells were examined. The expression of EGFR and phosphorylation of AKT and ERK2 (extracellular signal-regulated kinase 2) were reduced following treatment.

It has been shown in this Example that HNK induces apoptosis and slows the cell cycle of breast cancer cells, and it is systematically active against breast cancer in vivo. Moreover, HNK was well tolerated by the animals in therapeutically beneficial doses. These results suggest that HNK, either alone or in combination with other drugs, may be an effective therapeutic agent in the treatment of breast cancer.

### Example 10

### Honokiol Induces Apoptosis and Mitochondrial Hexokinase Dissociation

### Materials and Methods

Hd HEK293 cells were cultured in DMEM supplemented with 10% fetal bovine serum. The cells were treated with honokiol at 10, 20, or 40 ug/ml for 1 to 24 hours and harvested. Phosphorylation of S6K, S6, and AKT were determined by Western blotting with phosphospecific antibodies. Protein levels were also determined by respective antibodies. All antibodies were purchased from Cell Signaling Inc.

Cell Culture: Polyclonal Rat1a fibroblasts stably expressing mAkt or control vector (Kennedy et al., 1999) as well as SV40-immortalized polyclonal wt and Bax/Bak (Bax/Bak DKO) MEF cell lines (McClintock et al., 2002) were used for the experiment. Cells were routinely maintained in Dulbecco's Modified Eagle Media (DMEM) supplemented with 10% fetal calf serum (FCS), unless otherwise indicated.

Induction of apoptosis: Honokiol was prepared in DMSO and added to the cells in serum-free DMEM at the concentration indicated for a 5-hour time period.

DAPI staining: For 4,6-diamidino-2-phenylindole dihydrochloride hydrate (DAPI) staining, cells were fixed by the addition of formaldehyde solution directly to the medium on the plates. DAPI staining was performed as previously described (Kennedy et al., 1999).

HK activity assay: HK activity was measured by a standard G-6-P dehydrogenase-coupled spectrophotometric assay as described previously (Gottlob et al., 2001) with minor modifications. Whole-cell lysates were prepared by brief sonication (15 s) in homogenization buffer consisting of 45 mM Tris-HCl, 50 mM KH₂PO₄, 10 mM glucose, and 0.5 mM EGTA, pH 8.2. In parallel, mitochondrion-enriched fractions were prepared from identical, paired cells resuspended in 250 mM sucrose/20 mM Tris-Hcl/1 mM EGTA, pH 7.4, via mechanical lysis and differential centrifugation as described previously (Gottlob et al., 2001). Protein concentrations were uniformly determined for both whole-cell and mitochondrion-enriched samples by the method of Bradford using commercially available reagents and standards (Bio-Rad). HK activity was measured as the total glucose-phosphorylating capacity of whole-cell lysates or mitochondrion-enriched fractions in a final assay mixture containing 50 mM triethanolammine chloride, 7.5 mM MgCl₂, 0.5 mM EGTA, 11 mM monothioglycerol, 4 mM glucose, 6.6 mM ATP, 0.5 mg of NADP/ml, and 0.5 U of yeast G-6-P dehydrogenase (Sigma)/ml, pH 8.5. HK activity in each sample was calculated as the coupled rate of NADPH formation by the Lambert-Beer law: [(A340/t)/ε]×dilution factor/[protein], where ε (6.22 mM⁻¹cm⁻¹) is the extinction coefficient for NADPH at 340 nm, t is time, and [protein] is the protein concentration. Percent mtHK activity was calculated from the formula [(mtHK activity mitochondrial protein/total cellular protein)/whole-cell HK activity]×100.

### Results

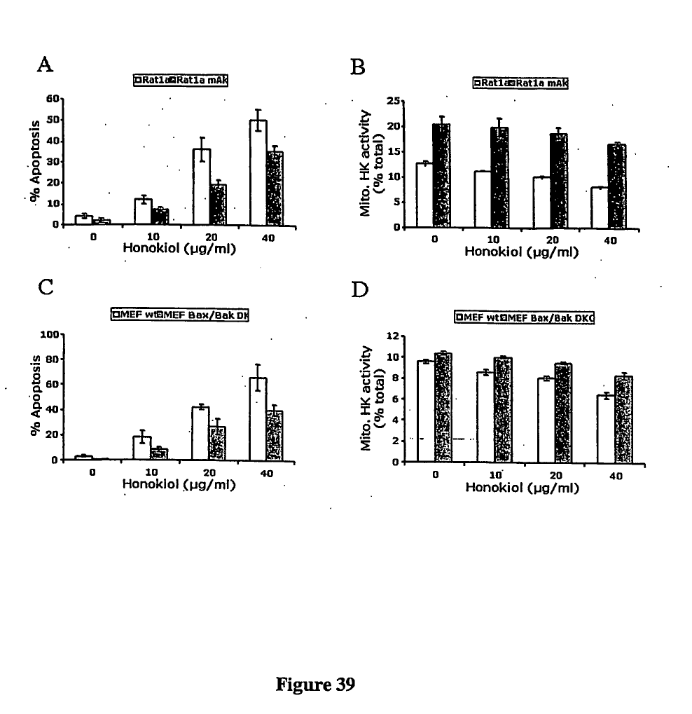

Kinetics of apoptosis in Rat1a and Rat1a-mAkt fibroblast (FIG. 39A) or wildtype and Bax/Bak DKO MEF (FIG. 39C) cell lines after treatment with 0 to 40 μg/ml honokiol in the absence of growth factors. Rat1a and Rat1a-mAkt fibroblast (FIG. 39B) or wildtype and Bax/Bak DKO MEF (FIG. 39D) cell lines were withdrawn from growth factors in the presence or absence of Honokiol and the percentage of total cellular hexokinase activity associated with the mitochondria was determined.

### Example 11

### Treatment of Arthritic Conditions with Honokiol

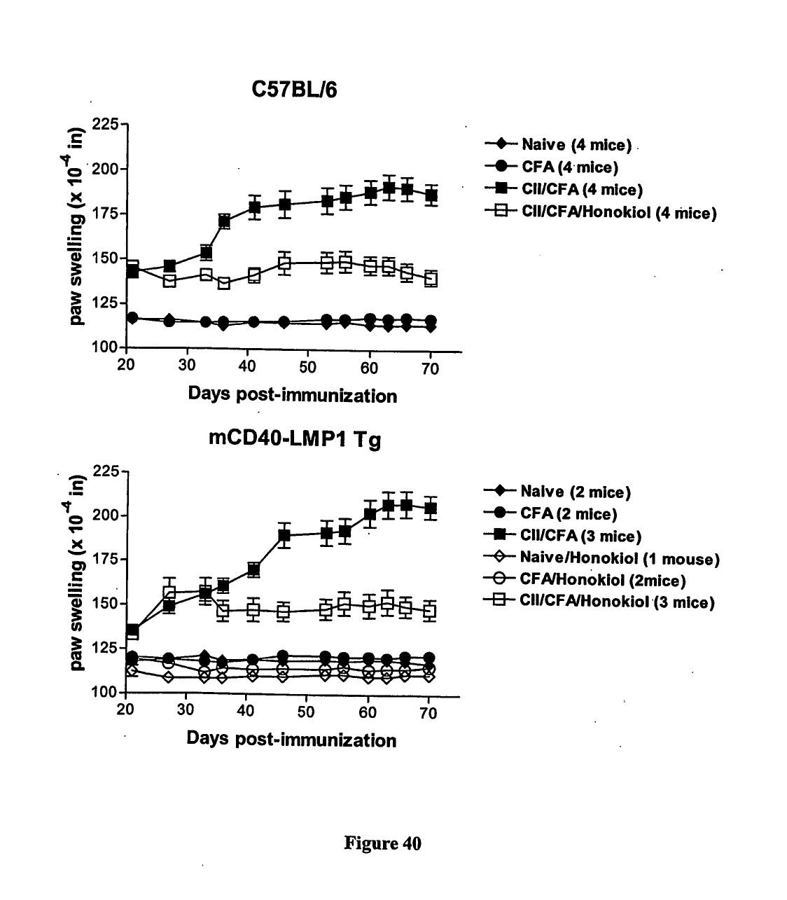

As shown in FIG. 40, Female C57Bl/6 mice were purchased at 5-8 weeks of age from the NCl. Female mice expressing the mCD40-LMP1 transgene (on a CD40−/− background) were bred in our transgenic mouse facility. Mice were either left naïve, immunized in the tail s.c. with 100 mg Type II Chicken Collagen (Sigma) dissolved in 10 mM acetic acid and emulsified in IFA (Sigma) containing 5 mg/ml H37 RA heat-killed mycobacteria (Difco) (CFA), or immunized with 10 mM acetic acid emulsified in CFA. Some of the mice were injected i.p. with 3 mg/mouse/day honokiol suspended in 20% Intralipid, starting at day 21 post-immunization. In vivo honokiol treatment stablizes collagen induced arthritis (CIA) pathology in both C57Bl/6 and LMP1 transgenic mice mice, but does not inhibit to level of negative control (FIG. 40).

In addition, antigen recall lymph node cultures from honokiol treated, CII immunized mice show decreased proliferation and IFN-g production, with unaltered IL-10 production. Inguinal and para-aortic lymph nodes from female C57BL/6 or mCD40-LMP1 Tg mice (4e5/well) were cultured with heat denatured Type II Collagen (CII) and assessed for proliferation (3H incorporation, CPM) and cytokine production (by ELISA). Mice were assessed 70 days post-immunization with CII/CF or CFA only (or naïve). Some mice received honokiol (3 mg/day) from day 21-70 post-immunization. Honokiol treated mice show decreased proliferation and IFN-g production, with unaltered IL-10 production.

CD40 mediated IL-6 and TNF-alpha production was also evaluated. Negatively selected splenic B cells from female C57BL/6 or mCD40-LMP1 Tg mice (1e5/well) were co-cultured with Hi5 insect cells (2.5e4well) infected with baculovirus (WT) expressing mouse CD154, the ligand for CD40. IL-6 in culture supernatants was assessed by ELISA. Mice were assessed 70 days post-immunization with CII/CF or CFA only (or naïve). Some mice received honokiol (3 mg/day) from day 21-70 post-immunization. CD40 mediated IL-6 production is decreased in splenic B cells from mice treated with honokiol. In addition, Negatively selected splenic B cells from female C57BL/6 or mCD40-LMP1 Tg mice (1e5/well) were co-cultured with Hi5 insect cells (2.5e4well) infected with baculovirus (WT) expressing mouse CD154, the ligand for CD40. TNF-a in culture supermatants was assessed by ELISA. Mice were assessed 70 days post-immunization with CII/CF or CFA only (or naïve). Some mice received honokiol (3 mg/day) from day 21-70 post-immunization. CD40 mediated TNF-alpha production is decreased in splenic B cells from mice treated with honokiol.

In LMP1 Tg mice, even negative control mice have lowered IL-6 and TNF-alpha responses. However, IL-10 was not affected, as determined by analyzing the role of Honokiol treatment on B cell IL-10 production. Negatively selected splenic B cells from female C57BL/6 or mCD40-LMP1 Tg mice (1e5/well) were co-cultured with Hi5 insect cells (2.5e4well) infected with baculovirus (WT) expressing mouse CD154, the ligand for CD40. IL-10 in culture supermatants was assessed by ELISA. Mice were assessed 70 days post-immunization with CII/CF or CFA only (or naïve). Some mice received honokiol (3 mg/day) from day 21-70 post-immunization.

Mouse B Cell Line Experiments (CH12.hCD40-LMP1 and M12.hCD40-LMP1)

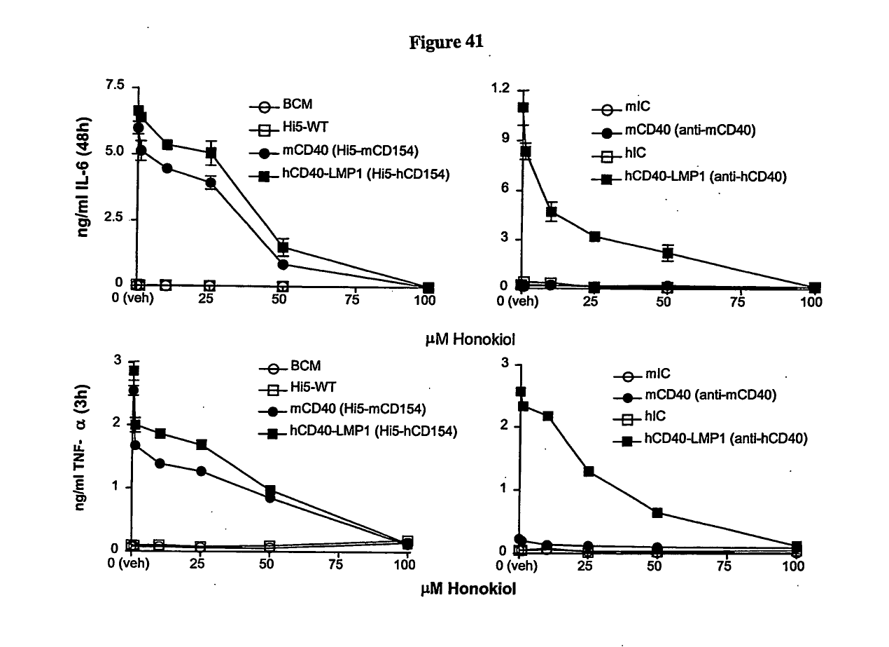

Honokiol inhibits CD40/LMP1 mediated IL-6, TNF-a production in a dose-dependent manner (FIG. 41), but not IL-10 or IL-4. As shown in FIG. 41, CH12.LX cells (1×105 for IL-6; 4×105 for TNF-a) stably transfected with hCD40-LMP1 were stimulated ±Honokiol for indicated times with culture medium (BCM), 1 mg/ml anti-CD40 or isotype control (IC), or Hi-5 insect cells expressing CD154 or WT baculovirus (2.5×104 for IL-6; 1×105 for TNF-a). IL-6 and TNF-a levels in culture supernatants were determined by ELISA. Subsequent IgM production by CH12.LX cells is also affected, as determined by assaying IgM secretion by CH12.hCD40-LMP1 cells. IgM secretion was measured by hemolytic plaque assay, as previously described (GA Bishop. 1991. J. Immunol. 147(4): 1107-1114). Honokiol inhibited IgM production.

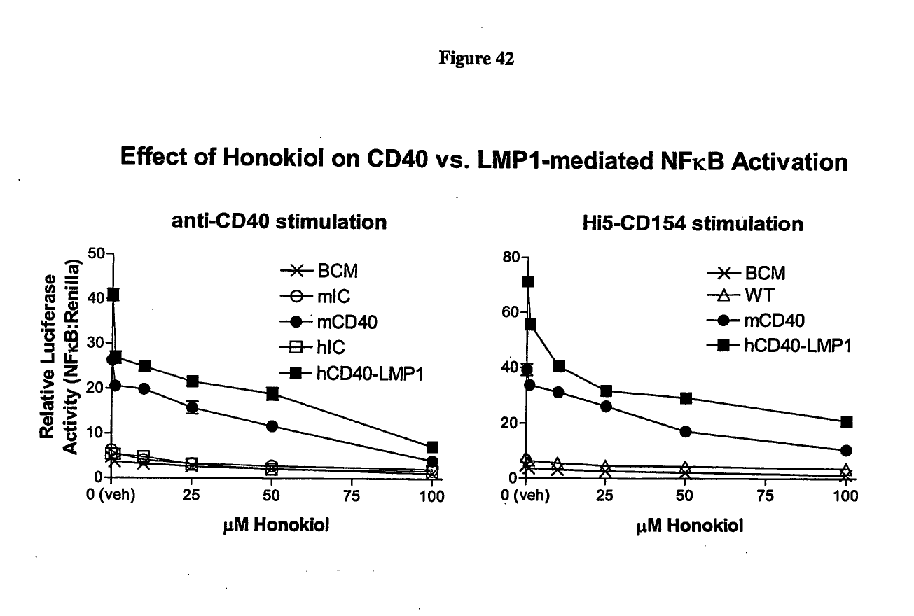

Further, data indicates that NFkB and JNK are two of the pathways which contribute to CD40 and LMP1 activation. NFkB activation (luciferase assay) was inhibited by honokiol in a dose dependent manner (FIG. 42), but not necessarily to baseline (especially via CD40-LMP1). Mouse M12.4.1 cells (1.5×107), stably transfected with hCD40-LMP1 chimeric molecule were transiently transfected with 20 mg 4×NFkB luciferase reporter plasmid and 1 mg Renilla luciferase vector (pRL-null; Promega, Madison, Wis.) by electroporation. Cells (2×106 cells/ml) were rested on ice for 15 minutes, treated with medium alone (BCM), 10 mg/ml anti-TNF-a (MP6-XT3; MP6-XT22), or 100 mM Honokiol for 30 minutes, then incubated an additional 6 hours in the presence of BCM, 10 mg/ml anti-(m)ouse or anti-(h)uman CD40 or isotype controls. After stimulation, cells were pelleted, lysed, and assayed for relative luciferase activity (NFkB: Renilla) per manufacturer's protocol (Promega) using a Turner Designs 20/20 luminometer, with settings of 2 second delay followed by 10 second read. (Note: anti-mCD40 reacts with endougenous CD40 and anti-hCD40 reacts with transfected hCD40-LMP1).

Further, IkBa phosphorylation is not completely inhibited by honokiol, but there is dose dependent inhibition of IkBa reaccumulation after degradation. In total and cytoplasmic fractions, less NFkB2 p100 is processed to p52 and RelB is activated less efficiently (increase and subsequent decrease) in the presence of honokiol. Although still present, honokiol treatment results in less movement of CD40/LMP1 mediated p52 (processed) and RelB (NFkB2 complex protein) into the nucleus (p52 more so than RelB).

JNK phosphorylation is also inhibited by honokiol in a dose dependent manner. CH12.LX cells (1×106) stably transfected with hCD40-LMP1 were stimulated ±honokiol for various times with culture medium (M), 1 mg/ml anti-CD40 or isotype control (IC). The cells were pelleted by centrifugation, lysed and analyzed by SDS PAGE and Western blotting. Peroxidase-labeled antibodies were visualized on Western blots using a chemilluminescent detection reagent to assay for JNK phosphorylation.

### Example 12

### Combination of TSA and Honokiol Treatment on Cancer Cells

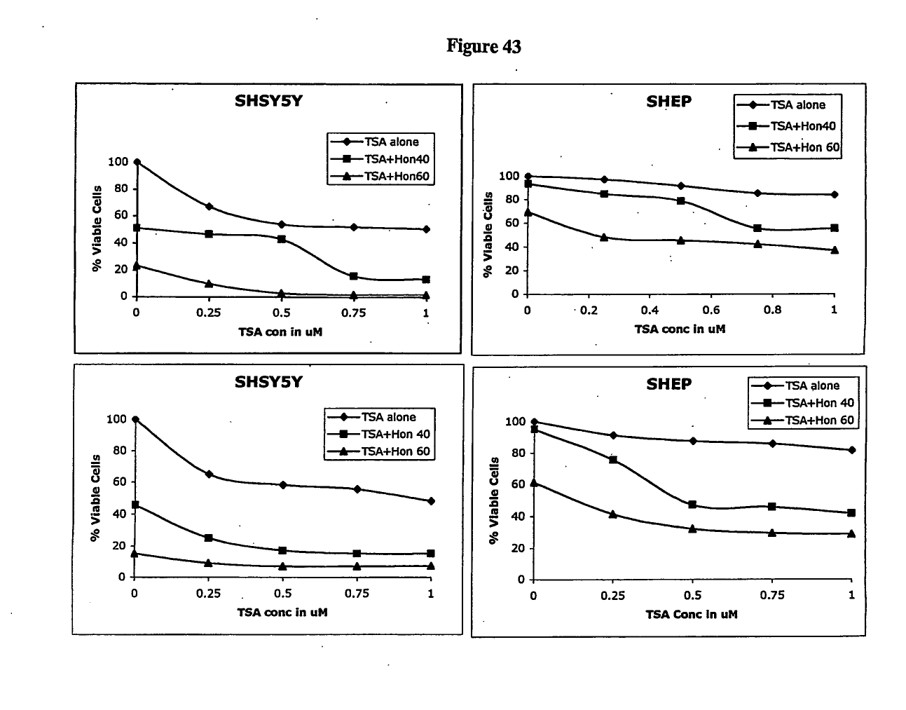

In FIG. 43, the effects on cancer cell viability after treatment with trichostatin A (TSA), a histone deacetylase inhibitor, in combination with honokiol were examined. SHSY5Y and SHEP neuroblastoma cells were treated with 0-1 uM TSA alone or in combination with honokiol. The results demonstrate that there is a significant decrease in the percent of viable cells after combination treatment of TSA and honokiol compared with TSA alone.

This invention has been described with reference to its preferred embodiments. Variations and modifications of the invention, will be obvious to those skilled in the art from the foregoing detailed description of the invention. It is intended that all of these variations and modifications be included within the scope of this invention.

TABLE 1
COMPOUNDS

| | | |
|---|---|---|
| 1. | Name: 2-AllylphenolIUPAC: 2-allylphenolMF: C₉H₁₀OCAS #: 1745-81-9MW: 134.18MDL #: MFCD00002250BP: 226-228° C. FP: 192d: 1.02 | 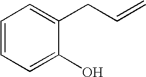 |
| 2. | Name: ChromoneIUPAC: 4H-chromen-4-oneMF: C₉H₈O₂CAS #: 491-38-3MW: 146.14MDL #: MFCD00024064MP: 55-60° C. | 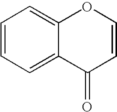 |
| 3. | Name:IUPAC: 2-[(2E)-2-butenyl]phenolMF: C₁₀H₁₂OCAS #:MW: 148.20MDL #: MFCD00020108 | 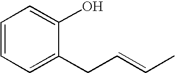 |
| 4. | Name:IUPAC: 2-allyl-4-methylphenolMF: C₁₀H₁₂OCAS #:MW: 148.20MDL #: MFCD00037177 | 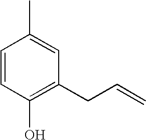 |
| 5. | Name:IUPAC: 2-allyl-6-methylphenolMF: C₁₀H₁₂OCAS #:MW: 148.20MDL #: MFCD00002341 | 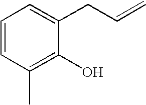 |
| 6. | Name:IUPAC: 2-methyl-4H-chromen-4-oneMF: C₁₀H₈O₂CAS #:MW: 160.17MDL #: MFCD00086982 | 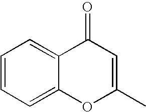 |
| 7. | Name: 6-FluorochromoneIUPAC: 6-fluoro-4H-chromen-4-oneMF: C₉H₅FO₂CAS #: 105300-38-7MW: 164.13MDL #: MFCD00094002MP: 166-169° C. | 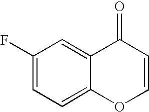 |
| 8. | Name: o-EugenolIUPAC: 2-allyl-6-methoxyphenolMF: C₁₀H₁₂O₂CAS #: 579-60-2MW: 164.20MDL #: MFCD00192169BP: 119-121° C. FP: 230d: 1.0680 | 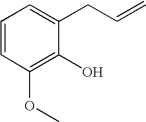 |
| 9. | Name: 3-CyanochromoneIUPAC: 4-oxo-4H-chromene-3-carbonitrileMF: C₁₀H₅NO₂CAS #: 50743-17-4MW: 171.15MDL #: MFCD00052604MP: 174-176° C. | 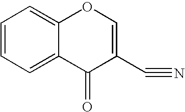 |
| 10. | Name: JugloneIUPAC: 5-hydroxynaphthoquinoneMF: C₁₀H₆O₃CAS #: 481-39-0MW: 174.15MDL #: MFCD00001684MP: 152-154° C. | 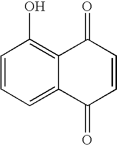 |
| 11. | Name: 3-FormylchromoneIUPAC: 4-oxo-4H-chromene-3-carbaldehydeMF: C₁₀H₈O₃CAS #: 17422-74-1MW: 174.15MDL #: MFCD00014667MP: 151-153° C. | 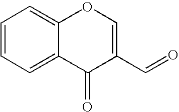 |
| 12. | Name:IUPAC: 2,6-dimethyl-4H-chromene-4-oneMF: C₁₁H₁₀O₂CAS #:MW: 174.20MDL #: MFCD00024069 | 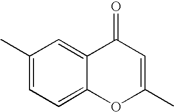 |
| 13. | Name:IUPAC: 6-fluoro-2-methyl-4H-chromen-4-oneMF: C₁₀H₇FO₂CAS #:MW: 178.16MDL #: MFCD03701513 | 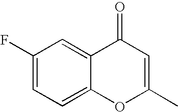 |
| 14. | Name: 6-Methylchromone hydrateIUPAC: 6-methyl-4H-chromen-4-one hydrateMF: C₁₀H₁₀O₃CAS #: 314041-54-8MW: 178.18MDL #: MFCD00209598MP: 76-78° C. | 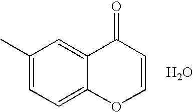 |
| 15. | Name: 6-ChlorochromoneIUPAC: 6-chloro-4H-chromen-4-oneMF: C₉H₅ClO₂CAS #: 33533-99-2MW: 180.59MDL #: MFCD00191904MP: 136-138° C. | 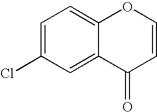 |
| 16. | Name: 3-Cyano-6-methylchromoneIUPAC: 6-methyl-4-oxo-4H-chromen-3-carbonitrileMF: C₁₁H₇NO₂CAS #: 50743-18-5MW: 185.18MDL #: MFCD00191962MP: 151-153° C. | 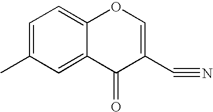 |
| 17. | Name: 3-Formyl-6-methylchromoneIUPAC: 6-methyl-4-oxo-4H-chromen-3-carbaldehydeMF: C₁₁H₈O₃CAS #: 42059-81-4MW: 188.18MDL #: MFCD00138943MP: 172-173° C. | 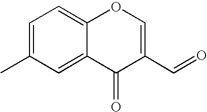 |
| 18. | Name: Plumbagin from Plumbago IndicaIUPAC: 5-hydroxy-2-methylnaphthoquinoneMF: C₁₁H₈O₃CAS #: 481-42-5MW: 188.18MDL #: MFCD00001682MP: 76-78° C. | 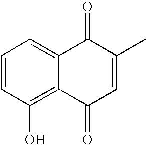 |
| 19. | Name:IUPAC: 1-(2-cyclohexen-1-yl-)-2-methoxybenzeneMF: C₁₃H₁₆OCAS #:MW: 188.27MDL #: MFCD00095230 | 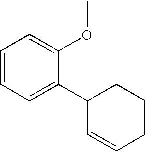 |
| 20. | Name:IUPAC: 2,6-diallyl-3-methylphenolMF: C₁₃H₁₆OCAS #:MW: 188.27MDL #: MFCD00086620 | 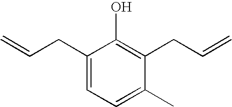 |
| 21. | Name: 6-Fluorochromone-3-carbonitrileIUPAC: 6-fluoro-4-oxo-4H-chromene-3-carbonitirleMF: C₁₀H₄FNO₂CAS #: 227202-21-3MW: 189.14MDL #: MFCD03094001MP: 174-178° C. | 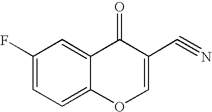 |
| 22. | Name: 2-Amino-3-formylchromoneIUPAC: 2-amino-4-oxo-4H-chromene-3-carbaldehydeMF: C₁₀H₇NO₃CAS #: 61424-76-8MW: 189.17MDL #: MFCD00191735MP: 249° C. | 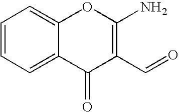 |
| 23. | Name: 4-Oxo-4H-1benzopyran-2-carboxylic acidIUPAC: 4-oxo-4H-chromene-2-carboxylic acidMF: C₁₀H₆O₄CAS #: 4940-39-0MW: 190.15MDL #: MFCD00006838MP: 260° C. | 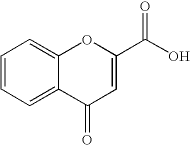 |
| 24. | Name: Chromone-3-carboxylic acidIUPAC: 4-oxo-4H-chromene-3-carboxylic acidMF: C₁₀H₆O₄CAS #: 39079-62-4MW: 190.15MDL #: MFCD00017338MP: 202-205° C. | 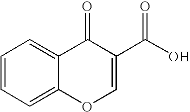 |
| 25. | Name: 5,8-Dihydroxy-1,4-naphthoquinoneIUPAC: 5,8-dihydroxynaphthoquinoneMF: C₁₀H₈O₄CAS #: 475-38-7MW: 190.15MDL #: MFCD00001685MP: 220-230° C. | 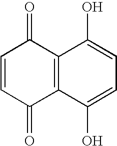 |
| 26. | Name:IUPAC: 2-allyl-4-tert-butylphenolMF: C₁₃H₁₈OCAS #:MW: 190.28MDL #: MFCD00225299 | 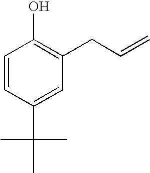 |
| 27. | Name: 6-NitrochromoneIUPAC: 6-nitro-4H-chromen-4-oneMF: C₉H₅NO₄CAS #: 51484-05-0MW: 191.14MDL #: MFCD02954226MP: 172-175° C. | 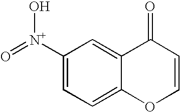 |
| 28. | Name:IUPAC: N-(3-allyl-2-hydroxyphenyl)acetamideMF: C₁₁H₁₃NO₂CAS #:MW: 191.23MDL #: MFCD0092563 | 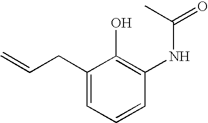 |
| 29. | Name: 6-Fluorochromone-3-carboxaldehydeIUPAC: 6-fluoro-4-oxo-4H-chromene-3-carbaldehydeMF: C₁₀H₁₅FO₃CAS #: 69155-76-6MW: 192.14MDL #: MFCD00139060MP: 155-160° C. | 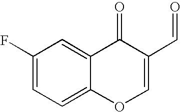 |
| 30. | Name:IUPAC: 2-allyl-3-hydroxy-4-methoxybenzaldehydeMF: C₁₁H₁₂O₃CAS #:MW: 192.21MDL #: MFCD00266470 | 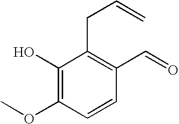 |
| 31. | Name:IUPAC: (2-allylphenoxy)acetic acidMF: C₁₁H₁₂O₃CAS #:MW: 192.21MDL #: MFCD00090833 | 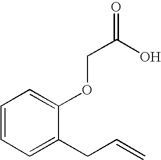 |
| 32. | Name:IUPAC: 1-(2-allyl-3,6-dihydroxyphenyl) ethanoneMF: C₁₁H₁₂O₃CAS #:MW: 192.21MDL #: MFCD00100489 | 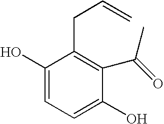 |
| 33. | Name: 6-Chloro-7-methylchromoneIUPAC: 6-chloro-7-methyl-4H-chromen-4-oneMF: C₁₀H₇ClO₂CAS #: 67029-84-9MW: 194.61MDL #: MFCD00239401MP: 171-173° C. | 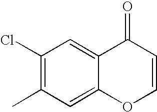 |
| 34. | Name: 3-Cyano-6-ethylchromoneIUPAC: 6-ethyl-4-oxo-4H-chromen-3-carbonitrileMF: C₁₂H₉NO₂CAS #: 50743-19-6MW: 199.21MDL #: MFCD00191959MP: 123-125° C. | 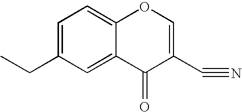 |
| 35. | Name: 3-Cyano-6,7-dimethylchromoneIUPAC: 6,7-dimethyl-4-oxo-4H-chromene-3-carbonitrileMF: C₁₂H₉NO₂CAS #: 94978-86-6MW: 199.21MDL #: MFCD00191957MP: 206-209° C. | 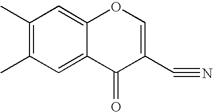 |
| 36. | Name: 3-Formyl-6,8-dimethylchromoneIUPAC: 6,8-dimethyl-4-oxo-4H-chromene-3-carbaldehydeMF: C₁₂H₁₀O₃CAS #: 42059-75-6MW: 202.21MDL #: MFCD00192182MP: 187-190° C. | 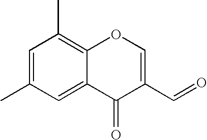 |
| 37. | Name: 6-Ethyl-3-formylchromoneIUPAC: 6-ethyl-4-oxo-4H-chromene-3-carbaldehydeMF: C₁₂H₁₀O₃CAS #: 42059-78-9MW: 202.21MDL #: MFCD00192155MP: 108-110° C. | 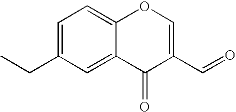 |
| 38. | Name:IUPAC: 3,4,6-trihydroxy-5H-benzo[a]cyclohepten-5-oneMF: C₁₁H₈O₄CAS #:MW: 204.18MDL #: MFCD00597904 | 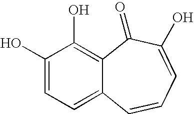 |
| 39. | Name: 6-Methylchromone-2-carboxylic acidIUPAC: 6-methyl-4-oxo-4H-chromene-2-carboxylic acidMF: C₁₁H₈O₄CAS #: 5006-44-0MW: 204.18MDL #: MFCD00239435MP: 267-270° C. | 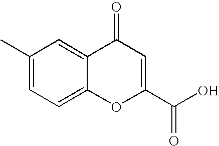 |
| 40. | Name:IUPAC: 1-(2-methoxy-4-methylphenyl)-3-methyl-2-buten-1-oneMF: C₁₃H₁₆O₂CAS #:MW: 204.26MDL #: MFCD00156752 | 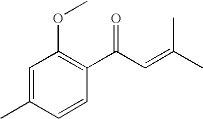 |
| 41. | Name: 6-Chloro-3-cyanochromoneIUPAC: 6-chloro-4-oxo-4H-chromene-3-carbonitrileMF: C₁₀H₄ClNO₂CAS #: 50743-20-9MW: 205.60MDL #: MFCD00191905MP: 209-211° C. | 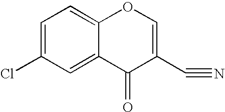 |
| 42. | Name: 6-Fluorochromone-2-carboxylic acidIUPAC: 6-fluoro-4-oxo-4H-chromene-2-carboxylic acidMF: C₁₀H₅FO₄CAS #: 99199-59-4MW: 208.14MDL #: MFCD03070543MP: 257-259° C. | 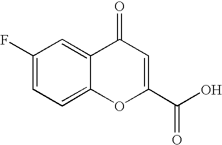 |
| 43. | Name: 6-Fluorochromone-3-carboxylic acidIUPAC: 6-fluoro-4-oxo-4H-chromene-3-carboxylic acidMF: C₁₀H₅FO₄CAS #: 71346-17-3MW: 208.14MDL #: MFCD03424505MP: 234-238° C. | 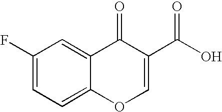 |
| 44. | Name: 6-Chloro-3-formylchromoneIUPAC: 6-chloro-4-oxo-4H-chromene-3-carbaldehydeMF: C₁₀H₅ClO₃CAS #: 42248-31-7MW: 208.60MDL #: MFCD00139138MP: 166-168° C. | 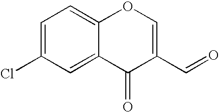 |
| 45. | Name:IUPAC: 8-allyl-2-imino-2H-chromene-3-carbonitrileMF: C₁₃H₁₀N₂OCAS #:MW: 210.23MDL #: MFCD00449726 | 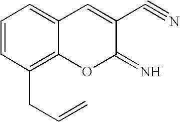 |
| 46. | Name: 3-Cyano-6-ispropylchromoneIUPAC: 6-ispropyl-4-oxo-4H-chromene-3-carbonitrileMF: C₁₃H₁₁NO₂CAS #: 50743-32-3MW: 213.23MDL #: MFCD00191961MP: 118-120° C. | 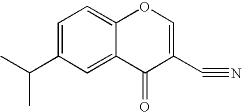 |
| 47. | Name:IUPAC: (2E)-3-(2-furyl)-1-(2-hydroxyphenyl)-2-propen-1-oneMF: C₁₃H₁₀O₃CAS #:MW: 214.22MDL #: MFCD00499139 | 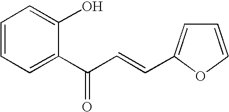 |
| 48. | Name: 3-Formyl-6-isopropylchromoneIUPAC: 6-isopropyl-4-oxo-4H-chromene-3-carbaldehydeMF: C₁₃H₁₂O₃CAS#: 49619-58-1MW: 216.23MDL #: MFCD00192183MP: 98-100° C. | 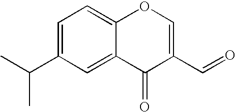 |
| 49. | Name: 2-Amino-3-formyl-6,7-dimethylchromoneIUPAC: 2-amino-6,7-dimethyl-4-oxo-4H-chromene-3-carbaldehydeMF: C₁₂H₁₁NO₃CAS #: 94978-87-7MW: 217.22MDL #: MFCD00191736MP: 300° C. | 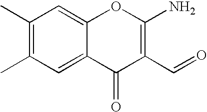 |
| 50. | Name: 3-Formyl-6-nitrochromoneIUPAC: 6-nitro-4-oxo-4H-chromene-3-carbaldehydeMF: C₁₀H₅NO₅CAS #: 42059-80-3MW: 219.15MDL #: MFCD00192184MP: 157-161° C. | 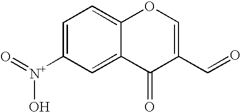 |
| 51. | Name: 3-(Diethylamine)-1-(2-hydroxyphenyl)-2-propen-1-oneIUPAC: (2E)-3-(diethylamino)-1-(2-hydroxyphenyl)-2-propen-1-oneMF: C₁₃H₁₇NO₂CAS #: 1776-33-6MW: 219.28MDL #: MFCD00274217MP: 77-81° C. | 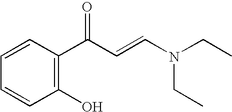 |
| 52. | Name: PurpurogallinIUPAC: 2,3,4,6-tetrahydroxy-5H-benzo[a]cyclohepten-5-oneMF: C₁₁H₈O₅CAS #: 569-77-7MW: 220.18MDL #: MFCD00004145MP: 275° C. | 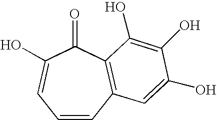 |
| 53. | Name:IUPAC: 5,8-dimethoxy-2-methyl-4H-chromen-4-oneMF: C₁₂H₁₂O₄CAS #:MW: 220.22MDL #: MFCD00024068 | 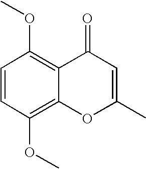 |
| 54. | Name: DillapioleIUPAC: 6-allyl-4,5-dimethoxy-1,3-benzodioxoleMF: C₁₂H₁₄O₄CAS #: 484-31-1MW: 222.24MDL #: MFCD000210045d: 1.1630 | 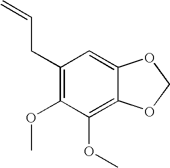 |
| 55. | Name: FlavoneIUPAC: 2-phenyl-4H-chromen-4-oneMF: C₁₅H₁₀O₂CAS #: 525-82-6MW: 222.24MDL #: MFCD00006825MP: 96-97° C. | 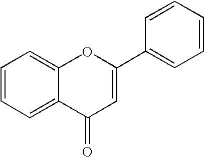 |
| 56. | Name:IUPAC: 2-chloro-5-methoxynaphthoquinoneMF: C₁₁H₇ClO₃CAS #:MW: 222.62MDL #: MFCD00184316 | 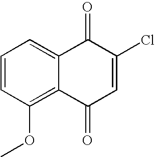 |
| 57. | Name: 6-Chloro-3-formyl-7-methylchromoneIUPAC: 6-chloro-7-methyl-4-oxo-4H-chromene-3-carbaldehydeMF: C₁₁H₇ClO₃CAS #: 64481-12-5MW: 222.62MDL #: MFCD00191919MP: 183-185° C. | 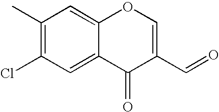 |
| 58. | Name: 2-Amino-6-chloro-3-formylchromoneIUPAC: 2-Amino-6-chloro-4-oxo-4H-chromene-3-carbaldehydeMF: C₁₀H₆ClNO₃CAS #: 68301-77-9MW: 223.61MDL #: MFCD00139140MP: 300° C. | 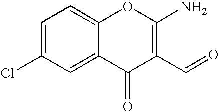 |
| 59. | Name:IUPAC: 3-allyldibenzo[b,d]furan-4-olMF: C₁₅H₁₂O₂CAS #:MW: 224.25MDL #: MFCD00094065 | 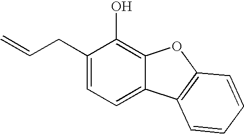 |
| 60. | Name: 1-(2-Hydroxyphenyl)-3-phenyl-2-propenoneIUPAC: (2E)-1-(2-hydroxyphenyl)-3-phenyl-2-propen-1-oneMF: C₁₅H₁₂O₂CAS #: 1214-47-7MW: 224.25MDL #: MFCD00016441MP: 88-92° C. | 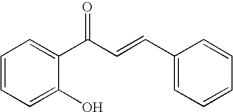 |
| 61. | Name: 3-BromochromoneIUPAC: 3-bromo-4H-chromen-4-oneMF: C₉H₅BrO₂CAS #: 49619-82-1MW: 225.04MDL #: MFCD00017337MP: 94-98° C. | 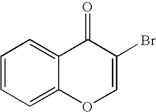 |
| 62. | Name: 6-BromochromoneIUPAC: 6-bromo-4H-chromen-4-oneMF: C₉H₅BrO₂CAS #: 51483-92-2MW: 225.04MDL #: MFCD00239369MP: 135-139° C. | 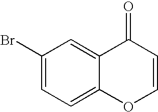 |
| 63. | Name:IUPAC: 3-allyl[1,1′-biphenyl]-2,2′-diolMF: C₁₅H₁₄O₂CAS #:MW: 226.27MDL #: MFCD00091148 | 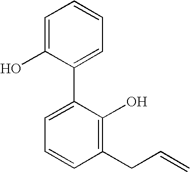 |
| 64. | Name:IUPAC: 2,4-di(2-cyclopenten-1-yl)phenolMF: C₁₆H₁₈OCAS #:MW: 226.31MDL #: MFCD00019306 | 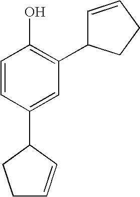 |
| 65. | Name:IUPAC: 8-allyl-2-imine-2H-chromene-3-carboxamideMF: C₁₃H₁₂N₂O₂CAS #:MW: 228.25MDL #: MFCD00988378 | 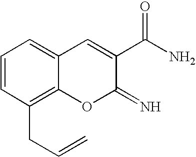 |
| 66. | Name:IUPAC: 4-(2-methoxy-4-methylphenyl)-4-methyl-2,5-cyclohexadien-1-oneMF: C₁₅H₁₆O₂CAS #:MW: 228.29MDL #: MFCD00266756 | 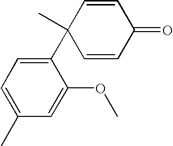 |
| 67. | Name:IUPAC: 1,3,5-triallyl-2-methoxybenzeneMF: C₁₆H₂₀OCAS #:MW: 228.33MDL #: MFCD00182587 | 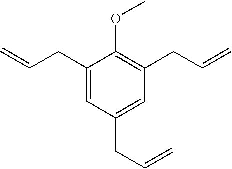 |
| 68. | Name: 6,8-Dichloro-3-methylchromoneIUPAC: 6,8-dichloro-3-methyl-4H-chromen-4-oneMF: C₁₀H₆Cl₂O₂CAS #: 57645-95-1MW: 229.06MDL #: MFCD00218605MP: 141-144° C. | 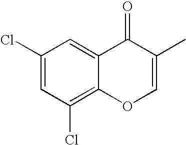 |
| 69. | Name: VisnaginIUPAC: 4-methoxy-7-methyl-5H-furo[3,2-g]chromen-5-oneMF: C₁₃H₁₀O₄CAS #: 82-57-5MW: 230.22MDL #: MFCD00005008MP: 140-142° C. | 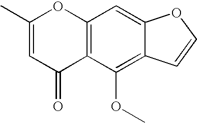 |
| 70. | Name:IUPAC: N-[(Z)-(2,4-dioxo-2H-chromen-3(4H)-ylidene)methyl]ureaMF: C₁₁H₈N₂O₄CAS #:MW: 232.19MDL #: MFCD00158209 | 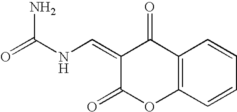 |
| 71. | Name:IUPAC: 1-(2-hydroxy-4,5-dimethoxyphenyl)-3-methyl-2-buten-1-oneMF: C₁₃H₁₆O₄CAS #:MW: 236.26MDL #: MFCD00094982 | 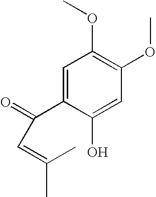 |
| 72. | Name: 6-MethylflavoneIUPAC: 6-methyl-2-phenyl-4H-chromen-4-oneMF: C₁₆H₁₂O₂CAS #: 29976-75-8MW: 236.27MDL #: MFCD00017461MP: 119-122° C. | 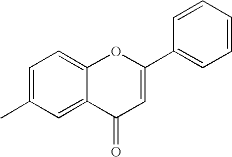 |
| 73. | Name: 6-Fluoro-8-nitrochromone-3-carboxaldehydeIUPAC: 6-fluoro-8-nitro-4-oxo-4H-chromene-3-carbaldehydeMF: C₁₀H₄FNO₅CAS #: 351003-07-1MW: 237.14MDL #: MFCD03094008MP: 110-114° C. | 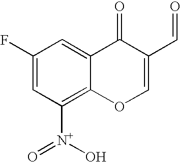 |
| 74. | Name: 3-HydroxyflavoneIUPAC: 3-hydroxy-2-phenyl-4H-chromen-4-oneMF: C₁₅H₁₀O₃CAS #: 577-85-5MW: 238.24MDL #: MFCD00006832MP: 171-172° C. | 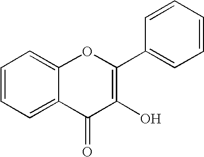 |
| 75. | Name:IUPAC: 7-hydroxy-3-phenyl-4H-chromen-4-oneMF: C₁₅H₁₀O₃CAS #:MW: 238.24MDL #: MFCD00017701 | 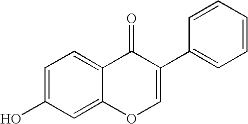 |
| 76. | Name: 6-HydroxyflavoneIUPAC: 6-hydroxy-2-phenyl-4H-chromen-4-oneMF: C₁₅H₁₀O₃CAS #: 6665-83-4MW: 238.24MDL #: MFCD00017329MP: 234-236° C. | 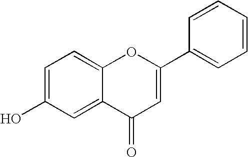 |
| 77. | Name:IUPAC: 9-methoxy-1,4-anthracenedioneMF: C₁₅H₁₀O₃CAS #:MW: 238.24MDL #: MFCD00666818 | 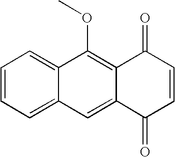 |
| 78. | Name: 7-HydroxyflavoneIUPAC: 7-hydroxy-2-phenyl-4H-chromen-4-oneMF: C₁₅H₁₀O₃CAS #: 6665-86-7MW: 238.24MDL #: MFCD00006835MP: 245-247° C. | 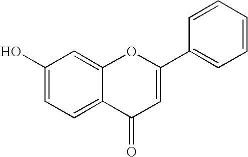 |
| 79. | Name:IUPAC: 2-allyl-4-(phenyldiazenyl)phenolMF: C₁₅H₁₄N₂OCAS #:MW: 238.28MDL #: MFCD00181339 | 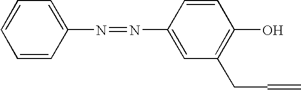 |
| 80. | Name:IUPAC: 3-bromo-2-methyl-4H-chromen-4-oneMF: C₁₀H₇BrO₂CAS #:MW: 239.07MDL #: MFCD02671414 | 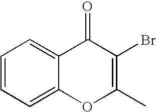 |
| 81. | Name:IUPAC: 6-bromo-2-methyl-4H-chromen-4-oneMF: C₁₀H₇BrO₂CAS #:MW: 239.07MDL #: MFCD03701517 | 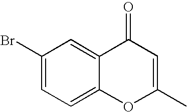 |
| 82. | Name: 6,8-Dichloro-3-cyanochromoneIUPAC: 6,8-dichloro-4-oxo-4H-chromene-3-carbonitrileMF: C₁₀H₃Cl₂NO₂CAS #: 72798-32-4MW: 240.04MDL #: MFCD00192004MP: 169-174° C. | 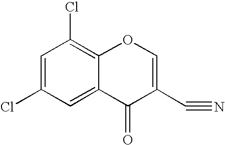 |
| 83. | Name:IUPAC: 1-(2,4-dihydroxyphenyl)-3-phenyl-2-propan-1-oneMF: C₁₅H₁₂ O₃CAS #:MW: 240.25MDL #: MFCD00180050 | 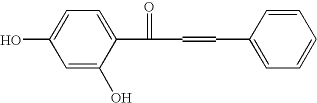 |
| 84. | Name:IUPAC: 5,6-dioxo-5,8-dihydro-1-naphthalenyl 2-methylacrylateMF: C₁₄H₁₀O₄CAS #:MW: 242.23MDL #: MFCD00184315 | 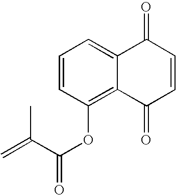 |
| 85. | Name:IUPAC: (2E)-3-(3-fluorophenyl)-1-(2-hydroxyphenyl)-2-propen-1-oneMF: C₁₅H₁₁FO₂CAS #:MW: 242.25MDL #: MFCD00087650 | 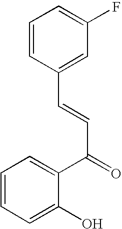 |
| 86. | Name: 3-Bromo-6-fluorochromoneIUPAC: 3-bromo-6-fluoro-4H-chromen-4-oneMF: C₉H₄BrFO₂CAS #: 179111-05-8MW: 243.03MDL #: MFCD03094003MP: 130-134° C. | 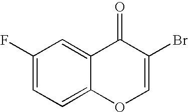 |
| 87. | Name:IUPAC: 2,3-dichloro-5-hydroxynaphthoquinoneMF: C₁₀H₄Cl₂O₃CAS #:MW: 243.04MDL #: MFCD00184322 | 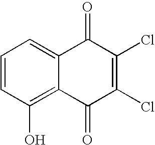 |
| 88. | Name: 6,8-Dichloro-3-formylchromoneIUPAC: 6,8-dichloro-4-oxo-4H-chromene-3-carbaldehydeMF: C₁₀H₄Cl₂O₃CAS #: 64481-10-3MW: 243.04MDL #: MFCD00051510MP: 171-173° C. | 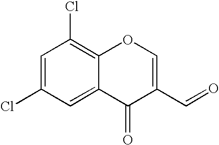 |
| 89. | Name:IUPAC: 3-(3-Isoxazolyl)-7-methoxy-4H-chromen-4-oneMF: C₁₃H₉NO₄CAS #:MW: 243.21MDL #: MFCD00991538 | 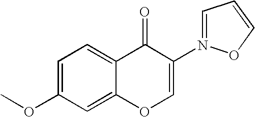 |
| 90. | Name:IUPAC: 7-hydroxy-3-(3-isoxazolyl)-5-methyl-4H-chromen-4-oneMF: C₁₃H₉NO₄CAS #:MW: 243.21MDL #: MFCD01082196 | 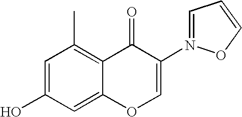 |
| 91. | Name:IUPAC: N′-(3-allyl-2-hydroxybenzylidene)-2-cyanoacetohydrazideMF: C₁₃H₁₃N₃O₂CAS #:MW: 243.26MDL #: MFCD00228589 | 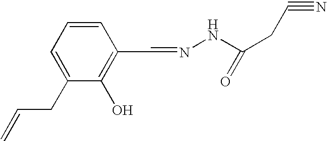 |
| 92. | Name:IUPAC: 3-acetyl-6,7-dimethoxy-4H-chromen-4-oneMF: C₁₃H₁₂O₅CAS #:MW: 248.23MDL #: MFCD00266750 | 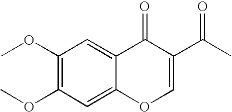 |
| 93. | Name:IUPAC: N-[(Z)-(2,4-dioxo-2H-chromen-3(4H)-ylidene)methyl]thioureaMF: C₁₁H₈N₂O₃SCAS #:MW: 248.26MDL #: MFCD02671782 | 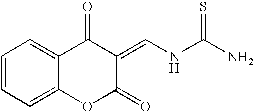 |
| 94. | Name: 6-Bromo-3-cyanochromoneIUPAC: 6-bromo-4-oxo-4H-chromene-3-carbonitrileMF: C₁₀H₄BrNO₂CAS #: 52817-13-7MW: 250.05MDL #: MFCD00191842MP: 224-226° C. | 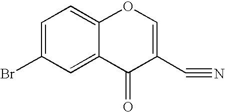 |
| 95. | Name: 7-MethoxyflavoneIUPAC: 7-methoxy-2-phenyl-4H-chromen-4-oneMF: C₁₆H₁₂O₃CAS #: 22395-22-8MW: 252.26MDL #: MFCD00017462MP: 110-112° C. | 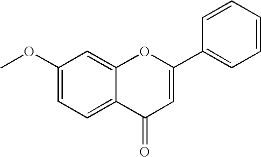 |
| 96. | Name: 6-MethoxyflavoneIUPAC: 6-methoxy-2-phenyl-4H-chromen-4-oneMF: C₁₆H₁₂O₃CAS #: 26964-24-9MW: 252.26MDL #: MFCD00017322MP: 163-165° C. | 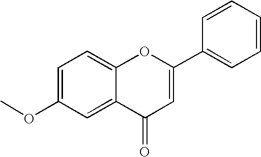 |
| 97. | Name:IUPAC: 7-hydroxy-8-methyl-3-phenyl-4H-chromen-4-oneMF: C₁₆H₁₂O₃CAS #:MW: 252.26MDL #: MFCD01995544 | 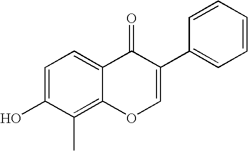 |
| 98. | Name:IUPAC: 7-methoxy-3-phenyl-4H-chromen-4-oneMF: C₁₆H₁₂O₃CAS #:MW: 252.26MDL #: MFCD00181416 | 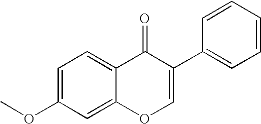 |
| 99. | Name: 3-MethoxyflavoneIUPAC: 3-methoxy-2-phenyl-4H-chromen-4-oneMF: C₁₆H₁₂O₃CAS #: 7245-02-5MW: 252.26MDL #: MFCD00017612MP: 114-115° C. | 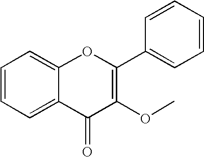 |
| 100. | Name:IUPAC: 5-hydroxy-7-methyl-2-phenyl-4H-chromen-4-oneMF: C₁₆H₁₂O₃CAS #:MW: 252.26MDL #: MFCD00047635 | 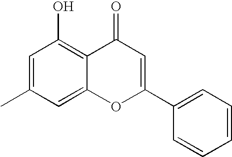 |
| 101. | Name: 2-(3-Allyl-2-hydroxy-5-methylphenyl)-2H-benzotriazoleSynonyms: 2-Allyl-6-(2H-benzotriazol-2-yl)-p-Cresol2-(2H-Benzotriazol-2-yl)-4-methyl-6-(2-propenyl)phenolMF: C₁₆H₁₅N₃OCAS #: 2170-39-0MW: 265.31MDL #: MFCD00236141MP: 93-101 oC | 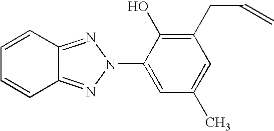 |
| 102. | Name: 2-benzotriaziol-1-yl-acetic acid (4-allyl-3-hydroxy-benzylidene)-hydrazideMF: C₁₈H₁₇N₅O₂CAS #:MW: 335.37MDL #: | 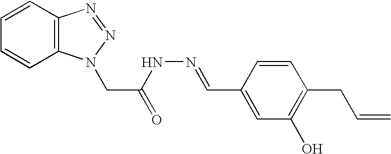 |
| 103. | Name: 2-allyl-1-hydroxy-3-methylpyrido(1,2-A)benzimidazole-4-carbonitrileMF: C₁₆H₁₃N₃OCAS #:MF: 263.30MDL #: | 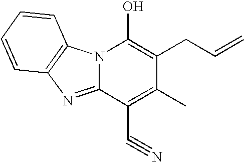 |
| 104. | Name: Pyridine-2-carboxylic acid (3-allyl-2-hydroxy-benzylidene)-hydrazideMF: C₁₆H₁₅N₃O₂CAS #:MW: 281.32MDL #: | 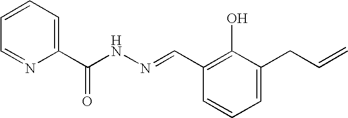 |
| 105. | Name: Thiophene-2-carboxylic acid (3-allyl-2-hydroxy-benzylidene)-hydrazideMF: C₁₅H₁₄N₂O₂SCAS #:MW: 286.36MDL #: | 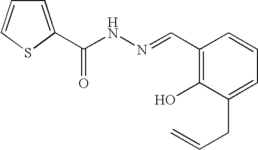 |

1. A compound of Formula Ia, or a salt, ester or prodrug thereof: 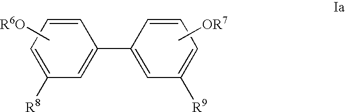
wherein R⁶ and R⁷ are independently H, alkyl, alkenyl, alkynyl, or aryl, which is optionally substituted; wherein R⁸ and R⁹ are independently alkyl, alkenyl, alkynyl, or aryl; and wherein at least one of R⁸ and R⁹ are alkyl, such as C₁₋₅ alkyl.

2. The compound of claim 1, wherein: R⁶ and R⁷ are independently H or C₁₋₅ alkyl; R⁸ and R⁹ are independently C₁₋₅ alkyl or alkenyl; and at least one of R⁸ and R⁹ are methyl, ethyl, propyl or butyl.

3. A compound of Formula Ib, or a salt, ester or prodrug thereof: 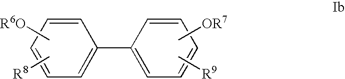
wherein R⁶ and R⁷ are independently H, alkyl, alkenyl, alkynyl, or aryl, which is optionally substituted; wherein R⁸ and R⁹ are independently alkyl, alkenyl, alkynyl, or aryl; and
wherein at least one of R⁶ and R⁷ are not H.

4. The compound of claim 3, wherein: R⁵ and R⁷ are independently methyl, ethyl or propyl: R⁸ and R⁹ are independently C₁₋₅ alkyl or alkenyl; and at least one of R⁶ and R⁷ are not H.

5. A compound of Formula Ic, Id, Ie or If, or a salt, ester or prodrug thereof: 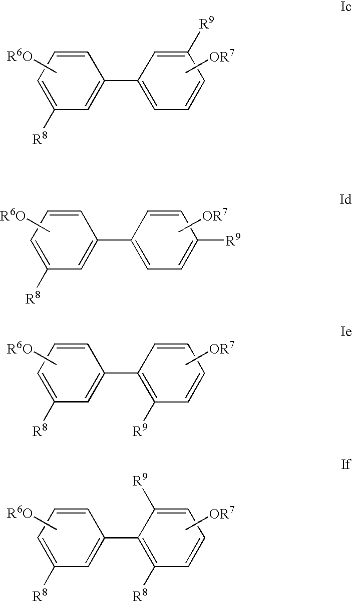
wherein R⁶ and R⁷ are independently H, alkyl, alkenyl, alkynyl, or aryl; and wherein R⁸ and R⁹ are independently alkyl, alkenyl, alkynyl, or aryl.

6. The compound of claim 5, wherein: R⁶ and R⁷ are independently H or C₁₋₅ alkyl; and R⁸ and R⁹ are independently C₁₋₅ alkyl or alkenyl.

7. A compound selected from the group consisting of:

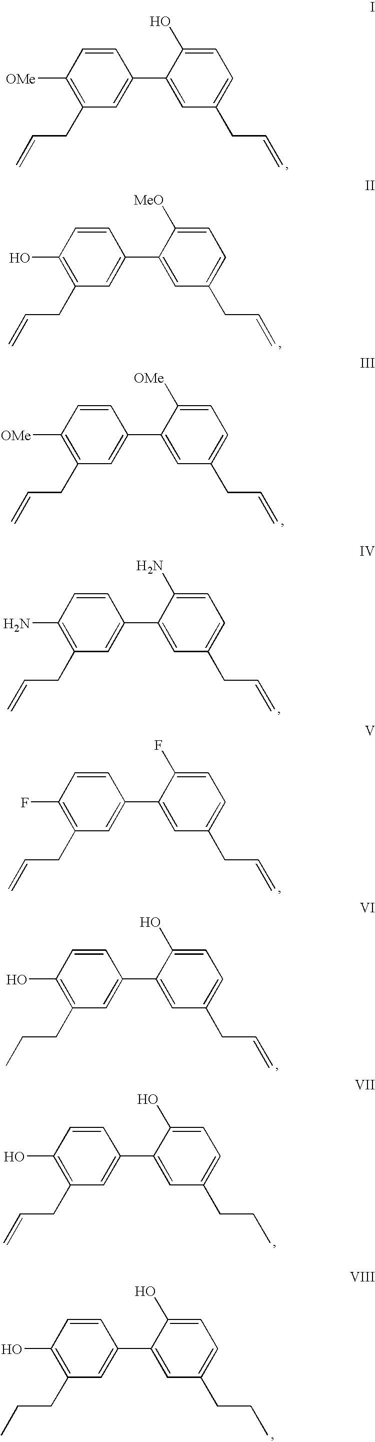

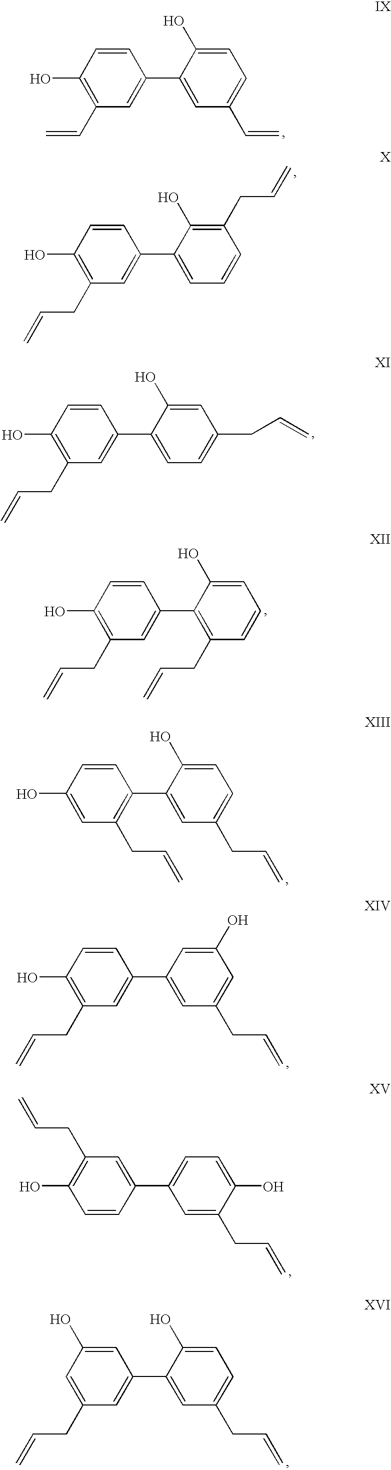

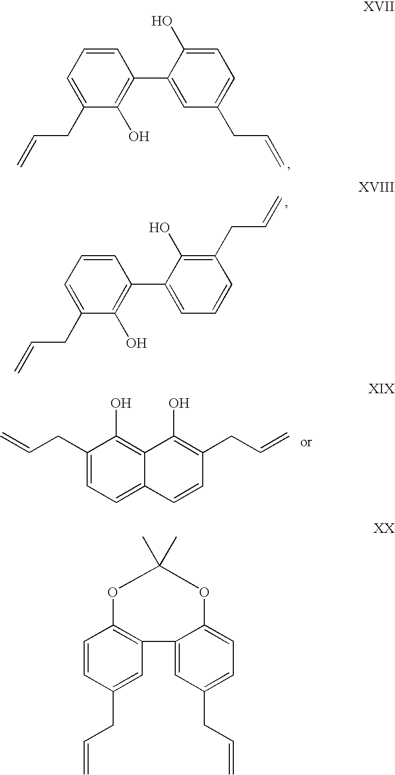

8. A method for the treatment of cancer in a host, the method comprising administering an effective amount of a compound of claim 1 to the host.

9. The method of claim 8, wherein the cancer is selected from the group consisting of carcinoma, sarcoma, lymphoma, leukemia, and myeloma.

10. The method of claim 8, wherein the cancer expresses or overexpresses one or more of phospholipase D, AMPK, or NFKB.

11. A method for the treatment of myeloma in a host, the method comprising administering to the host an effective amount of a compound of claim 1, or honokiol or magnoliol, or a derivative thereof.

12. A method for the treatment of drug resistant cancer in a host, the method comprising administering to the host an effective amount of claim 1, or honokiol or magnoliol, or a derivative thereof.

13. The method of claim 11, wherein the myeloma expresses or overexpresses one or more of phospholipase D, AMPK, or NFKB.

14. The method of claim 12, wherein the cancer is myeloma.

15. The method of claim 12, wherein the cancer expresses or overexpresses one or more of phospholipase D, AMPK, or NFKB.

16. The method of claim 14, wherein the myeloma is selected from the group consisting of multiple myeloma, macroglobulinemia, isolated plasmacytoma of bone, extramedullary plasmacytoma, Waldenstrom's macroglobulinemia, monoclonal gammapathy, and refractory plasma cell neoplasm.

17. The method of claim 8, wherein the compound is administered in combination or alternation with at least one additional therapeutic agent for the treatment of cancer.

18. The method of claim 17, wherein the additional therapeutic agent is a histone deacetylase inhibitor.

19. The method of claim 11, wherein the compound is a compound of claim 1.

20. A method for treating a tumor or cancer in a host comprising (i) obtaining a biological sample from the tumor or cancer; (ii) determining whether the tumor or cancer expresses or overexpresses phospholipase D, (iii) if the tumor or cancer expresses or overexpresses phospholipase D, treating the tumor or cancer with an effective amount of a compound of claim 1, honokiol, magnoliol or a derivative thereof.

21. The method of claim 20, wherein the cancer is selected from the group consisting of carcinoma, sarcoma, lymphoma, leukemia and myeloma.

22. A method for the treatment of an inflammatory condition or osteoporosis in a host the method comprising administering to the host an effect amount of a compound of claim 1, honokiol, or magnoliol or a derivative thereof optionally in combination in a pharmaceutically acceptable carrier.

23. A composition comprising a compound of claim 1 and a pharmaceutically acceptable carrier.

24. Use of a compound of claim 1 in a method for the treatment of a disorder associated with abnormal cell proliferation.

25. The use of claim 24 wherein the disorder is myeloma.

26. The method of claim 12, wherein the compound is administered in combination or alternation with at least one additional therapeutic agent for the treatment of cancer.

27. The method of claim 12, wherein the compound is a compound of claim 1.
